(19) United States
(12) Patent Application Publication
(10) Pub. No.: US 2008/0004302 A1
(43) Pub. Date: Jan. 3, 2008

(54) Novel Compounds

(75) Inventors: Maria-Elena Theoclitou, Macclesfield (GB); David Buttar, Macclesfield (GB); Kevin Michael Foote, Macclesfield (GB); Thorsten Nowak, Macclesfield (GB); David Alan Rudge, Macclesfield (GB); Andrew Peter Thomas, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/769,417

(22) Filed: Jun. 27, 2007

Related U.S. Application Data
(60) Provisional application No. 60/908,428, filed on Mar. 28, 2007.
(60) Provisional application No. 60/818,259, filed on Jun. 30, 2006.

(51) Int. Cl. A61K 31/506; C07D 413/14

(52) U.S. Cl. 514/275; 544/324

(57) ABSTRACT

There is provided a compound of formula (I):

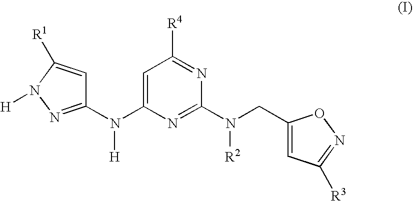

processes for the manufacture thereof, pharmaceutical compositions thereof and uses in therapy.

This application claims the benefit under 35 U.S.C. §119(e) of Application No U.S. 60/818259 filed on 30^{th} June 2006 and Application No U.S. 60/908428 filed on 28^{th} Mar. 2007. The present invention relates to pyrimidine derivatives, a process for their preparation, pharmaceutical compositions containing them, a process for preparing the pharmaceutical compositions, and their use in therapy.

Protein kinases are a class of proteins (enzymes) that regulate a variety of cellular functions. This is accomplished by the phosphorylation of specific amino acids on protein substrates resulting in conformational alteration of the substrate protein. The conformational change modulates the activity of the substrate or its ability to interact with other binding partners. The enzyme activity of the protein kinase refers to the rate at which the kinase adds phosphate groups to a substrate. It can be measured, for example, by determining the amount of a substrate that is converted to a product as a function of time. Phosphorylation of a substrate occurs at the active-site of a protein kinase. Tyrosine kinases are a subset of protein kinases that catalyze the transfer of the terminal phosphate of adenosine triphosphate (ATP) to tyrosine residues on protein substrates. These kinases play an important part in the propagation of growth factor signal transduction that leads to cellular proliferation, differentiation and migration.

Fibroblast growth factor (FGF) has been recognized as an important mediator of many physiological processes, such as morphogenesis during development and angiogenesis. There are currently over 25 known members of the FGF family. The fibroblast growth factor receptor (FGFR) family consists of four members with each composed of an extracellular ligand binding domain, a single transmembrane domain and an intracellular cytoplasmic protein tyrosine kinase domain. Upon stimulation with FGF, FGFRs undergo dimerisation and transphosphorylation, which results in receptor activation. Receptor activation is sufficient for the recruitment and activation of specific downstream signalling partners that participate in the regulation of diverse process such as cell growth, cell metabolism and cell survival (Reviewed in Eswarakumar, V. P. et. al., Cytokine & Growth Factor Reviews 2005, 16, p 139-149). Consequently, FGF and FGFRs have the potential to initiate and/or promote tumorigenesis. There is now considerable evidence directly linking FGF signalling to human cancer. The elevated expression of various FGFs has been reported in a diverse range of tumour types such as bladder, renal cell and prostate (amongst others). FGF has also been described as a powerful angiogenic factor. The expression of FGFRs in endothelial cells has also been reported. Activating mutations of various FGFRs have been associated with bladder cancer and multiple myeloma (amongst others) whilst receptor expression has also been documented in prostate and bladder cancer amongst others (Reviewed in Grose, R. et. al., Cytokine & Growth Factor Reviews 2005, 16, p 179-186 and Kwabi-Addo, B. et. al., Endocrine-Related Cancer 2004, 11, p709-724). For these reasons, the FGF signalling system is an attractive therapeutic target, particularly since therapies targeting FGFRs and/or FGF signalling may affect both the tumour cells directly and tumour angiogenesis.

In accordance with the present invention, there is provided a compound of formula (I):

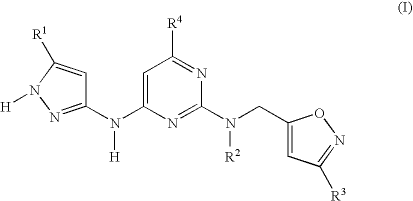

wherein
R¹ represents a C₁-C₆alkyl group optionally substituted by one or more substituents selected from C₁-C₆alkoxy, C₃-C₆cycloalkyl, C₁-C₆alkylthio, —NR⁵R⁶, —C(O)NR⁷R⁸, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), cyano and hydroxyl, a C₃-C₅cycloalkyl group optionally substituted by one or more substituents selected from C₁-C₆alkoxy, C₃-C₆cycloalkyl, C₁-C₆alkylthio, —NR⁹R¹⁰, —C(O)NR¹¹R¹² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a C₂-C₆alkenyl group optionally substituted by one or more substituents selected from C₁-C₆alkoxy, C₃-C₆cycloalkyl, C₁-C₆alkylthio, —NR¹³R¹⁴, —C(O)NR¹⁵R¹⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a 4- to 6-membered heterocyclyl group optionally substituted with by one or more substituents selected from C₁-C₆alkyl, C₁-C₆alkoxy, C₃-C₆cycloalkyl, C₁-C₆alkylthio, —NR¹⁷R¹⁸, —C(O)NR¹⁹R²⁰, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₘC₁-C₆alkyl, —NR²¹R²², —C(O)NR²³R²⁴, —SO₂NR²⁵R²⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₁-C₆alkoxy group optionally substituted by one or more substituents selected from C₁-C₆alkoxy, C₆-aryloxy, C₃-C₆cycloalkyl, —NR²⁷R²⁸, —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —OSO₂C₁₋₆alkyl, —NR³¹R³², —C(O)NR³³R³⁴, —NHC(O)OC₁₋₆alkyl, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₃-C₁₂carbocyclyloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₚC₁-C₆alkyl, —NR³⁷R³⁸, —C(O)NR³⁹R⁴⁰, —SO₂NR⁴¹R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a 5- to 6-membered heterocyclyloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl, a —S(O)ₓR⁴⁹ group, a —S(O)₂NR⁵⁰R⁵¹ group, or -A-B;
R² represents hydrogen or a C₁-C₃alkyl group optionally substituted by one or more substituents selected from C₁-C₃alkoxy, cyano, hydroxyl, amino (—NH₂), mono-C₁-C₃alkylamino and di-(C₁-C₃alky)amino;
R⁴ represents hydrogen, a C₁-C₆alkyl group optionally substituted by one or more substituents selected from C₁-C₃alkoxy, hydroxyl, amino (—NH₂), mono-C₁-C₃alkylamino and di-(C₁-C₃alkyl)amino, a C₁-C₆alkenyl group optionally substituted with C₁-C₃alkoxy, a C₁-C₆alkynyl group optionally substituted with C₁-C₃alkoxy, a C₃-C₅cycloalkyl group optionally substituted with C₁-C₃alkoxy, a C₁-C₆alkoxy group optionally substituted with C₁-C₃alkoxy, hydroxyl, amino (—NH₂), mono-C₁-C₃alkylamino and di-(C₁-C₃alkyl)amino, —C(O)NR⁵²R⁵³, —NR⁵⁴R⁵⁵, —S(O)_{y}R⁵⁶;
A represents a C₂-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or a C₁-alkyleneoxy optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl;
B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, C₁-C₆alkyloxycarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₃₋₅cycloalkyl, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring;
m is 0, 1 or 2;
n is 0, 1 or 2;
p is 0, 1 or 2;
r is 0, 1 or 2;
s is 0, 1 or 2
x is 0, 1 or 2;
y is 0, 1 or 2;
R⁵ and R⁶ each independently represent hydrogen, C₁-C₄alkyl or C₃-C₆cycloalkyl, or R⁵ and R⁶ together with the nitrogen atom to which they are attached form a 4- to 6-membered saturated heterocycle;
R⁷ and R⁸ each independently represent hydrogen, C₁-C₄alkyl or C₃-C₆cycloalkyl, or R⁷ and R⁸ together with the nitrogen atom to which they are attached form a 4- to 6-membered saturated heterocycle;
R⁹ and R¹⁰ each independently represent hydrogen, C₁-C₄alkyl or C₃-C₆cycloalkyl, or R⁹ and R¹⁰ together with the nitrogen atom to which they are attached form a 4- to 6-membered saturated heterocycle;
R¹¹ and R¹² each independently represent hydrogen, C₁-C₄alkyl or C₃-C₆cycloalkyl, or R¹¹ and R¹² together with the nitrogen atom to which they are attached form a 4- to 6-membered saturated heterocycle;
R¹³ and R¹⁴ each independently represent hydrogen, C₁-C₄alkyl or C₃-C₆cycloalkyl, or R¹³ and R¹⁴ together with the nitrogen atom to which they are attached form a 4- to 6-membered saturated heterocycle;
R¹⁵ and R¹⁶ each independently represent hydrogen, C₁-C₄alkyl or C₃-C₆cycloalkyl, or R¹⁵ and R¹⁶ together with the nitrogen atom to which they are attached form a 4- to 6-membered saturated heterocycle;
R¹⁷ and R¹⁸ each independently represent hydrogen, C₁-C₄alkyl or C₃-C₆cycloalkyl, or R¹⁷ and R¹⁸ together with the nitrogen atom to which they are attached form a 4- to 6-membered saturated heterocycle;
R¹⁹ and R²⁰ each independently represent hydrogen, C₁-C₄alkyl or C₃-C₆cycloalkyl, or R¹⁹ and R²⁰ together with the nitrogen atom to which they are attached form a 4- to 6-membered saturated heterocycle;
R²¹ and R²² each independently represent hydrogen, C₁-C₄alkyl or C₃-C₆cycloalkyl, or R21 and R²² together with the nitrogen atom to which they are attached form a 4- to 6-membered saturated heterocycle;
R²³ and R²⁴ each independently represent hydrogen, C₁-C₄alkyl or C₃-C₆cycloalkyl, or R23 and R²⁴ together with the nitrogen atom to which they are attached form a 4- to 6-membered saturated heterocycle;
R²⁵ and R²⁶ each independently represent hydrogen, C₁-C₄alkyl or C₃-C₆cycloalkyl, or R²⁵ and R²⁶ together with the nitrogen atom to which they are attached form a 4- to 6-membered saturated heterocycle;
R²⁷ and R²⁸ each independently represent hydrogen, C₁-C₄alkyl or C₃-C₆cycloalkyl, or R²⁷ and R²⁸ together with the nitrogen atom to which they are attached form a 4- to 6-membered saturated heterocycle;
R²⁹ and R³⁰ each independently represent hydrogen, C₁-C₄alkyl or C₃-C₆cycloalkyl, or R²⁹ and R³⁰ together with the nitrogen atom to which they are attached form a 4- to 6-membered saturated heterocycle;
R³¹ and R³² each independently represent hydrogen, C₁-C₆alkyl or C₃-C₆cycloalkyl, or R³¹ and R³² together with the nitrogen atom to which they are attached form a 4- to 6-membered saturated heterocycle optionally comprising an additional heteratom selected from oxygen, sulphur or nitrogen;
R³³ and R³⁴ each independently represent hydrogen, C₁-C₄alkyl or C₃-C₆cycloalkyl, or R³³ and R³⁴ together with the nitrogen atom to which they are attached form a 4- to 6-membered saturated heterocycle optionally comprising an additional heteratom selected from oxygen, sulphur or nitrogen;
R³⁵ and R³⁶ each independently represent hydrogen, C₁-C₄alkyl or C₃-C₆cycloalkyl, or R³⁵ and R³⁶ together with the nitrogen atom to which they are attached form a 4- to 6-membered saturated heterocycle;
R³⁷ and R³⁸ each independently represent hydrogen, C₁-C₄alkyl or C₃-C₆cycloalkyl, or R³⁷ and R³⁸ together with the nitrogen atom to which they are attached form a 4- to 6-membered saturated heterocycle;
R³⁹ and R⁴⁰ each independently represent hydrogen, C₁-C₄alkyl or C₃-C₆cycloalkyl, or R³⁹ and R⁴⁰ together with the nitrogen atom to which they are attached form a 4- to 6-membered saturated heterocycle;
R⁴¹ and R⁴² each independently represent hydrogen, C₁-C₄alkyl or C₃-C₆cycloalkyl, or R⁴¹ and R⁴² together with the nitrogen atom to which they are attached form a 4- to 6-membered saturated heterocycle;
R⁴³ and R⁴⁴ each independently represent hydrogen, C₁-C₄alkyl or C₃-C₆cycloalkyl, or R⁴³ and R⁴⁴ together with the nitrogen atom to which they are attached form a 4- to 6-membered saturated heterocycle;
R⁴⁵ and R⁴⁶ each independently represent hydrogen, C₁-C₄alkyl or C₃-C₆cycloalkyl, or R⁴⁵ and R⁴⁶ together with the nitrogen atom to which they are attached form a 4- to 6-membered saturated heterocycle;
R⁴⁷ and R⁴⁸ each independently represent hydrogen, C₁-C₄alkyl or C₃-C₆cycloalkyl, or R⁴⁷ and R⁴⁸ together with the nitrogen atom to which they are attached form a 4- to 6-membered saturated heterocycle; ‘R⁴⁹ represents C₁-C₆alkyl, C₃-C₆cycloalkyl or —CH₂Ar wherein Ar represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring;
R⁵⁰ and R⁵¹ each independently represent hydrogen, C₁-C₄alkyl or C₃-C₆cycloalkyl, or R⁵⁰ and R⁵¹ together with the nitrogen atom to which they are attached form a 4- to 6-membered saturated heterocycle;
R⁵² and R⁵³ each independently represent hydrogen, C₁-C₄alkyl or C₃-C₆cycloalkyl, or R⁵² and R⁵³ together with the nitrogen atom to which they are attached form a 4- to 6-membered saturated heterocycle;
R⁵⁴ and R⁵⁵ each independently represent hydrogen, C₁-C₄alkyl or C₃-C₆cycloalkyl, or R⁵⁴ and R⁵⁵ together with the nitrogen atom to which they are attached form a 4- to 6-membered saturated heterocycle;
R⁵⁶ represents C₁-C₆alkyl or C₃-C₆cycloalkyl;
R⁵⁷ and R⁵⁸ each independently represent hydrogen, C₁-C₄alkyl or C₃-C₆cycloalkyl, or R⁵⁷ and R⁵⁸ together with the nitrogen atom to which they are attached form a 4- to 6-membered saturated heterocycle;
R⁵⁹ and R⁶⁰ each independently represent hydrogen, C₁-C₄alkyl or C₃-C₆cycloalkyl, or R⁵⁹ and R⁶⁰ together with the nitrogen atom to which they are attached form a 4- to 6-membered saturated heterocycle;
R⁶¹ and R⁶² each independently represent hydrogen, C₁-C₄alkyl or C₃-C₆cycloalkyl, or R⁶¹ and R⁶² together with the nitrogen atom to which they are attached form a 4- to 6-membered saturated heterocycle optionally comprising an additional heteratom selected from oxygen, sulphur or nitrogen;
R⁶³ and R⁶⁴ each independently represent hydrogen, C₁-C₄alkyl or C₃-C₆cycloalkyl, or R⁶³ and R⁶⁴ together with the nitrogen atom to which they are attached form a 4- to 6-membered saturated heterocycle;
R⁶⁵ and R⁶⁶ each independently represent hydrogen, C₁-C₄alkyl or C₃-C₆cycloalkyl, or R⁶⁵ and R⁶⁶ together with the nitrogen atom to which they are attached form a 4- to 6-membered saturated heterocycle; and
   wherein
(i) when R¹ is an optionally substituted C₂-C₆alkenyl, 4- to 6-membered heterocyclyl group, C₁-C₆alkoxy group, C₃-C₁₂carbocyclyloxy, 5- to 6-membered heterocyclyloxy, —S(O)ₓR⁴⁹, —S(O)₂NR⁵⁰R⁵¹ or -A-B group,
   R³ represents a C₁-C₅alkyl group optionally substituted by one or more substituents selected from C₁-C₃alkoxy, cyano, hydroxyl, amino (—NH₂), mono-C₁-C₃alkylamino and di-(C₁-C₃alkyl)amino, a C₃-C₅cycloalkyl group optionally substituted by one or more substituents selected from C₁-C₃alkyl and C₁-C₃alkoxy, a 3- to 5-membered saturated heterocyclyl group optionally substituted with by one or more substituents selected from C₁-C₃alkyl, C₁-C₃alkoxy and C₃cycloalkyl, a 5- or 6-membered aromatic ring optionally comprising at least one ring heteroatom selected from nitrogen, oxygen and sulphur, a mono-C₁-C₃alkylaminocarbonyl group, a di-(C₁-C₃alkyl)aminocarbonyl group, a C₁-C₃alkoxy carbonyl group, a —CONH₂ group, a —CN group, or a —CO₂H group;
or (ii) when R¹ is an optionally substituted C₁-C₆alkyl or a C₃-C₅cycloalkyl group,
   R³ represents a C₁-C₅alkyl group optionally substituted by one or more substituents selected from C₁-C₃alkoxy, cyano, hydroxyl, amino (—NH₂), mono-C₁-C₃alkylamino and di-(C₁-C₃alkyl)amino, a C₃-C₅cycloalkyl group optionally substituted with C₁-C₃alkoxy, a 3- to 5-membered saturated heterocyclyl group optionally substituted with by one or more substituents selected from C₁-C₃alkyl, C₁-C₃alkoxy and C₃cycloalkyl, a —CONH₂ group, a —CN group, or a —CO₂H group;
   or a pharmaceutically acceptable salt thereof

In accordance with the present invention, there is provided a compound of formula (I):

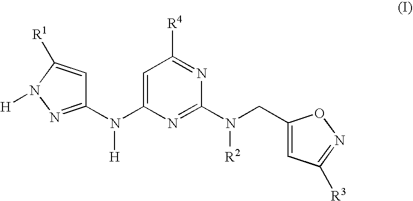

wherein
R¹ represents a C₁-C₆alkyl group optionally substituted by one or more substituents selected from C₁-C₆alkoxy, C₃-C₆cycloalkyl, C₁-C₆alkylthio, —NR⁵R⁶, —C(O)NR⁷R⁸, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), cyano and hydroxyl, a C₃-C₅cycloalkyl group optionally substituted by one or more substituents selected from C₁-C₆alkoxy, C₃-C₆cycloalkyl, C₁-C₆alkylthio, —NR⁹R¹⁰, —C(O)NR¹¹R¹² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a C₂-C₆alkenyl group optionally substituted by one or more substituents selected from C₁-C₆alkoxy, C₃-C₆cycloalkyl, C₁-C₆alkylthio, —NR¹³R¹⁴, —C(O)NR¹⁵R¹⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a 4- to 6-membered heterocyclyl group optionally substituted with by one or more substituents selected from C₁-C₆alkyl, C₁-C₆alkoxy, C₃-C₆cycloalkyl, C₁-C₆alkylthio, —NR¹⁷R¹⁸, —C(O)NR¹⁹R²⁰, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₘC₁-C₆alkyl, —NR²¹R²², —C(O)NR²³R²⁴, —SO₂NR²⁵R²⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₁-C₆alkoxy group optionally substituted by one or more substituents selected from C₁-C₆alkoxy, C₆-aryloxy, C₃-C₆cycloalkyl, —NR²⁷R²⁸, —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —OSO₂C₁₋₆alkyl, —NR³¹R³², —C(O)NR³³R³⁴, —NHC(O)OC₁₋₆alkyl, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₆aryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₚC₁-C₆alkyl, —NR³⁷R³⁸, —C(O)NR³⁹R⁴⁰, —SO₂NR⁴¹R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a 5- to 6-membered heteroaryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl, a —S(O)ₓR⁴⁹ group, a —S(O)₂NR⁵⁰R⁵¹ group, or -A-B;
R² represents hydrogen or a C₁-C₃alkyl group optionally substituted by one or more substituents selected from C₁-C₃alkoxy, cyano, hydroxyl, amino (—NH₂), mono-C₁-C₃alkylamino and di-(C₁-C₃alky)amino;
R⁴ represents hydrogen, a C₁-C₆alkyl group optionally substituted by one or more substituents selected from C₁-C₃alkoxy, hydroxyl, amino (—NH₂), mono-C₁-C₃alkylamino and di-(C₁-C₃alkyl)amino, a C₁-C₆alkenyl group optionally substituted with C₁-C₃alkoxy, a C₁-C₆alkynyl group optionally substituted with C₁-C₃alkoxy, a C₃-C5cycloalkyl group optionally substituted with C₁-C₃alkoxy, a C₁-C₆alkoxy group optionally substituted with C₁-C₃alkoxy, hydroxyl, amino (—NH₂), mono-C₁-C₃alkylamino and di-(C₁-C₃alkyl)amino,
—C(O)NR⁵²R⁵³
—NR⁵⁴R⁵⁵
—S(O)_{y}R⁵⁶;
A represents a C₂-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or a C₁-alkyleneoxy optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl;
B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, C₁-C₆alkyloxycarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁵ (each of which may be optionally substituted by one or more substituents selected from halogen, C₁-C₆alkyl, C₁-C₆alkoxy, C₃₋₅cycloalkyl, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring;
m is 0, 1 or 2;
n is 0, 1 or 2;
p is 0, 1 or 2;
r is 0, 1 or 2;
s is 0, 1 or 2
x is 0, 1 or 2;
y is 0, 1 or 2;
R⁵ and R⁶ each independently represent hydrogen, C₁-C₄alkyl or C₃-C₆cycloalkyl, or R⁵ and R⁶ together with the nitrogen atom to which they are attached form a 4- to 6-membered saturated heterocycle;
R⁷ and R⁸ each independently represent hydrogen, C₁-C₄alkyl or C₃-C₆cycloalkyl, or R⁷ and R⁸ together with the nitrogen atom to which they are attached form a 4- to 6-membered saturated heterocycle;
R⁹ and R¹⁰ each independently represent hydrogen, C₁-C₄alkyl or C₃-C₆cycloalkyl, or R⁹ and R¹⁰ together with the nitrogen atom to which they are attached form a 4- to 6-membered saturated heterocycle;
R¹¹ and R¹² each independently represent hydrogen, C₁-C₄alkyl or C₃-C₆cycloalkyl, or R¹l and R¹² together with the nitrogen atom to which they are attached form a 4- to 6-membered saturated heterocycle;
R¹³ and R¹⁴ each independently represent hydrogen, C₁-C₄alkyl or C₃-C₆cycloalkyl, or R¹³ and R¹⁴ together with the nitrogen atom to which they are attached form a 4- to 6-membered saturated heterocycle;
R¹⁵ and R¹⁶ each independently represent hydrogen, C₁-C₄alkyl or C₃-C₆cycloalkyl, or R¹⁵ and R¹⁶ together with the nitrogen atom to which they are attached form a 4- to 6-membered saturated heterocycle;
R¹⁷ and R¹⁸ each independently represent hydrogen, C₁-C₄alkyl or C₃-C₆cycloalkyl, or R¹⁷ and R¹⁸ together with the nitrogen atom to which they are attached form a 4- to 6-membered saturated heterocycle;
R¹⁹ and R²⁰ each independently represent hydrogen, C₁-C₄alkyl or C₃-C₆cycloalkyl, or R¹⁹ and R²⁰ together with the nitrogen atom to which they are attached form a 4- to 6-membered saturated heterocycle;
R²¹ and R²² each independently represent hydrogen, C₁-C₄alkyl or C₃-C₆cycloalkyl, or R²¹ and R²² together with the nitrogen atom to which they are attached form a 4- to 6-membered saturated heterocycle;
R²³ and R²⁴ each independently represent hydrogen, C₁-C₄alkyl or C₃-C₆cycloalkyl, or R²³ and R²⁴ together with the nitrogen atom to which they are attached form a 4- to 6-membered saturated heterocycle;
R²⁵ and R²⁶ each independently represent hydrogen, C₁-C₄alkyl or C₃-C₆cycloalkyl, or R²⁵ and R²⁶ together with the nitrogen atom to which they are attached form a 4- to 6-membered saturated heterocycle;
R²⁷ and R²⁸ each independently represent hydrogen, C₁-C₄alkyl or C₃-C₆cycloalkyl, or R²⁷ and R²⁸ together with the nitrogen atom to which they are attached form a 4- to 6-membered saturated heterocycle;
R²⁹ and R³⁰ each independently represent hydrogen, C₁-C₄alkyl or C₃-C₆cycloalkyl, or R²⁹ and R³⁰ together with the nitrogen atom to which they are attached form a 4- to 6-membered saturated heterocycle;
R³¹ and R³² each independently represent hydrogen, C₁-C₆alkyl or C₃-C₆cycloalkyl, or R³¹ and R³² together with the nitrogen atom to which they are attached form a 4- to 6-membered saturated heterocycle optionally comprising an additional heteratom selected from oxygen, sulphur or nitrogen;
R³³ and R³⁴ each independently represent hydrogen, C₁-C₄alkyl or C₃-C₆cycloalkyl, or R³³ and R³⁴ together with the nitrogen atom to which they are attached form a 4- to 6-membered saturated heterocycle optionally comprising an additional heteratom selected from oxygen, sulphur or nitrogen;
R³⁵ and R³⁶ each independently represent hydrogen, C₁-C₄alkyl or C₃-C₆cycloalkyl, or R³⁵ and R³⁶ together with the nitrogen atom to which they are attached form a 4- to 6-membered saturated heterocycle;
R³⁷ and R³⁸ each independently represent hydrogen, C₁-C₄alkyl or C₃-C₆cycloalkyl, or R³⁷ and R³⁸ together with the nitrogen atom to which they are attached form a 4- to 6-membered saturated heterocycle;
R³⁹ and R⁴⁰ each independently represent hydrogen, C₁-C₄alkyl or C₃-C₆cycloalkyl, or R³⁹ and R⁴⁰ together with the nitrogen atom to which they are attached form a 4- to 6-membered saturated heterocycle;
R⁴¹ and R⁴² each independently represent hydrogen, C₁-C₄alkyl or C₃-C₆cycloalkyl, or R⁴¹ and R⁴² together with the nitrogen atom to which they are attached form a 4- to 6-membered saturated heterocycle;
R⁴³ and R⁴⁴ each independently represent hydrogen, C₁-C₄alkyl or C₃-C₆cycloalkyl, or R⁴³ and R⁴⁴ together with the nitrogen atom to which they are attached form a 4- to 6-membered saturated heterocycle;
R⁴⁵ and R⁴⁶ each independently represent hydrogen, C₁-C₄alkyl or C₃-C₆cycloalkyl, or R⁴⁵ and R⁴⁶ together with the nitrogen atom to which they are attached form a 4- to 6-membered saturated heterocycle;
R⁴⁷ and R⁴⁸ each independently represent hydrogen, C₁-C₄alkyl or C₃-C₆cycloalkyl, or R⁴⁷ and R⁴⁸ together with the nitrogen atom to which they are attached form a 4- to 6-membered saturated heterocycle;
R⁴⁹ represents C₁-C₆alkyl, C₃-C₆cycloalkyl or -CH₂Ar wherein Ar represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), -CH₂OCO₂H, halogen, nitro, cyano, carboxyl and hydroxyl, and optionally wherein two or more adjacent substituents together with the atoms to which they are attached form a partially or fully unsaturated 4- to 6-membered ring;
R⁵⁰ and R⁵¹ each independently represent hydrogen, C₁-C₄alkyl or C₃-C₆cycloalkyl, or R⁵⁰ and R⁵¹ together with the nitrogen atom to which they are attached form a 4- to 6-membered saturated heterocycle;
R⁵² and R⁵³ each independently represent hydrogen, C₁-C₄alkyl or C₃-C₆cycloalkyl, or R⁵² and R⁵³ together with the nitrogen atom to which they are attached form a 4- to 6-membered saturated heterocycle;
R⁵⁴ and R⁵⁵ each independently represent hydrogen, C₁-C₄alkyl or C₃-C₆cycloalkyl, or R⁵⁴ and R⁵⁵ together with the nitrogen atom to which they are attached form a 4- to 6-membered saturated heterocycle;
R⁵⁶ represents C₁-C₆alkyl or C₃-C₆cycloalkyl;

R⁵⁷ and R⁵⁸ each independently represent hydrogen, C₁-C₄alkyl or C₃-C₆cycloalkyl, or R⁵⁷ and R⁵⁸ together with the nitrogen atom to which they are attached form a 4- to 6-membered saturated heterocycle;
R⁵⁹ and R⁶⁰ each independently represent hydrogen, C₁-C₄alkyl or C₃-C₆cycloalkyl, or R⁵⁹ and R⁶⁰ together with the nitrogen atom to which they are attached form a 4- to 6-membered saturated heterocycle;
R⁶¹ and R⁶² each independently represent hydrogen, C₁-C₄alkyl or C₃-C₆cycloalkyl, or R⁶¹ and R⁶² together with the nitrogen atom to which they are attached form a 4- to 6-membered saturated heterocycle optionally comprising an additional heteratom selected from oxygen, sulphur or nitrogen;
R⁶³ and R⁶⁴ each independently represent hydrogen, C₁-C₄alkyl or C₃-C₆cycloalkyl, or R⁶³ and R⁶⁴ together with the nitrogen atom to which they are attached form a 4- to 6-membered saturated heterocycle;
R⁶⁵ and R⁶⁶ each independently represent hydrogen, C₁-C₄alkyl or C₃-C₆cycloalkyl, or R⁶⁵ and R⁶⁶ together with the nitrogen atom to which they are attached form a 4- to 6-membered saturated heterocycle; and
   wherein
(i) when R¹ is an optionally substituted C₂-C₆alkenyl, 4- to 6-membered heterocyclyl group, C₁-C₆alkoxy group, C₆aryloxy group, 5- to 6-membered heteroaryloxy, —S(O)ₓR⁴⁹, —S(O)₂NR⁵OR⁵¹ or -A-B group,
   R³ represents a C₁-C₅alkyl group optionally substituted by one or more substituents selected from C₁-C₃alkoxy, cyano, hydroxyl, amino (—NH₂), mono-C₁-C₃alkylamino and di-(C₁-C₃alkyl)amino, a C₃-C₅cycloalkyl group optionally substituted by one or more substituents selected from C₁-C₃alkyl and C₁-C₃alkoxy, a 3- to 5-membered saturated heterocyclyl group optionally substituted with by one or more substituents selected from C₁-C₃alkyl, C₁-C₃alkoxy and C₃cycloalkyl, a 5- or 6-membered aromatic ring optionally comprising at least one ring heteroatom selected from nitrogen, oxygen and sulphur, a mono-C₁-C₃alkylaminocarbonyl group, a di-(C₁-C₃alkyl)aminocarbonyl group, a C₁-C₃alkoxy carbonyl group, a —CONH₂ group, a —CN group, or a —CO₂H group;
or (ii) when R¹ is an optionally substituted C₁-C₆alkyl or a C₃-C₅cycloalkyl group,
   R³ represents a C₁-C₅alkyl group optionally substituted by one or more substituents selected from C₁-C₃alkoxy, cyano, hydroxyl, amino (—NH₂), mono-C₁-C₃alkylamino and di-(C₁-C₃alkyl)amino, a C₃-C5cycloalkyl group optionally substituted by one or more substituents selected from C₁-C₃alkyl and C₁-C₃alkoxy, a 3- to 5-membered saturated heterocyclyl group optionally substituted with by one or more substituents selected from C₁-C₃alkyl, C₁-C₃alkoxy and C₃cycloalkyl, a —CONH₂ group, a —CN group, or a —CO₂H group;
or a pharmaceutically acceptable salt thereof

In accordance with the present invention, there is provided a compound of formula (I):

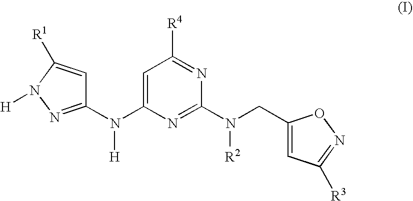

wherein
R¹ represents a C₁-C₆alkyl group optionally substituted by one or more substituents selected from C₁-C₆alkoxy, C₃-C₆cycloalkyl, C₁-C₆alkylthio, —NR⁵R⁶, —C(O)NR⁷R⁸, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), cyano and hydroxyl, a C₃-C₅cycloalkyl group optionally substituted by one or more substituents selected from C₁-C₆alkoxy, C₃-C₆cycloalkyl, C₁-C₆alkylthio, —NR⁹R¹⁰, —C(O)NR¹¹R¹² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a C₂-C₆alkenyl group optionally substituted by one or more substituents selected from C₁-C₆alkoxy, C₃-C₆cycloalkyl, C₁-C₆alkylthio, —NR¹³R¹⁴, —C(O)NR¹⁵R¹⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a 4- to 6-membered heterocyclyl group optionally substituted with by one or more substituents selected from C₁-C₆alkyl, C₁-C₆alkoxy, C₃-C₆cycloalkyl, C₁-C₆alkylthio, —NR¹⁷R¹⁸, —C(O)NR¹⁹R²⁰, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₘC₁-C₆alkyl, —NR²¹R²², —C(O)NR²³R²⁴, —SO₂NR²⁵R²⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₁-C₆alkoxy group optionally substituted by one or more substituents selected from C₁-C₆alkoxy, C₆-aryloxy, C₃-C₆cycloalkyl, —NR²⁷R²⁸, —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —OSO₂C₁₋₆alkyl, —NR³¹R³², —C(O)NR³³R³⁴, —NHC(O)OC₁₋₆alkyl, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₆aryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₚC₁-C₆alkyl, —NR³⁷R³⁸, —C(O)NR³⁹R⁴⁰, —SO₂NR⁴¹R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a 5- to 6-membered heteroaryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl, a —S(O)ₓR⁴⁹ group, a —S(O)₂NR⁵⁰R⁵¹ group, or -A-B;
R² represents hydrogen or a C₁-C₃alkyl group optionally substituted by one or more substituents selected from C₁-C₃alkoxy, cyano, hydroxyl, amino (—NH₂), mono-C₁-C₃alkylamino and di-(C₁-C₃alky)amino;
R⁴ represents hydrogen, a C₁-C₆alkyl group optionally substituted with C₁-C₃alkoxy, hydroxyl, amino (—NH₂), mono-C₁-C₃alkylamino and di-(C₁-C₃alkyl)amino, a C₁-C₆alkenyl group optionally substituted with C₁-C₃alkoxy, a C₁-C₆alkynyl group optionally substituted with C₁-C₃alkoxy, a C₃-C₅cycloalkyl group optionally substituted with C₁-C₃alkoxy, a C₁-C₆alkoxy group optionally substituted with C₁-C₃alkoxy, hydroxyl, amino (—NH₂), mono-C₁-C₃alkylamino and di-(C₁-C₃alkyl)amino, —C(O)NR⁵²R⁵³, —NR⁵⁴R⁵⁵, —S(O)_{y}R⁵⁶;
A represents a C₂-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or a C₁-alkyleneoxy optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or
an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl;
B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, C₁-C₆alkyloxycarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₃₋₅cycloalkyl, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring;
m is 0, 1 or 2;
n is 0, 1 or 2;
p is 0, 1 or 2;
r is 0, 1 or 2;
s is 0, 1 or 2
x is 0, 1 or 2;
y is 0, 1 or 2;
R⁵ and R⁶ each independently represent hydrogen, C₁-C₄alkyl or C₃-C₆cycloalkyl, or R⁵ and R⁶ together with the nitrogen atom to which they are attached form a 4- to 6-membered saturated heterocycle;
R⁷ and R⁸ each independently represent hydrogen, C₁-C₄alkyl or C₃-C₆cycloalkyl, or R⁷ and R⁸ together with the nitrogen atom to which they are attached form a 4- to 6-membered saturated heterocycle;
R⁹ and R¹⁰ each independently represent hydrogen, C₁-C₄alkyl or C₃-C₆cycloalkyl, or R⁹ and R¹⁰ together with the nitrogen atom to which they are attached form a 4- to 6-membered saturated heterocycle;
R¹¹ and R¹² each independently represent hydrogen, C₁-C₄alkyl or C₃-C₆cycloalkyl, or R¹¹ and R¹² together with the nitrogen atom to which they are attached form a 4- to 6-membered saturated heterocycle;
R¹³ and R¹⁴ each independently represent hydrogen, C₁-C₄alkyl or C₃-C₆cycloalkyl, or R¹³ and R¹⁴ together with the nitrogen atom to which they are attached form a 4- to 6-membered saturated heterocycle;
R¹⁵ and R¹⁶ each independently represent hydrogen, C₁-C₄alkyl or C₃-C₆cycloalkyl, or R¹⁵ and R¹⁶ together with the nitrogen atom to which they are attached form a 4- to 6-membered saturated heterocycle;
R¹⁷ and R¹⁸ each independently represent hydrogen, C₁-C₄alkyl or C₃-C₆cycloalkyl, or R¹⁷ and R¹⁸ together with the nitrogen atom to which they are attached form a 4- to 6-membered saturated heterocycle;
R¹⁹ and R²⁰ each independently represent hydrogen, C₁-C₄alkyl or C₃-C₆cycloalkyl, or R¹⁹ and R²⁰ together with the nitrogen atom to which they are attached form a 4- to 6-membered saturated heterocycle;
R²¹ and R²² each independently represent hydrogen, C₁-C₄alkyl or C₃-C₆cycloalkyl, or R²¹ and R²² together with the nitrogen atom to which they are attached form a 4- to 6-membered saturated heterocycle;
R²³ and R²⁴ each independently represent hydrogen, C₁-C₄alkyl or C₃-C₆cycloalkyl, or R²³ and R²⁴ together with the nitrogen atom to which they are attached form a 4- to 6-membered saturated heterocycle;
R²⁵ and R²⁶ each independently represent hydrogen, C₁-C₄alkyl or C₃-C₆cycloalkyl, or R²⁵ and R²⁶ together with the nitrogen atom to which they are attached form a 4- to 6-membered saturated heterocycle;
R²⁷ and R²⁸ each independently represent hydrogen, C₁-C₄alkyl or C₃-C₆cycloalkyl, or R²⁷ and R²⁸ together with the nitrogen atom to which they are attached form a 4- to 6-membered saturated heterocycle;
R²⁹ and R³⁰ each independently represent hydrogen, C₁-C₄alkyl or C₃-C₆cycloalkyl, or R²⁹ andR³⁰ together with the nitrogen atom to which they are attached form a 4- to 6-membered saturated heterocycle;
R³¹ and R³² each independently represent hydrogen, C₁-C₆alkyl or C₃-C₆cycloalkyl, or R³¹ and R³² together with the nitrogen atom to which they are attached form a 4- to 6-membered saturated heterocycle optionally comprising an additional heteratom selected from oxygen, sulphur or nitrogen;
R³³ and R³⁴ each independently represent hydrogen, C₁-C₄alkyl or C₃-C₆cycloalkyl, or R³³ and R³⁴ together with the nitrogen atom to which they are attached form a 4- to 6-membered saturated heterocycle optionally comprising an additional heteratom selected from oxygen, sulphur or nitrogen;
R³⁵ and R³⁶ each independently represent hydrogen, C₁-C₄alkyl or C₃-C₆cycloalkyl, or R³⁵ and R³⁶ together with the nitrogen atom to which they are attached form a 4- to 6-membered saturated heterocycle;
R³⁷ and R³⁸ each independently represent hydrogen, C₁-C₄alkyl or C₃-C₆cycloalkyl, or R³⁷ and R³⁸ together with the nitrogen atom to which they are attached form a 4- to 6-membered saturated heterocycle;
R³⁹ and R⁴⁰ each independently represent hydrogen, C₁-C₄alkyl or C₃-C₆cycloalkyl, or R³⁹ and R⁴⁰ together with the nitrogen atom to which they are attached form a 4- to 6-membered saturated heterocycle;
R⁴¹ and R⁴² each independently represent hydrogen, C₁-C₄alkyl or C₃-C₆cycloalkyl, or R⁴¹ and R⁴² together with the nitrogen atom to which they are attached form a 4- to 6-membered saturated heterocycle;
R⁴³ and R⁴⁴ each independently represent hydrogen, C₁-C₄alkyl or C₃-C₆cycloalkyl, or R⁴³ and R⁴⁴ together with the nitrogen atom to which they are attached form a 4- to 6-membered saturated heterocycle;
R⁴⁵ and R⁴⁶ each independently represent hydrogen, C₁-C₄alkyl or C₃-C₆cycloalkyl, or R⁴⁵ and R⁴⁶ together with the nitrogen atom to which they are attached form a 4- to 6-membered saturated heterocycle;
R⁴⁷ and R⁴⁸ each independently represent hydrogen, C₁-C₄alkyl or C₃-C₆cycloalkyl, or R⁴⁷ and R⁴⁸ together with the nitrogen atom to which they are attached form a 4- to 6-membered saturated heterocycle;
R⁴⁹ represents C₁-C₆alkyl, C₃-C₆cycloalkyl or -CH₂Ar wherein Ar represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), -CH₂OCO₂H, halogen, nitro, cyano, carboxyl and hydroxyl, and optionally wherein two or more adjacent substituents together with the atoms to which they are attached form a partially or fully unsaturated 4- to 6-membered ring;
R⁵⁰ and R⁵¹ each independently represent hydrogen, C₁-C₄alkyl or C₃-C₆cycloalkyl, or R⁵⁰ and R⁵¹ together with the nitrogen atom to which they are attached form a 4- to 6-membered saturated heterocycle;
R⁵² and R⁵³ each independently represent hydrogen, C₁-C₄alkyl or C₃-C₆cycloalkyl, or R⁵² and R⁵³ together with the nitrogen atom to which they are attached form a 4- to 6-membered saturated heterocycle;
R⁵⁴ and R⁵⁵ each independently represent hydrogen, C₁-C₄alkyl or C₃-C₆cycloalkyl, or R⁵⁴ and R⁵⁵ together with the nitrogen atom to which they are attached form a 4- to 6-membered saturated heterocycle;
R⁵⁶ represents C₁-C₆alkyl or C₃-C₆cycloalkyl;
R⁵⁷ and R⁵⁸ each independently represent hydrogen, C₁-C₄alkyl or C₃-C₆cycloalkyl, or R⁵⁷ and R⁵⁸ together with the nitrogen atom to which they are attached form a 4- to 6-membered saturated heterocycle;
R⁵⁹ and R⁶⁰ each independently represent hydrogen, C₁-C₄alkyl or C₃-C₆cycloalkyl, or R⁵⁹ and R⁶⁰ together with the nitrogen atom to which they are attached form a 4- to 6-membered saturated heterocycle;
R⁶¹ and R⁶² each independently represent hydrogen, C₁-C₄alkyl or C₃-C₆cycloalkyl, or R⁶¹ and R⁶² together with the nitrogen atom to which they are attached form a 4- to 6-membered saturated heterocycle optionally comprising an additional heteratom selected from oxygen, sulphur or nitrogen;
R⁶³ and R⁶⁴ each independently represent hydrogen, C₁-C₄alkyl or C₃-C₆cycloalkyl, or R⁶³ and R⁶⁴ together with the nitrogen atom to which they are attached form a 4- to 6-membered saturated heterocycle;
R⁶⁵ and R⁶⁶ each independently represent hydrogen, C₁-C₄alkyl or C₃-C₆cycloalkyl, or R⁶⁵ and R⁶⁶ together with the nitrogen atom to which they are attached form a 4- to 6-membered saturated heterocycle; and
   wherein
(i) when R¹ is an optionally substituted C₂-C₆alkenyl, 4- to 6-membered heterocyclyl group, C₁-C₆alkoxy group, C₆aryloxy group, 5- to 6-membered heteroaryloxy, —S(O)ₓR⁴⁹, —S(O)₂NR⁵OR⁵¹ or -A-B group,
   R³ represents a C₁-C₅alkyl group optionally substituted with C₁-C₃alkoxy, cyano, hydroxyl, amino (—NH₂), mono-C₁-C₃alkylamino and di-(C₁-C₃alkyl)amino, a C₃-C₅cycloalkyl group optionally substituted with C₁-C₃alkoxy, a 3- to 5-membered saturated heterocyclyl group optionally substituted with by one or more substituents selected from C₁-C₃alkyl, C₁-C₃alkoxy and C₃cycloalkyl, a 5- or 6-membered aromatic ring optionally comprising at least one ring heteroatom selected from nitrogen, oxygen and sulphur, a mono-C₁-C₃alkylaminocarbonyl group, a di-(C₁-C₃alkyl)aminocarbonyl group, a C₁-C₃alkoxy carbonyl group, a —CONH₂ group, a —CN group, or a —CO₂H group;
or (ii) when R¹ is an optionally substituted C₁-C₆alkyl or a C₃-C₅cycloalkyl group,
   R³ represents a C₁-C₅alkyl group optionally substituted with C₁-C₃alkoxy, cyano, hydroxyl, amino (—NH₂), mono-C₁-C₃alkylamino and di-(C₁-C₃alkyl)amino, a C₃-C₅cycloalkyl group optionally substituted with C₁-C₃alkoxy, a 3- to 5-membered saturated heterocyclyl group optionally substituted with by one or more substituents selected from C₁-C₃alkyl, C₁-C₃alkoxy and C₃cycloalkyl, a —CONH₂ group, a —CN group, or a —CO₂H group;
or a pharmaceutically acceptable salt thereof

It will be understood that the invention also encompasses all stereoisomeric forms, optical isomers, including racemates, tautomers, mixtures thereof and solvates.

In accordance a further aspect of the present invention, there is provided a compound of formula (I):

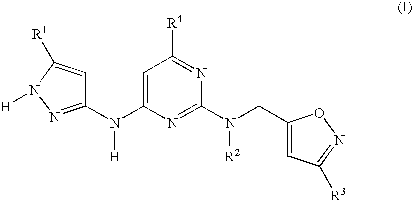

wherein
R¹ represents a C₁-C₆alkyl group optionally substituted by one or more substituents selected from C₁-C₆alkoxy, C₃-C₆cycloalkyl, C₁-C₆alkylthio, —NR⁵R⁶, —C(O)NR⁷R⁸, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), cyano and hydroxyl, a C₃-C₅cycloalkyl group optionally substituted by one or more substituents selected from C₁-C₆alkoxy, C₃-C₆cycloalkyl, C₁-C₆alkylthio, —NR⁹R¹⁰, —C(O)NR¹¹R¹² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a C₂-C₆alkenyl group optionally substituted by one or more substituents selected from C₁-C₆alkoxy, C₃-C₆cycloalkyl, C₁-C₆alkylthio, —NR¹³R¹⁴, —C(O)NR¹⁵R¹⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a 4- to 6-membered heterocyclyl group optionally substituted with by one or more substituents selected from C₁-C₆alkyl, C₁-C₆alkoxy, C₃-C₆cycloalkyl, C₁-C₆alkylthio, —NR¹⁷R¹⁸, —C(O)NR¹⁹R²⁰, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₘC₁-C₆alkyl, —NR²¹R²², —C(O)NR²³R²⁴, —SO₂NR²⁵R²⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₁-C₆alkoxy group optionally substituted by one or more substituents selected from C₁-C₆alkoxy, C₆-aryloxy, C₃-C₆cycloalkyl, —NR²⁷R²⁸ —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —OSO₂C₁₋₆alkyl, —NR³¹R³², —C(O)NR³³R³⁴, —NHC(O)OC₁₋₆alkyl, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₆aryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₚC₁-C₆alkyl, —NR³⁷R³⁸, —C(O)NR³⁹R⁴⁰, —SO₂NR⁴¹R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a 5- to 6-membered heteroaryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl, a —S(O)ₓR⁴⁹ group, a —S(O)₂NR⁵⁰R⁵¹ group, or -A-B;
R² represents hydrogen or a C₁-C₃alkyl group optionally substituted by one or more substituents selected from C₁-C₃alkoxy, cyano, hydroxyl, amino (—NH₂), mono-C₁-C₃alkylamino and di-(C₁-C₃alky)amino;
R⁴ represents hydrogen, a C₁-C₆alkyl group optionally substituted with C₁-C₃alkoxy, hydroxyl, amino (—NH₂), mono-C₁-C₃alkylamino and di-(C₁-C₃alkyl)amino, a C₁-C₆alkenyl group optionally substituted with C₁-C₃alkoxy, a C₁-C₆alkynyl group optionally substituted with C₁-C₃alkoxy, a C₃-C₅cycloalkyl group optionally substituted with C₁-C₃alkoxy, a C₁-C₆alkoxy group optionally substituted with C₁-C₃alkoxy, hydroxyl, amino (—NH₂), mono-C₁-C₃alkylamino and di-(C₁-C₃alkyl)amino, —C(O)NR⁵²R⁵³, NR⁵⁴R⁵⁵, —S(O)_{y}R⁵⁶;
A represents a C₂-alkylene optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or a C₁-alkyleneoxy optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or an oxyC₁-alkylene optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl;
B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring;
is m is 0, 1 or 2;
n is 0, 1 or 2;
p is 0, 1 or 2;
r is 0, 1 or 2;
s is 0, 1 or 2
x is 0, 1 or 2;
y is 0, 1 or 2;
R⁵ and R⁶ each independently represent hydrogen, C₁-C₄alkyl or C₃-C₆cycloalkyl, or R⁵ and R⁶ together with the nitrogen atom to which they are attached form a 4- to 6-membered saturated heterocycle;
R⁷ and R⁸ each independently represent hydrogen, C₁-C₄alkyl or C₃-C₆cycloalkyl, or R⁷ and R⁸ together with the nitrogen atom to which they are attached form a 4- to 6-membered saturated heterocycle;
R⁹ and R¹⁰ each independently represent hydrogen, C₁-C₄alkyl or C₃-C₆cycloalkyl, or R⁹ and R¹⁰ together with the nitrogen atom to which they are attached form a 4- to 6-membered saturated heterocycle;
R¹¹ and R¹² each independently represent hydrogen, C₁-C₄alkyl or C₃-C₆cycloalkyl, or R¹¹ and R¹² together with the nitrogen atom to which they are attached form a 4- to 6-membered saturated heterocycle;
R¹³ and R¹⁴ each independently represent hydrogen, C₁-C₄alkyl or C₃-C₆cycloalkyl, or R¹³ and R¹⁴ together with the nitrogen atom to which they are attached form a 4- to 6-membered saturated heterocycle;
R¹⁵ and R¹⁶ each independently represent hydrogen, C₁-C₄alkyl or C₃-C₆cycloalkyl, or R¹⁵ and R¹⁶ together with the nitrogen atom to which they are attached form a 4- to 6-membered saturated heterocycle;
R¹⁷ and R¹⁸ each independently represent hydrogen, C₁-C₄alkyl or C₃-C₆cycloalkyl, or R¹⁷ and R¹⁸ together with the nitrogen atom to which they are attached form a 4- to 6-membered saturated heterocycle;
R¹⁹ and R²⁰ each independently represent hydrogen, C₁-C₄alkyl or C₃-C₆cycloalkyl, or R¹⁹ and R²⁰ together with the nitrogen atom to which they are attached form a 4- to 6-membered saturated heterocycle;
R²¹ and R²² each independently represent hydrogen, C₁-C₄alkyl or C₃-C₆cycloalkyl, or R²¹ and R²² together with the nitrogen atom to which they are attached form a 4- to 6-membered saturated heterocycle;
R²³ and R²⁴ each independently represent hydrogen, C₁-C₄alkyl or C₃-C₆cycloalkyl, or R²³ and R²⁴ together with the nitrogen atom to which they are attached form a 4- to 6-membered saturated heterocycle;
R²⁵ and R²⁶ each independently represent hydrogen, C₁-C₄alkyl or C₃-C₆cycloalkyl, or R²⁵ and R²⁶ together with the nitrogen atom to which they are attached form a 4- to 6-membered saturated heterocycle;
R²⁷ and R²⁸ each independently represent hydrogen, C₁-C₄alkyl or C₃-C₆cycloalkyl, or R²⁷ and R²⁸ together with the nitrogen atom to which they are attached form a 4- to 6-membered saturated heterocycle;
R²⁹ and R³⁰ each independently represent hydrogen, C₁-C₄alkyl or C₃-C₆cycloalkyl, or R²⁹ and R³⁰ together with the nitrogen atom to which they are attached form a 4- to 6-membered saturated heterocycle;
R³¹ and R³² each independently represent hydrogen, C₁-C₆alkyl or C₃-C₆cycloalkyl, or R³¹ and R³² together with the nitrogen atom to which they are attached form a 4- to 6-membered saturated heterocycle optionally comprising an additional heteratom selected from oxygen, sulphur or nitrogen;
R³³ and R³⁴ each independently represent hydrogen, C₁-C₄alkyl or C₃-C₆cycloalkyl, or R³³ and R³⁴ together with the nitrogen atom to which they are attached form a 4- to 6-membered saturated heterocycle optionally comprising an additional heteratom selected from oxygen, sulphur or nitrogen;
R³⁵ and R³⁶ each independently represent hydrogen, C₁-C₄alkyl or C₃-C₆cycloalkyl, or R³⁵ and R³⁶ together with the nitrogen atom to which they are attached form a 4- to 6-membered saturated heterocycle;
R³⁷ and R³⁸ each independently represent hydrogen, C₁-C₄alkyl or C₃-C₆cycloalkyl, or R³⁷ and R³⁸ together with the nitrogen atom to which they are attached form a 4- to 6-membered saturated heterocycle;
R³⁹ and R⁴⁰ each independently represent hydrogen, C₁-C₄alkyl or C₃-C₆cycloalkyl, or R³⁹ and R⁴⁰ together with the nitrogen atom to which they are attached form a 4- to 6-membered saturated heterocycle;
R⁴¹ and R⁴² each independently represent hydrogen, C₁-C₄alkyl or C₃-C₆cycloalkyl, or R⁴¹ and R⁴² together with the nitrogen atom to which they are attached form a 4- to 6-membered saturated heterocycle;
R⁴³ and R⁴⁴ each independently represent hydrogen, C₁-C₄alkyl or C₃-C₆cycloalkyl, or R⁴³ and R⁴⁴ together with the nitrogen atom to which they are attached form a 4- to 6-membered saturated heterocycle;
R⁴⁵ and R⁴⁶ each independently represent hydrogen, C₁-C₄alkyl or C₃-C₆cycloalkyl, or R⁴⁵ and R⁴⁶ together with the nitrogen atom to which they are attached form a 4- to 6-membered saturated heterocycle;
R⁴⁷ and R⁴⁸ each independently represent hydrogen, C₁-C₄alkyl or C₃-C₆cycloalkyl, or R⁴⁷ and R⁴⁸ together with the nitrogen atom to which they are attached form a 4- to 6-membered saturated heterocycle;
R⁴⁹ represents C₁-C₆alkyl, C₃-C₆cycloalkyl or -CH₂Ar wherein Ar represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring;
R⁵⁰ and R⁵¹ each independently represent hydrogen, C₁-C₄alkyl or C₃-C₆cycloalkyl, or R⁵⁰ and R⁵¹ together with the nitrogen atom to which they are attached form a 4- to 6-membered saturated heterocycle;
R⁵² and R⁵³ each independently represent hydrogen, C₁-C₄alkyl or C₃-C₆cycloalkyl, or R⁵² and R⁵³ together with the nitrogen atom to which they are attached form a 4- to 6-membered saturated heterocycle;
R⁵⁴ and R⁵⁵ each independently represent hydrogen, C₁-C₄alkyl or C₃-C₆cycloalkyl, or R⁵⁴ and R⁵⁵ together with the nitrogen atom to which they are attached form a 4- to 6-membered saturated heterocycle;
R⁵⁶ represents C₁-C₆alkyl or C₃-C₆cycloalkyl;
R⁵⁷ and R⁵⁸ each independently represent hydrogen, C₁-C₄alkyl or C₃-C₆cycloalkyl, or R⁵⁷ and R⁵⁸ together with the nitrogen atom to which they are attached form a 4- to 6-membered saturated heterocycle;
R⁵⁹ and R⁶⁰ each independently represent hydrogen, C₁-C₄alkyl or C₃-C₆cycloalkyl, or R⁵⁹ and R⁶⁰ together with the nitrogen atom to which they are attached form a 4- to 6-membered saturated heterocycle;
R⁶¹ and R⁶² each independently represent hydrogen, C₁-C₄alkyl or C₃-C₆cycloalkyl, or R⁶¹ and R⁶² together with the nitrogen atom to which they are attached form a 4- to 6-membered saturated heterocycle optionally comprising an additional heteratom selected from oxygen, sulphur or nitrogen;
R⁶³ and R⁶⁴ each independently represent hydrogen, C₁-C₄alkyl or C₃-C₆cycloalkyl, or R⁶³ and R⁶⁴ together with the nitrogen atom to which they are attached form a 4- to 6-membered saturated heterocycle;
R⁶⁵ and R⁶⁶ each independently represent hydrogen, C₁-C₄alkyl or C₃-C₆cycloalkyl, or R⁶⁵ and R⁶⁶ together with the nitrogen atom to which they are attached form a 4- to 6-membered saturated heterocycle; and
   wherein
(i) when R¹ is an optionally substituted C₂-C₆alkenyl, 4- to 6-membered heterocyclyl group, C₁-C₆alkoxy group, C₆aryloxy group, 5- to 6-membered heteroaryloxy, —S(O)ₓR⁴⁹, —S(O)₂NR⁵⁰R⁵¹ or -A-B group,
   R³ represents a C₁-C₅alkyl group optionally substituted with C₁-C₃alkoxy, cyano, hydroxyl, amino (—NH₂), mono-C₁-C₃alkylamino and di-(C₁-C₃alkyl)amino, a C₃-C₅cycloalkyl group optionally substituted with C₁-C₃alkoxy, a 3- to 5-membered saturated heterocyclyl group optionally substituted with by one or more substituents selected from C₁-C₃alkyl, C₁-C₃alkoxy and C₃cycloalkyl, a 5- or 6-membered aromatic ring optionally comprising at least one ring heteroatom selected from nitrogen, oxygen and sulphur, a mono-C₁-C₃alkylaminocarbonyl group, a di-(C₁-C₃alkyl)aminocarbonyl group, a C₁-C₃alkoxy carbonyl group, a —CONH₂ group, a —CN group, or a —CO₂H group;
or (ii) when R¹ is an optionally substituted C₁-C₆alkyl or a C₃-C₅cycloalkyl group,
   R³ represents a C₁-C₅alkyl group optionally substituted with C₁-C₃alkoxy, cyano, hydroxyl, amino (—NH₂), mono-C₁-C₃alkylamino and di-(C₁-C₃alkyl)amino, a C₃-C₅cycloalkyl group optionally substituted with C₁-C₃alkoxy, a 3- to 5-membered saturated heterocyclyl group optionally substituted with by one or more substituents selected from C₁-C₃alkyl, C₁-C₃alkoxy and C₃cycloalkyl, a —CONH₂ group, a —CN group, or a —CO₂H group;
or a pharmaceutically acceptable salt thereof

It will be understood that the invention also encompasses all stereoisomeric forms, optical isomers, including racemates, tautomers, mixtures thereof and solvates.

Excluded Compound List 1

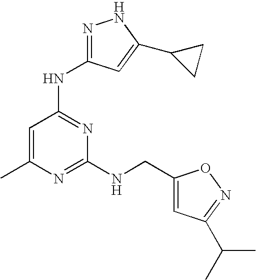

### N′-(5-cyclopropyl-1H-pyrazol-3-yl)-6-methyl-N-[(3-propan-2-yl-1,2-oxazol-5-yl)methyl]pyrimidine-2,4-diamine

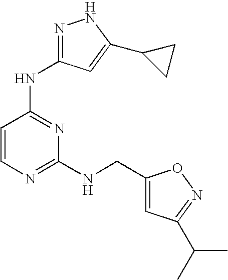

### N′-(5-cyclopropyl-1H-pyrazol-3-yl)-N-[(3-propan-2-yl-1,2-oxazol-5-yl)methyl]pyrimidine-2,4-diamine

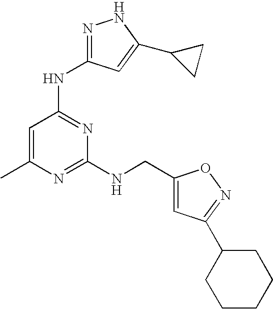

### N-[(3-cyclohexyl-1,2-oxazol-5-yl)methyl]-N′-(5-cyclopropyl-1H-pyrazol-3-yl)-6-methyl-pyrimidine-2,4-diamine

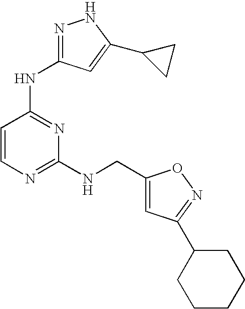

### N-[(3-cyclohexyl-1,2-oxazol-5-yl)methyl]-N′-(5-cyclopropyl-1H-pyrazol-3-yl)pyrimidine-2,4-diamine

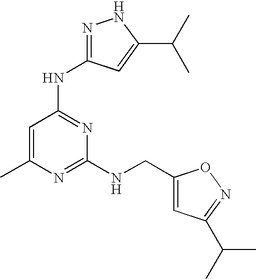

### 6-methyl-N-[(3-propan-2-yl-1,2-oxazol-5-yl)methyl]-N′-(5-propan-2-yl-1H-pyrazol-3-yl)pyrimidine-2,4-diamine

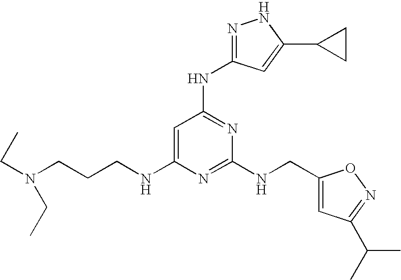

### N4-(5-cyclopropyl-1H-pyrazol-3-yl)-N6-(3-diethylaminopropyl)-N2-[(3-propan-2-yl-1,2-oxazol-5-yl)methyl]pyrimidine-2,4,6-triamine

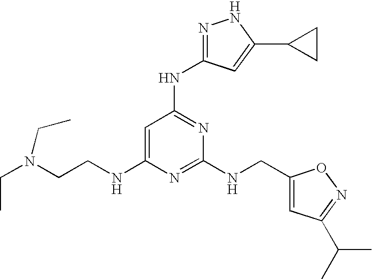

### N4-(5-cyclopropyl-1H-pyrazol-3-yl)-N6-(2-diethylaminoethyl)-N2-[(3-propan-2-yl-1,2-oxazol-5-yl)methyl]pyrimidine-2,4,6-triamine

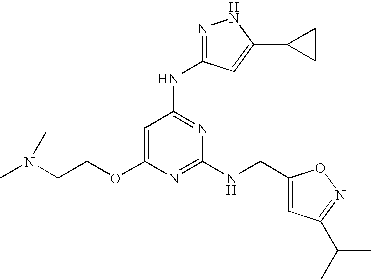

### N′-(5-cyclopropyl-1H-pyrazol-3-yl)-6-(2-dimethylaminoethoxy)-N-[(3-propan-2-yl-1,2-oxazol-5-yl)methyl]pyrimidine-2,4-diamine

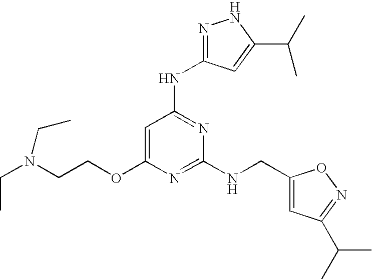

### 6-(2-diethylaminoethoxy)-N-[(3-propan-2-yl-1,2-oxazol-5-yl)methyl]-N′-(5-propan-2-yl-1H-pyrazol-3-yl)pyrimidine-2,4-diamine

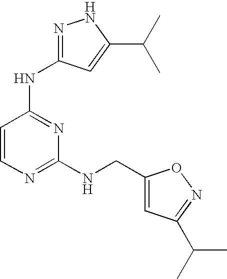

### N-[(3-propan-2-yl-1,2-oxazol-5-yl)methyl]-N′-(5-propan-2-yl-1H-pyrazol-3-yl)pyrimidine-2,4-diamine

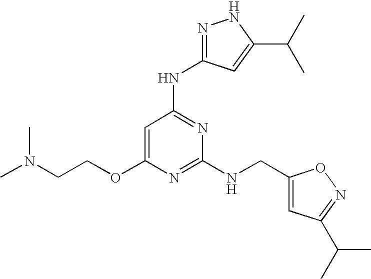

### 6-(2-dimethylaminoethoxy)-N-[(3-propan-2-yl-1,2-oxazol-5-yl)methyl]-N′-(5-propan-2-yl-1H-pyrazol-3-yl)pyrimidine-2,4-diamine

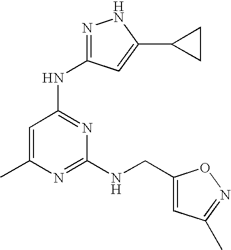

### N′-(5-cyclopropyl-1H-pyrazol-3-yl)-6-methyl-N-[(3-methyl-1,2-oxazol-5-yl)methyl]pyrimidine-2,4-diamine

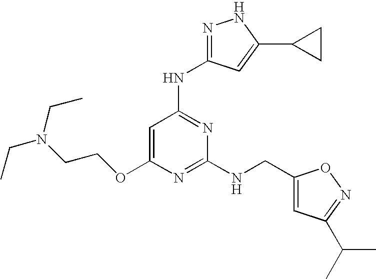

### N′-(5-cyclopropyl-1H-pyrazol-3-yl)-6-(2-diethylaminoethoxy)-N-[(3-propan-2-yl-1,2-oxazol-5-yl)methyl]pyrimidine-2,4-diamine

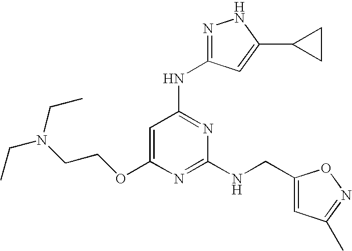

### N′-(5-cyclopropyl-1H-pyrazol-3-yl)-6-(2-diethylaminoethoxy)-N-[(3-methyl-1,2-oxazol-5-yl)methyl]pyrimidine-2,4-diamine

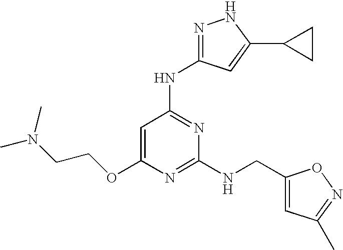

### N′-(5-cyclopropyl-1H-pyrazol-3-yl)-6-(2-dimethylaminoethoxy)-N-[(3-methyl-1,2-oxazol-5-yl)methyl]pyrimidine-2,4-diamine

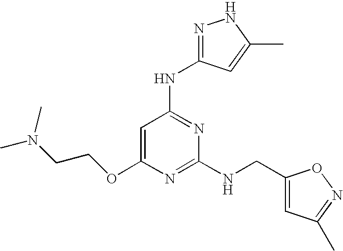

### 6-(2-dimethylaminoethoxy)-N-[(3-methyl-1,2-oxazol-5-yl)methyl]-N′-(5-methyl-1H-pyrazol-3-yl)pyrimidine-2,4-diamine

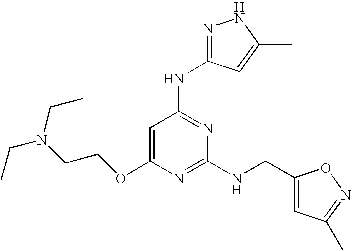

### 6-(2-diethylaminoethoxy)-N-[(3-methyl-1,2-oxazol-5-yl)methyl]-N′-(5-methyl-1H-pyrazol-3-yl)pyrimidine-2,4-diamine

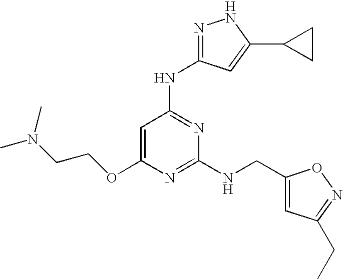

### N′-(5-cyclopropyl-1H-pyrazol-3-yl)-6-(2-dimethylaminoethoxy)-N-[(3-ethyl-1,2-oxazol-5-yl)methyl]pyrimidine-2,4-diamine

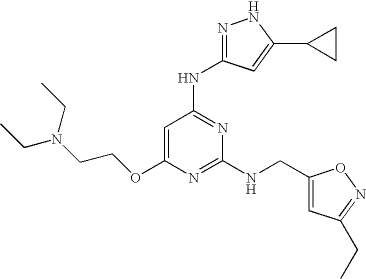

### N′-(5-cyclopropyl-1H-pyrazol-3-yl)-6-(2-diethylaminoethoxy)-N-[(3-ethyl-1,2-oxazol-5-yl)methyl]pyrimidine-2,4-diamine

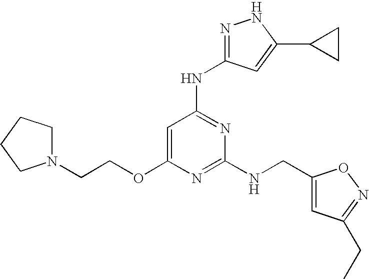

### N′-(5-cyclopropyl-1H-pyrazol-3-yl)-N-[(3-ethyl-1,2-oxazol-5-yl)methyl]-6-(2-pyrrolidin-1-ylethoxy)pyrimidine-2,4-diamine

In accordance with a second aspect of the present invention, there is provided a compound of formula (I):

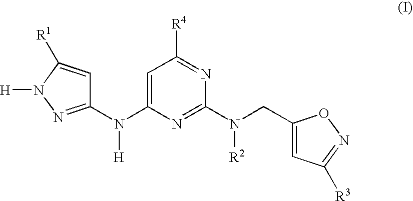

wherein
R¹ represents a C₁-C₆alkyl group optionally substituted by one or more substituents selected from C₁-C₆alkoxy, C₃-C₆cycloalkyl, C₁-C₆alkylthio, —NR⁵R⁶, —C(O)NR⁷R⁸, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), cyano and hydroxyl, a C₃-C₅cycloalkyl group optionally substituted by one or more substituents selected from C₁-C₆alkoxy, C₃-C₆cycloalkyl, C₁-C₆alkylthio, —NR⁹R¹⁰, —C(O)NR¹¹R¹² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a C₂-C₆alkenyl group optionally substituted by one or more substituents selected from C₁-C₆alkoxy, C₃-C₆cycloalkyl, C₁-C₆alkylthio, —NR¹³R¹⁴, —C(O)NR¹⁵R¹⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a 4- to 6-membered heterocyclyl group optionally substituted with by one or more substituents selected from C₁-C₆alkyl, C₁-C₆alkoxy, C₃-C₆cycloalkyl, C₁-C₆alkylthio, —NR¹⁷R¹⁸, —C(O)NR¹⁹R²⁰, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₘC₁-C₆alkyl, —NR²¹R²², —C(O)NR²³R²⁴, —SO₂NR²⁵R²⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₁-C₆alkoxy group optionally substituted by one or more substituents selected from C₁-C₆alkoxy, C₃-C₆cycloalkyl, —NR²⁷R²⁸, —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —NR³¹R³², —C(O)NR³³R³⁴, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₆aryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₚC₁-C₆alkyl, —NR³⁷R³⁸, —C(O)NR³⁹R⁴⁰, —SO₂NR⁴¹R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a 5- to 6-membered heteroaryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl, a —S(O)ₓR⁴⁹ group, a —S(O)₂NR⁵⁰R⁵¹ group, or -A-B;
R² represents hydrogen or a C₁-C₃alkyl group optionally substituted by one or more substituents selected from C₁-C₃alkoxy, cyano, hydroxyl, amino (—NH₂), mono-C₁-C₃alkylamino and di-(C₁-C₃alky)amino;
R³ represents a C₁-C₅alkyl group optionally substituted with C₁-C₃alkoxy, cyano, hydroxyl, amino (—NH₂), mono-C₁-C₃alkylamino and di-(C ₁-C₃alky)amino,
a C₃-C₅cycloalkyl group optionally substituted with C₁-C₃alkoxy, a 3- to 5-membered saturated heterocyclyl group optionally substituted with by one or more substituents selected from C₁-C₃alkyl, C₁-C₃alkoxy and C₃cycloalkyl, a —CONH₂ group, a —CN group, or a —CO₂H group;
R⁴ represents hydrogen, a C₁-C₆alkyl group optionally substituted with C₁-C₃alkoxy, hydroxyl, amino (—NH₂), mono-C₁-C₃alkylamino and di-(C₁-C₃alky)amino, a C₁-C₆alkenyl group optionally substituted with C₁-C₃alkoxy, a C₁-C₆alkynyl group optionally substituted with C₁-C₃alkoxy, a C₃-C₅cycloalkyl group optionally substituted with C₁-C₃alkoxy, a C₁-C₆alkoxy group optionally substituted with C₁-C₃alkoxy, hydroxyl, amino (—NH₂), mono-C₁-C₃alkylamino and di-(C₁-C₃alky)amino, —C(O)NR⁵²R⁵³, —NR⁵⁴R⁵⁵, —S(O)_{y}R⁵⁶;
A represents a C₂-alkylene optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl;
B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring;
m is 0, 1 or 2;
n is 0, 1 or 2;
p is 0, 1 or 2;
r is 0, 1 or 2;
s is 0, 1 or 2
x is 0, 1 or 2;
y is 0, 1 or 2;
R⁵ and R⁶ each independently represent hydrogen, C₁-C₄alkyl or C₃-C₆cycloalkyl, or R⁵ and R⁶ together with the nitrogen atom to which they are attached form a 4- to 6-membered saturated heterocycle;
R⁷ and R⁸ each independently represent hydrogen, C₁-C₄alkyl or C₃-C₆cycloalkyl, or R⁷ and R⁸ together with the nitrogen atom to which they are attached form a 4- to 6-membered saturated heterocycle;
R⁹ and R¹⁰ each independently represent hydrogen, C₁-C₄alkyl or C₃-C₆cycloalkyl, or R⁹ and R¹⁰ together with the nitrogen atom to which they are attached form a 4- to 6-membered saturated heterocycle;
R¹¹ and R¹² each independently represent hydrogen, C₁-C₄alkyl or C₃-C₆cycloalkyl, or R¹¹ and R¹² together with the nitrogen atom to which they are attached form a 4- to 6-membered saturated heterocycle;
R¹³ and R¹⁴ each independently represent hydrogen, C₁-C₄alkyl or C₃-C₆cycloalkyl, or R¹³ and R¹⁴ together with the nitrogen atom to which they are attached form a 4- to 6-membered saturated heterocycle;
R¹⁵ and R¹⁶ each independently represent hydrogen, C₁-C₄alkyl or C₃-C₆cycloalkyl, or R¹⁵ and R¹⁶ together with the nitrogen atom to which they are attached form a 4- to 6-membered saturated heterocycle;
R¹⁷ and R¹⁸ each independently represent hydrogen, C₁-C₄alkyl or C₃-C₆cycloalkyl, or R¹⁷ and R¹⁸ together with the nitrogen atom to which they are attached form a 4- to 6-membered saturated heterocycle;
R¹⁹ and R²⁰ each independently represent hydrogen, C₁-C₄alkyl or C₃-C₆cycloalkyl, or R¹⁹ and R²⁰ together with the nitrogen atom to which they are attached form a 4- to 6-membered saturated heterocycle;
R²¹ and R²² each independently represent hydrogen, C₁-C₄alkyl or C₃-C₆cycloalkyl, or R²¹ and R²² together with the nitrogen atom to which they are attached form a 4- to 6-membered saturated heterocycle;
R²³ and R²⁴ each independently represent hydrogen, C₁-C₄alkyl or C₃-C₆cycloalkyl, or R²³ and R²⁴ together with the nitrogen atom to which they are attached form a 4- to 6-membered saturated heterocycle;
R²⁵ and R²⁶ each independently represent hydrogen, C₁-C₄alkyl or C₃-C₆cycloalkyl, or R ²⁵ and R²⁶ together with the nitrogen atom to which they are attached form a 4- to 6-membered saturated heterocycle;
R²⁷ and R²⁸ each independently represent hydrogen, C₁-C₄alkyl or C₃-C₆cycloalkyl, or R²⁷ and R²⁸ together with the nitrogen atom to which they are attached form a 4- to 6-membered saturated heterocycle;
R²⁹ and R³⁰ each independently represent hydrogen, C₁-C₄alkyl or C₃-C₆cycloalkyl, or R²⁹ andR³⁰ together with the nitrogen atom to which they are attached form a 4- to 6-membered saturated heterocycle;
R³¹ and R³² each independently represent hydrogen, C₁-C₆alkyl or C₃-C₆cycloalkyl, or R³¹ and R³² together with the nitrogen atom to which they are attached form a 4- to 6-membered saturated heterocycle;
R³³ and R³⁴ each independently represent hydrogen, C₁-C₄alkyl or C₃-C₆cycloalkyl, or R³³ and R³⁴ together with the nitrogen atom to which they are attached form a 4- to 6-membered saturated heterocycle;
R³⁵ and R³⁶ each independently represent hydrogen, C₁-C₄alkyl or C₃-C₆cycloalkyl, or R³⁵ and R³⁶ together with the nitrogen atom to which they are attached form a 4- to 6-membered saturated heterocycle;

R³⁷ and R³⁸ each independently represent hydrogen, C₁-C₄alkyl or C₃-C₆cycloalkyl, or R³⁷ and R³⁸ together with the nitrogen atom to which they are attached form a 4- to 6-membered saturated heterocycle;
R³⁹ and R⁴⁰ each independently represent hydrogen, C₁-C₄alkyl or C₃-C₆cycloalkyl, or R³⁹ and R⁴⁰ together with the nitrogen atom to which they are attached form a 4- to 6-membered saturated heterocycle;
R⁴¹ and R⁴² each independently represent hydrogen, C₁-C₄alkyl or C₃-C₆cycloalkyl, or R⁴¹ and R⁴² together with the nitrogen atom to which they are attached form a 4- to 6-membered saturated heterocycle;
R⁴³ and R⁴⁴ each independently represent hydrogen, C₁-C₄alkyl or C₃-C₆cycloalkyl, or R⁴³ and R⁴⁴ together with the nitrogen atom to which they are attached form a 4- to 6-membered saturated heterocycle;
R⁴⁵ and R⁴⁶ each independently represent hydrogen, C₁-C₄alkyl or C₃-C₆cycloalkyl, or R⁴⁵ and R⁴⁶ together with the nitrogen atom to which they are attached form a 4- to 6-membered saturated heterocycle;
R⁴⁷ and R⁴⁸ each independently represent hydrogen, C₁-C₄alkyl or C₃-C₆cycloalkyl, or R⁴⁷ and R⁴⁸ together with the nitrogen atom to which they are attached form a 4- to 6-membered saturated heterocycle;
R⁴⁹ represents C₁-C₆alkyl, C₃-C₆cycloalkyl or —CH₂Ar wherein Ar represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring;
R⁵⁰ and R⁵¹ each independently represent hydrogen, C₁-C₄alkyl or C₃-C₆cycloalkyl, or R⁵⁰ and R⁵¹ together with the nitrogen atom to which they are attached form a 4- to 6-membered saturated heterocycle;
R⁵² and R⁵³ each independently represent hydrogen, C₁-C₄alkyl or C₃-C₆cycloalkyl, or R⁵² and R⁵³ together with the nitrogen atom to which they are attached form a 4- to 6-membered saturated heterocycle;
R⁵⁴ and R⁵⁵ each independently represent hydrogen, C₁-C₄alkyl or C₃-C₆cycloalkyl, or R⁵⁴ and R⁵⁵ together with the nitrogen atom to which they are attached form a 4- to 6-membered saturated heterocycle;
R⁵⁶ represents C₁-C₆alkyl or C₃-C₆cycloalkyl;
R⁵⁷ and R⁵⁸ each independently represent hydrogen, C₁-C₄alkyl or C₃-C₆cycloalkyl, or R⁵⁷ and R⁵⁸ together with the nitrogen atom to which they are attached form a 4- to 6-membered saturated heterocycle;
R⁵⁹ and R⁶⁰ each independently represent hydrogen, C₁-C₄alkyl or C₃-C₆cycloalkyl, or R⁵⁹ and R⁶⁰ together with the nitrogen atom to which they are attached form a 4- to 6-membered saturated heterocycle;
R⁶¹ and R⁶² each independently represent hydrogen, C₁-C₄alkyl or C₃-C₆cycloalkyl, or R⁶¹ and R⁶² together with the nitrogen atom to which they are attached form a 4- to 6-membered saturated heterocycle;
R⁶³ and R⁶⁴ each independently represent hydrogen, C₁-C₄alkyl or C₃-C₆cycloalkyl, or R⁶³ and R⁶⁴ together with the nitrogen atom to which they are attached form a 4- to 6-membered saturated heterocycle;
R⁶⁵ and R⁶⁶ each independently represent hydrogen, C₁-C₄alkyl or C₃-C₆cycloalkyl, or R⁶⁵ and R⁶⁶ together with the nitrogen atom to which they are attached form a 4- to 6-membered saturated heterocycle;
or a pharmaceutically acceptable salt thereof

In the context of the present specification, unless otherwise indicated, an alkyl substituent group or an alkyl moiety in a substituent group may be linear or branched. When R⁵ and R⁶, or R⁷ and R⁸, or R⁹ and R¹⁰, or R¹¹ and R¹² or R¹³ and R¹⁴ or R¹⁵ and R¹⁶, or R¹⁷ and R¹⁸ or R¹⁹ and R²⁰ or R²¹ and R²² or R²³ and R²⁴, or R²⁵ and R²⁶ or R²⁷ and R²⁸, or R²⁹ and R³⁰, or R³¹ and R³², or R³³ and R³⁴, or R³⁵ and R³⁶, or R³⁷ and R³⁸, or R³⁹ and R⁴⁰, or R⁴¹ and R⁴², or R⁴³ and R⁴⁴, or R⁴⁵ and R⁴⁶, or R⁴⁷ and R⁴⁸, or R⁵⁰ and R⁵¹, or R⁵² and R⁵³, or R⁵⁴ and R⁵⁵, or R⁵⁷ and R⁵⁸, or R⁵⁹ and R⁶⁰, or R⁶¹ and R⁶², or R⁶³ and R⁶⁴, or R⁶⁵ and R⁶⁶ represent a saturated heterocycle, it should be understood that unless otherwise stated the only heteroatom present is the nitrogen atom to which R⁵ and R⁶, or R⁷ and R⁸, or R⁹ and R¹⁰, or R¹¹ and R¹², or R¹³ and R¹⁴, or R¹⁵ and R¹⁶, or R¹⁷ and R¹⁸, or R¹⁹ and R²⁰, or R²¹ and R²², or R²³ and R²⁴, or R²⁵ and R²⁶, or R²⁷ and R²⁸, or R²⁹ and R³⁰, or R³¹ and R³², or R³³ and R³⁴, or R³⁵ and R³⁶, or R³⁷ and R³⁸, or R³⁹ and R⁴⁰, or R⁴¹ and R⁴², or R⁴³ and R⁴⁴, or R⁴⁵ and R⁴⁶, or R⁴⁷ and R⁴⁸, or R⁵⁰ and R⁵¹, or R⁵² and R⁵³, or R⁵⁴ and R⁵⁵, or R⁵⁷ and R⁵⁸, or R⁵⁹ and R⁶⁰, or R⁶¹ and R⁶², or R⁶³ and R⁶⁴, or R⁶⁵ and R⁶⁶ are attached.

Examples of “C₁-C₆alkyl” and “C₁-C₄alkyl” include methyl, ethyl, n-propyl, i-propyl, n-butyl, i-butyl and t-butyl. Examples of “C₁-C₆alkoxycarbonyl” include methoxycarbonyl, ethoxycarbonyl, n-butoxycarbonyl and t-butoxycarbonyl. Examples of “C₁-C₆alkoxy” and “C₁-C₃alkoxy” include methoxy, ethoxy, n-propoxy and i-propoxy. Examples of “C₁-C₆alkylcarbonylamino” include formamido, acetamido and propionylamino. Examples of “S(O)ₘC₁-C₆alkyl, S(O)ₙC₁-C₆alkyl, S(O)ₚC₁-C₆alkyl S(O)ᵣC₁-C₆alkyl S(O)ₛC₁-C₆alkyl S(O)ₓC₁-C₆alkyl and S(O)_{y}C₁-C₆alkyl” wherein m is 0, 1 or 2 include methylthio, ethylthio, methylsulphinyl, ethylsulphinyl, mesyl and ethylsulphonyl. Examples of “C₁-C₆alkylcarbonyl” include propionyl and acetyl. Examples of “C₂-C₆alkenyl” include vinyl, allyl and 1-propenyl. Examples of “C₃-C₆cycloalkyl” include cyclopropyl, cyclopentyl 1o and cyclohexyl. Example of “mono- and di-C₁ C₆alkylamino” include methylamino, dimethylamino, ethylamino, diethylamino and ethylmethylamino. Examples of “C₁-C₆alkylthio” include methylthio, ethylthio and propylthio.

Examples of halogen include fluorine, chlorine, bromine and iodine.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl.

A “5- or 6-membered aromatic ring optionally comprising at least one ring heteroatom selected from nitrogen, oxygen and sulphur” is a fully unsaturated, aromatic monocyclic ring containing 5 or 6 atoms of which at least one is a heteroatom selected from nitrogen, oxygen and sulphur, which may, unless otherwise specified, be carbon or nitrogen linked. Suitably a “5- or 6-membered aromatic ring optionally comprising at least one ring heteroatom selected from nitrogen, oxygen and sulphur” is furyl, imidazolyl, isothiazolyl, isoxazolyl, oxaxolyl, phenyl, pyrazinyl, pyrazolyl, pyridazinyl, pyridyl, pyrimidinyl, pyrrolyl, thiazolyl, thienyl and triazolyl rings.

A “4- to 6-membered heterocyclic group”, unless otherwise stated, includes saturated and fully or partially unsaturated, monocyclic rings containing 4, 5 or 6 atoms of which at 30 least one is a heteroatom selected from nitrogen, oxygen and sulphur, and which may, unless otherwise specified, be carbon or nitrogen linked. Suitable “4- to 6-membered heterocyclic group” which may comprise at least one ring heteroatom selected from nitrogen, oxygen and sulphur” include tetrahydrofuran, tetrahydrofuranone, gamma-butyrolactone, alpha-pyran, gamma-pyran, dioxolane, tetrahydropyran, dioxane, dihydrothiophene, thiolan, dithiolan, pyrroline, pyrrolidine, pyrazoline, pyrazolidine, imidazoline, imidazolidine, tetrazole, piperidine, pyridazine, pyrimidine, pyrazine, piperazine, triazine, tetrazine, morpholine, thiomorpholine, thiomorpholine S,S-dioxide, diazepan, oxazine, tetrahydro-oxazinyl, isothiazole, oxetane, azetidine, and pyrazolidine.

A “C₃-C₁₂carbocyclyloxy group” and “5- to 6-membered heterocyclyloxy” denotes an -OR group wherein R is either a 3- to 10-membered carbocyclyl group or a 5- to 6-membered heterocyclyl group.

A “C₆aryloxy group” and “5- to 6-membered heteroaryloxy” denotes an —OR group wherein R is a 6-membered aromatic ring, for example phenyl, or a 5- or 6-membered heteroaromatic ring comprising at least one ring heteroatom selected from nitrogen, oxygen and sulphur for example furyl, imidazolyl, isothiazolyl, isoxazolyl, oxaxolyl, phenyl, is pyrazinyl, pyrazolyl, pyridazinyl, pyridyl, pyrimidinyl, pyrrolyl, thiazolyl, thienyl or triazolyl.

A “C₂-alkylene” denotes a two carbon saturated linking group. For example, an unsubstituted C₂-alkylene group is a —CH₂CH₂— linking group.

A “C₁-alkyleneoxy” denotes a two atom saturated linking group comprising one carbon and one oxygen atom. For example, an unsubstituted Cl-alkyleneoxy group is a —CH₂O— linking group (and for example the group -A-B is —CH₂O—B).

An “oxyC₁-alkylene” denotes a two atom saturated linking group comprising one carbon and one oxygen atom. For example, an unsubstituted C₁-alkyleneoxy group is a —OCH₂— linking group (and for example the group -A-B is —OCH₂—B).

When R¹ represents a C₁-C₆alkyl group (such as methyl, ethyl, propyl, i-propyl, butyl, i-butyl, t-butyl pentyl, i-pentyl, neopentyl, hexyl), the C₁-C₆alkyl group is optionally substituted by one or more substituents selected from C₁-C₆alkoxy (such as methoxy, ethoxy, propoxy, i-propoxy, butoxy, i-butoxy, t-butoxy pentoxy, i-pentoxy, neopentoxy, hexoxy), C₃-C₆cycloalkyl (such as cyclopropyl, cyclobutyl, cyclopentyl, cyclohexyl), C₁-C₆alkylthio (such as methylthio, ethylthio, propylthio, i-propylthio, butylthio, i-butylthio, t-butylthio, pentylthio, i-pentylthio, neopentylthio, hexylthio), —NR⁵R⁶, —C(O)NR⁷R⁸, (each of which may be optionally substituted by one or more substituents selected from halogen [such as fluorine, chlorine, bromine or iodine], C₁-C₆alkyl [such as methyl, ethyl, propyl, i-propyl, butyl, i-butyl, t-butyl pentyl, i-pentyl, neopentyl, hexyl], C₁-C₆alkoxy [such as methoxy, ethoxy, propoxy, i-propoxy, butoxy, i-butoxy, t-butoxy pentoxy, i-pentoxy, neopentoxy, hexoxy], C₁-C₆alkylthio [such as methylthio, ethylthio, propylthio, i-propylthio, butylthio, i-butylthio, t-butylthio, pentylthio, i-pentylthio, neopentylthio, hexylthio], amino [—NH₂], mono- and di-C₁-C₆alkylamino [such as methylamino, ethylamino, propylamino, i-propylamino, butylamino, i-butylamino, t-butylamino, pentylamino, i-pentylamino, neopentylamino, hexylamino], cyano, hydroxyl and trifluoromethyl) cyano and hydroxyl.

When R¹ represents a C₃-C₅cycloalkyl group (such as cyclopropyl, cyclobutyl, cyclopentyl), the C₃-C₅cycloalkyl group is optionally substituted by one or more substituents selected from C₁-C₆alkoxy (such as methoxy, ethoxy, propoxy, i-propoxy, butoxy, i-butoxy, t-butoxy pentoxy, i-pentoxy, neopentoxy, hexoxy), C₃-C₆cycloalkyl (such as cyclopropyl, cyclobutyl, cyclopentyl, cyclohexyl), C₁-C₆alkylthio (such as methylthio, ethylthio, propylthio, i-propylthio, butylthio, i-butylthio, t-butylthio, pentylthio, i-pentylthio, neopentylthio, hexylthio), —NR⁹R¹⁰, —C(O)NR¹¹R¹², (each of which may be optionally substituted by one or more substituents selected from halogen, C₁-C₆alkyl [such as methyl, ethyl, propyl, i-propyl, butyl, i-butyl, t-butyl pentyl, i-pentyl, neopentyl, hexyl], C₁-C₆alkoxy [such as methoxy, ethoxy, propoxy, i-propoxy, butoxy, i-butoxy, t-butoxy pentoxy, i-pentoxy, neopentoxy, hexoxy], C₁-C₆alkylthio [such as methylthio, ethylthio, propylthio, i-propylthio, butylthio, i-butylthio, t-butylthio, pentylthio, i-pentylthio, neopentylthio, hexylthio], amino [—NH₂], mono- and di-C₁-C₆alkylamino [such as methylamino, ethylamino, propylamino, i-propylamino, butylamino, i-butylamino, t-butylamino, pentylamino, i-pentylamino, neopentylamino, hexylamino], hydroxyl and trifluoromethyl), and hydroxyl.

When R¹ represents a C₂-C₆alkenyl group, the C₂-C₆alkenyl is optionally substituted by one or more substituents selected from C₁-C₆alkoxy (such as methoxy, ethoxy, propoxy, i-propoxy, butoxy, i-butoxy, t-butoxy pentoxy, i-pentoxy, neopentoxy, hexoxy), C₃-C₆cycloalkyl (such as cyclopropyl, cyclobutyl, cyclopentyl, cyclohexyl), C₁-C₆alkylthio (such as methylthio, ethylthio, propylthio, i-propylthio, butylthio, i-butylthio, t-butylthio, pentylthio, i-pentylthio, neopentylthio, hexylthio), —NR¹³R¹⁴, —C(O)NR¹⁵R¹⁶ (each of which may be optionally substituted by one or more substituents selected from halogen, C₁-C₆alkyl [such as methyl, ethyl, propyl, i-propyl, butyl, i-butyl, t-butyl pentyl, i-pentyl, neopentyl, hexyl], C₁-C₆alkoxy [such as methoxy, ethoxy, propoxy, i-propoxy, butoxy, i-butoxy, t-butoxy pentoxy, i-pentoxy, neopentoxy, hexoxy], C₁-C₆alkylthio [such as methylthio, ethylthio, propylthio, i-propylthio, butylthio, i-butylthio, t-butylthio, pentylthio, i-pentylthio, neopentylthio, hexylthio], amino [—NH₂], mono- and di-C₁-C₆alkylamino [such as methylamino, ethylamino, propylamino, i-propylamino, butylamino, i-butylamino, t-butylamino, pentylamino, i-pentylamino, neopentylamino, hexylamino], hydroxyl and trifluoromethyl) and hydroxyl.

When R¹ represents a 4- to 6-membered heterocyclyl group, the 4- to 6-membered heterocyclyl group is optionally substituted with by one or more substituents selected from C₁-C₆alkyl (such as methyl, ethyl, propyl, i-propyl, butyl, i-butyl, t-butyl pentyl, i-pentyl, neopentyl, hexyl), C₁-C₆alkoxy (such as methoxy, ethoxy, propoxy, i-propoxy, butoxy, i-butoxy, t-butoxy pentoxy, i-pentoxy, neopentoxy, hexoxy), C₃-C₆cycloalkyl (such as cyclopropyl, cyclobutyl, cyclopentyl, cyclohexyl), C₁-C₆alkylthio (such as methylthio, ethylthio, propylthio, i-propylthio, butylthio, i-butylthio, t-butylthio, pentylthio, i-pentylthio, neopentylthio, hexylthio), —NR¹⁷R¹⁸, —C(O)NR¹⁹R²⁰, (each of which may be optionally substituted by one or more substituents selected from halogen, C₁-C₆alkyl [such as methyl, ethyl, propyl, i-propyl, butyl, i-butyl, t-butyl pentyl, i-pentyl, neopentyl, hexyl], C₁-C₆alkoxy [such as methoxy, ethoxy, propoxy, i-propoxy, butoxy, i-butoxy, t-butoxy pentoxy, i-pentoxy, neopentoxy, hexoxy], C₁-C₆alkylthio [such as methylthio, ethylthio, propylthio, i-propylthio, butylthio, i-butylthio, t-butylthio, pentylthio, i-pentylthio, neopentylthio, hexylthio], amino [—NH₂], mono- and di-C₁-C₆alkylamino [such as methylamino, ethylamino, propylamino, i-propylamino, butylamino, i-butylamino, t-butylamino, pentylamino, i-pentylamino, neopentylamino, hexylamino], hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl (such as methyl, ethyl, propyl, i-propyl, butyl, i-butyl, t-butyl pentyl, i-pentyl, neopentyl, hexyl), C₁-C₆alkoxy (such as methoxy, ethoxy, propoxy, i-propoxy, butoxy, i-butoxy, t-butoxy pentoxy, i-pentoxy, neopentoxy, hexoxy), C₂-C₆alkenyl, C₃-C₆cycloalkyl (such as cyclopropyl, cyclobutyl, cyclopentyl, cyclohexyl), C₁-C₆alkoxycarbonyl (such as methoxycarbonyl, ethoxycarbonyl, propoxycarbonyl, i-propoxycarbonyl, butoxycarbonyl, i-butoxycarbonyl, t-butoxycarbonyl, pentoxycarbonyl, i-pentoxycarbonyl, neopentoxycarbonyl, hexoxycarbonyl), C₁-C₆alkylcarbonyl (such as methylcarbonyl, ethylcarbonyl, propylcarbonyl, i-propylcarbonyl, butylcarbonyl, i-butylcarbonyl, t-butylcarbonyl, pentylcarbonyl, i-pentylcarbonyl, neopentylcarbonyl, hexylcarbonyl), C₁-C₆alkylcarbonylamino (such as methylamino, ethylamino, propylamino, i-propylamino, butylamino, i-butylamino, t-butylamino, pentylamino, i-pentylamino, neopentylamino, hexylamino), phenylcarbonyl, —S(O)ₘC₁-C₆alkyl, —NR²¹R²², —C(O)NR²³R²⁴, —SO₂NR²⁵R²⁶ (each of which may be optionally substituted by one or more substituents selected from halogen, C₁-C₆alkyl [such as methyl, ethyl, propyl, i-propyl, butyl, i-butyl, t-butyl pentyl, i-pentyl, neopentyl, hexyl], C₁-C₆alkoxy [such as methoxy, ethoxy, propoxy, i-propoxy, butoxy, i-butoxy, t-butoxy pentoxy, i-pentoxy, neopentoxy, hexoxy], C₁-C₆alkylthio [such as methylthio, ethylthio, propylthio, i-propylthio, butylthio, i-butylthio, t-butylthio, pentylthio, i-pentylthio, neopentylthio, hexylthio], amino (—NH₂), mono- and di-C₁-C₆alkylamino [such as methylamino, ethylamino, propylamino, i-propylamino, butylamino, i-butylamino, t-butylamino, pentylamino, i-pentylamino, neopentylamino, hexylamino], phenylcarbonyl, hydroxyl and trifluoromethyl), halogen, nitro, cyano, carboxyl and hydroxyl.

When R¹ represents a C₁-C₆alkoxy group (such as methoxy, ethoxy, propoxy, i-propoxy, butoxy, i-butoxy, t-butoxy pentoxy, i-pentoxy, neopentoxy, hexoxy), the C₁-C₆alkoxy group is optionally substituted by one or more substituents selected from C₁-C₆alkoxy (such as methoxy, ethoxy, propoxy, i-propoxy, butoxy, i-butoxy, t-butoxy pentoxy, i-pentoxy, neopentoxy, hexoxy), C₃-C₆cycloalkyl (such as cyclopropyl, cyclobutyl, cyclopentyl, cyclohexyl), C₁-C₆alkylthio (such as methylthio, ethylthio, propylthio, i-propylthio, butylthio, i-butylthio, t-butylthio, pentylthio, i-pentylthio, neopentylthio, hexylthio), —NR²⁷R²⁸, —C(O)NR²⁹R³⁰ (each of which may be optionally substituted by one or more substituents selected from halogen, C₁-C₆alkyl [such as methyl, ethyl, propyl, i-propyl, butyl, i-butyl, t-butyl pentyl, i-pentyl, neopentyl, hexyl], C₁-C₆alkoxy [such as methoxy, ethoxy, propoxy, i-propoxy, butoxy, i-butoxy, t-butoxy pentoxy, i-pentoxy, neopentoxy, hexoxy], C₁-C₆alkylthio [such as methylthio, ethylthio, propylthio, i-propylthio, butylthio, i-butylthio, t-butylthio, pentylthio, i-pentylthio, neopentylthio, hexylthio], amino [—NH₂], mono- and di-C₁-C₆alkylamino [such as methylamino, ethylamino, propylamino, i-propylamino, butylamino, i-butylamino, t-butylamino, pentylamino, i-pentylamino, neopentylamino, hexylamino], hydroxyl and trifluoromethyl) hydroxyl and a 5- or 6-membered aromatic ring optionally comprising at least one ring heteroatom selected from nitrogen, oxygen and sulphur, the ring being optionally substituted by one or more substituents selected from C₁-C₆alkyl (such as methyl, ethyl, propyl, i-propyl, butyl, i-butyl, t-butyl pentyl, i-pentyl, neopentyl, hexyl), C₁-C₆alkoxy (such as methoxy, ethoxy, propoxy, i-propoxy, butoxy, i-butoxy, t-butoxy pentoxy, i-pentoxy, neopentoxy, hexoxy, C₂-C₆alkenyl, C₃-C₆cycloalkyl (such as cyclopropyl, cyclobutyl, cyclopentyl, cyclohexyl), C₁-C₆alkoxycarbonyl (such as methoxycarbonyl, ethoxycarbonyl, propoxycarbonyl, i-propoxycarbonyl, butoxycarbonyl, i-butoxycarbonyl, t-butoxycarbonyl, pentoxycarbonyl, i-pentoxycarbonyl, neopentoxycarbonyl, hexoxycarbonyl), C₁-C₆alkylcarbonyl (such as methylcarbonyl, ethylcarbonyl, propylcarbonyl, i-propylcarbonyl, butylcarbonyl, i-butylcarbonyl, t-butylcarbonyl, pentylcarbonyl, i-pentylcarbonyl, neopentylcarbonyl, hexylcarbonyl), C₁-C₆alkylcarbonylamino (such as methylamino, ethylamino, propylamino, i-propylamino, butylamino, i-butylamino, t-butylamino, pentylamino, i-pentylamino, neopentylamino, hexylamino), phenylcarbonyl, —S(O)ₙC₁-C₆alkyl, —NR³¹R³², —C(O)NR³³R³⁴, —SO₂NR³⁵R³⁶ (each of which may be optionally substituted by one or more substituents selected from halogen, C₁-C₆alkyl [such as methyl, ethyl, propyl, i-propyl, butyl, i-butyl, t-butyl pentyl, i-pentyl, neopentyl, hexyl], C₁-C₆alkoxy [such as methoxy, ethoxy, propoxy, i-propoxy, butoxy, i-butoxy, t-butoxy pentoxy, i-pentoxy, neopentoxy, hexoxy], C₁-C₆alkylthio [such as methylthio, ethylthio, propylthio, i-propylthio, butylthio, i-butylthio, t-butylthio, pentylthio, i-pentylthio, neopentylthio, hexylthio], amino (—NH₂), mono- and di-C₁-C₆alkylamino [such as methylamino, ethylamino, propylamino, i-propylamino, butylamino, i-butylamino, t-butylamino, pentylamino, i-pentylamino, neopentylamino, hexylamino], hydroxyl and trifluoromethyl), halogen, nitro, cyano, carboxyl and hydroxyl.

When R¹ represents a C₆aryloxy group, the C₆aryloxy group is optionally substituted by one or more substituents selected from C₁-C₆alkyl (such as methyl, ethyl, propyl, i-propyl, butyl, i-butyl, t-butyl pentyl, i-pentyl, neopentyl, hexyl), C₁-C₆alkoxy (such as methoxy, ethoxy, propoxy, i-propoxy, butoxy, i-butoxy, t-butoxy pentoxy, i-pentoxy, neopentoxy, hexoxy), C₂-C₆alkenyl, C₃-C₆cycloalkyl (such as cyclopropyl, cyclobutyl, cyclopentyl, cyclohexyl), C₁-C₆alkoxycarbonyl (such as methoxycarbonyl, ethoxycarbonyl, propoxycarbonyl, i-propoxycarbonyl, butoxycarbonyl, i-butoxycarbonyl, t-butoxycarbonyl, pentoxycarbonyl, i-pentoxycarbonyl, neopentoxycarbonyl, hexoxycarbonyl), C₁-C₆alkylcarbonyl (such as methylcarbonyl, ethylcarbonyl, propylcarbonyl, i-propylcarbonyl, butylcarbonyl, i-butylcarbonyl, t-butylcarbonyl, pentylcarbonyl, i-pentylcarbonyl, neopentylcarbonyl, hexylcarbonyl), C₁-C₆alkylcarbonylamino (such as methylamino, ethylamino, propylamino, i-propylamino, butylamino, i-butylamino, t-butylamino, pentylamino, i-pentylamino, neopentylamino, hexylamino), phenylcarbonyl, —S(O)ₚC₁-C₆alkyl, —NR³⁷R³⁸, —C(O)NR³⁹R⁴⁰, —SO₂NR⁴¹R⁴² (each of which may be optionally substituted by one or more substituents selected from halogen, C₁-C₆alkyl [such as methyl, ethyl, propyl, i-propyl, butyl, i-butyl, t-butyl pentyl, i-pentyl, neopentyl, hexyl], C₁-C₆alkoxy [such as methoxy, ethoxy, propoxy, i-propoxy, butoxy, i-butoxy, t-butoxy pentoxy, i-pentoxy, neopentoxy, hexoxy], C₁-C₆alkylthio [such as methylthio, ethylthio, propylthio, i-propylthio, butylthio, i-butylthio, t-butylthio, pentylthio, i-pentylthio, neopentylthio, hexylthio], amino [—NH₂], mono- and di-C₁-C₆alkylamino [such as methylamino, ethylamino, propylamino, i-propylamino, butylamino, i-butylamino, t-butylamino, pentylamino, i-pentylamino, neopentylamino, hexylamino], hydroxyl and trifluoromethyl), halogen, nitro, cyano, carboxyl and hydroxyl.

When R¹ represents a 5- to 6-membered heteroaryloxy group, the 5- to 6-membered heteroaryloxy group is optionally substituted by one or more substituents selected from C₁-C₆alkyl (such as methyl, ethyl, propyl, i-propyl, butyl, i-butyl, t-butyl pentyl, i-pentyl, neopentyl, hexyl), C₁-C₆alkoxy (such as methoxy, ethoxy, propoxy, i-propoxy, butoxy, i-butoxy, t-butoxy pentoxy, i-pentoxy, neopentoxy, hexoxy), C₂-C₆alkenyl, C₃-C₆cycloalkyl (such as cyclopropyl, cyclobutyl, cyclopentyl, cyclohexyl), C₁-C₆alkoxycarbonyl (such as methoxycarbonyl, ethoxycarbonyl, propoxycarbonyl, i-propoxycarbonyl, butoxycarbonyl, i-butoxycarbonyl, t-butoxycarbonyl, pentoxycarbonyl, i-pentoxycarbonyl, neopentoxycarbonyl, hexoxycarbonyl), C₁-C₆alkylcarbonyl (such as methylcarbonyl, ethylcarbonyl, propylcarbonyl, i-propylcarbonyl, butylcarbonyl, i-butylcarbonyl, t-butylcarbonyl, pentylcarbonyl, i-pentylcarbonyl, neopentylcarbonyl, hexylcarbonyl), C₁-C₆alkylcarbonylamino (such as methylamino, ethylamino, propylamino, i-propylamino, butylamino, i-butylamino, t-butylamino, pentylamino, i-pentylamino, neopentylamino, hexylamino), phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl [such as methyl, ethyl, propyl, i-propyl, butyl, i-butyl, t-butyl pentyl, i-pentyl, neopentyl, hexyl], C₁-C₆alkoxy [such as methoxy, ethoxy, propoxy, i-propoxy, butoxy, i-butoxy, t-butoxy pentoxy, i-pentoxy, neopentoxy, hexoxy], C₁-C₆alkylthio [such as methylthio, ethylthio, propylthio, i-propylthio, butylthio, i-butylthio, t-butylthio, pentylthio, i-pentylthio, neopentylthio, hexylthio], amino [—NH₂], mono-C₁-C₆alkylamino, di-(C₁-C₆alky)amino [such as methylamino, ethylamino, propylamino, i-propylamino, butylamino, i-butylamino, t-butylamino, pentylamino, i-pentylamino, neopentylamino, hexylamino], hydroxyl and trifluoromethyl), halogen, nitro, cyano, carboxyl and hydroxyl.

When R¹ represents a —S(O)ₓR⁴⁹ group, R⁴⁹ represents C₁-C₆alkyl (such as methyl, ethyl, propyl, i-propyl, butyl, i-butyl, t-butyl pentyl, i-pentyl, neopentyl, hexyl), C₃-C₆cycloalkyl (such as cyclopropyl, cyclobutyl, cyclopentyl, cyclohexyl) or —CH₂Ar wherein Ar represents a 5- or 6-membered aromatic ring optionally comprising at least one ring heteroatom selected from nitrogen, oxygen and sulphur, the ring being optionally substituted by one or more substituents selected from C₁-C₆alkyl (such as methyl, ethyl, is propyl, i-propyl, butyl, i-butyl, t-butyl pentyl, i-pentyl, neopentyl, hexyl), C₁-C₆alkoxy (such as methoxy, ethoxy, propoxy, i-propoxy, butoxy, i-butoxy, t-butoxy pentoxy, i-pentoxy, neopentoxy, hexoxy), C₂-C₆alkenyl, C₃-C₆cycloalkyl (such as cyclopropyl, cyclobutyl, cyclopentyl, cyclohexyl), C₁-C₆alkoxycarbonyl (such as methoxycarbonyl, ethoxycarbonyl, propoxycarbonyl, i-propoxycarbonyl, butoxycarbonyl, i-butoxycarbonyl, t-butoxycarbonyl, pentoxycarbonyl, i-pentoxycarbonyl, neopentoxycarbonyl, hexoxycarbonyl), C₁-C₆alkylcarbonyl (such as methylcarbonyl, ethylcarbonyl, propylcarbonyl, i-propylcarbonyl, butylcarbonyl, i-butylcarbonyl, t-butylcarbonyl, pentylcarbonyl, i-pentylcarbonyl, neopentylcarbonyl, hexylcarbonyl), C₁-C₆alkylcarbonylamino (such as methylamino, ethylamino, propylamino, i-propylamino, butylamino, i-butylamino, t-butylamino, pentylamino, i-pentylamino, neopentylamino, hexylamino), phenylcarbonyl, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl [such as methyl, ethyl, propyl, i-propyl, butyl, i-butyl, t-butyl pentyl, i-pentyl, neopentyl, hexyl], C₁-C₆alkoxy [such as methoxy, ethoxy, propoxy, i-propoxy, butoxy, i-butoxy, t-butoxy pentoxy, i-pentoxy, neopentoxy, hexoxy], C₁-C₆alkylthio [such as methylthio, ethylthio, propylthio, i-propylthio, butylthio, i-butylthio, t-butylthio, pentylthio, i-pentylthio, neopentylthio, hexylthio], amino (—NH₂), mono- and di-C₁-C₆alkylamino [such as methylamino, ethylamino, propylamino, i-propylamino, butylamino, i-butylamino, t-butylamino, pentylamino, i-pentylamino, neopentylamino, hexylamino], hydroxyl and trifluoromethyl), —CH₂OCO₂H, halogen, nitro, cyano, carboxyl and hydroxyl.

When R¹ represents a —S(O)₂NR⁵⁰R⁵¹ group, R⁵⁰ and R⁵¹ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁵⁰ and R⁵¹ together with the nitrogen atom to which they are attached form a 4- to 6-membered saturated heterocycle (such as pyrrolidinyl or piperidinyl).

When R¹ represents -A-B, A represents a C₂-alkylene optionally substituted by one or more substituents selected from C₁-C₆alkoxy (such as methoxy, ethoxy, propoxy, i-propoxy, butoxy, i-butoxy, t-butoxy pentoxy, i-pentoxy, neopentoxy, hexoxy), C₃-C₆cycloalkyl (such as cyclopropyl, cyclobutyl, cyclopentyl, cyclohexyl), C₁-C₆alkylthio,- —NR⁵⁷R⁵⁸, —C(O)NR⁵⁹R⁶⁰ (each of which may be optionally substituted by one or more substituents i selected from halogen, C₁-C₆alkyl [such as methyl, ethyl, propyl, i-propyl, butyl, i-butyl, t-butyl pentyl, i-pentyl, neopentyl, hexyl], C₁-C₆alkoxy [such as methoxy, ethoxy, propoxy, i-propoxy, butoxy, i-butoxy, t-butoxy pentoxy, i-pentoxy, neopentoxy, hexoxy], C₁-C₆alkylthio [such as methylthio, ethylthio, propylthio, i-propylthio, butylthio, i-butylthio, t-butylthio, pentylthio, i-pentylthio, neopentylthio, hexylthio], amino (—NH₂), mono- and di-C₁-C₆alkylamino [such as methylamino, ethylamino, propylamino, i-propylamino, butylamino, i-butylamino, t-butylamino, pentylamino, i-pentylamino, neopentylamino, hexylamino], hydroxyl and trifluoromethyl), and hydroxyl, and B represents a 5- or 6-membered aromatic ring optionally comprising at least one ring heteroatom selected from nitrogen, oxygen and sulphur, the ring being optionally substituted by one or more substituents selected from C₁-C₆alkyl (such as methyl, ethyl, propyl, i-propyl, butyl, i-butyl, t-butyl pentyl, i-pentyl, neopentyl, hexyl), C₁-C₆alkoxy (such as methoxy, ethoxy, propoxy, i-propoxy, butoxy, i-butoxy, t-butoxy pentoxy, i-pentoxy, neopentoxy, hexoxy), C₂-C₆alkenyl, C₃-C₆cycloalkyl (such as cyclopropyl, cyclobutyl, cyclopentyl, cyclohexyl), C₁-C₆alkoxycarbonyl (such as methoxycarbonyl, ethoxycarbonyl, propoxycarbonyl, i-propoxycarbonyl, butoxycarbonyl, i-butoxycarbonyl, t-butoxycarbonyl, pentoxycarbonyl, i-pentoxycarbonyl, neopentoxycarbonyl, hexoxycarbonyl), C₁-C₆alkylcarbonyl (such as methylcarbonyl, ethylcarbonyl, propylcarbonyl, i-propylcarbonyl, butylcarbonyl, i-butylcarbonyl, t-butylcarbonyl, pentylcarbonyl, i-pentylcarbonyl, neopentylcarbonyl, hexylcarbonyl), C₁-C₆alkylcarbonylamino (such as methylamino, ethylamino, propylamino, i-propylamino, butylamino, i-butylamino, t-butylamino, pentylamino, i-pentylamino, neopentylamino, hexylamino), phenylcarbonyl, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl [such as methyl, ethyl, propyl, i-propyl, butyl, i-butyl, t-butyl pentyl, i-pentyl, neopentyl, hexyl], C₁-C₆alkoxy [such as methoxy, ethoxy, propoxy, i-propoxy, butoxy, i-butoxy, t-butoxy pentoxy, i-pentoxy, neopentoxy, hexoxy], C₁-C₆alkylthio [such as methylthio, ethylthio, propylthio, i-propylthio, butylthio, i-butylthio, t-butylthio, pentylthio, i-pentylthio, neopentylthio, hexylthio], amino (—NH₂), mono- and di-C₁-C₆alkylamino [such as methylamino, ethylamino, propylamino, i-propylamino, butylamino, i-butylamino, t-butylamino, pentylamino, i-pentylamino, neopentylamino, hexylamino], hydroxyl and trifluoromethyl), -CH₂OCO₂H, halogen, nitro, is cyano, carboxyl, hydroxyl and optionally wherein two or more adjacent substituents together with the atoms to which they are attached form a partially or fully unsaturated 4- to 6-membered ring.

When B represents a 5- or 6-membered aromatic ring optionally comprising at least one ring heteroatom selected from nitrogen, oxygen and sulphur, the ring being optionally substituted by at least two adjacent substituents and wherein the two or more adjacent substituents together with the atoms to which they are attached form a partially or fully unsaturated 4- to 6-membered ring, examples of B include indole, indoline, benzothiophen, benzofuran, benzimidazole and benzodioxole.

When R² represents a C₁-C₃alkyl group (such as methyl, ethyl, propyl, i-propyl) the C₁-C₃alkyl group is optionally substituted by one or more substituents selected from C₁-C₃alkoxy (such as methoxy, ethoxy, propoxy, i-propoxy), cyano, hydroxyl, amino (—NH₂), mono-C₁-C₃alkylamino and di-(C₁-C₃alky)amino (such as methylamino, ethylamino, propylamino, i-propylamino).

When R³ represents a C₁-C₅alkyl group (such as methyl, ethyl, propyl, i-propyl, butyl, i-butyl, t-butyl pentyl, i-pentyl, neopentyl), the C₁-C₅alkyl group is optionally substituted with C₁-C₃alkoxy (such as methoxy, ethoxy, propoxy, i-propoxy), cyano, hydroxyl, amino (—NH₂), mono-C₁-C₃alkylamino and di-(C₁-C₃alky)amino (such as methylamino, ethylamino, propylamino, i-propylamino).

When R³ represents a C₃-C₅cycloalkyl group (such as cyclopropyl, cyclobutyl, cyclopentyl), the C₃-C₅cycloalkyl group is optionally substituted with C₁-C₃alkoxy (such as methoxy, ethoxy, propoxy, i-propoxy).

When R³ represents a 3- to 5-membered saturated heterocyclyl group, the 3- to 5-membered saturated heterocyclyl group is optionally substituted with by one or more substituents selected from C₁-C₃alkyl (such as methyl, ethyl, propyl, i-propyl), C₁-C₃alkoxy (such as methoxy, ethoxy, propoxy, i-propoxy) and C₃cycloalkyl (such as cyclopropyl).

When R⁴ represents a C₁-C₆alkyl group (such as methyl, ethyl, propyl, i-propyl, butyl, i-butyl, t-butyl pentyl, i-pentyl, neopentyl, hexyl), the C₁-C₆alkyl group is optionally substituted with C₁-C₃alkoxy (such as methoxy, ethoxy, propoxy, i-propoxy), hydroxyl, amino (—NH₂), mono-C₁-C₃alkylamino and di-(C₁-C₃alky)amino (such as methylamino, ethylamino, propylamino, i-propylamino).

When R⁴ represents a C₁-C₆alkenyl group, the C₁-C₆alkenyl group is optionally substituted with C₁-C₃alkoxy (such as methoxy, ethoxy, propoxy, i-propoxy).

When R⁴ represents a C₁-C₆alkynyl group, the C₁-C₆alkynyl group is optionally substituted with C₁-C₃alkoxy (such as methoxy, ethoxy, propoxy, i-propoxy).

When R⁴ represents a C₃-C₅cycloalkyl group (such as cyclopropyl, cyclobutyl, cyclopentyl), the C₃-C₅cycloalkyl group is optionally substituted with C₁-C₃alkoxy (such as methoxy, ethoxy, propoxy, i-propoxy).

When R⁴ represents a C₁-C₆alkoxy group (such as methoxy, ethoxy, propoxy, i-propoxy, butoxy, i-butoxy, t-butoxy pentoxy, i-pentoxy, neopentoxy, hexoxy), the C₁-C₆alkoxy group is optionally substituted with C₁-C₃alkoxy (such as methoxy, ethoxy, propoxy, i-propoxy), hydroxyl, amino (—NH₂), mono-C₁-C₃alkylamino and di-(C₁-C₃alky)amino (such as methylamino, ethylamino, propylamino, i-propylamino).

When R⁴ represents -CONR⁵²R⁵³, R⁵² and R⁵³ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (such as cyclopropyl, cyclobutyl, cyclopentyl and cyclohexyl), or R⁵² and R⁵³ together with the nitrogen atom to which they are attached form a 4- to 6-membered saturated heterocycle (such as pyrrolidinyl or piperidinyl).

When R⁴ represents —NR⁵⁴R⁵⁵, R⁵⁴ and R⁵⁵ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁵⁴ and R⁵⁵ together with the nitrogen atom to which they are attached form a 4- to 6-membered saturated heterocycle (such as pyrrolidinyl or piperidinyl).

When R⁴ represents —S(O)_{y}R⁵⁶, R⁵⁶ represents C₁-C₆alkyl (such as methyl, ethyl, propyl, i-propyl, butyl, i-butyl, t-butyl pentyl, i-pentyl, neopentyl, hexyl) or C₃-C₆cycloalkyl(such as cyclopropyl, cyclobutyl, cyclopentyl and cyclohexyl).

R⁵ and R⁶ each independently represent hydrogen, C₁-C₄, particularly C₁-C₂alkyl 1o (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁵ and R⁶ together with the nitrogen atom to which they are attached form a 4- to 6-membered saturated heterocycle (such as pyrrolidinyl or piperidinyl).

R⁷ and R⁸ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁷ and R⁸ together with the nitrogen atom to which they are attached form a 4- to 6-membered saturated heterocycle (such as pyrrolidinyl or piperidinyl).

R⁹ and R¹⁰ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁹ and R¹⁰ together with the nitrogen atom to which they are attached form a 4- to 6-membered saturated heterocycle (such as pyrrolidinyl or piperidinyl).

R¹l and R¹² each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R¹¹ and R¹² together with the nitrogen atom to which they are attached form a 4- to 6-membered saturated heterocycle (such as pyrrolidinyl or piperidinyl).

R¹³ and R¹⁴ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R¹³ and R¹⁴ together with the nitrogen atom to which they are attached form a 4- to 6-membered saturated heterocycle (such as pyrrolidinyl or piperidinyl).

R¹⁵ and R¹⁶ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R¹⁵ and R¹⁶ together with the nitrogen atom to which they are attached form a 4- to 6-membered saturated heterocycle (such as pyrrolidinyl or piperidinyl).

R¹⁷ and R¹⁸ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R¹⁷ and R¹⁸ together with the nitrogen atom to which they are attached form a 4- to 6-membered saturated heterocycle (such as pyrrolidinyl or piperidinyl).

R¹⁹ and R²⁰ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R¹⁹ and R²⁰ together with the nitrogen atom to which they are attached form a 4- to 6-membered saturated heterocycle (such as pyrrolidinyl or piperidinyl).

R²¹ and R²² each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R²¹ and R²² together with the nitrogen atom to which they are attached form a 4- to 6-membered saturated heterocycle (such as pyrrolidinyl or piperidinyl).

R²³ and R²⁴ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R²³ and R²⁴ together with the nitrogen atom to which they are attached form a 4- to 6-membered saturated heterocycle (such as pyrrolidinyl or piperidinyl).

R²⁵ and R²⁶ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R²⁵ and R²⁶ together with the nitrogen atom to which they are attached form a 4- to 6-membered saturated heterocycle (such as pyrrolidinyl or piperidinyl).

R²⁷ and R²⁸each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R²⁷ and R²⁸ together with the nitrogen atom to which they are attached form a 4- to 6-membered saturated heterocycle (such as pyrrolidinyl or piperidinyl).

R²⁹ and R³⁰ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R²⁹ and R³⁰ together with the nitrogen atom to which they are attached form a 4- to 6-membered saturated heterocycle (such as pyrrolidinyl or piperidinyl).

R³¹ and R³² each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R³¹ and R³² together with the nitrogen atom to which they are attached form a 4- to 6-membered saturated heterocycle (such as pyrrolidinyl or piperidinyl).

R³³ and R³⁴ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R³³ and R³⁴ together with the nitrogen atom to which they are attached form a 4- to 6-membered saturated heterocycle (such as pyrrolidinyl or piperidinyl).

R³⁵ and R³⁶ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R³⁵ and R³⁶ together with the nitrogen atom to which they are attached form a 4- to 6-membered saturated heterocycle (such as pyrrolidinyl or piperidinyl).

R³⁷ and R³⁸ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R³⁷ and R³⁸ together with the nitrogen atom to which they are attached form a 4- to 6-membered saturated heterocycle (such as pyrrolidinyl or piperidinyl).

R³⁹ and R⁴⁰ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R³⁹ and R⁴⁰ together with the nitrogen atom to which they are attached form a 4- to 6-membered saturated heterocycle (such as pyrrolidinyl or piperidinyl).

R⁴¹ and R⁴² each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁴¹ and R⁴² together with the nitrogen atom to which they are attached form a 4- to 6-membered saturated heterocycle (such as pyrrolidinyl or piperidinyl).

R⁴³ and R⁴⁴ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁴³ and R⁴⁴ together with the nitrogen atom to which they are attached form a 4- to 6-membered saturated heterocycle (such as pyrrolidinyl or piperidinyl).

R⁴⁵ and R⁴⁶ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁴⁵ and R⁴⁶ together with the nitrogen atom to which they are attached form a 4- to 6-membered saturated heterocycle (such as pyrrolidinyl or piperidinyl).

R⁴⁷ and R⁴⁸ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁴⁷ and R⁴⁸ together with the nitrogen atom to which they are attached form a 4- to 6-membered saturated heterocycle (such as pyrrolidinyl or piperidinyl).

R⁵⁷ and R ⁵⁸ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁵⁷ and R⁵⁸ together with the nitrogen atom to which they are attached form a 4- to 6-membered saturated heterocycle (such as pyrrolidinyl or piperidinyl).

R⁵⁹ and R⁶⁰ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁵⁹ and R⁶⁰ together with the nitrogen atom to which they are attached form a 4- to 6-membered saturated heterocycle (such as pyrrolidinyl or piperidinyl).

R⁶¹ and R⁶² each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁶¹ and R⁶² together with the nitrogen atom to which they are attached form a 4- to 6-membered saturated heterocycle (such as pyrrolidinyl or piperidinyl).

R⁶³ and R⁶⁴ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁶³ and R⁶⁴ together with the nitrogen atom to which they are attached form a 4- to 6-membered saturated heterocycle (such as pyrrolidinyl or piperidinyl).

R⁶⁵ and R⁶⁶ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁶⁵ and R⁶⁶ together with the nitrogen atom to which they are attached form a 4- to 6-membered saturated heterocycle (such as pyrrolidinyl or piperidinyl).

Particular values of variable groups are as follows. Such values may be used where appropriate with any of the definitions, claims or embodiments defined hereinbefore or hereinafter.

In one embodiment of the invention, R¹ represents
a C₁-C₆alkoxy group optionally substituted by one or more substituents selected from C₁-C₆alkoxy, C₆-aryloxy, C₃-C₆cycloalkyl, —NR²⁷R²⁸, —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —OSO₂C₁₋₆alkyl, —NR³¹R³², —C(O)NR³³R³⁴, —NHC(O)OC₁₋₆alkyl, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl;

a C₆aryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₚC₁-C₆alkyl, —NR³⁷R³⁸, —C(O)N³⁹R⁴⁰, —SO₂NR⁴¹R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl; or a 5- to 6-membered heteroaryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl.

In another embodiment of the invention, R¹ represents a C₁-C₆alkoxy group optionally substituted by one or more substituents selected from C₁-C₆alkoxy.

In another embodiment of the invention, R¹ represents a C₁-C₆alkoxy group.

In another embodiment of the invention, R¹ represents a C₁-C₃alkoxy group.

In another embodiment of the invention, R¹ represents a i-propoxy group.

In another embodiment of the invention, R¹ represents a C₁-C₆alkyl group optionally substituted by one or more substituents selected from C₁-C₆alkoxy, C₃-C₆cycloalkyl, C₁-C₆alkylthio, —NR⁵R⁶, —C(O)NR⁷R⁸, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), cyano and hydroxyl.

In a further embodiment of the invention, R¹ represents a C₁-C₆alkyl group substituted by one or more substituents selected from C₁-C₆alkoxy, —NR⁵R⁶, —C(O)NR⁷R⁸, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), and hydroxyl.

In a further embodiment of the invention, R¹ represents a C₁-C₆alkyl group substituted by one or more substituents selected from C₁-C₆alkoxy (which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl) and hydroxyl.

In a further embodiment of the invention R¹ represents a C₃-C₅cycloalkyl group optionally substituted by one or more substituents selected from C₁-C₆alkoxy, C₃-C₆cycloalkyl, C₁-C₆alkylthio, —NR⁹R¹⁰, —C(O)NR¹¹R¹² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl.

In one embodiment of the invention R¹ represents a 4- to 6-membered heterocyclyl group optionally substituted with by one or more substituents selected from C₁-C₆alkyl, C₁-C₆alkoxy, C₃-C₆cycloalkyl, C₁-C₆alkylthio, —NR¹⁷R¹⁸, —C(O)NR¹⁹R²⁰, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₘC₁-C₆alkyl, —NR²¹R²², —C(O)NR²³R²⁴, —SO₂NR²⁵R²⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl.

In one embodiment of the invention R¹ represents -A-B wherein
A represents a C₂-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a C₁-alkyleneoxy optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; and
B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, C₁-C₆alkyloxycarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring.

In one embodiment of the invention R¹ represents -A-B wherein
A represents a C₂-alkylene optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a C₁-alkyleneoxy optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or an oxyC₁-alkylene optionally substituted by one or more substituents selected from C₁-C₆alkoxy, C₃-C₆cycloalkyl, C₁-C₆alkylthio, —NR⁵⁷R⁵3 —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; and
B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring.

In another embodiment of the invention R¹ represents -A-B wherein
A represents a C₂-alkylene optionally substituted by one or more substituents selected from C₁-C₆alkyl, C₁-C₆alkoxy, C₃-C₆cycloalkyl, C₁-C₆alkylthio,- —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; or an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; and
B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring.

In another embodiment of the invention R¹ represents -A-B wherein
A represents a C₂-alkylene optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; or an oxyC,-alkylene optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; and
B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring.

In a further embodiment of the invention R¹ represents -A-B wherein
A represents a C₂-alkylene optionally substituted by one or more substituents selected from C₁-C₆alkyl, C₁-C₆alkoxy, C₃-C₆cycloalkyl, C₁-C₆alkylthio,- —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; or an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; and
B represents a phenyl ring or a pyridin-4-yl ring each optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered nng.

In a further embodiment of the invention R¹ represents -A-B wherein
A represents a C₂-alkylene optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; or an oxyC,-alkylene optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; and
B represents a phenyl ring optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring.

In a further embodiment of the invention R¹ represents -A-B wherein
A represents a C₂-alkylene optionally substituted by one or more substituents selected from C₁-C₆alkyl, C₁-C₆alkoxy, C₃-C₆cycloalkyl, C₁-C₆alkylthio,- —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; and
B represents a phenyl ring or a pyridin-4-yl ring each optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring.

In a further embodiment of the invention R¹ represents -A-B wherein
A represents a C₂-alkylene optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; and
B represents a phenyl ring optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring.

In a further embodiment of the invention R¹ represents -A-B wherein
A represents an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; and
B represents a phenyl ring or a pyridin-4-yl ring each optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring.

In a further embodiment of the invention R¹ represents -A-B wherein
A represents a —CH₂CH₂— or a —OCH₂—; and
B represents a phenyl ring or a pyridin-4-yl ring each optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered nng.

In a further embodiment of the invention R¹ represents -A-B wherein
A represents a —CH₂CH₂— or a —OCH₂—; and
B represents a phenyl ring optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring.

In a further embodiment of the invention R¹ represents -A-B wherein
A represents a —CH₂CH₂— or a —OCH₂—; and

B represents a phenyl ring or a pyridin-4-yl ring each optionally substituted by one or more substituents selected from C₁-C₆alkyl, C₁-C₆alkoxy, C₁-C₆alkoxycarbonyl, C₁-C₆alkylcarbonylamino, phenyl, —NR⁶¹R⁶², —C(O)NR⁶³R⁶⁴, (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring.

In a further embodiment of the invention R¹ represents -A-B wherein
A represents a —CH₂CH₂— or a —OCH₂—; and
B represents a phenyl ring or a pyridin-4-yl ring each optionally substituted by one or more substituents selected from C₁-C₆alkyl, C₁-C₆alkoxy, C₁-C₆alkoxycarbonyl, C₁-C₆alkylcarbonylamino, phenyl, —NR⁶¹R⁶², —C(O)NR⁶³R⁶⁴, (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), halogen, cyano, carboxyl and hydroxyl, and optionally wherein two or more adjacent substituents together with the atoms to which they are attached form a partially or fully unsaturated 4- to 6-membered ring.
R⁶¹ and R⁶² each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁶¹ and R⁶² together with the nitrogen atom to which they are attached form a 4- to 6-membered saturated heterocycle (such as pyrrolidinyl, morpholiny or piperidinyl).

R⁶³ and R⁶⁴ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁶³ and R⁶⁴ together with the nitrogen atom to which they are attached form a 4- to 6-membered saturated heterocycle (such as pyrrolidinyl, morpholiny or piperidinyl).

In one embodiment of the invention, R¹ represents a C₁-C₃alkyl group (such as methyl, ethyl, propyl and i-propyl) optionally substituted by one or more substituents selected from C₁-C₃alkoxy (such as methoxy, ethoxy, propoxy and i-propoxy), C₃-C₄cycloalkyl (such as cyclopropyl and cyclobutyl) [each of which may be optionally substituted by one or more substituents selected from halogen (such as fluorine, chlorine, bromine or iodine), C₁-C₃alkyl (such as methyl, ethyl, propyl and i-propyl), C₁-C₃alkoxy (such as methoxy, ethoxy, propoxy and i-propoxy)], and hydroxyl; a cyclopropyl group optionally substituted by C₁-C₃alkoxy (such as methoxy, ethoxy, propoxy and i-propoxy); a C₁-C₃alkoxy group (such as methoxy, ethoxy, propoxy and i-propoxy) optionally substituted by one or more substituents selected from C₁-C₃alkoxy (such as methoxy, ethoxy, propoxy and i-propoxy) and cyclopropyl; a phenyloxy group optionally substituted by one or more substituents selected from C₁-C₃alkyl (such as methyl, ethyl, propyl and i-propyl), C₁-C₃alkoxy(such as methoxy, ethoxy, propoxy and i-propoxy) and cyclopropyl; or -A-B wherein A represents a C₂-alkylene, and B represents a phenyl ring optionally substituted by one or more substituents selected from C₁-C₃alkyl, C₁-C₃alkoxy or cyclopropyl.

In another embodiment of the invention, R¹ represents a C₁-C₃alkyl group (such as methyl, ethyl, propyl and i-propyl) substituted by one or more substituents selected from C₁-C₃alkoxy (such as methoxy, ethoxy, propoxy and i-propoxy) [which may be optionally substituted by one or more substituents selected from halogen (such as fluorine, chlorine, bromine or iodine), C₁-C₃alkyl (such as methyl, ethyl, propyl and i-propyl), C₁-C₃alkoxy (such as methoxy, ethoxy, propoxy and i-propoxy)], and hydroxyl; a C₁-C₃alkoxy group (such as methoxy, ethoxy, propoxy and i-propoxy) optionally substituted by one or more substituents selected from C₁-C₃alkoxy (such as methoxy, ethoxy, propoxy and i-propoxy) and cyclopropyl; a phenyloxy group optionally substituted by one or more substituents selected from C₁-C₃alkyl (such as methyl, ethyl, propyl and i-propyl), C₁-C₃alkoxy(such as methoxy, ethoxy, propoxy and i-propoxy) and cyclopropyl; or -A-B wherein A represents a C₂-alkylene or oxyC₁-alkylene, and B represents a phenyl ring optionally substituted by one or more substituents selected from halogen, C₁-C₃alkyl, C₁-C₃alkoxy or C(O)NR⁶³R⁶⁴.

In a further additional aspect of the invention R¹ represents a methyl, ethyl, propyl, i-propyl, hydroxymethyl, cyclopropyl, methoxypropyl, ethoxypropyl, phenylethyl, p-methoxyphenylethyl, m-methoxyphenylethyl, 3,5-dimethoxyphenylethyl, i-propoxy, benzyloxy, or a (3,5-dimethoxyphenyl)methoxy group.

In a further additional aspect of the invention R¹ represents a hydroxymethyl, methoxypropyl, ethoxypropyl, phenylethyl, 2-(3-methoxyphenyl)ethyl, 2-(3,5-dimethoxyphenyl)ethyl, i-propoxy, benzyloxy, (3,5-dimethoxyphenyl)methoxy, 2-(3-hydroxyphenyl)ethyl, 2-(3,5-dihydroxyphenyl)ethyl, (3-methoxyphenyl)methoxy, [3-(methylcarbamoyl)phenyl]methoxy, [3-methoxy-5-(methylcarbamoyl)phenyl]methoxy, 2-[3-(methylcarbamoyl)phenyl]ethyl, 2-[3-methoxy-5-(methylcarbamoyl)phenyl]ethyl, (3-hydroxyphenyl)methoxy, (3,5-dihydroxyphenyl)methoxy, (3-chloro-5-methoxy-phenyl)methoxy, 2-(2,6-dimethoxypyridin-4-yl)ethyl, (5-fluoro-2-methoxy-pyridin-4-yl)methoxy, 2-(5-fluoro-2-methoxy-pyridin-4-yl)ethyl, (3-methoxy-5-methyl-phenyl)methoxy, (3-fluorophenyl)methoxy, (3-chlorophenyl)methoxy, 2-(3-aminophenyl)ethyl, 2-(5-methoxythiophen-2-yl)ethyl, 2-(2-furyl)ethyl, (2,6-dimethoxypyridin-4-yl)methoxy or a 2-(3-chloro-5-methoxy-phenyl)ethyl group.

In a further additional aspect of the invention R¹ represents a hydroxymethyl, methoxypropyl, ethoxypropyl, phenylethyl, 2-(3-methoxyphenyl)ethyl, 2-(3,5-dimethoxyphenyl)ethyl, i-propoxy, benzyloxy, (3,5-dimethoxyphenyl)methoxy, 2-(3-hydroxyphenyl)ethyl, 2-(3,5-dihydroxyphenyl)ethyl, (3-methoxyphenyl)methoxy, [3-(methylcarbamoyl)phenyl]methoxy, [3-methoxy-5-(methylcarbamoyl)phenyl]methoxy, 2-[3-(methylcarbamoyl)phenyl]ethyl, 2-[3-methoxy-5-(methylcarbamoyl)phenyl]ethyl, (3-hydroxyphenyl)methoxy, (3,5-dihydroxyphenyl)methoxy, (3-chloro-5-methoxy-phenyl)methoxy, 2-(2,6-dimethoxypyridin-4-yl)ethyl, (5-fluoro-2-methoxy-pyridin-4-yl)methoxy, 2-(5-fluoro-2-methoxy-pyridin-4-yl)ethyl, (3-methoxy-5-methyl-phenyl)methoxy, (3-fluorophenyl)methoxy, (3-chlorophenyl)methoxy, 2-(3-aminophenyl)ethyl, 2-(5-methoxythiophen-2-yl)ethyl, 2-(2-furyl)ethyl or a 2-(3-chloro-5-methoxy-phenyl)ethyl group.

In a further additional aspect of the invention R¹ represents a hydroxymethyl, methoxypropyl, ethoxypropyl, phenylethyl, 2-(3-methoxyphenyl)ethyl, 2-(3,5-dimethoxyphenyl)ethyl, i-propoxy, benzyloxy, (3,5-dimethoxyphenyl)methoxy, 2-(3-hydroxyphenyl)ethyl, 2-(3,5-dihydroxyphenyl)ethyl, (3-methoxyphenyl)methoxy, [3-(methylcarbamoyl)phenyl]methoxy, 2-[3-(methylcarbamoyl)phenyl]ethyl, 2-[3-methoxy-5-(methylcarbamoyl)phenyl]ethyl, 2-(2,6-dimethoxypyridin-4-yl)ethyl, (5-fluoro-2-methoxy-pyridin-4-yl)methoxy, 2-(5-fluoro-2-methoxy-pyridin-4-yl)ethyl, (3-methoxy-5-methyl-phenyl)methoxy, (3-fluorophenyl)methoxy, (3-chlorophenyl)methoxy, 2-(3-aminophenyl)ethyl, 2-(5-methoxythiophen-2-yl)ethyl, 2-(2-furyl)ethyl or a 2-(3-chloro-5-methoxy-phenyl)ethyl group.

In a further additional aspect of the invention R¹ represents a hydroxymethyl, methoxypropyl, ethoxypropyl, phenylethyl, 2-(3-methoxyphenyl)ethyl, 2-(3,5-dimethoxyphenyl)ethyl, i-propoxy, benzyloxy, (3,5-dimethoxyphenyl)methoxy, 2-(3-hydroxyphenyl)ethyl, 2-(3,5-dihydroxyphenyl)ethyl, (3-methoxyphenyl)methoxy, [3-(methylcarbamoyl)phenyl]methoxy, [3-methoxy-5-(methylcarbamoyl)phenyl]methoxy, 2-[3-(methylcarbamoyl)phenyl]ethyl, 2-[3-methoxy-5-(methylcarbamoyl)phenyl]ethyl, (3-hydroxyphenyl)methoxy, (3,5-dihydroxyphenyl)methoxy, (3-chloro-5-methoxy-phenyl)methoxy, or a 2-(3-chloro-5-methoxy-phenyl)ethyl group.

In another embodiment of the invention, R² represents hydrogen or a C₁-C₃alkyl group (such as methyl, ethyl, n-propyl, or isopropyl).

In a further aspect of the invention, R² represents hydrogen or methyl.

In a further aspect of the invention, R² represents hydrogen.

In a further embodiment of the invention, R³ represents a C₁-C₅alkyl group; a C₃-C₅cycloalkyl group; a oxolan-2-yl group; a CH₂N(CH₃)₂ group; a —CONHMe group or a —CONH₂ group.

In a further embodiment of the invention, R³ represents a C₁-C₅alkyl group; a C₃-C₅cycloalkyl group; or a —CONH₂ group.

In a further aspect of the invention, R³ represents methyl, ethyl, propyl, i-propyl, cyclopropyl, cyclobutyl or —CONH₂.

In a further aspect of the invention, R³ represents methyl, ethyl, propyl, i-propyl, cyclopropyl or —CONH₂.

In a further aspect of the invention R³ represents methyl, cyclopropyl, cyclobutyl or —CONH₂.

In a further aspect of the invention R³ represents methyl, cyclopropyl or —CONH₂.

In a further embodiment of the invention R⁴ hydrogen, a C₁-C₆alkyl group; a C₃-C₅cycloalkyl; a C₁-C₆alkoxy group.

In a further aspect of the invention, R⁴ represents hydrogen, methyl or methoxy.

In a further aspect R⁴ represents hydrogen.

In an embodiment of the invention, there is provided a subset of compounds of formula (I), and pharmaceutically acceptable salts thereof, in which:
R¹ represents a C₁-C₆alkyl group optionally substituted by one or more substituents selected from C₁-C₆alkoxy, C₃-C₆cycloalkyl, C₁-C₆alkylthio, —NR⁵R⁶, —C(O)NR⁷R⁸, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), cyano and hydroxyl, a C₃-C₅cycloalkyl group optionally substituted by one or more substituents selected from C₁-C₆alkoxy, C₃-C₆cycloalkyl, C₁-C₆alkylthio, —NR⁹R¹⁰, —C(O)NR¹¹R¹² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a 4- to 6-membered heterocyclyl group optionally substituted with by one or more substituents selected from C₁-C₆alkyl, C₁-C₆alkoxy, C₃-C₆cycloalkyl, C₁-C₆alkylthio, —NR¹⁷R¹⁸, —C(O)NR¹⁹R²⁰, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₘC₁-C₆alkyl, —NR²¹R²², —C(O)NR²³R²⁴, —SO₂NR²⁵R²⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₁-C₆alkoxy group optionally substituted by one or more substituents selected from C₁-C₆alkoxy, C₃-C₆cycloalkyl, —NR²⁷R²⁸, —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —NR³¹R³², —C(O)NR³³R³⁴, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₆aryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C ₁-C₆alkylcarbonylamino, phenylcarbonyl, —S(O)ₚC₁-C₆alkyl, —NR³⁷R³⁸, —C(O)NR³⁹R⁴⁰, —SO₂NR⁴¹R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a 5- to 6-membered heteroaryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl, or -A-B wherein A represents a C₂-alkylene optionally substituted by
   one or more substituents selected from C₁-C₆alkyl, C₁-C₆alkoxy, C₃-C₆cycloalkyl, C₁-C₆alkylthio, —NR⁵⁷ R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or a C₁-alkyleneoxy optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or an oxyCI-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring;
R² represents hydrogen;
R⁴ represents hydrogen; and
   wherein
(i) when R¹ is an optionally substituted 4- to 6-membered heterocyclyl group, C₁-C₆alkoxy group, C₆aryloxy group, 5- to 6-membered heteroaryloxy or -A-B group,
   R³ represents methyl, ethyl, propyl, i-propyl, cyclopropyl, cyclobutyl, —CONH₂ or —CONHMe,
or (ii) when R¹ is an optionally substituted C₁-C₆alkyl or a C₃-C₅cycloalkyl group,
R³ represents methyl, ethyl, propyl, i-propyl, cyclopropyl, cyclobutyl or —CONH₂.

In an embodiment of the invention, there is provided a subset of compounds of formula (I), and pharmaceutically acceptable salts thereof, in which:
R¹ represents a C₁-C₆alkyl group optionally substituted by one or more substituents selected from C₁-C₆alkoxy, C₃-C₆cycloalkyl, C₁-C₆alkylthio, —NR⁵R⁶, —C(O)NR⁷R⁸, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), cyano and hydroxyl, a C₃-C₅cycloalkyl group optionally substituted by one or more substituents selected from C₁-C₆alkoxy, C₃-C₆cycloalkyl, C₁-C₆alkylthio, —NR⁹R¹⁰, —C(O)NR¹¹R¹² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a 4- to 6-membered heterocyclyl group optionally substituted with by one or more substituents selected from C₁-C₆alkyl, C₁-C₆alkoxy, C₃-C₆cycloalkyl, C₁-C₆alkylthio, —NR¹⁷R¹⁸, —C(O)NR¹⁹R²⁰, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₘC₁-C₆alkyl, —NR²¹R²², —C(O)NR²³R²⁴, —SO₂NR²⁵R²⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₁-C₆alkoxy group optionally substituted by one or more substituents selected from C₁-C₆alkoxy, C₃-C₆cycloalkyl, —NR²⁷R²⁸, —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —NR³¹R³², —C(O)NR³³R³⁴, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₆aryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₚC₁-C₆alkyl, —NR³⁷R³⁸, —C(O)NR³⁹R⁴⁰, —SO₂NR⁴ R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a 5- to 6-membered heteroaryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl, or -A-B wherein A represents a C₂-alkylene optionally substituted by
   one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or a C₁-alkyleneoxy optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or an oxyCI-alkylene optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring;

R² represents hydrogen;

R⁴ represents hydrogen; and

wherein
(i) when R¹ is an optionally substituted 4- to 6-membered heterocyclyl group, C₁-C₆alkoxy group, C₆aryloxy group, 5- to 6-membered heteroaryloxy or -A-B group,
   R³ represents methyl, ethyl, propyl, i-propyl, cyclopropyl, cyclobutyl, -CONH₂ or —CONHMe,
or (ii) when R¹ is an optionally substituted C₁-C₆alkyl or a C₃-C₅cycloalkyl group,
   R³ represents methyl, ethyl, propyl, i-propyl, cyclopropyl, cyclobutyl or —CONH₂.

In another embodiment of the invention, there is provided a subset of compounds of formula (I), and pharmaceutically acceptable salts thereof, in which:
R¹ represents a C₁-C₆alkyl group substituted by one or more substituents selected from C₁-C₆alkoxy (which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), and hydroxyl, a C₁-C₆alkoxy group optionally substituted by one or more substituents selected from C₁-C₆alkoxy, C₃-C₆cycloalkyl, —NR²⁷R²⁸, —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —NR⁵¹R⁵², —C(O)NR³³R³⁴, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₆aryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, -S(O)pC₁-C₆alkyl, —NR³⁷R³3,-C(O)NR³⁹R⁴⁰, —SO₂NR⁴¹R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a 5- to 6-membered heteroaryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl, or -A-B wherein A represents a C₂-alkylene optionally substituted by
   one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring;
R² represents hydrogen;
R⁴ represents hydrogen; and
   wherein
(i) when R¹ is an optionally substituted C₁-C₆alkoxy group, C₆aryloxy group, 5- to 6-membered heteroaryloxy or -A-B group,
   R³ represents methyl, ethyl, propyl, i-propyl, cyclopropyl, cyclobutyl, —CONH₂ or —CONHMe,
or (ii) when R¹ is an optionally substituted C₁-C₆alkyl group,
   R³ represents methyl, ethyl, propyl, i-propyl, cyclopropyl, cyclobutyl or —CONH₂.

In another embodiment of the invention, there is provided a subset of compounds of formula (I), and pharmaceutically acceptable salts thereof, in which:
R¹ represents a C₁-C₆alkyl group substituted by one or more substituents selected from C₁-C₆alkoxy (which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), and hydroxyl, a C₁-C₆alkoxy group optionally substituted by one or more substituents selected from C₁-C₆alkoxy, C₃-C₆cycloalkyl, —NR²⁷R²⁸, —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —NR³¹R³², —C(O)NR³³R³⁴, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₆aryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C ₁-C₆alkylcarbonylamino, phenylcarbonyl, —S(O)ₚC₁-C₆alkyl, —NR³⁷R³⁸, —C(O)NR³⁹R⁴⁰, —SO₂NR⁴¹R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a 5- to 6-membered heteroaryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl, or -A-B wherein A represents a C₂-alkylene optionally substituted by
   one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or an oxyCI-alkylene optionally substituted by one or more substituents selected from C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring;
R² represents hydrogen;
R⁴ represents hydrogen; and
   wherein
(i) when R¹ is an optionally substituted C₁-C₆alkoxy group, C₆aryloxy group, 5- to 6-membered heteroaryloxy or -A-B group,
   R³ represents methyl, ethyl, propyl, i-propyl, cyclopropyl, cyclobutyl, —CONH₂ or
   CONHMe,
or (ii) when R¹ is an optionally substituted C₁-C₆alkyl group,
   R³ represents methyl, ethyl, propyl, i-propyl, cyclopropyl, cyclobutyl or —CONH₂.

In an embodiment of the invention, there is provided a subset of compounds of formula (I), and pharmaceutically acceptable salts thereof, in which:
R¹ represents a C₁-C₃alkyl group (such as methyl, ethyl, propyl and i-propyl) optionally substituted by one or more substituents selected from C₁-C₃alkoxy (such as methoxy, ethoxy, propoxy and i-propoxy), C₃-C₄cycloalkyl (such as cyclopropyl and cyclobutyl) [each of which may be optionally substituted by one or more substituents selected from halogen (such as fluorine, chlorine, bromine or iodine), C₁-C₃alkyl (such as methyl, ethyl, propyl and i-propyl), C₁-C₃alkoxy (such as methoxy, ethoxy, propoxy and i-propoxy)], and hydroxyl, a cyclopropyl group optionally substituted by C₁-C₃alkoxy (such as methoxy, ethoxy, propoxy and i-propoxy), a C₁-C₃alkoxy group (such as methoxy, ethoxy, propoxy and i-propoxy) optionally substituted by one or more substituents selected from C₁-C₃alkoxy (such as methoxy, ethoxy, propoxy and i-propoxy) and cyclopropyl, a phenyloxy group optionally substituted by one or more substituents selected from C₁-C₃alkyl (such as methyl, ethyl, propyl and i-propyl), C₁-C₃alkoxy(such as methoxy, ethoxy, propoxy and i-propoxy) and cyclopropyl, ore -A-B wherein A represents a C₂-alkylene, and B represents a phenyl ring optionally substituted by one or more substituents selected from C₁-C₃alkyl, C₁-C₃alkoxy or cyclopropyl;
R² represents hydrogen or methyl;
R³ represents methyl, ethyl, propyl, i-propyl, cyclopropyl or —CONH₂; and
R⁴ represents hydrogen, methyl or methoxy,
or a pharmaceutically acceptable salt thereof

In an embodiment of the invention, there is provided a subset of compounds of formula (I), and pharmaceutically acceptable salts thereof, in which:
R¹ represents a C₁-C₃alkyl group (such as methyl, ethyl, propyl and i-propyl) optionally substituted by one or more substituents selected from C₁-C₃alkoxy (such as methoxy, ethoxy, propoxy and i-propoxy), C₃-C₄cycloalkyl (such as cyclopropyl and cyclobutyl) [each of which may be optionally substituted by one or more substituents selected from halogen (such as fluorine, chlorine, bromine or iodine), C₁-C₃alkyl (such as methyl, ethyl, propyl and i-propyl), C₁-C₃alkoxy (such as methoxy, ethoxy, propoxy and i-propoxy)], and hydroxyl, a cyclopropyl group optionally substituted by C₁-C₃alkoxy (such as methoxy, ethoxy, propoxy and i-propoxy), a C₁-C₃alkoxy group (such as methoxy, ethoxy, propoxy and i-propoxy) optionally substituted by one or more substituents selected from C₁-C₃alkoxy (such as methoxy, ethoxy, propoxy and i-propoxy) and cyclopropyl, a phenyloxy group optionally substituted by one or more substituents selected from C₁-C₃alkyl (such as methyl, ethyl, propyl and i-propyl), C₁-C₃alkoxy(such as methoxy, ethoxy, propoxy and i-propoxy) and cyclopropyl, or -A-B wherein A represents a C₂-alkylene, and B represents a pyridin-4-yl ring optionally substituted by one or more substituents selected from C₁-C₃alkyl, C₁-C₃alkoxy or cyclopropyl;
R² represents hydrogen or methyl;
R³ represents methyl, ethyl, propyl, i-propyl, cyclopropyl or —CONH₂; and
R⁴ represents hydrogen, methyl or methoxy,
or a pharmaceutically acceptable salt thereof

In an embodiment of the invention, there is provided a subset of compounds of formula (I), and pharmaceutically acceptable salts thereof, in which:
R¹ represents a C₁-C₃alkyl group (such as methyl, ethyl, propyl and i-propyl) optionally substituted by one or more substituents selected from C₁-C₃alkoxy (such as methoxy, ethoxy, propoxy and i-propoxy), C₃-C₄cycloalkyl (such as cyclopropyl and cyclobutyl) [each of which may be optionally substituted by one or more substituents selected from halogen (such as fluorine, chlorine, bromine or iodine), C₁-C₃alkyl (such as methyl, ethyl, propyl and i-propyl), C₁-C₃alkoxy (such as methoxy, ethoxy, propoxy and i-propoxy)], and hydroxyl, a cyclopropyl group optionally substituted by C₁-C₃alkoxy (such as methoxy, ethoxy, propoxy and i-propoxy), a C₁-C₃alkoxy group (such as methoxy, ethoxy, propoxy and i-propoxy) optionally substituted by one or more substituents selected from C₁-C₃alkoxy (such as methoxy, ethoxy, propoxy and i-propoxy) and cyclopropyl, a phenyloxy group optionally substituted by one or more substituents selected from C₁-C₃alkyl (such as methyl, ethyl, propyl and i-propyl), C₁-C₃alkoxy(such as methoxy, ethoxy, propoxy and i-propoxy) and cyclopropyl, or -A-B wherein A represents an oxyC₁-alkylene, and B represents a phenyl ring or a pyridin-4-yl ring each optionally substituted by one or more substituents selected from C₁-C₃alkyl, C₁-C₃alkoxy or cyclopropyl;
R² represents hydrogen or methyl;
R³ represents methyl, ethyl, propyl, i-propyl, cyclopropyl or —CONH₂; and
R⁴ represents hydrogen, methyl or methoxy,
or a pharmaceutically acceptable salt thereof

In another embodiment of the invention, there is provided a subset of compounds of formula (I), and pharmaceutically acceptable salts thereof, in which:
R¹ represents a C₁-C₃alkyl group (such as methyl, ethyl, propyl and i-propyl) substituted by one or more substituents selected from C₁-C₃alkoxy (such as methoxy, ethoxy, propoxy and i-propoxy) [which may be optionally substituted by one or more substituents selected from halogen (such as fluorine, chlorine, bromine or iodine), C₁-C₃alkyl (such as methyl, ethyl, propyl and i-propyl), C₁-C₃alkoxy (such as methoxy, ethoxy, propoxy and i-propoxy)], and hydroxyl, a C₁-C₃alkoxy group (such as methoxy, ethoxy, propoxy and i-propoxy) optionally substituted by one or more substituents selected from C₁-C₃alkoxy (such as methoxy, ethoxy, propoxy and i-propoxy) and cyclopropyl, or -A-B wherein A represents a C₂-alkylene or oxyC₁-alkylene, and B represents a phenyl ring optionally substituted by one or more substituents selected from halogen, C₁-C₃alkyl, C₁-C₃alkoxy or CONR⁶³R⁶⁴;
R² represents hydrogen;
R⁴ represents hydrogen; and
   wherein
(i) when R¹ is an optionally substituted C₁-C₃alkoxy group, phenoxyoxy group, or -A-B group,
   R³ represents methyl, ethyl, propyl, i-propyl, cyclopropyl, cyclobutyl, —CONH₂ or —CONHMe,
or (ii) when R¹ is an optionally substituted C₁-C₃alkyl group,
R³ represents methyl, ethyl, propyl, i-propyl, cyclopropyl, cyclobutyl or —CONH₂.

In another embodiment of the invention, there is provided a subset of compounds of formula (I), and pharmaceutically acceptable salts thereof, in which:
R¹ represents a C₁-C₃alkyl group (such as methyl, ethyl, propyl and i-propyl) substituted by one or more substituents selected from C₁-C₃alkoxy (such as methoxy, ethoxy, propoxy and i-propoxy) [which may be optionally substituted by one or more substituents selected from halogen (such as fluorine, chlorine, bromine or iodine), C₁-C₃alkyl (such as methyl, ethyl, propyl and i-propyl), C₁-C₃alkoxy (such as methoxy, ethoxy, propoxy and i-propoxy)], and hydroxyl, a C₁-C₃alkoxy group (such as methoxy, ethoxy, propoxy and i-propoxy) optionally substituted by one or more substituents selected from C₁-C₃alkoxy (such as methoxy, ethoxy, propoxy and i-propoxy) and cyclopropyl, or -A-B wherein A represents a C₂-alkylene or oxyC₁-alkylene, and B represents a pyridine-4-yl ring optionally substituted by one or more substituents selected from halogen, C₁-C₃alkyl, C₁-C₃alkoxy or CONR⁶³R⁶⁴;
R² represents hydrogen;
R⁴ represents hydrogen; and
   wherein
(i) when R¹ is an optionally substituted C₁-C₃alkoxy group, phenoxyoxy group, or -A-B group,
   R³ represents methyl, ethyl, propyl, i-propyl, cyclopropyl, cyclobutyl, —CONH₂ or —CONHMe,
or (ii) when R¹ is an optionally substituted C₁-C₃alkyl group,
R³ represents methyl, ethyl, propyl, i-propyl, cyclopropyl, cyclobutyl or —CONH₂.

In a further aspect of the invention, there is provided a compound of formula (I) (as depicted above) wherein:
R¹ represents a methyl, ethyl, propyl, i-propyl, hydroxymethyl, cyclopropyl, methoxypropyl, ethoxypropyl, phenylethyl, p-methoxyphenylethyl, m-methoxyphenylethyl, or (3,5-dimethoxyphenyl)methoxy;
R² represents hydrogen or methyl;
R³ represents methyl, cyclopropyl or —CONH₂; and
R⁴ represents hydrogen, methyl or methoxy, or a pharmaceutically acceptable salt thereof

In a further aspect of the invention, there is provided a compound of formula (I) (as depicted above) wherein:
R¹ represents hydroxymethyl, methoxypropyl, ethoxypropyl, phenylethyl, 2-(3-methoxyphenyl)ethyl, 2-(3,5-dimethoxyphenyl)ethyl, i-propoxy, benzyloxy, (3,5-dimethoxyphenyl)methoxy, 2-(3-hydroxyphenyl)ethyl, 2-(3,5-dihydroxyphenyl)ethyl, (3-methoxyphenyl)methoxy, [3-(methylcarbamoyl)phenyl]methoxy, [3-methoxy-5-(methylcarbamoyl)phenyl]methoxy, 2-[3-(methylcarbamoyl)phenyl]ethyl, 2-[3-methoxy-5-(methylcarbamoyl)phenyl]ethyl, (3-hydroxyphenyl)methoxy, (3,5-dihydroxyphenyl)methoxy,(3-chloro-5-methoxy-phenyl)methoxy, or a 2-(3-chloro-5-methoxy-phenyl)ethyl group;
R² represents hydrogen;
R³ represents methyl, cyclopropyl, cyclobutyl or —CONH₂; and
R⁴ represents hydrogen,
or a pharmaceutically acceptable salt thereof

In a further aspect of the invention, there is provided a compound of formula (I) (as depicted above) wherein:
R¹ represents a hydroxymethyl, methoxypropyl, ethoxypropyl, phenylethyl, 2-(3-methoxyphenyl)ethyl, 2-(3,5-dimethoxyphenyl)ethyl, i-propoxy, benzyloxy, (3,5-dimethoxyphenyl)methoxy, 2-(3-hydroxyphenyl)ethyl, 2-(3,5-dihydroxyphenyl)ethyl, (3-methoxyphenyl)methoxy, [3-(methylcarbamoyl)phenyl]methoxy, [3-methoxy-5-(methylcarbamoyl)phenyl]methoxy, 2-[3-(methylcarbamoyl)phenyl]ethyl, 2-[3-methoxy-5-(methylcarbamoyl)phenyl]ethyl, (3-hydroxyphenyl)methoxy, (3,5-dihydroxyphenyl)methoxy, (3-chloro-5-methoxy-phenyl)methoxy, 2-(2,6-dimethoxypyridin-4-yl)ethyl, (5-fluoro-2-methoxy-pyridin-4-yl)methoxy, 2-(5-fluoro-2-methoxy-pyridin-4-yl)ethyl, (3-methoxy-5-methyl-phenyl)methoxy, (3-fluorophenyl)methoxy, (3-chlorophenyl)methoxy, 2-(3-aminophenyl)ethyl, 2-(5-methoxythiophen-2-yl)ethyl, 2-(2-furyl)ethyl, (2,6-dimethoxypyridin-4-yl)methoxy or a 2-(3-chloro-5-methoxy-phenyl)ethyl group;
R² represents hydrogen;
R³ represents methyl, cyclopropyl, cyclobutyl or —CONH₂; and
R⁴ represents hydrogen,
or a pharmaceutically acceptable salt thereof

In a further aspect of the invention, there is provided a compound of formula (I) (as depicted above) wherein:
R¹ represents a hydroxymethyl, methoxypropyl, ethoxypropyl, phenylethyl, 2-(3-methoxyphenyl)ethyl, 2-(3,5-dimethoxyphenyl)ethyl, i-propoxy, benzyloxy, (3,5-dimethoxyphenyl)methoxy, 2-(3-hydroxyphenyl)ethyl, 2-(3,5-dihydroxyphenyl)ethyl, (3-methoxyphenyl)methoxy, [3-(methylcarbamoyl)phenyl]methoxy, [3-methoxy-5-(methylcarbamoyl)phenyl]methoxy, 2-[3-(methylcarbamoyl)phenyl]ethyl, 2-[3-methoxy-5-(methylcarbamoyl)phenyl]ethyl, (3-hydroxyphenyl)methoxy, (3,5-dihydroxyphenyl)methoxy, (3-chloro-5-methoxy-phenyl)methoxy, 2-(2,6-dimethoxypyridin-4-yl)ethyl, (5-fluoro-2-methoxy-pyridin-4-yl)methoxy, 2-(5-fluoro-2-methoxy-pyridin-4-yl)ethyl, (3-methoxy-5-methyl-phenyl)methoxy, (3-fluorophenyl)methoxy, (3-chlorophenyl)methoxy, 2-(3-aminophenyl)ethyl, 2-(5-methoxythiophen-2-yl)ethyl, 2-(2-furyl)ethyl or a 2-(3-chloro-5-methoxy-phenyl)ethyl group;
R² represents hydrogen;
R³ represents methyl, cyclopropyl, cyclobutyl or —CONH₂; and
R⁴ represents hydrogen,
or a pharmaceutically acceptable salt thereof.

Examples of compounds of the invention include:
N-[(3-methylisoxazol-5-yl)methyl]-N′-(5-methyl-2H-pyrazol-3-yl)pyrimidine-2,4-diamine,
N-methyl-N-[(3-methylisoxazol-5-yl)methyl]-N′-(5-methyl-2H-pyrazol-3-yl)pyrimidine-2,4-diamine,
N-[(3-cyclopropylisoxazol-5-yl)methyl]-N′-(5-methyl-2H-pyrazol-3-yl)pyrimidine-2,4-diamine,
5-[[[4-[(5-methyl-2H-pyrazol-3-yl)amino]pyrimidin-2-yl]amino]methyl]isoxazole-3-carboxamide,
[5-[[2-[(3-methylisoxazol-5-yl)methylamino]pyrimidin-4-yl]amino]-1H-pyrazol-3-yl]methanol,
N-[(3-methylisoxazol-5-yl)methyl]-N′-(5-propyl-2H-pyrazol-3-yl)pyrimidine-2,4-diamine,
N-[(3-cyclopropylisoxazol-5-yl)methyl]-N′-(5-propyl-2H-pyrazol-3-yl)pyrimidine-2,4-diamine,
5-[[[4-[(5-propyl-1H-pyrazol-3-yl)amino]pyrimidin-2-yl]amino]methyl]isoxazole-3-carboxamide,
N′-(5-cyclopropyl-2H-pyrazol-3-yl)-N-[(3-methylisoxazol-5-yl)methyl]pyrimidine-2,4-diamine,
N-[(3-cyclopropylisoxazol-5-yl)methyl]-N′-(5-cyclopropyl-2H-pyrazol-3-yl)pyrimidine-2,4-diamine,
5-[[[4-[(5-cyclopropyl-2H-pyrazol-3-yl)amino]pyrimidin-2-yl]amino]methyl]isoxazole-3-carboxamide,
N′-[5-(3-methoxypropyl)-2H-pyrazol-3-yl]-N-[(3-methylisoxazol-5-yl)methyl]pyrimidine-2,4-diamine,
N-[(3-cyclopropylisoxazol-5-yl)methyl]-N′-[5-(3-methoxypropyl)-2H-pyrazol-3-yl]pyrimidine-2,4-diamine,
5-[[[4-[[5-(3-methoxypropyl)-2H-pyrazol-3-yl]amino]pyrimidin-2-yl]amino]methyl]isoxazole-3-carboxamide,
N′-[5-(3-ethoxypropyl)-2H-pyrazol-3-yl]-N-[(3-methylisoxazol-5-yl)methyl]pyrimidine-2,4-diamine,
N-[(3-cyclopropylisoxazol-5-yl)methyl]-N′-[5-(3-ethoxypropyl)-2H-pyrazol-3-yl]pyrimidine-2,4-diamine,
5-[[[4-[[5-(3-ethoxypropyl)-2H-pyrazol-3-yl]amino]pyrimidin-2-yl]amino]methyl]isoxazole-3-carboxamide,
N′-[5-[2-(4-methoxyphenyl)ethyl]-2H-pyrazol-3-yl]-N-[(3-methylisoxazol-5-yl)methyl]pyrimidine-2,4-diamine,
N-[(3-cyclopropylisoxazol-5-yl)methyl]-N′-[5-[2-(4-methoxyphenyl)ethyl]-2H-pyrazol-3-yl]pyrimidine-2,4-diamine,
5-[[[4-[[5-[2-(4-methoxyphenyl)ethyl]-2H-pyrazol-3-yl]amino]pyrimidin-2-yl]amino]methyl]isoxazole-3-carboxamide,
N′-[5-[2-(3-methoxyphenyl)ethyl]-2H-pyrazol-3-yl]-N-[(3-methylisoxazol-5-yl)methyl]pyrimidine-2,4-diamine,
N′-[5-[2-(3-methoxyphenyl)ethyl]-2H-pyrazol-3-yl]-N-[(3-methylisoxazol-5-yl)methyl]pyrimidine-2,4-diamine,
5-[[[4-[[5-[2-(3-methoxyphenyl)ethyl]-2H-pyrazol-3-yl]amino]pyrimidin-2-yl]amino]methyl]isoxazole-3-carboxamide,
N-[(3-methylisoxazol-5-yl)methyl]-N′-(5-phenethyl-1H-pyrazol-3-yl)pyrimidine-2,4-diamine,
N′-(5-isopropoxy-2H-pyrazol-3-yl)-N-[(3-methylisoxazol-5-yl)methyl]pyrimidine-2,4-diamine,
N-[(3-cyclopropylisoxazol-5-yl)methyl]-N′-(5-isopropoxy-2H-pyrazol-3-yl)pyrimidine-2,4-diamine,
N′-(5-isopropoxy-1H-pyrazol-3-yl)-6-methyl-N-[(3-methylisoxazol-5-yl)methyl]pyrimidine-2,4-diamine,
N-[(3-cyclopropylisoxazol-5-yl)methyl]-N′-(5-isopropoxy-2H-pyrazol-3-yl)-6-methyl-pyrimidine-2,4-diamine,
N′-(5-isopropoxy-2H-pyrazol-3-yl)-6-methoxy-N-[(3-methylisoxazol-5-yl)methyl]pyrimidine-2,4-diamine,
N-[(3-cyclopropylisoxazol-5-yl)methyl]-N′-(5-isopropoxy-2H-pyrazol-3-yl)-6-methoxy-pyrimidine-2,4-diamine,
N′-(5-benzyloxy-1H-pyrazol-3-yl)-N-[(3-methylisoxazol-5-yl)methyl]pyrimidine-2,4-diamine,
N′-[5-[(3,5-dimethoxyphenyl)methoxy]-1H-pyrazol-3-yl]-N-[(3-methylisoxazol-5-yl)methyl]pyrimidine-2,4-diamine,
5-[[[4-[[5-(hydroxymethyl)-1H-pyrazol-3-yl]amino]pyrimidin-2-yl]amino]methyl]-1,2-oxazole-3-carboxamide,
N-[(3-Cyclobutyl 1,2-oxazol-5-yl)methyl]-N′-[5-(3-methoxypropyl)-1H-pyrazol-3-yl]pyrimidine-2,4-diamine,
N′-[5-[2-(2-methoxyphenyl)ethyl]-1H-pyrazol-3-yl]-N-[(3-methyl 1,2-oxazol-5-yl)methyl]pyrimidine-2,4-diamine hydrochloride,
N′-[5-[2-(4-methoxyphenyl)ethyl]-2H-pyrazol-3-yl]-N-[(3-pyrimidin-2-yl-1,2-oxazol-5-yl)methyl]pyrimidine-2,4-diamine,
N′-[5-[2-(3-methoxyphenyl)ethyl]-2H-pyrazol-3-yl]-N-[(3-pyrimidin-2-yl1,2-oxazol-5-yl)methyl]pyrimidine-2,4-diamine,
N-[(3-methyl-1,2-oxazol-5-yl)methyl]-N′-[5-(phenoxymethyl)-2H-pyrazol-3-yl]pyrimidine-2,4-diamine,
N-[(3-cyclopropyl 1,2-oxazol-5-yl)methyl]-N′-[5-(phenoxymethyl)-2H-pyrazol-3-yl]pyrimidine-2,4-diamine,
5-[[[4-[[5-(phenoxymethyl)-2H-pyrazol-3-yl]amino]pyrimidin-2-yl]amino]methyl]1,2-oxazole-3-carboxamide,
N-[(3-methyl 1,2-oxazol-5-yl)methyl]-N′-[5-[2-(4-phenylmethoxyphenyl)ethyl]-2H-pyrazol-3-yl]pyrimidine-2,4-diamine,
N-[(3-methyl 1,2-oxazol-5-yl)methyl]-N′-[5-[2-(3-phenylmethoxyphenyl)ethyl]-2H-pyrazol-3yl]pyrimidine-2,4-diamine,
N N-[(3-methyl 1,2-oxazol-5-yl)methyl]-N′-[5-[2-(2-phenylmethoxyphenyl)ethyl]-1H-pyrazol-3-yl]pyrimidine-2,4-diamine hydrochloride,
N′-[5-[2-[3-(2-methoxyethoxy)phenyl]ethyl]-2H-pyrazol-3-yl]-N-[(3-methyl1,2-oxazol-5-yl)methyl]pyrimidine-2,4-diamine,
3-[2-[5-[[2-[(3-methyl 1,2-oxazol-5-yl)methylamino]pyrimidin-4-yl]amino]-1H-pyrazol-3-yl]ethyl]phenol,
N′-[5-[2-(3,5-dimethoxyphenyl)ethyl]-2H-pyrazol-3-yl]-N-[(3-methyl1,2-oxazol-5-yl)methyl]pyrimidine-2,4-diamine,
5-[2-[5-[[2-[(3-methyl 1,2-oxazol-5-yl)methylamino]pyrimidin-4-yl]amino]-1H-pyrazol-3-yl]ethyl]benzene-1,3-diol,
N′-[5-[(3,5-Dimethoxyphenoxy)methyl]-2H-pyrazol-3-yl]-N-[(3-methyl1,2-oxazol-5-yl)methyl]pyrimidine-2,4-diamine,
N′-[5-[2-(2,5-dimethoxyphenyl)ethyl]-2H-pyrazol-3-yl]-N-[(3-methyl1,2-oxazol-5-yl)methyl]pyrimidine-2,4-diamine,
N′-[5-[2-(3,4-dimethoxyphenyl)ethyl]-1H-pyrazol-3-yl]-N-[(3-methyl1,2-oxazol-5-yl)methyl]pyrimidine-2,4-diamine hydrochloride,
N′-[5-[2-(4-methoxy-2-methyl-phenyl)ethyl]-2H-pyrazol-3-yl]-N-[(3-methyl1,2-oxazol-5-yl)methyl]pyrimidine-2,4-diamine,
3-[2-[5-[[2-[(3-methyl-1,2-oxazol-5-yl)methylamino]pyrimidin-4-yl]amino]-2H-pyrazol-3-yl]ethyl]benzonitrile,
N′-[5-[2-(3-fluoro-5-methyl-phenyl)ethyl]-1H-pyrazol-3-yl]-N-[(3-methyl1,2-oxazol-5-yl)methyl]pyrimidine-2,4-diamine,
5-[[[4-[(5-phenethyl-2H-pyrazol-3-yl)amino]pyrimidin-2-yl]amino]methyl]-1,2-oxazole-3-carboxamide,
N-[(3-methyl1,2-oxazol-5-yl)methyl]-N′-[5-[2-[3-(trifluoromethoxy)phenyl]ethyl]-1H-pyrazol-3-yl]pyrimidine-2,4-diamine,
N-[(3-methyl1,2-oxazol-5-yl)methyl]-N′-[5-[2-(3-methylphenyl)ethyl]-1H-pyrazol-3-yl]pyrimidine-2,4-diamine hydrochloride,
N′-[5-[2-(3-bromophenyl)ethyl]-1H-pyrazol-3-yl]-N-[(3-methyl1,2-oxazol-5-yl)methyl]pyrimidine-2,4-diamine hydrochloride,
N′-[5-(2-benzo[1,3]dioxol-5-ylethyl)-2H-pyrazol-3-yl]-N-[(3-methyl1,2-oxazol-5-yl)methyl]pyrimidine-2,4-diamine,
N-[(3-methyl1,2-oxazol-5-yl)methyl]-N′-[5-[2-(3-morpholin-4-ylphenyl)ethyl]-1H-pyrazol-3-yl]pyrimidine-2,4-diamine,
N′-[5-[(3-ethylphenyl)methoxy]-2H-pyrazol-3-yl]-N-[(3-methyl1,2-oxazol-5-yl)methyl]pyrimidine-2,4-diamine,
N⁴-[5-(2-methoxy-1-methylethoxy)-1H-pyrazol-3-yl]-N²-[(3-methylisoxazol-5-yl)methyl]pyrimidine-2,4-diamine,
N²-[(3-cyclopropylisoxazol-5-yl)methyl]-N4-[5-(2-methoxy-1-methylethoxy)-1H-pyrazol-3-yl]pyrimidine-2,4-diamine,
Ethyl 5-[[[4-[(5-propan-2-yloxy-2H-pyrazol-3-yl)amino]pyrimidin-2-yl]amino]methyl]1,2- oxazole-3-carboxylate,
5-[[[4-[(5-propan-2-yloxy-2H-pyrazol-3-yl)amino]pyrimidin-2-yl]amino]methyl]-1,2-oxazole-3-carboxamide,
N-methyl-5-[[[4-[(5-propan-2-yloxy-2H-pyrazol-3-yl)amino]pyrimidin-2-yl]amino]methyl]1,2-oxazole-3-carboxamide,
N,N-dimethyl-5-[[[4-[(5-propan-2-yloxy-2H-pyrazol-3-yl)amino]pyrimidin-2-yl]amino]methyl]1,2-oxazole-3-carboxamide,
N′-(5-propan-2-yloxy-2H-pyrazol-3-yl)-N-[(3-pyrimidin-5-yl 1,2-oxazol-5-yl)methyl]pyrimidine-2,4-diamine,
N′-(5-propan-2-yloxy-2H-pyrazol-3-yl)-N-[(3-pyrimidin-2-yl-1,2-oxazol-5-yl)methyl]pyrimidine-2,4-diamine,
5N-[[3-(oxolan-3-yl)1,2-oxazol-5-yl]methyl]-N′-(5-propan-2-yloxy-2H-pyrazol-3-yl)pyrimidine-2,4-diamine,
N-[[3-(oxolan-2-yl)1,2-oxazol-5-yl]methyl]-N′-(5-propan-2-yloxy-2H-pyrazol-3-yl)pyrimidine-2,4-diamine,
N-[[3-(oxan-4-yl)1,2-oxazol-5-yl]methyl]-N′-(5-propan-2-yloxy-2H-pyrazol-3-yl)pyrimidine-2,4-diamine,
N′-(5-ethoxy-1H-pyrazol-3-yl)-N-[(3-methyl1,2-oxazol-5-yl)methyl]pyrimidine-2,4-diamine,
N-[(3-methyl1,2-oxazol-5-yl)methyl]-N′-[5-[(3-morpholin-4-ylphenyl)methoxy]-2H-pyrazol-3-yl]pyrimidine-2,4-diamine,
N-[(3-methyl1,2-oxazol-5-yl)methyl]-N′-[5-[(3-methylsulfonyloxyphenyl)methoxy]-2H-pyrazol-3-yl]pyrimidine-2,4-diamine,
tert-Butyl N-[3-[[5-[[2-[(3-methyl1,2-oxazol-5-yl)methylamino]pyrimidin-4-yl]amino]-1H-pyrazol-3-yl]oxymethyl]phenyl]carbamate,
[3-[[5-[[2-[(3-methyl1,2-oxazol-5-yl)methylamino]pyrimidin-4-yl]amino]-1H-pyrazol-3-yl]oxymethyl]phenyl]-morpholin-4-yl-methanone,
N-methyl-3-[[5-[[2-[(3-methyl1,2-oxazol-5-yl)methylamino]pyrimidin-4-yl]amino]-1H-pyrazol-3-yl]oxymethyl]benzamide,
3-[[5-[[2-[(3-methyl1,2-oxazol-5-yl)methylamino]pyrimidin-4-yl]amino]-2H-pyrazol-3-yl]oxymethyl]benzonitrile hydrochloride,
N′-[5-[(3-chlorophenyl)methoxy]-1H-pyrazol-3-yl]-N-[(3-methyl1,2-oxazol-5- yl)methyl]pyrimidine-2,4-diamine hydrochloride,
N′-[5-[(3-fluorophenyl)methoxy]-1H-pyrazol-3-yl]-N-[(3-methyl1,2-oxazol-5-yl)methyl]pyrimidine-2,4-diamine hydrochloride,
N-[(3-methyl1,2-oxazol-5-yl)methyl]-N′-[5-[[3-(trifluoromethyl)phenyl]methoxy]-1H-pyrazol-3-yl]pyrimidine-2,4-diamine hydrochloride,
N-[(3-methyl1,2-oxazol-5-yl)methyl]-N′-[5-[[4-(trifluoromethyl)phenyl]methoxy]-1H-pyrazol-3-yl]pyrimidine-2,4-diamine hydrochloride,
Methyl 3-[[5-[[2-[(3-methyl1,2-oxazol-5-yl)methylamino]pyrimidin-4-yl]amino]-1H-pyrazol- 3-yl]oxymethyl]benzoate hydrochloride,
3-[[5-[[2-[(3-methyl1,2-oxazol-5-yl)methylamino]pyrimidin-4-yl]amino]-1H-pyrazol-3-yl]oxymethyl]benzoic acid,
N′-[5-[(4-fluoro-3-methoxy-phenyl)methoxy]-1H-pyrazol-3-yl]-N-[(3-methyl1,2-oxazol-5-yl)methyl]pyrimidine-2,4-diamine hydrochloride,
N-[(3-methyl1,2-oxazol-5-yl)methyl]-N′-[5-(2-phenoxyethoxy)-2H-pyrazol-3-yl]pyrimidine-2,4-diamine,
N-[(3-methyl1,2-oxazol-5-yl)methyl]-N′-(5-thiophen-2-yl-1H-pyrazol-3-yl)pyrimidine-2,4-diamine,
N′-[5-(2-furyl)-1H-pyrazol-3-yl]-N-[(3-methyl1,2-oxazol-5-yl)methyl]pyrimidine-2,4-diamine,
N-[(3-methyl1,2-oxazol-5-yl)methyl]-N′-[5-[2-(3-phenyl-1,2,4-oxadiazol-5-yl)ethyl]-2H-pyrazol-3-yl]pyrimidine-2,4-diamine,
N′-[5-[2-(2-furyl)ethyl]-2H-pyrazol-3-yl]-N-[(3-methyl1,2-oxazol-5-yl)methyl]pyrimidine-2,4-diamine,
N′-[5-(3-furylmethoxy)-1H-pyrazol-3-yl]-N-[(3-methyl1,2-oxazol-5-yl)methyl]pyrimidine-2,4-diamine,
N-[(3-methyl1,2-oxazol-5-yl)methyl]-N′-[5-[2-(oxolan-3-yl)ethyl]-1H-pyrazol-3-yl]pyrimidine-2,4-diamine,
N′-[5-[2-(3-furyl)ethyl]-1H-pyrazol-3-yl]-N-[(3-methyl1,2-oxazol-5-yl)methyl]pyrimidine-2,4-diamine,
N-[(3-cyclopropyl 1,2-oxazol-5-yl)methyl]-N′-[5-[2-(2-furyl)ethyl]-2H-pyrazol-3-yl]pyrimidine-2,4-diamine,
5-[[[4-[[5-[2-(2-furyl)ethyl]-2H-pyrazol-3-yl]amino]pyrimidin-2-yl]amino]methyl]1,2-oxazole-3-carboxamide,
N′-[5-[2-(2-furyl)ethyl]-2H-pyrazol-3-yl]-N-[(3-pyrimidin-2-yl 1,2-oxazol-5-yl)methyl]pyrimidine-2,4-diamine,
N-[(3-methyl1,2-oxazol-5-yl)methyl]-N′-[5-(oxan-4-yl)-1H-pyrazol-3-yl]pyrimidine-2,4-diamine hydrochloride,
N-[(3-methyl1,2-oxazol-5-yl)methyl]-N′-[5-(2-pyridin-3-ylethyl)-2H-pyrazol-3-yl]pyrimidine-2,4-diamine,
N-[(3-methyl-1,2-oxazol-5-yl)methyl]-N′-[5-(2-pyridin-4-ylethyl)-2H-pyrazol-3-yl]pyrimidine-2,4-diamine, and
N-[(3-methyl1,2-oxazol-5-yl)methyl]-N′-[5-[2-(4-methylthiophen-2-yl)ethyl]-2H-pyrazol-3-yl]pyrimidine-2,4-diamine,
N′-[5-[2-(2,5-dimethylpyrazol-3-yl)ethyl]-1H-pyrazol-3-yl]-N-[(3-methyl-1,2-oxazol-5-yl)methyl]pyrimidine-2,4-diamine
N′-[5-[2-(1-methylimidazol-4-yl)ethyl]-1H-pyrazol-3-yl]-N-[(3-methyl-1,2-oxazol-5-yl)methyl]pyrimidine-2,4-diamine
N′-(5-cyclopentyl-1H-pyrazol-3-yl)-N-[(3-methyl-1,2-oxazol-5-yl)methyl]pyrimidine-2,4-diamine
N′-(5-cyclopentyl-2H-pyrazol-3-yl)-N-[(3-cyclopropyl-1,2-oxazol-5-yl)methyl]pyrimidine-2,4-diamine
N-[(3-cyclopropyl-1,2-oxazol-5-yl)methyl]-N′-[5-(2-furyl)-2H-pyrazol-3-yl]pyrimidine-2,4-diamine
3-[2-[5-[[2-[(3-cyclopropyl-1,2-oxazol-5-yl)methylamino]pyrimidin-4-yl]amino]-1H-pyrazol-3-yl]ethyl]phenol
N′-[5-[2-[5-(dimethylaminomethyl)-2-furyl]ethyl]-1H-pyrazol-3-yl]-N-[(3-methyl-1,2-oxazol-5-yl)methyl]pyrimidine-2,4-diamine
N-[(3-cyclobutyl-1,2-oxazol-5-yl)methyl]-N′-(5-propan-2-yloxy-1H-pyrazol-3-yl)pyrimidine-2,4-diamine
N′-(5-cyclopentyl-2H-pyrazol-3-yl)-N-[[3-(oxolan-2-yl)-1,2-oxazol-5-yl]methyl]pyrimidine-2,4-diamine
N-[(3-cyclopropyl-1,2-oxazol-5-yl)methyl]-N′-[5-(2-methylpropyl)-2H-pyrazol-3-yl]pyrimidine-2,4-diamine
N-[(3-cyclopropyl-1,2-oxazol-5-yl)methyl]-N′-(5-phenylmethoxy-2H-pyrazol-3-yl)pyrimidine-2,4-diamine
N′-[5-[2-(3-chloro-5-fluoro-phenyl)ethyl]-2H-pyrazol-3-yl]-N-[(3-methyl-1,2-oxazol-5-yl)methyl]pyrimidine-2,4-diamine
N′-[5-[2-[3-(aminomethyl)phenyl]ethyl]-1H-pyrazol-3-yl]-N-[(3-methyl-1,2-oxazol-5-yl)methyl]pyrimidine-2,4-diamine
N,N-dimethyl-3-[2-[5-[[2-[(3-methyl-1,2-oxazol-5-yl)methylamino]pyrimidin-4-yl]amino]-2H-pyrazol-3-yl]ethyl]benzamide
N′-[5-[2-(2,6-dimethoxypyrimidin-4-yl)ethyl]-1H-pyrazol-3-yl]-N-[(3-methyl-1,2-oxazol-5-yl)methyl]pyrimidine-2,4-diamine
[5-[[[4-[[5-[2-(3-methoxyphenyl)ethyl]-2H-pyrazol-3-yl]amino]pyrimidin-2-yl]amino]methyl]-1,2-oxazol-3-yl]methanol
N′-[5-[2-(5-fluoro-2-methoxy-pyridin-4-yl)ethyl]-1H-pyrazol-3-yl]-N-[(3-methyl-1,2-oxazol-5-yl)methyl]pyrimidine-2,4-diamine
3-[2-[5-[[2-[[3-(dimethylaminomethyl)-1,2-oxazol-5-yl]methylamino]pyrimidin-4yl]amino]-1H-pyrazol-3-yl]ethyl]phenol
5-[[[4-[[5-[2-(3-methoxyphenyl)ethyl]-2H-pyrazol-3-yl]amino]pyrimidin-2-yl]amino]methyl]-N-methyl-1,2-oxazole-3-carboxamide
5-[[[4-[[5-[2-(3-hydroxyphenyl)ethyl]-2H-pyrazol-3-yl]amino]pyrimidin-2-yl]amino]methyl]-N-methyl-1,2-oxazole-3-carboxamide
N-[(3-methyl-1,2-oxazol-5-yl)methyl]-N′-[5-[2-(3-propan-2-yloxyphenyl)ethyl]-1H-pyrazol-3-yl]pyrimidine-2,4-diamine
5-[[[4-[[5-[2-[3-(cyclopropylmethoxy)phenyl]ethyl]-1H-pyrazol-3-yl]amino]pyrimidin-2-yl]amino]methyl]-1,2-oxazole-3-carboxamide
N′-[5-[2-(2,6-dimethoxypyridin-4-yl)ethyl]-1H-pyrazol-3-yl]-N-[(3-methyl-1,2-oxazol-5-yl)methyl]pyrimidine-2,4-diamine
N′-[5-[2-(3-aminophenyl)ethyl]-1H-pyrazol-3-yl]-N-[(3-methyl-1,2-oxazol-5-yl)methyl]pyrimidine-2,4-diamine
5-[[[4-[[5-[2-(3-chloro-5-methoxy-phenyl)ethyl]-2H-pyrazol-3-yl]amino]pyrimidin-2-yl]amino]methyl]-1,2-oxazole-3-carboxamide
N-[[3-(dimethylaminomethyl)-1,2-oxazol-5-yl]methyl]-N′-[5-[2-(5-methoxypyridin-3-yl)ethyl]-1H-pyrazol-3-yl]pyrimidine-2,4-diamine
3-[2-[5-[[2-[[3-(dimethylaminomethyl)-1,2-oxazol-5-yl]methylamino]pyrimidin-4-yl]amino]-1H-pyrazol-3-yl]ethyl]phenol
3-Methoxy-N-methyl-5-[2-[5-[[2-[(3-methyl-1,2-oxazol-5-yl)methylamino]pyrimidin-4-yl]amino]-1H-pyrazol-3-yl]ethyl]benzamide
N-[(3-methyl-1,2-oxazol-5-yl)methyl]-N′-[5-[2-(3-pyrimidin-2-yloxyphenyl)ethyl]-1H-pyrazol-3-yl]pyrimidine-2,4-diamine hydrochloride
6-[2-[5-[[2-[(3-methyl-1,2-oxazol-5-yl)methylamino]pyrimidin-4-yl]amino]-2H-pyrazol-3-yl]ethyl]-1H-pyridin-2-one dihydrochloride
N-[[3-(dimethylaminomethyl)-1,2-oxazol-5-yl]methyl]-N′-[5-[2-(5-fluoro-2-methoxy-pyridin-4-yl)ethyl]-1H-pyrazol-3-yl]pyrimidine-2,4-diamine
N′-[5-[2-(5-methoxypyridin-3-yl)ethyl]-1H-pyrazol-3-yl]-N-[(3-methyl-1,2-oxazol-5-yl)methyl]pyrimidine-2,4-diamine
N-[3-methoxy-5-[2-[5-[[2-[(3-methyl-1,2-oxazol-5-yl)methylamino]pyrimidin-4-yl]amino]-2H-pyrazol-3-yl]ethyl]phenyl]acetamide
5-[[[4-[[5-[2-(3-propan-2-yloxyphenyl)ethyl]-1H-pyrazol-3-yl]amino]pyrimidin-2-yl]amino]methyl]-1,2-oxazole-3-carboxamide

N-methyl-3-[2-[5-[[2-[(3-methyl-1,2-oxazol-5-yl)methylamino]pyrimidin-4-yl]amino]-1H-pyrazol-3-yl]ethyl]benzamide
N,3-dimethyl-5-[2-[5-[[2-[(3-methyl-1,2-oxazol-5-yl)methylamino]pyrimidin-4-yl]amino]-1H-pyrazol-3-yl]ethyl]benzamide
4-Methoxy-N-methyl-6-[2-[5-[[2-[(3-methyl-1,2-oxazol-5-yl)methylamino]pyrimidin-4-yl]amino]-1H-pyrazol-3-yl]ethyl]pyridine-2-carboxamide
N′-[5-[(3-methoxy-5-methyl-phenyl)methoxy]-1H-pyrazol-3-yl]-N-[(3-methyl-1,2-oxazol-5-yl)methyl]pyrimidine-2,4-diamine
N′-[5-[(5-fluoro-2-methoxy-pyridin-4-yl)methoxy]-1H-pyrazol-3-yl]-N-[(3-methyl-1,2-oxazol-5-yl)methyl]pyrimidine-2,4-diamine
N′-[5-[(4-methoxypyridin-2-yl)methoxy]-1H-pyrazol-3-yl]-N-[(3-methyl-1,2-oxazol-5-yl)methyl]pyrimidine-2,4-diamine
N′-[5-[2-(5-methoxythiophen-2-yl)ethyl]-1H-pyrazol-3-yl]-N-[(3-methyl-1,2-yl)methyl]pyrimidine-2,4-diamine
N′-[5-[2-(2-methoxy-1,3-thiazol-5-yl)ethyl]-1H-pyrazol-3-yl]-N-[(3-methyl-1,2-oxazol-5-yl)methyl]pyrimidine-2,4-diamine
N-[(3-propan-2-yl-1,2-oxazol-5-yl)methyl]-N′-(5-propan-2-yloxy-2H-pyrazol-3-yl)pyrimidine-2,4-diamine
N-[[3-(3-methyloxetan-3-yl)-1,2-oxazol-5-yl]methyl]-N′-(5-propan-2-yloxy-2H-pyrazol-3-yl)pyrimidine-2,4-diamine
N-[[3-(1-methylcyclopropyl)-1,2-oxazol-5-yl]methyl]-N′-(5-propan-2-yloxy-2H-pyrazol-3-yl)pyrimidine-2,4-diamine
N′-(5-methoxy-2H-pyrazol-3-yl)-N-[(3-methyl-1,2-oxazol-5-yl)methyl]pyrimidine-2,4-diamine
or pharmaceutically acceptable salts of any one thereof.

In another aspect of the invention, particular compounds of the invention are any one of the Examples or pharmaceutically acceptable salts of any one thereof.

In a further aspect of the invention, there is provided a compound selected from any one of the Examples.

In a further aspect of the invention, particular compounds of the invention are any one of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 or 121, or pharmaceutically acceptable salts of any one thereof.

In a further aspect of the invention, there is provide a compound selected from any one of Examples 3, 6, 7, 9, 10, 13, 14, 15, 16, 21, 28, 29, 41, 42, 43, 44, 56, 57, 66, 67, 68, 69, 71, 73, 84, 91, 93, 94, 97, 102, 103, 111, 124, 126, 128, 129, 131, 132, 135, 141, 27, 52, 53, 54, 61, 62, 70, 72, 107, 120, 1, 2, 4, 8, 12, 17, 18, 19, 1 20, 23, 24, 25, 26, 31, 32, 33, 34, 35, 37, 38, 39, 40, 45, 46, 47, 48, 49, 50, 51, 55, 63, 64, 65, 74, 76, 77, 78, 79, 80, 81, 82, 83, 85, 86, 88, 89, 90, 92, 95, 96, 98, 100, 104, 105, 106, 108, 109, 110, 112, 113, 114, 115, 116, 117, 121, 122, 123, 125, 130, 133, 136, 137, 138, 139, 140, 142, 143 5, 22, 36, 58, 59, 60, 75, 87, 99, 101, 118, 119, 127 and 134.

In a further aspect of the invention, there is provide a compound selected from any one of Examples 3, 6, 7, 9, 10, 13, 14, 15, 16, 21, 28, 29, 41, 42, 43, 44, 56, 57, 66, 67, 68, 69, 71, 73, 84, 91, 93, 94, 97, 102, 103, 111, 124, 126, 128, 129, 131, 132, 135, 141, 27, 30, 52, 53, 54, 61, 62, 70, 72, 107, 120, 1, 2, 4, 8, 12, 17, 18, 19, 1 20, 23, 24, 25, 26, 31, 32, 33, 34, 35, 37, 38, 39, 40, 45, 46, 47, 48, 49, 50, 51, 55, 63, 64, 65, 74, 76, 77, 78, 79, 80, 81, 82, 83, 85, 86, 88, 89, 90, 92, 95, 96, 98, 100, 104, 105, 106, 108, 109, 110, 112, 113, 114, 115, 116, 117, 121, 122, 123, 125, 130, 133, 136, 137, 138, 139, 140, 142 and 143.

In a further aspect of the invention, there is provide a compound selected from any one of Examples 3, 6, 7, 9, 10, 13, 14, 15, 16, 21, 28, 29, 41, 42, 43, 44, 56, 57, 66, 67, 68, 69, 71, 73, 84, 91, 93, 94, 97, 102, 103, 111, 124, 126, 128, 129, 131, 132, 135, 141, 27, 30, 52, 53, 54, 61, 62, 70, 72, 107, and 120.

In a further aspect of the invention, there is provide a compound selected from any one of Examples 3, 6, 7, 9, 10, 13, 14, 15, 16, 21, 28, 29, 41, 42, 43, 44, 56, 57, 66, 67, 68, 69, 71, 73, 84, 91, 93, 94, 97, 102, 103, 111, 124, 126, 128, 129, 131, 132, 135 and 141.

In a further aspect of the invention, there is provide a compound selected from any one of Examples 66, 67, 68, 69, 71, 84, 102, 70, 76, 77, 78, 79, 80, 81, 82, 83, 85, 86, and 75.

In a further aspect of the invention, there is provide a compound selected from any one of Examples 66, 67, 68, 69, 71, 84, 102, 70, 76, 77, 78, 79, 80, 81, 82, 83, 85 and 86.

In a further aspect of the invention, there is provide a compound selected from any one of Examples 66, 67, 68, 69, 71, 84, 102 and 70.

In a further aspect of the invention, there is provide a compound selected from any one of Examples 66, 67, 68, 69, 71, 84 and 102.

In a further aspect of the invention, there is provide a compound selected from any one of Examples 28, 29, 41, 42, 43, 44, 56, 57, 111, 124, 126, 128, 129, 132, 141, 73, 91, 93, 94, 97, 103, 131, 135, 27, 30, 52, 53, 54, 61, 62, 107, 135, 72, 24, 25, 26, 31, 32, 33, 34, 35, 37, 38, 39, 40, 45, 46, 47, 48, 49, 50, 51, 55, 63, 64, 65, 106, 109, 110, 112, 113, 115, 116, 117, 121, 122, 123, 125, 130, 133, 136, 138, 139, 140, 142, 143, 74, 88, 89, 90, 92, 95, 96, 98, 100, 108, 137, 58, 59, 60, 118, 119, 127, 134, 36, 87, 99 and 101.

In a further aspect of the invention, there is provide a compound selected from any one of Examples 28, 29, 41, 42, 43, 44, 56, 57, 111, 124, 126, 128, 129, 132, 141, 73, 91, 93, 94, 97, 103, 131, 135, 27, 30, 52, 53, 54, 61, 62, 107, 135, 72, 73, 91, 93, 94, 97, 103, 131, 135, 24, 25, 26, 31, 32, 33, 34, 35, 37, 38, 39, 40, 45, 46, 47, 48, 49, 50, 51, 55, 63, 64, 65, 106, 109, 110, 112, 113, 115, 116, 117, 121, 122, 123, 125, 130, 133, 136, 138, 139, 140, 142, 143, 74, 88, 89, 90, 92, 95, 96, 98, 100, 108 and 137.

In a further aspect of the invention, there is provide a compound selected from any one of Examples 28, 29, 41, 42, 43, 44, 56, 57, 111, 124, 126, 128, 129, 132, 141, 73, 91, 93, 94, 97, 103, 131, 135, 27, 30, 52, 53, 54, 61, 62, 107, 111, 124, 126, 128, 129, 132, 135 and 72.

In a further aspect of the invention, there is provide a compound selected from any one of Examples 28, 29, 41, 42, 43, 44, 56, 57, 111, 124, 126, 128, 129, 132, 141, 73, 91, 93, 94, 97, 103, 131 and 135.

The present invention further provides a process for the preparation of a compound of formula (I) as defined hereinbefore above, or a pharmaceutically acceptable salt thereof, which comprises:
i) reacting a compound of formula (IV)

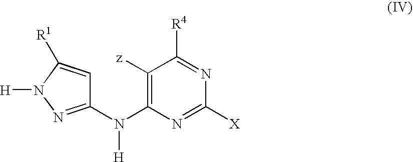

wherein X represents a leaving group (e.g. halogen or sulfanyl such as methanesulfanyl or sulphonyloxy such as methanesulphonyloxy or toluene-4-sulphonyloxy), Z represents hydrogen or a halogen, and R¹ and R⁴ are as hereinbefore defined for a compound formula (I)
with a compound of formula (V)

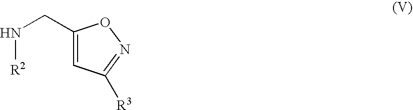

wherein R² and R³ are as defined hereinbefore for a compound of formula (I) to give,
when Z is hydrogen, a compound of formula (I) or,
when Z is halogen, a compound of formula (VI)

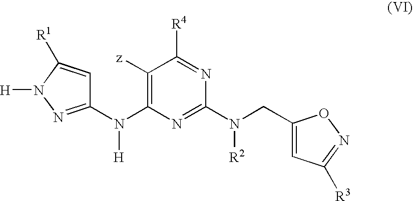

and (ii) when Z is a halogen, optionally reacting a compound of formula (VI) with a de-halogenating reagent to give a compound of formula (I);
and optionally after (i) or (ii) carrying out one or more of the following:
   converting the compound obtained to a further compound of the invention
   forming a pharmaceutically acceptable salt of the compound.

Step (i) may conveniently be carried out in a suitable solvent such as 2-methoxyethanol, 1-methylpyrrolidinone, butanol or dimethylacetamide at a temperature in the range from 90-200° C., optionally with microwave irradiation. The reaction can be carried out in the presence or absence of a suitable acid or base for example an inorganic acid such as hydrochloric acid or sulphuric acid, or an organic acid such as acetic acid or formic acid (or a suitable Lewis acid) or an inorganic base such as sodium carbonate, or an organic base such as N,N-diisopropylethylamine.

Optional dehalogenation may conveniently be carried out in a suitable solvent such as ethanol in the presence of a suitable catalyst such as 5-20% palladium on carbon under an atmosphere of hydrogen.

Compounds of formula (IV) may be prepared by reacting a compound of formula (II)

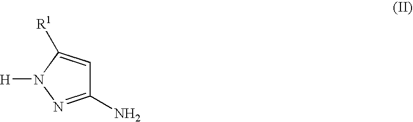

wherein R¹ is as defined hereinbefore for a compound of formula (I), with a compound of formula (III),

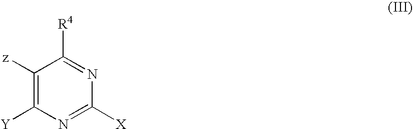

wherein X and Y each independently represents a leaving group (e.g. halogen or sulfanyl such as methanesulfanyl or sulphonyloxy such as methanesulphonyloxy or toluene-4-sulphonyloxy), Z represents hydrogen or a halogen, and R⁴ is as defined hereinbefore for a compound of formula (I)
to give a compound of formula (IV)

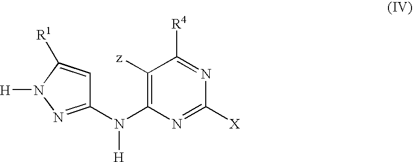

This reaction may conveniently be carried out in the presence of a suitable solvent such as ethanol, butanol, toluene or 1-methylpyrrolid-2-one, optionally in the presence of a suitable acid or base for example an inorganic acid such as hydrochloric acid or sulphuric acid, or an organic acid such as acetic acid or formic acid (or a suitable Lewis acid) or an inorganic base such as sodium carbonate, or an organic base such as N,N-diisopropylethylamine and at a temperature in the range from 0° C. to reflux.

In a further aspect of the present invention there is provide a process for the preparation of a compound of formula (I) as defined hereinbefore above, or a pharmaceutically acceptable salt thereof, which comprises: reacting a compound of formula (IX),

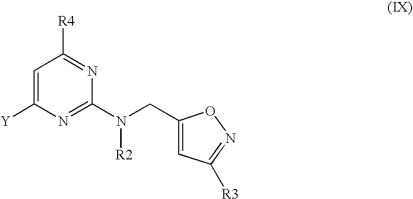

wherein Y is a leaving group such as chloro, and R², R³ and R⁴ are as defined hereinbefore for a compound of formula (I),
with a compound of formula (II)

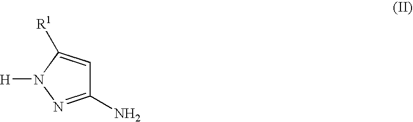

wherein R¹ is as defined hereinbefore for a compound of formula (I) and optionally carrying out one or more of the following:
converting the compound obtained to a further compound of the invention
forming a pharmaceutically acceptable salt of the compound.

The process may conveniently be carried out in a suitable solvent such as 1-methylpyrrolidinone or dimethylacetamide in the presence of a suitable acid such as hydrogen chloride in dioxane at a temperature in the range from 90 to 120° C.

Compounds of Formula (IX) may be prepared by
(a) reacting a compound of formula (VII)

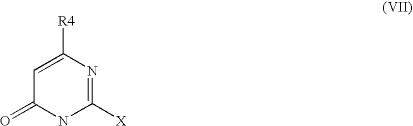

wherein R⁴ is as defined hereinbefore for a compound of formula (I) and X represents a leaving group (e.g. halogen or sulfanyl such as methanesulfanyl or sulphonyloxy such as methanesulphonyloxy or toluene-4-sulphonyloxy),
with a compound of formula (V)

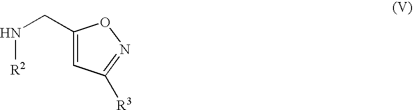

wherein R² and R³ are as defined hereinbefore for a compound of formula (I) to give a compound of formula (VIII)

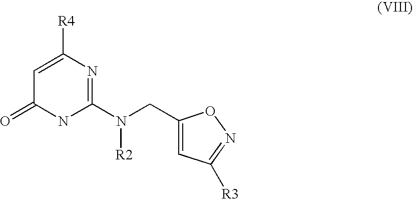

and,
(b) by reacting a compound of formula (VIII) with a chlorinating agent to a compound of formula (IX)

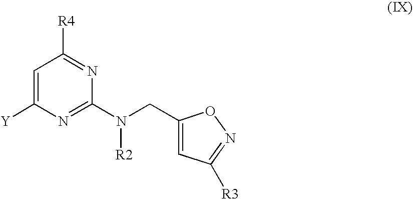

wherein Y is a leaving group such as chloro.

Step (a) may conveniently be carried out in a suitable solvent such as diglyme in the presence of a suitable base such as N,N-diisopropylethylamine at a temperature in the range from 120 to 180° C.

Step (b) may conveniently be carried out in a suitable solvent such as toluene with a suitable chlorinating agent such as phosphorus oxychloride in the presence of a suitable base such as N,N-diisopropylethylamine at a temperature in the range from 60 to 100° C.

In a still further aspect of the present invention there is provided a process for the preparation of a compound of formula (I) as hereinbefore defined but wherein R⁴ represent a C₁-C₆alkoxy group optionally substituted with C₁-C₃alkoxy, hydroxyl, amino (—NH₂), mono-C₁-C₃alkylamino and di-(C₁-C₃alky)amino, —NR⁵⁴R⁵⁵, or —S(O)_{y}R⁵⁶, or a pharmaceutically acceptable salt thereof, which comprises: reacting a compound of formula (XII)

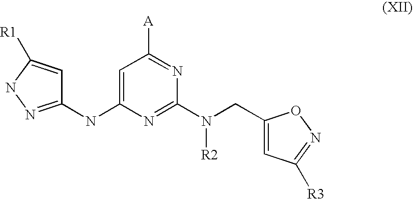

with a compound of formula (XIII)

H—R⁴ (XIII)
wherein R⁴ represents a C₁-C₆alkoxy group optionally substituted with C₁-C₃alkoxy, hydroxyl, amino (—NH₂), mono-C₁-C₃alkylamino and di-(C₁-C₃alky) amino, —NR⁵⁴R⁵⁵, or —S(O)_{y}R⁵⁶ wherein y=0,
and when R⁴ is —S(O)_{y}R⁵⁶ wherein y=0, optionally reacting with an oxidising agent, and optionally carrying out one or more of the following:
   converting the compound obtained to a further compound of the invention
   forming a pharmaceutically acceptable salt of the compound.

The reaction may conveniently be carried out in a suitable solvent such as 1-methylpyrrolidinone, dimethylacetamide or a compound of formula (XIII) used as solvent in the presence of a suitable base such as N,N-diisopropylethylamine or sodium hydride at a temperature in the range from 80 to 200° C., optionally with microwave irradiation.

The compound of formula (XII) may be obtained by:
(1) reacting a compound of formula (X)

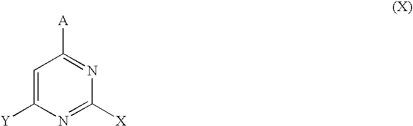

wherein X, Y and A each independently represents a leaving group (such as halogen or sulfanyl such as methanesulfanyl or sulphonyloxy such as methanesulphonyloxy or toluene-4-sulphonyloxy), with a compound of formula (II),

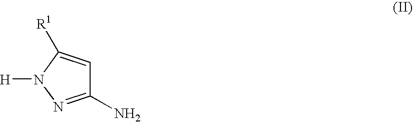

wherein R¹ is as defined hereinbefore for a compound of formula (I) to give a compound of formula (XI)

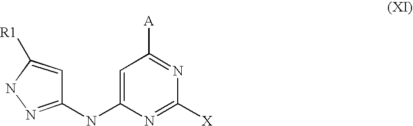

and,
(2) reacting a compound of formula (XI) with a compound of formula (V)

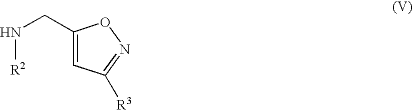

wherein R² and R³ are as defined hereinbefore for a compound of formula (I) to give a compound of formula (XII)

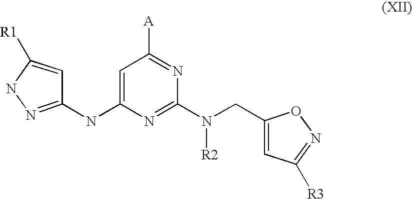

Step (1) may conveniently be carried out in a suitable solvent such as ethanol in the presence of a suitable base such as sodium carbonate or N,N-diisopropylethylamine at a temperature in the range from 0 to 25° C.

Step (2) may conveniently be carried out in a suitable solvent such as butanol, hexanol, 1-methylpyrrolidinone or dimethylacetamide in the presence of a suitable base such as N,N-diisopropylethylamine at a temperature in the range from 80 to 120° C.

Compounds of formulae (II), (III), (V), (VII), (X) and (XIII) are either commercially 1o available, are known in the literature or may be prepared using known techniques.

In a still further aspect of the present invention there is provided a process for the preparation of a compound of formula (I) as hereinbefore defined but wherein R³ represent a C₁-C₆alkyl group optionally substituted with mono-C₁-C₃alkylamino and di-(C₁-C₃alky)amino, —NR⁵⁴R⁵⁵, or a pharmaceutically acceptable salt thereof, which comprises: is reacting a compound of formula (XIV)

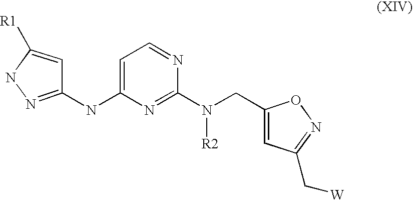

wherein W represents a leaving group (or can be converted into a leaving group)(such as halogen or sulfanyl such as methanesulfanyl or sulphonyloxy such as methanesulphonyloxy), with a compound selected from a mono-C₁-C₃alkylamine, di-(C₁-C₃alky)amine and a compound of formula (XV)

H—NR⁵⁴R⁵⁵ (XV)
and optionally carrying out one or more of the following:
   converting the compound obtained to a further compound of the invention
   forming a pharmaceutically acceptable salt of the compound.
The reaction may conveniently be carried out in a suitable solvent such as dichloromethane or tetrahydrofuran at room temperature.
The compound of formula (XIV) may be obtained by any of the procedures outlined previously for synthesis of compounds of the formula (I).

Compounds of formulae (XV) are either commercially available, are known in the literature or may be prepared using known techniques.

Compounds of formula (I) can be converted into further compounds of formula (I) using standard procedures. Examples of the types of conversion reactions that may be used include introduction of a substituent by means of an aromatic substitution reaction, reduction of substituents, alkylation of substituents, de-alkylation of substituents and oxidation of substituents. The reagents and reaction conditions for such procedures are well known in the chemical art. Particular examples of aromatic substitution reactions include the introduction of a nitro group using concentrated nitric acid; the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of reduction reactions include the reduction of a nitro group to an amino group by catalytic hydrogenation with a nickel catalyst or by treatment with iron in the presence of hydrochloric acid with heating or the reduction of a cyano group to an amino group by treatment with lithium aluminium hydride; particular examples of de-alkylation reactions include the conversion of a methoxy group to a hydroxyl by treatment with boron tribromide; and particular examples of oxidation reactions include oxidation of alkylthio to alkylsulphinyl or alkylsulphonyl.

It will be appreciated by those skilled in the art that in the processes of the present invention certain functional groups such as hydroxyl or amino groups in the starting reagents or intermediate compounds may need to be protected by protecting groups. Thus, the preparation of the compounds of formula (I) may involve, at various stages, the addition and removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 2nd edition, T. W. Greene and P. G. M. Wuts, Wiley-Interscience (1991).

The compounds of formula (I) above may be converted to a pharmaceutically acceptable salt thereof, preferably an acid addition salt such as a hydrochloride, hydrobromide, phosphate, acetate, fumarate, maleate, tartrate, citrate, oxalate, methanesulphonate or p-toluenesulphonate, or an alkali metal salt such as a sodium or potassium salt.

Certain compounds of formula (I) are capable of existing in stereoisomeric forms. It will be understood that the invention encompasses the use of all geometric and optical isomers (including atropisomers) of the compounds of formula (I) and mixtures thereof including racemates.

Certain compounds of formula (I) are capable of existing in tatomeric forms. For example, 5-[[[4-[[5-(hydroxymethyl)-1H-pyrazol-3-yl]amino]pyrimidin-2-yl]amino]methyl]-1,2-oxazole-3-carboxamide

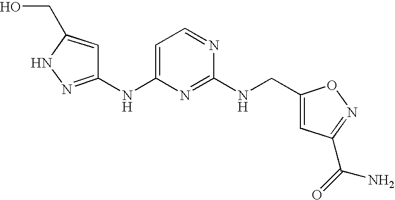

may also exist as the corresponding tautomer 5-[[[4-[[5-(hydroxymethyl)-2H-pyrazol-3-yl]amino]pyrimidin-2-yl]amino]methyl]-1,2-oxazole-3-carboxamide

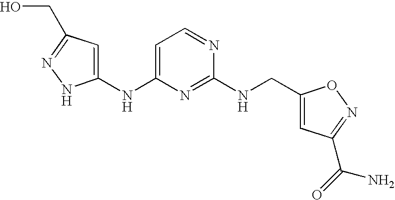

It is understood that compounds referred to by name, unless otherwise stated, include all tautomers of the compound.

The use of tautomers and mixtures thereof also form an aspect of the present invention.

The compounds of formula (I) have activity as pharmaceuticals, in particular as modulators or inhibitors of FGFR activity, and may be used in the treatment of proliferative and hyperproliferative diseases/conditions, examples of which include the following cancers:
(1) carcinoma, including that of the bladder, brain, breast, colon, kidney, liver, lung, ovary, pancreas, prostate, stomach, cervix, colon, thyroid and skin;
(2) hematopoietic tumors of lymphoid lineage, including acute lymphocytic leukaemia, B-cell lymphoma and Burketts lymphoma;
(3) hematopoietic tumours of myeloid lineage, including acute and chronic myelogenous leukaemias and promyelocytic leukaemia;
(4) tumours of mesenchymal origin, including fibrosarcoma and rhabdomyosarcoma; and
(5) other tumours, including melanoma, seminoma, tetratocarcinoma, neuroblastoma and glioma.

The compounds of the invention are especially useful in the treatment of tumors of the breast and prostate.

Thus, the present invention provides a compound of formula (I), or a pharmaceutically-acceptable salt thereof, as hereinbefore defined for use in therapy.

In a further aspect, the present invention provides the use of a compound of formula (I), or a pharmaceutically acceptable salt thereof, as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

The invention also provides a method of treating cancer which comprises administering to a patient in need thereof a therapeutically effective amount of a compound of formula (I), or a pharmaceutically acceptable salt thereof, as hereinbefore defined.

The invention still further provides a method of modulating FGFR activity which comprises administering to a patient in need thereof a therapeutically effective amount of a compound of formula (I), or a pharmaceutically acceptable salt thereof, as hereinbefore defined.

We have found that the compounds defined in the present invention, or a pharmaceutically acceptable salt thereof, are effective anti-cancer agents which property is believed to arise from their FGFR inhibitory properties. Accordingly the compounds of the present invention are expected to be useful in the treatment of diseases or medical conditions mediated alone or in part by FGFR, i.e. the compounds may be used to produce a FGFR inhibitory effect in a warm-blooded animal in need of such treatment.

Thus the compounds of the present invention provide a method for treating cancer characterised by inhibition of FGFR, i.e. the compounds may be used to produce an anti-cancer effect mediated alone or in part by the inhibition of FGFR.

Such a compound of the invention is expected to possess a wide range of anti-cancer properties as activating mutations in FGFR have been observed in many human cancers, including but not limited to, melanoma, papillary thyroid tumours, cholangiocarcinomas, colon, ovarian and lung cancers. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, bladder, prostate, breast and pancreas. In particular such compounds of the invention are expected to slow advantageously the growth of primary and recurrent solid tumours of, for example, the breast and prostate. More particularly such compounds of the invention, or a pharmaceutically acceptable salt thereof, are expected to inhibit the growth of those primary and recurrent solid tumours which are associated with FGFR, especially those tumours which are significantly dependent on FGFR for their growth and spread, including for example, certain tumours of the breast and prostate.

Thus according to this aspect of the invention there is provided a compound of the formula (I), or a pharmaceutically acceptable salt thereof, as defined hereinbefore for use as a medicament.

According to a further aspect of the invention there is provided the use of a compound of the formula (I), or a pharmaceutically acceptable salt thereof, as defined hereinbefore in the manufacture of a medicament for use in the production of a FGFR inhibitory effect in a warm-blooded animal such as man.

According to this aspect of the invention there is provided the use of a compound of the formula (I), or a pharmaceutically acceptable salt thereof, as defined hereinbefore in the manufacture of a medicament for use in the production of an anti-cancer effect in a warm-blooded animal such as man.

According to a further feature of the invention, there is provided the use of a compound of the formula (I), or a pharmaceutically acceptable salt thereof, as defined herein before in the manufacture of a medicament for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further aspect of the invention there is provided the use of a compound of the formula (I), or a pharmaceutically acceptable salt thereof, as defined hereinbefore in the production of a FGFR inhibitory effect in a warm-blooded animal such as man.

According to this aspect of the invention there is provided the use of a compound of the formula (I), or a pharmaceutically acceptable salt thereof, as defined hereinbefore in the production of an anti-cancer effect in a warm-blooded animal such as man.

According to a further feature of the invention, there is provided the use of a compound of the formula (I), or a pharmaceutically acceptable salt thereof, as defined herein before in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a FGFR inhibitory effect in a warm-blooded animal, such as man, in need of such treatment which comprises administering to said animal an effective amount of a compound of formula (I), or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined above.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof as defined herein before.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a FGFR inhibitory effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.

The compounds of formula (I) and pharmaceutically acceptable salts thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (per cent by weight), more preferably from 0.05 to 80% w, still more preferably from 0. 10 to 70% w, and even more preferably from 0. 10 to 50% w, of active ingredient, all percentages by weight being based on total composition.

The present invention also provides a pharmaceutical composition comprising a compound of formula (I), or a pharmaceutically acceptable salt thereof, as hereinbefore defined, in association with a pharmaceutically acceptable adjuvant, diluent or carrier.

The invention further provides a process for the preparation of a pharmaceutical composition of the invention which comprises mixing a compound of formula (I), or a pharmaceutically acceptable salt thereof, as hereinbefore defined, with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane aerosols and dry powder formulations; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or 10 condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The size of the dose for therapeutic purposes of a compound of the invention will naturally vary according to the nature and severity of the conditions, the age and sex of the animal or patient and the route of administration, according to well known principles of medicine.

In general, a compound of the invention will be administered so that a daily dose in the range, for example, from 0.5 mg to 75 mg active ingredient per kg body weight is received, given if required in divided doses. In general lower doses will be administered when a parenteral route is employed. Thus, for example, for intravenous administration, a dose in the range, for example, from 0.5 mg to 30 mg active ingredient per kg body weight will generally be used. Similarly, for administration by inhalation, a dose in the range, for example, from 0.5 mg to 25 mg active ingredient per kg body weight will generally be used. Oral administration is however preferred. For example, a formulation intended for oral administration to humans will generally contain, for example, from 0.5 mg to 2 g of active ingredient.

For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The anti cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:—
(i) other antiproliferative/antineoplastic drugs and combinations thereof, as used in medical oncology, such as alkylating agents (for example cis platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of
5*-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZDO530; International Patent Application WO 01/94341) and N-(2-chloro-6-methylphenyl)-2- {6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);
(iv) inhibitors of growth factor function: for example such inhibitors include growth factor antibodies and growth factor receptor antibodies (for example the anti erbB2 antibody trastuzumab [Herceptin™], the anti-EGFR antibody panitumumab, the anti erbB1 antibody cetuximab [Erbitux, C225]and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD 1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD 1152, PH739358, VX-680, MLN8054, R^{763,} MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin avb3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and
(ix) immunotherapy approaches, including for example ex vivo and in vivo approaches to increase the immunogenicity of patient tumour cells, such as transfection with cytokines such as interleukin 2, interleukin 4 or granulocyte macrophage colony stimulating factor, approaches to decrease T cell anergy, approaches using transfected immune cells such as cytokine transfected dendritic cells, approaches using cytokine transfected tumour cell lines and approaches using anti idiotypic antibodies.

### EXAMPLES

The invention will now be further described with reference to the following illustrative examples in which, unless stated otherwise:
(i) temperatures are given in degrees Celsius (° C.); operations were carried out at room or ambient temperature, that is, at a temperature in the range of 18-25° C.;
(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required; (vii) when given, NMR data is in the form of delta values for major diagnostic protons, given in parts per million (ppm) relative to tetramethylsilane (TMS) as an internal standard, determined at 300 MHz, in DMSO-d₆ unless otherwise indicated; Alternatively, NMR data may also be in the form of delta values for major diagnostic protons, given in parts per million (ppm) relative to tetramethylsilane (TMS) as an internal standard, determined at 300 MHz, in DMSO-d₆+CD₃COOD unless otherwise indicated;
(viii) chemical symbols have their usual meanings; SI units and symbols are used;
(ix) solvent ratios are given in volume:volume (v/v) terms; and
(x) mass spectra (MS) data was generated on an LC/MS system where the HPLC component comprised generally either a Agilent 1100 or Waters Alliance HT (2790 & 2795) equipment and was run on a Phemonenex Gemini C18 5 μm, 50×2 mm column (or similar) eluting with either acidic eluent (for example, using a gradient between 0-95% water/acetonitrile with 5% of a 1% formic acid in 50:50 water:acetonitrile (v/v) mixture; or using an equivalent solvent system with methanol instead of acetonitrile), or basic eluent (for example, using a gradient between 0-95% water/acetonitrile with 5% of a 0. 1% 880 Ammonia in acetonitrile mixture); and the MS component comprised generally a Waters ZQ spectrometer. 10 Chromatograms for Electrospray (ESI) positive and negative Base Peak Intensity, and UV Total Absorption Chromatogram from 220-300 nm, are generated and values for m/z are given; generally, only ions which indicate the parent mass are reported and unless otherwise stated the value quoted is the (M+H)+ for positive ion mode and (M−H)⁻ for negative ion mode;
Alternatively, mass spectra may be run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;(xi) Preparative HPLC was performed on C18 reversed-phase silica, for example on a Waters ‘Xterra’ preparative reversed-phase column (5 microns silica, 19 mm diameter, 100 mm length) using decreasingly polar mixtures as eluent, for example decreasingly polar mixtures of water (containing 1% acetic acid or 1% aqueous ammonium hydroxide (d=0.88) and acetonitrile;
(xii) the following abbreviations have been used:
   THF tetrahydrofuran;
   DMF N,N-dimethylformamide;
   EtOAc ethyl acetate;
   DMS dimethylsulphide;
   DIPEA N,N-diisopropylethylamine (also known as N-ethyl-N-propan-2-yl-propan-2-amine)
   DCM dichloromethane; and
   DMSO dimethylsulphoxide.
   PBS phosphate buffered saline
   HEPES N-[2-Hydroxyethyl]piperazine-N′-[2-ethanesulfonic acid]DTT dithiothreitol
   ATP Adenosine Triphosphate
   BSA bovine serum albumin
   DMEM Dulbecco's modified Eagle's Medium
   OptiMEM is a reduced serum free media used to grow mammalian cells, commercially available from Invitrogen
(xii) compounds are named using C-lab naming software: Openeye Lexichem version 1.4; using IUPAC naming convention;
(xiii) unless otherwise specified, starting materials are commercially available.

TABLE 1

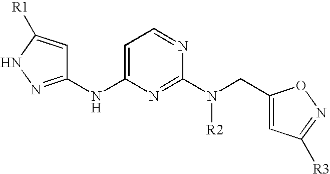

| Example | R1 | R2 | R3 |
|---|---|---|---|
| 1 | Me | H | Me |
| 2 | Me | Me | Me |
| 3 | Me | H | 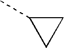 |
| 4 | Me | H | 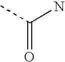 |
| 5 | 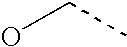 | H | Me |
| 6 | 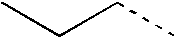 | H | Me |
| 7 | 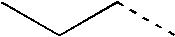 | H | 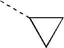 |
| 8 | 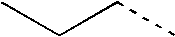 | H | 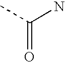 |
| 9 | 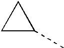 | H | Me |
| 10 | 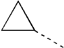 | H | 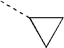 |
| 11 | 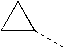 | H | 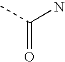 |
| 12 | 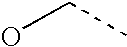 | H | 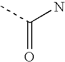 |
| 13 | 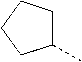 | H | Me |
| 14 | 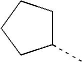 | H | 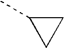 |
| 15 | 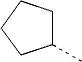 | H | 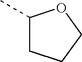 |
| 16 | 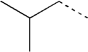 | H | 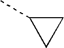 |

### Example 1

### N-[(3-methylisoxazol-5-yl)methyl]-N′-(5-methyl-2H-pyrazol-3-yl)pyrimidine-2,4-diamine (also known as N-[(3-methyl-1,2-oxazol-5-yl)methyl]-N′-(5-methyl-2H-pyrazol-3-yl)pyrimidine-2,4-diamine)

A mixture of 2-chloro-N-(5-methyl-1H-pyrazol-3-yl)pyrimidin-4-amine (0.209 g, 1.0 mmol), (3-methylisoxazol-5-yl)methanamine hydrochloride (also known as (3-methyl-1,2-oxazol-5-yl)methanamine hydrochloride; 0.446 g, 3.0 mmol) and N,N-diisopropylethylamine (0.693 ml, 4.0 mmol) in n-butanol (10 ml) was heated at 115° C. for 18 hours. The mixture was evaporated under vacuum and the residue was then partitioned between water (20 ml) and diethyl ether (20 ml). The mixture was filtered and the residue washed with water and then allowed to dry to leave compound 1 in table 1 (0.264 g, 93% yield).

¹H NMR (300 MHz, DMSO): 2.17 (s, 3H), 2.18 (s, 3H), 4.53 (d, 2H), 6.11 (s, 1H), 6.14-6.42 (m, 2H), 7.19 (s, 1H), 7.83 (d, 1H), 9.32 (s, 1H), 11.84 (s, 1H).

MS: m/z 286 (MH⁺).

2-chloro-N-(5-methyl-1H-pyrazol-3-yl)pyrimidin-4-amine and (3-methyl-1,2-oxazol-5-yl)methanamine hydrochloride, used as starting materials, can be prepared by the method described in the literature (Barlaam, Bernard; Pape, Andrew; Thomas, Andrew. Preparation of pyrimidine derivatives as modulators of insulin-like growth factor-1 receptor (IGF-1). WO2003048133).

### Example 2

### N-methyl-N-[(3-methylisoxazol-5-yl)methyl]-N′-(5-methyl-2H-pyrazol-3-yl)pyrimidine-2,4-diamine (also known as N-methyl-N-[(3-methyl-1,2-oxazol-5-yl)methyl]-N′-(5-methyl-2H-pyrazol-3-yl)pyrimidine-2,4-diamine)

Prepared using an analogous method to example 1 but starting with N-[(3-methylisoxazol-5-yl)methyl]methanamine hydrochloride (also known as N-methyl-1-(3-methyl-1,2-oxazol-5-yl)methanamine hydrochloride; 0.489 g, 3.0 mmol) to give example 2 in table 1 (0.127 g, 42% yield).

¹H NMR (300 MHz, DMSO): 2.18 (s, 3H), 2.19 (s, 3H), 3.13 (s, 3H), 4.89 (s, 2H), 6.01-6.23 (m, 2H), 6.33 (s, 1H), 7.90 (d, 1H), 9.39 (s, 1H), 11.86 (s, 1H).

MS: m/z 300 (MH⁺).

### Example 3

### N-[(3-cyclopropylisoxazol-5-yl)methyl]-N ′-(5-methyl-2H-pyrazol-3-yl)pyrimidine-2,4-diamine (also known as N-[(3-cyclopropyl-1,2-oxazol-5-yl)methyl]-N′-(5-methyl-2H-pyrazol-3-yl)pyrimidine-2,4-diamine)

A mixture of 2-chloro-N-(5-methyl-1H-pyrazol-3-yl)pyrimidin-4-amine (0. 105 g, 0.5 mmol), (3-cyclopropylisoxazol-5-yl)methanamine hydrochloride (also known as (3-cyclopropyl-1,2-oxazol-5-yl)methanamine hydrochloride; 0.11 4 g, 0.65 mmol) and N,N-diisopropylethylamine (0.218 ml, 1.25 mmol) in 2-methoxyethanol (4 ml) was heated at 200° C. in a Emrys Optimiser microwave for 2 hours. The mixture was concentrated and the residue purified by preparative hplc eluting with a gradient of acetonitrile in water (containing 1% ammonia). The fractions containing product were combined and evaporated to leave compound 3 in table 1 (0.028 g, 18% yield).

¹H NMR (300 MHz, DMSO): 0.61-0.75 (m, 2H), 0.89-1.01 (m, 2H), 1.87-2.01 (m, 1H), 2.18 (s, 3H), 4.50 (s, 2H), 6.01 (s, 1H), 6.07-6.37 (m, 2H), 7.13 (s, 1H), 7.82 (s, 1H), 9.31 (s, 1H), 11.84 (s, 1H).

MS: m/z 312 (MH⁺).

(3-cyclopropylisoxazol-5-yl)methanamine hydrochloride (also known as (3-cyclopropyl-1,2-oxazol-5-yl)methanamine hydrochloride), used as starting material, can be prepared by the method described in the literature (Nowak, Thorsten; Thomas, Andrew Peter. Preparation of 4-(pyrazol-3-ylamino)pyrimidines for use in the treatment of cancer. WO2005040159).

### Example 4

### 5-[[[4-[(5-methyl-2H-pyrazol-3-yl)amino]pyrimidin-2-yl]amino]methyl]isoxazole-3-carboxamide (also known as 5-[[[4-[(5-methyl-2H-pyrazol-3-yl)amino]pyrimidin-2-yl]amino]methyl]-1,2-oxazole-3-carboxamide)

Prepared in an analogous way to example 3 but using 5-(aminomethyl)isoxazole-3-carboxamide (also known as 5-(aminomethyl)-1,2-oxazole-3-carboxamide; 0. 124 g, 0.88 mmol) to give compound 4 in table 1 (0.048 g, 31% yield).

¹H NMR(300 MHz, DMSO): 2.18 (s, 3H), 4.61 (d, 2H), 6.19 (s, 1H), 6.31 (s, 1H), 6.52 (s, 1H), 7.26 (s, 1H), 7.73 (s, 1H), 7.83 (d, 1H), 8.03 (s, 1H), 9.34 (s, 1H), 11.84 (s, 1H).

MS: m/z 315 (MH⁺).

5-(aminomethyl)isoxazole-3-carboxamide (also known as 5-(aminomethyl)-1,2-oxazole-3-carboxamide), used as starting material, can be prepared by the method described in the literature (Baucke, Dorit; Lange, Udo; Mack, Helmut; Seitz, Werner; Zierke, Thomas; Hoffken, Hans Wolfgang; Homberger, Wilfried. Preparation of amidino-substituted peptides as thrombin inhibitors. WO9806741).

### Example 5

### [5-[[2-[(3-methylisoxazol-5-yl)methylamino]pyrimidin-4-yl]amino]-1H-pyrazol-3-yl]methanol (also known as [5-[[2-[(3-methyl-1,2-oxazol-5-yl)methylamino]pyrimidin-4-yl]amino]-1H-pyrazol-3-yl]methanol)

Prepared in an analogous way to example 3 but starting with [5-[(2-chloropyrimidin-4-yl)amino]-2H-pyrazol-3-yl]methanol (0.095 g, 0.42 mmol) and (3-methylisoxazol-5-yl)methanamine hydrochloride (also known as (3-methyl-1,2-oxazol-5-yl)methanamine hydrochloride; 0.088 g, 0.59 mmol) to give compound 5 in table 1 (0.044 g, 35% yield).

¹H NMR (300 MHz, DMSO): 2.17 (s, 3H), 4.42 (s, 2H), 4.53 (s, 2H), 5.19 (s, 1H), 6.12 (s, 1H), 6.26-6.43 (m, 2H), 7.17 (s, 1H), 7.83 (d, 1H), 9.35 (s, 1H), 12.04 (s, 1H).

MS: m/z 302 (MH⁺).

[5-[(2-chloropyrimidin-4-yl)amino]-2H-pyrazol-3-yl]methanol, used as starting material, was prepared as follows:
a) A mixture of (5-amino-2H-pyrazol-3-yl)methanol (2.5 1 g, 22.2 mmol) and 2,4-dichloropyrimidine (3.0 g, 20.1 mmol) and di-iso-propylethylamine (4.21 ml, 24.2 mmol) in ethanol (60 ml) was stirred at 40° C. for 4 days. The resultant precipitate was filtered, washed with ethanol and then with diethyl ether and then dried under vacuum to leave [5-[(2-chloropyrimidin-4-yl)amino]-2H-pyrazol-3-yl]methanol (3.1 g, 68% yield).

¹H NMR (300 MHz, DMSO): 4.46 (d, 2H), 5.28 (d, 1H), 6.25 (s, 1H), 7.15 (s, 1H), 8.16 (s, 1H), 10.32 (s, 1H), 12.32 (s, 1H).

MS: m/z 226 (MH⁺).

(5-amino-2H-pyrazol-3-yl)methanol, used as starting material, was prepared as follows:
i) A solution of 5-nitro-1H-pyrazole-3-carboxylic acid (15.0 g, 95.5 mmol) in tetrahydrofuran (150 ml) was cooled to 0° C. (ice bath). Dimethylformamide (I drop) and then oxalyl chloride (10.83 ml, 124 mmol) were added dropwise and the resulting solution was allowed to warm to room temperature and then stirred under argon for 2 hours. The mixture was evaporated and the residue was dissolved in tetrahydrofuran (200 ml) and then added dropwise to a solution of 2M lithium borohydride (in tetrahydrofuran, 71.6 ml, 143 mmol) cooled to -15° C., under argon (internal temperature kept between −15° C. and −10° C., during addition). The mixture was allowed to warm to room temperature over 2 hours and then left to stir at room temperature overnight. The mixture was added dropwise to a mixture of ice/water (200 ml ice/200 ml water) and then extracted into ethyl acetate (2×). The organic fractions were combined and washed with brine, dried over magnesium sulfate and then evaporated to leave (5-nitro-1H-pyrazol-3-yl)methanol (10.26 g, 75% yield).

¹H NMR (500 MHz, CDCl₃): 4.52 (s, 2H), 6.85 (s, 1H), 13.87 (s, 1H).
ii) Ammonium formate (0.551 g, 8.74 mmol) was added, in one portion, to a solution of (5-nitro-1H-pyrazol-3-yl)methanol (0.50 g, 3.49 mmol) in ethanol (14 ml). The mixture was blanketed with argon and 10% palladium on carbon (50 mg) was added. The vial was then sealed and heated in a microwave to 140° C. for 10 minutes. The mixture was filtered and the residue was washed with a 1:1 mixture of ethyl acetate:ethanol (20 ml). The filtrate was evaporated and the residue purified by chromatography on silica eluting with a 0-30% mixture of methanol in ethyl acetate to give (5-amino-2H-pyrazol-3-yl)methanol (0.225 g, 57% yield).

¹H NMR (400 MHz, DMSO): 4.27 (d, 2H), 4.53 (s, 2H), 4.95 (t, 1H), 5.29 (s, 1H), 11.20 (s, 1H).

(3-methyl-1,2-oxazol-5-yl)methanamine hydrochloride, used as starting material, was prepared as outlined in Example 1.

### Example 6

### N-[(3-methylisoxazol-5-yl)methyl]-N ′-(5-propyl-2H-pyrazol-3-yl)pyrimidine-2,4-diamine (also known as N-[(3-methyl-1,2-oxazol-5-yl)methyl]-N ′-(5-propyl-2H-pyrazol-3-yl)pyrimidine-2,4-diamine)

Prepared in an analogous way to example 3 but starting with 2-chloro-N-(5-propyl-1H-pyrazol-3-yl)pyrimidin-4-amine (0.10 g, 0.42 mmol) and (3-methylisoxazol-5-yl)methanamine hydrochloride (also known as (3-methyl-1,2-oxazol-5-yl)methanamine hydrochloride; 0.088 g, 0.59 mmol) to give compound 6 in table 1 (0.068 g, 52% yield).

¹H NMR (300 MHz, DMSO): 0.90 (t, 3H), 1.53-1.65 (m, 2H), 2.17 (s, 3H), 4.53 (d, 2H), 6.11 (s, 1H), 6.14-6.46 (m, 2H), 7.19 (s, 1H), 7.82 (d, 1H), 9.34 (s, 1H), 11.85 (s, 1H); (2 Protons under DMSO).

MS: m/z 314 (MH⁺).

2-chloro-N-(5-propyl-1H-pyrazol-3-yl)pyrimidin-4-amine, used as starting material, was prepared as follows:
a) A mixture of 5-propyl-1H-pyrazol-3-amine (1.6 g, 12.78 mmol), 2,4-dichloropyrimidine (1.71 g, 11.5 mmol) and N,N-diisopropylethylamine (2.45 ml, 14.1 mmol) in ethanol (40 ml) was heated at 40° C. for 3 days. The mixture was poured into water and the resulting precipitate was filtered and washed with water and then with ice-cold diethyl ether. The residue was dried under vacuum to leave 2-chloro-N-(5-propyl-1H-pyrazol-3-yl)pyrimidin-4-amine (2.12 g, 78% yield).

¹H NMR (300 MHz, DMSO): 0.91 (t, 3H), 1.54-1.67 (m, 2H), 2.55 (t, 2H), 6.08 (s, 1H), 7.20 (s, 1H), 8.15 (d, 1H), 10.27 (s, 1H), 12.14 (s, 1H).

MS: m/z 238 (MH⁺).

5-propyl-1H-pyrazol-3-amine, used as starting material, can be prepared by the method described in the literature (Barlaam, Bernard; Pape, Andrew; Thomas, Andrew. Preparation of pyrimidine derivatives as modulators of insulin-like growth factor-1 receptor (IGF-1). WO2003048133).

(3-methyl-1,2-oxazol-5-yl)methanamine hydrochloride, used as starting material, was prepared as outlined in Example 1.

### Example 7

### N-[(3-cyclopropylisoxazol-5-yl)methyl]-N ′-(5-propyl-2H-pyrazol-3-yl)pyrimidine-2,4-diamine (also known as N-[(3-cyclopropyl-1,2-oxazol-5-yl)methyl]-N′-(5-propyl-2H-pyrazol-3-yl)pyrimidine-2,4-diamine)

Prepared in an analogous way to example 6 but starting with (3-cyclopropylisoxazol-5-yl)methanamine hydrochloride (also known as (3-cyclopropyl-1,2-oxazol-5-yl)methanamine hydrochloride; 0. 1 14 g, 0.65 mmol) to give compound 7 in table 1 (0.058 g, 34% yield).

¹H NMR (300 MHz, DMSO): 0.63-0.75 (m, 2H), 0.82-1.01 (m, 5H), 1.50-1.67 (m, 2H), 1.86-2.01 (m, 1H), 4.51 (s, 2H), 5.99 (s, 1H), 6.05-6.41 (m, 2H), 7.15 (s, 1H), 7.82 (s, 1H), 9.33 (s, 1H), 11.85 (s, 1H); 2 Protons under DMSO.

MS: m/z 340 (MH⁺).

(3-cyclopropyl-1,2-oxazol-5-yl)methanamine hydrochloride, used as starting material, was prepared as in Example 3.

### Example 8

### 5-[[[4-[(5-propyl-1H-pyrazol-3-yl)amino]pyrimidin-2-yl]amino]methyl]isoxazole-3-carboxamide (also known as 5-[[[4-[(5-propyl-1H-pyrazol-3-yl)amino]pyrimidin-2-yl]amino]methyl]-1,2-oxazole-3-carboxamide)

Prepared in an analogous way to example 6 but starting with 5-(aminomethyl)isoxazole-3-carboxamide (also known as 5-(aminomethyl)-1,2-oxazole-3-carboxamide) to give compound 8 in table 1 (0.040 g, 23% yield).

¹H NMR (300 MHz, DMSO): 0.90 (t, 3H), 1.55-1.62 (m, 2H), 4.62 (d, 2H), 6.23 (s, 1H), 11.86 (s, 1H); 2 protons under DMSO.

MS: m/z 343 (MH⁺).

5-(aminomethyl)-1,2-oxazole-3-carboxamide, used as starting material, can be prepared as described in Example 4.

### Example 9

### N′-(5-cyclopropyl-2H-pyrazol-3-yl)-N-[(3-methylisoxazol-5-yl)methyl]pyrimidine-2,4-diamine (also known as N′-(5-cyclopropyl-2H-pyrazol-3-yl)-N-[(3-methyl-1,2-oxazol-5-yl)methyl]pyrimidine-2,4-diamine)

Prepared in an analogous way to example 3 but starting with 2-chloro-N-(5-cyclopropyl-1H-pyrazol-3-yl)pyrimidin-4-amine (0. 11 8 g, 0.5 mmol) and (3-methylisoxazol-5-yl)methanamine hydrochloride (also known as (3-methyl-1,2-oxazol-5-yl)methanamine hydrochloride; 0.097 g, 0.65 mmol) to give example 9 in table 1 (0.020 g, 10% yield).

¹H NMR (300 MHz, DMSO): 0.60-0.71 (m, 2H), 0.80-0.95 (m, 2H), 1.77-1.88 (m, 1H), 2.18 (s, 3H), 4.52 (s, 2H), 6.02-6.20 (m, 2H), 6.26 (s, 1H), 7.20 (s, 1H), 7.81 (s, 1H), 9.33 (s, 1H), 11.90 (s, 1H).

MS: m/z 312 (MH⁺).

2-chloro-N-(5-cyclopropyl-1H-pyrazol-3-yl)pyrimidin-4-amine, used as starting material, can be prepared by the method described in the literature (Nowak, Thorsten; Thomas, Andrew Peter. Preparation of 4-(pyrazol-3-ylamino)pyrimidines for use in the treatment of cancer. WO2005040159).

(3-methyl-1,2-oxazol-5-yl)methanamine hydrochloride, used as starting material, was prepared as outlined in Example 1.

### Example 10

### N-[(3-cyclopropylisoxazol-5-yl)methyl]-N ′-(5-cyclopropyl-2H-pyrazol-3-yl)pyrimidine-2,4-diamine (also known as N-[(3-cyclopropyl-1,2-oxazol-5-yl)methyl]-N′-(5-cyclopropyl-2H-pyrazol-3-yl)pyrimidine-2,4-diamine)

Prepared in an analogous way to example 9 but starting with (3-cyclopropylisoxazol-5-yl)methanamine hydrochloride (also known as (3-cyclopropyl-1,2-oxazol-5-yl)methanamine hydrochloride; 0.097 g, 0.55 mmol). After the reaction was complete the mixture was concentrated and the residue triturated with water. The resultant precipitate was filtered and the residue washed first with water and then with diethyl ether and then allowed to dry under vacuum to give example 10 in table 1 (0.086 g, 52% yield).

¹H NMR (300 MHz, DMSO): 0.65-0.72 (m, 4H), 0.89-0.99 (m, 4H), 1.79-1.88 (m, 1H), 1.90-1.99 (m, 1H), 4.54 (d, 2H), 6.02 (s, 1H), 6.13 (s, 1H), 6.28 (s, 1H), 6.72 (s, 1H), 7.82 (d, 1H), 9.64 (s, 1H), 11.99 (s, 1H).

MS: m/z 338 (MH⁺).

(3-cyclopropyl-1,2-oxazol-5-yl)methanamine hydrochloride, used as starting material, was prepared as in Example 3.

### Example 11

### 5-[[[4-[(5-cyclopropyl-2H-pyrazol-3-yl)amino]pyrimidin-2-yl]amino]methyl]isoxazole-3-carboxamide (also known as 5-[[[4-[(5-cyclopropyl-2H-pyrazol-3-yl)amino]pyrimidin-2-yl]amino]methyl]-1,2-oxazole-3-carboxamide)

Prepared in an analogous way to example 9 but starting with 5-(aminomethyl)isoxazole-3-carboxamide (also known as 5-(aminomethyl)-1,2-oxazole-3-carboxamide; 0.124 g, 0.88 mmol) to give example 11 in table 1 (0.014 g, 8% yield).

¹H NMR (300 MHz, DMSO): 0.63-0.68 (m, 2H), 0.84-0.94 (m, 2H), 1.79-1.88 (m, 1H), 4.62 (d, 2H), 6.13 (s, 1H), 6.27 (s, 1H), 6.51 (s, 1H), 7.28 (s, 1H), 7.74 (s, 1H), 7.83 (d, 1H), 8.03 (s, 1H), 9.36 (s, 1H), 11.91 (s, 1H).

MS: m/z 341 (MH⁺).

5-(aminomethyl)-1,2-oxazole-3-carboxamide, used as starting material, can be prepared as described in Example 4.

### Example 12

### 5-[[[4-[[5-(hydroxymethyl)-1H-pyrazol-3-yl]amino]pyrimidin-2-yl]amino]methyl]-1,2-oxazole-3-carboxamide

Prepared in an analogous way to Example 3, from [5-[(2-chloropyrimidin-4-yl)amino]-2H-pyrazol-3-yl]methanol (113mg, 0.50 mmol) and 5-(aminomethyl)isoxazole-3-carboxamide (99mg, 0.70 mmol) to give the title compound as a solid (6.5 mg, 4% yield).

MS: m/z 331 (MH).

[5-[(2-chloropyrimidin-4-yl)amino]-2H-pyrazol-3-yl]methanol used as a starting material was prepared as described in Example 5.

5-(aminomethyl)isoxazole-3-carboxamide, used as starting material, can be prepared by the method described in Example 4.

### Example 13

### N′-(5-cyclopentyl-2H-pyrazol-3-yl)-N-[(3-methyl-1,2-oxazol-5-yl)methyl]pyrimidine-2,4-diamine

4-chloro-N-[(3-methyl-1,2-oxazol-5-yl)methyl]pyrimidin-2-amine (200 mg, 0.890 mmol) was dissolved in ethanol (5 ml) and 5-cyclopentyl-2H-pyrazol-3-amine (135 mg, 0.890 mmol) was added. The solution was heated to 80° C. for 18 h. The solution was allowed to cool to room temperature and then filtered. The solid was added to water (10 ml) and concentrated ammonia solution (3 drops) was added. The precipitate was collected by filtration, washed with water (2 ml) and dried in vacuo to yield the title compound as a colourless solid (180.8 mg, 60% yield).

¹H NMR (399.902 MHz, DMSO with D-4 AcOD) δ 1.55 (m, 6H), 1.87 (m, 2H), 2.09 (s, 3H), 2.90 (m, 1H), 4.47 (d, J=5.2 Hz, 2H), 6.03 (s, 1H), 6.13 (bs, 1H), 6.18 (bs, 1H), 7.75 (d, J=5.9 Hz, 1H)

MS: m/z 340 (MH+)

(4-chloro-N-[(3-methyl-1,2-oxazol-5-yl)methyl]pyrimidin-2-amine used as a starting material was prepared as follows:—

To a solution containing 2-[(3-methyl1,2-oxazol-5-yl)methylamino]pyrimidin-4-ol (8.8 g) and diisopropylethylamine (9.6 ml) in toluene (40 ml) was added phosphorous oxychloride (4.8 ml) dropwise. The gummy suspension was heated at 80° C. for 2 h. The reaction was allowed to cool to r.t and then poured portionwise into saturated sodium bicarbonate solution The product was extracted with ethyl acetate (×2), washed with brine, dried (MgSO₄), filtered and evaporated to give a cream solid. The solid was washed with ethyl acetate and dichloromethane (plus few drops of methanol) in an attempt to dissolve it. The suspension was heated to reflux. After filtration, a cream solid was obtained (1.6 g). The filtrate was loaded onto a silica column and after elution with ethyl acetate the crude product was obtained. Trituration with diethyl ether gave the desired compound as a pale yellow solid (3.28 g). Total yield=4.88 g (50%).

¹H NMR (400.13 MHz DMSO) 2.19 (s, 3H), 4.56 (d, 2H), 6.15 (s, 1H), 6.77 (d, 1H), 8.22 (t, 1H), 8.29 (d, 1H)

MS: m/z 225 (MH+)

2-[(3-methyl1,2-oxazol-5-yl)methylamino]pyrimidin-4-ol was prepared as follows:—

(3-Methylisoxazol-5-yl)methanamine (9.3 g, 83 mmoles) and 2-methylsulfonylpyrimidin-4-ol (9.8 g, 69 mmoles) were heated together at 160° C. for 4 h. The mixture was allowed to cool then dissolved in dichloromethane and purified by chromatography (silica) eluting with 5-15% methanol in dichloromethane to give the product as a brown gum (8.88 g, 62%).

1H NMR (DMSO) δ 2.19 (s, 3H), 4.57 (s, 2H), 5.6 (d, 1H), 6.19 (s, 1H), 7.03 (bs, 1H), 7.61 (d, 1H), 11 (bs, 1H)

MS: m/z 207 (MH+)

5-cyclopentyl-2H-pyrazol-3-amine used as a starting material was prepared as follows:—

To an argon flushed reaction vessel was added 1,4-dioxane (100 ml, anhydrous) and to this was added sodium hydride (3.60 g, 60% dispersion in mineral oil, 90 mmoles). Acetonitrile (4.7 ml, 90 mmole, anhydrous) was added to the slurry and the mixture was stirred at room temperature for 30 mins. Methyl cyclopentanecarboxylate was added (9.6 g, 75 mmole) via syringe. The mixture was stirred at room temperature for 30 mins, then slowly heated to 105° C. overnight. The mixture was evaporated to dryness and the resulting solid dissolved in water (250 ml). The aqueous solution was extracted with DCM (3×75 ml). The aqueous layer was then acidified to pH 1-3 with concentrated hydrochloric acid (5-6 ml). The product was extracted into DCM (5×75 ml) and the combined organic extracts were dried over magnesium sulphate and filtered. The filtrate was evaporated at 600 mbar and 60° C. on a rotary evaporator, to avoid loss of any volatile product. The resulting oil was dissolved in ethanol (100 ml) and hydrazine hydrate (2 eq., 7.50 g, 150 mmoles) was added and the mixture was refluxed overnight. The solution was evaporated to dryness and then purified by silica column chromatography, eluting with a 0-10% MeOH in DCM gradient to give the desired compound (7.6 g, 67%)

MS: m/z 152 (MH+)

### Example 14

### N′-(5-cyclopentyl-2H-pyrazol-3-yl)-N-[(3-cyclopropyl-1,2-oxazol-5-yl)methyl]pyrimidine-2,4-diamine

To a reaction tube was added 4-chloro-N-[(3-cyclopropyl-1,2-oxazol-5-yl)methyl]pyrimidin-2-amine (100 mg, 0.40 mmoles), ethanol (2 ml), and 5-cyclopentyl-2H-pyrazol-3-amine (64mg, 0.42 mmoles). The mixture was heated overnight at 80° C. The cooled mixture was filtered and washed with ethanol. The sample was dissolved in methanol, poured onto a SCX-2 column and washed with methanol. The product eluted with 2N ammonia in methanol and the solvent was evaporated to give a gum. The gum was triturated with ether, filtered, dried in a vacuum oven at 45° C. overnight to yield the title product as a white solid (80mg, 55%).

1H NMR (DMSO 400.13 MHz) 0.68 (m, 2H), 0.94 (m, 2H), 1.48-1.75 (m, 6H), 1.95 (m, 3H), 2.96 (m, 1H), 4.52 (d, 2H), 5.99 (s, 1H), 6.25 (bm, 2H), 7.15 (bs, 1H), 7.82 (d, 1H), 9.34 (s, 1H), 11.88 (s, 1H)

MS: m/z 366 (MH+)

5-cyclopentyl-2H-pyrazol-3-amine amine used as a starting material was prepared as in Example 13.

4-chloro-N-[(3-cyclopropyl-1,2-oxazol-5-yl)methyl]pyrimidin-2-amine was prepared in analogous manner to (4-chloro-N-[(3-methyl-1,2-oxazol-5-yl)methyl]pyrimidin-2-amine in Example 13 except using 2-[(3-cyclopropyl-1,2-oxazol-5-yl)methylamino]pyrimidin-4-ol as starting material (3.17 g, 13.65 mmoles). Yield was 1.79 g (52%).

### Example 15

### N′-(5-cyclopentyl-2H-pyrazol-3-yl)-N-[[3-(oxolan-2-yl)-1,2-oxazol-5-yl]ethyl]pyrimidine-2,4-diamine

2-chloro-N-(5-cyclopentyl-2H-pyrazol-3-yl)pyrimidin-4-amine (150 mg, 0.569 mmol) was dissolved in 2-methoxy ethanol (5 ml) and [3-(oxolan-2-yl)-1,2-oxazol-5-ylmethanamine (192 mg, 1.138 mmol) and di-isopropylethylamine (148 mg, 199 μl, 1.138 mmol) were added. The mixture was heated to 160° C. for 30 mins in a microwave reactor, then to 180° C. for 20 mins and then to 200° C. for 80 mins. The solvent was evaporated under reduced pressure and the crude product was purified by reverse-phase preparative HPLC (basic) using a 25-45% gradient of acetonitrile in water containing 1% ammonium hydroxide solution. The clean fractions were combined and evaporated to give the title compound as a colourless solid (52 mg, 23% yield).

¹H NMR (399.902 MHz, DMSO and d-4 AcOD) δ 1.62 (m, 6H), 1.91 (m, 5H), 2.21 (m, 1H), 2.98 (m, 1H), 3.79 (m, 2H), 4.58 (d, J =5.4 Hz, 2H), 4.87 (t, J =6.7 Hz, 1H), 6.21 (s, 1H), 6.25 (s, 1H), 7.28 (t, J=5.5 Hz, 1H), 7.83 (d, J=5.7 Hz, 1H), 9.43 (s, 1H). MS: m/z 396 (MH+)

2-chloro-N-(5-cyclopentyl-2H-pyrazol-3-yl)pyrimidin-4-amine, used as starting material was prepared as follows:

2,4-Dichloropyrimidine (500 mg, 3.356 mmol) was dissolved in ethanol (10 ml) and di-isopropylethylamine (702 μl, 4.027 mmol) and 5-cyclopentyl-2H-pyrazol-3-amine (559 mg, 3.692 mmol) were added. The mixture was stirred at 40° C. for 3 days then allowed to cool to room temperature. The solution was concentrated to approximately half of the initial volume under reduced pressure, then added dropwise to water. The mixture was left to stand for 18 h and then the precipitate was collected by filtration, washed with water and dried in vacuo to yield 2-chloro-N-(5-cyclopentyl-2H-pyrazol-3-yl)pyrimidin-4-amine as a cream solid (644.2 mg, 73% yield)

¹H NMR (399.902 MHz, DMSO) δ 1.65 (m, 6H), 2.02 (s, 2H), 3.04 (m, 1H), 6.08 (bs, 1H), 8.17 (s, 1H), 10.27 (s, 1H), 12.17 (s, 1H) MS: m/z 264 (MH+)

5-cyclopentyl-2H-pyrazol-3-amine amine used as a starting material was prepared as in Example 13.

[3-(oxolan-2-yl)-1,2-oxazol-5-ylmethanamine, used as a starting material was prepared in an analogous manner to that described for (3-cyclopropylisoxazol-5-yl)methanamine hydrochloride (Example 3) by the method described in the literature (Nowak, Thorsten; Thomas, Andrew Peter. Preparation of 4-(pyrazol-3-ylamino)pyrimidines for use in the treatment of cancer. WO2005040159). Oxolane-2-carbaldehyde was used as starting material.

### Example 16

### N-[(3-cyclopropyl-1,2-oxazol-5-yl)methyl]-N′-[5-(2-methylpropyl)-2H-pyrazol-3-yl]pyrimidine-2,4-diamine

To a reaction tube was added 4-chloro-N-[(3-cyclopropyl-1,2-oxazol-5-yl)methyl]pyrimidin-2-amine (100 mg, 0.40 mmoles), ethanol (2 ml), and 5-(2-methylpropyl)-2H-pyrazol-3-amine (59mg, 0.42 mmoles). The mixture was heated overnight at 80° C. The cooled mixture was filtered and the solid was washed with ethanol. The sample was dissolved in methanol, poured onto a SCX-2 column and washed with methanol. The product eluted with 2N ammonia in methanol and the solvent was evaporated to give a gum. The gum was triturated with ether, filtered, dried in a vacuum oven at 45° C. overnight to yield the title product as a white solid (65mg, 47%).

1H NMR (DMSO 400.13 MHz) 0.69 (m, 2H), 0.87 (m, 6H), 0.95 (m, 2H), 1.85 (m, 1H), 1.93 (m, 1H), 2.39 (d, 2H), 4.51 (d, 2H), 5.99 (s, 1H), 6.2-6.35 (bs, 2H), 7.17 (bs, 1H), 7.82 (d, 1H), 9.38 (bs, 1H), 11.85 (s,1 H)

MS: m/z 354 (MH+)

4-chloro-N-[(3-cyclopropyl-1,2-oxazol-5-yl)methyl]pyrimidin-2-amine material was prepared as in Example 14.

5-(2-methylpropyl)-2H-pyrazol-3-amine, used as starting material, can be prepared in an analogous method to that described for 5-propyl-1H-pyrazol-3-amine (Example 6) by the method described in the literature (Barlaam, Bernard; Pape, Andrew; Thomas, Andrew. Preparation of pyrimidine derivatives as modulators of insulin-like growth factor-1 receptor (IGF-1). WO2003048133).

TABLE 2

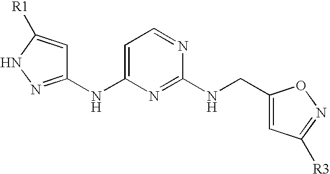

| Example | R1 | R3 |
|---|---|---|
| 17 | 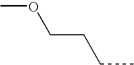 | Me |
| 18 | 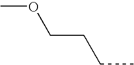 | 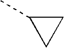 |
| 19 | 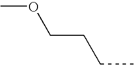 | 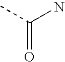 |
| 20 | 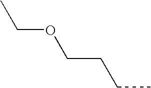 | Me |
| 21 | 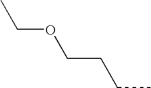 | 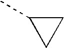 |
| 22 | 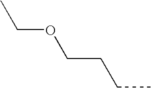 | 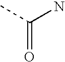 |
| 23 | 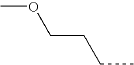 | 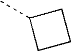 |

### Example 17

### N′-[5-(3-methoxypropyl)-2H-pyrazol-3-yl]-N-[(3-methylisoxazol-5-yl)methyl]pyrimidine-2,4-diamine (also known as N′-[5-(3-methoxypropyl)-2H-pyrazol-3-yl]-N-[(3-methyl-1,2-oxazol-5-yl)m ethyl]pyrimidine-2,4-diamine)

Prepared in an analogous way to example 3 but starting with 2-chloro-N-[5-(3-methoxypropyl)-1H-pyrazol-3-yl]pyrimidin-4-amine (0.10 g, 0.37 mmol) and (3-methylisoxazol-5-yl)methanamine hydrochloride (also known as (3-methyl-1,2-oxazol-5-yl)methanamine hydrochloride; 0.084 g, 0.56 mmol) to give example 17 in table 2 (0.033 g, 26% yield).

1H NMR (300 MHz, DMSO): 1.76-1.85 (m, 2H), 2.17 (s, 3H), 2.57 (t, 2H), 3.24 (s, 3H), 3.34 (t, 2H), 4.53 (d, 2H), 6.10 (s, 1H), 6.14-6.39 (m, 2H), 7.18 (s, 1H), 7.82 (d, 1H), 9.34 (s, 1H), 11.87 (s, 1H).

MS: m/z 344 (MH⁺).

2-chloro-N-[5-(3-methoxypropyl)-1H-pyrazol-3-yl]pyrimidin-4-amine, used as starting material, was prepared as follows:
a) Acetonitrile (6.3 ml, 120 mmol) was added to a slurry of sodium hydride (4.8 g dispersion in mineral oil, 120 mmol) in anhydrous 1,4-dioxane (135 ml) and the mixture was stirred at room temperature for 30 minutes. Methyl 4-methoxybutyrate (13.23 ml, 100 mmol) was added and the mixture was stirred at room temperature for 30 minutes and then heated at 105° C. overnight. Water (3 drops) was added and the mixture was then evaporated. The residue was dissolved in water (350 ml) and extracted with dichloromethane (3×). The aqueous layer was acidified to pH 1-3 with concentrated hydrochloric acid and then extracted into dichloromethane (5×). The combined extracts were dried over magnesium sulfate and then evaporated. To the residue in ethanol (135 ml) was added hydrazine hydrate (9.7 ml, 200 mmol) and the mixture heated at reflux overnight. The mixture was evaporated and then co-evaporated with ethanol (2×). The residue was purified by chromatography on silica eluting with a mixture of 0-10% methanol in dichloromethane. Fractions containing product were combined and evaporated to leave 5-(3-methoxypropyl)-1H-pyrazol-3-amine.

¹H NMR (300 MHz, CDCl3): 1.75-1.84 (m, 2H), 2.56 (t, 2H), 3.27 (s, 3H), 3.33 (t, 2H), 5.36 (s, 1H).
b) A mixture of 2,4-dichloropyrimidine (1. 845 g, 12.3 8 mmol), 5-(3-methoxypropyl)-1H-pyrazol-3-amine (2.405 g, 15.48 mmol) and N,N-diisopropylethylamine (4.32 ml, 24.8 mmol) in ethanol was allowed to stand for 6 days at room temperature. The mixture was concentrated and the residue dissolved in dichloromethane (60 ml) and then washed with water (2×50 ml) followed by brine (2×50 ml). The organic phase was dried over sodium sulfate and then purified directly by chromatography on silica eluting with a mixture of 50-75% ethyl acetate in isohexane. Fractions containing product were combined and evaporated to leave a solid which was triturated with diethyl ether to give 2-chloro-N-[5-(3-methoxypropyl)-1H-pyrazol-3-yl]pyrimidin-4-amine (2.45 g, 74% yield).

¹H NMR (300 MHz, DMSO): 1.76-1.86 (m, 2H), 2.62 (t, 2H), 3.24 (s, 3H), 3.34 (t, 2H), 6.11 (s, 1H), 7.19 (s, 1H), 8.16 (d, 1H), 10.28 (s, 1H), 12.17 (s, 1H).

MS: m/z 268 (MH⁺).

(3-methyl-1,2-oxazol-5-yl)methanamine hydrochloride, used as starting material, was prepared as outlined in Example 1.

### Example 18

### N-[(3-cyclopropylisoxazol-5-yl)methyl]-N′-[5-(3-methoxypropyl)-2H-pyrazol-3-yl]pyrimidine-2,4-diamine (also known as N-[(3-cyclopropyl-1,2-oxazol-5-yl)methyl]-N′-[5-(3-methoxypropyl)-2H-pyrazol-3-yl]pyrimidine-2,4-diamine)

Prepared in an analogous way to example 17 but starting with (3-cyclopropylisoxazol-5-yl)methanamine hydrochloride (also known as (3-cyclopropyl-1,2-oxazol-5-yl)methanamine hydrochloride; 0.098 g, 0.56 mmol) to give example 18 in table 2 (0.054 g, 39% yield).

1H NMR (300 MHz, DMSO): 0.67-0.72 (m, 2H), 0.93-0.99 (m, 2H), 1.76-1.85 (m, 2H), 1.89-1.99 (m, 1H), 2.57 (t, 2H), 3.24 (s, 3H), 3.34 (t, 2H), 4.50 (s, 2H), 5.99 (s, 1H), 6.13-6.39 (m, 2H), 7.15 (s, 1H), 7.82 (d, 1H), 9.34 (s, 1H), 11.88 (s, 1H).

MS: m/z 370 (MH⁺).

(3-cyclopropyl-1,2-oxazol-5-yl)methanamine hydrochloride, used as starting material, was prepared as in Example 3.

### Example 19

### 5-[[[4-[[5-(3-methoxypropyl)-2H-pyrazol-3-yl]amino]pyrimidin-2-yl]amino]methyl]isoxazole-3-carboxamide (also known as 5-[[[4-[[5-(3-methoxypropyl)-2H-pyrazol-3-yl]amino]pyrimidin-2-yl]amino]methyl]-1,2-oxazole-3-carboxamide)

Prepared in an analogous way to example 17 but starting with 5-(aminomethyl)isoxazole-3-carboxamide hydrochloride (also known as 5-(aminomethyl)-1,2-oxazole-3-carboxamide hydrochloride; 0.10 g, 0.56 mmol) to give example 19 in table 2 (0.007 g, 5% yield).

1H NMR (300 MHz, DMSO): 1.76-1.85 (m, 2H), 2.57 (t, 2H), 3.24 (s, 3H), 3.34 (t, 2H), 4.62 (d, 2H), 6.16-6.36 (m, 2H), 6.52 (s, 1H), 7.27 (s, 1H), 7.73 (s, 1H), 7.84 (d, 1H), 8.02 (s, 1H), 9.38 (s, 1H), 11. 89 (s, 1H).

MS: m/z 373 (MH⁺).

5-(aminomethyl)-1,2-oxazole-3-carboxamide hydrochloride, used as starting material, can be prepared as described in Example 4.

### Example 20

### N′-[5-(3-ethoxypropyl)-2H-pyrazol-3-yl]-N-[(3-methylisoxazol-5-yl)methyl]pyrimidine-2,4-diamine (also known as N′-[5-(3-ethoxypropyl)-2H-pyrazol-3-yl]-N-[(3-methyl-1,2-oxazol-5-yl)methyl]pyrimidine-2,4-diamine)

Prepared in an analogous way to example 3 but starting with 2-chloro-N-[5-(3-ethoxypropyl)-1H-pyrazol-3-yl]pyrimidin-4-amine (0.10 g, 0.35 mmol) and (3-methylisoxazol-5-yl)methanamine hydrochloride (also known as (3-methyl-1,2-oxazol-5-yl)methanamine hydrochloride; 0.080 g, 0.53 mmol) to give example 20 in table 2 (0.024 g, 19% yield).

¹H NMR (300 MHz, DMSO): 1.11 (t, 3H), 1.75-1.84 (m, 2H), 2.17 (s, 3H), 2.57 (t, 2H), 3.35-3.45 (m, 4H), 4.53 (d, 2H), 6.10 (s, 1H), 6.15-6.41 (m, 2H), 7.18 (s, 1H), 7.82 (d, 1H), 9.34 (s, 1H), 11.87 (s, 1H).

MS: m/z 358 (MH⁺).

2-chloro-N-[5-(3-ethoxypropyl)-1H-pyrazol-3-yl]pyrimidin-4-amine, used as starting material, was prepared as follows:
a) Prepared in an analogous reaction to that described in example 17a but starting with ethyl 4-ethoxybutyrate (20.0 g, 125 mmol) to give 5-(3-ethoxypropyl)-1H-pyrazol-3-amine (13.9 g, 66% yield).

MS: m/z 170 (MH⁺).
b) Prepared in an analogous reaction to that described in example 17b but starting with 5-(3-ethoxypropyl)-1H-pyrazol-3-amine (5.0 g, 29.6 mmol) to give 2-chloro-N-[5-(3-ethoxypropyl)-1H-pyrazol-3-yl]pyrimidin-4-amine (4.2 g, 51% yield).

¹H NMR (300 MHz, DMSO): 1.12 (t, 3H), 1.76-1.85 (m, 2H), 2.62 (t, 2H), 3.35-3.45 (m, 4H), 5.88-6.33 (m, 1H), 7.19 (s, 1H), 8.16 (d, 1H), 10.27 (s, 1H), 12.16 (s, 1H).

MS: m/z 282 (MH⁺).

(3-methyl-1,2-oxazol-5-yl)methanamine hydrochloride, used as starting material, was prepared as outlined in Example 1.

### Example 21

### N-[(3-cyclopropylisoxazol-5-yl)methyl]-N ′-[5-(3-ethoxypropyl)-2H-pyrazol-3-yl]pyrimidine-2,4-diamine (also known as N-[(3-cyclopropyl-1,2-oxazol-5-yl)methyl]-N′-[5-(3-ethoxypropyl)-2H-pyrazol-3-yl]pyrimidine-2,4-diamine)

Prepared in an analogous way to example 20 but starting with (3-cyclopropylisoxazol-5-yl)methanamine hydrochloride (also known as (3-cyclopropyl-1,2-oxazol-5-yl)methanamine hydrochloride; 0.093 g, 0.53 mmol) to give example 21 in table 2 (0.032 g, 24% yield).

¹H NMR (300 MHz, DMSO): 0.67-0.72 (m, 2H), 0.92-0.99 (m, 2H), 1. 11 (t, 3H), 1.75-1.84 (m, 2H), 1.90-1.99 (m, 1H), 2.57 (t, 2H), 3.35-3.44 (m, 4H), 4.51 (d, 2H), 5.99 (s, 1H), 6.07-6.49 (m, 2H), 7.15 (s, 1H), 7.82 (d, 1H), 9.33 (s, 1H), 11.87 (s, 1H).

MS: m/z 384 (MH⁺).

(3-cyclopropyl-1,2-oxazol-5-yl)methanamine hydrochloride, used as starting material, was prepared as in Example 3.

### Example 22

### 5-[[[4-[[5-(3-ethoxypropyl)-2H-pyrazol-3-yl]amino]pyrimidin-2-yl]amino]methyl]isoxazole-3-carboxamide (also known as 5-[[[4-[[5-(3-ethoxypropyl)-2H-pyrazol-3-yl]amino]pyrimidin-2-yl]amino]methyl]-1,2-oxazole-3-carboxamide)

Prepared in an analogous way to example 20 but starting with 5-(aminomethyl)isoxazole-3-carboxamide (also known as 5-(aminomethyl)-1,2-oxazole-3-carboxamide; 0.095 g, 0.53 mmol) to give example 22 in table 2 (0.038 g, 28% yield).

¹H NMR (300 MHz, DMSO): 1.11 (t, 3H), 1.74-1.84 (m, 2H), 2.58 (t, 2H), 3.36-3.45 (m, 4H), 4.62 (d, 2H), 6.23 (s, 1H), 6.31 (s, 1H), 6.51 (s, 1H), 7.26 (s, 1H), 7.74 (s, 1H), 7.83 (d, 1H), 8.02 (s, 1H), 9.38 (s, 1H), 11.88 (s, 1H).

MS: m/z 387 (MH⁺).

5-(aminomethyl)-1,2-oxazole-3-carboxamide, used as starting material, can be prepared as described in Example 4.

### Example 23

### N-[(3-Cyclobutyl1,2-oxazol-5-yl)methyl]-N′-[5-(3-methoxypropyl)-1H-pyrazol-3-yl]pyrimidine-2,4-diamine

Prepared in an analogous way to Example 3, from 2-chloro-N-[5-(3-methoxypropyl)-1H-pyrazol-3-yl]pyrimidin-4-amine (75mg, 0.28 mmol) and (3-cyclobutyl1,2-oxazol-5-yl)methanamine (95mg, 0.56 mmol) to yield the title compound (51 mg, 48%) as a white solid.

1H NMR (300.132 MHz, DMSO) 6 1.79 (t, 2H), 1.86-1.88 (m, 2H), 2.05-2.14 (m, 2H), 2.20-2.29 (m, 2H), 2.56 (t, 2H), 3.22 (s, 3H), 3.32 (t, 2H), 3.44-3.55 (m, 1H), 4.57 (s, 2H), 6.18 (s, 1H), 6.22 (s, 1H), 6.27 (s, 1H), 7.82 (d, 1H). MS: m/z 384 (MH+)

2-Chloro-N-[5-(3-methoxypropyl)-1H-pyrazol-3-yl]pyrimidin-4-amine, used as starting material, can be prepared by the method described in Example 17.

(3-Cyclobutyl1,2-oxazol-5-yl)methanamine, used as starting material, can be prepared by the method described in the literature (Nowak, Thorsten; Thomas, Andrew Peter. Preparation of 4-(pyrazol-3-ylamino)pyrimidines for use in the treatment of cancer. WO2005040159). Starting from cyclobutanecarbaldehyde (14.64 g, 174 mmol) afforded (3-cyclobutylisoxazol-5-yl)methanamine as an oil (8.8 g, 27% over 3 steps). ¹H NMR (399.9 MHz, CDCl₃) δ1.52 (2H, s), 1.82-1.94 (1H, m), 1.96-2.07 (1H, m), 2.09-2.06 (1H, m), 2.09-2.21(2H, m), 2.23-2.35 (2H, m), 3.49-3.57 (1H, m), 3.89(2h, s), 5.98 (1H, s).MS: m/z 153 (MH+).

TABLE 3

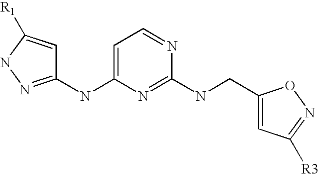

| Example | R1 | R3 |
|---|---|---|
| 24 | 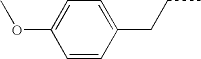 | Me |
| 25 | 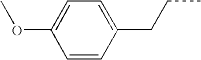 | 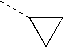 |
| 26 | 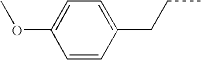 | 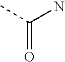 |
| 27 | 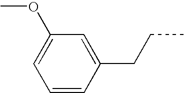 | Me |
| 28 | 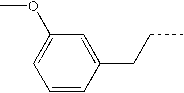 | 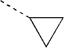 |
| 29 | 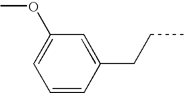 | 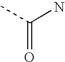 |
| 30 | 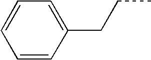 | Me |
| 31 | 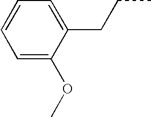 | Me |
| 32 | 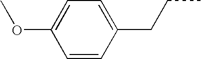 | 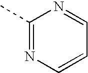 |
| 33 | 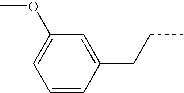 | 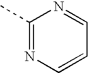 |
| 34 | 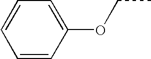 | Me |
| 35 | 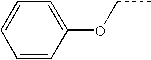 | 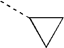 |
| 36 | 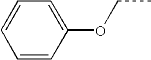 | 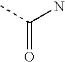 |
| 37 | 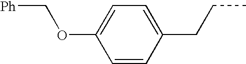 | Me |
| 38 | 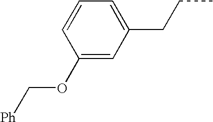 | Me |
| 39 | 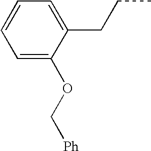 | Me |
| 40 | 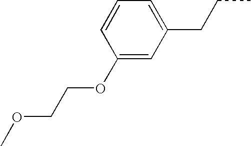 | Me |
| 41 | 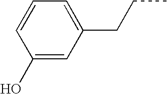 | Me |
| 42 | 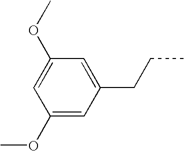 | Me |
| 43 | 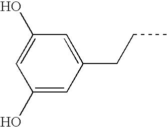 | Me |
| 44 | 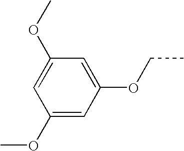 | Me |
| 45 | 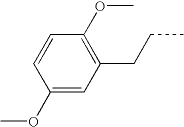 | Me |
| 46 | 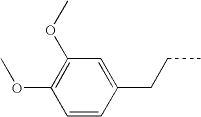 | Me |
| 47 | 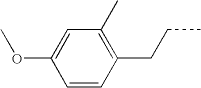 | Me |
| 48 | 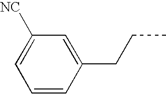 | Me |
| 49 | 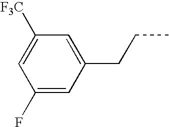 | Me |
| 50 | 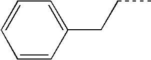 | 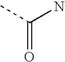 |
| 51 | 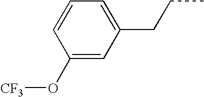 | Me |
| 52 | 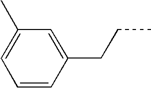 | Me |
| 53 | 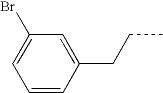 | Me |
| 54 | 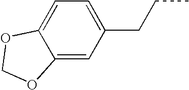 | Me |
| 55 | 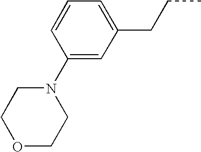 | Me |
| 56 | 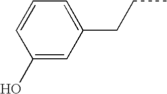 | 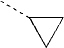 |
| 57 | 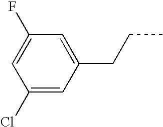 | Me |
| 58 | 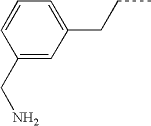 | Me |
| 59 | 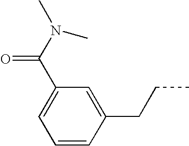 | Me |
| 60 | 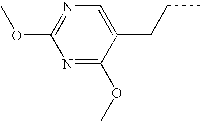 | Me |
| 61 | 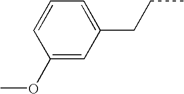 | 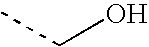 |
| 62 | 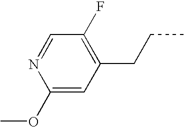 | Me |
| 63 | 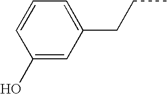 | 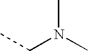 |
| 64 | 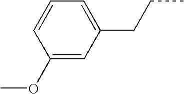 | 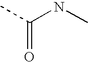 |
| 65 | 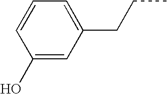 | 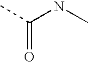 |
| 122 | 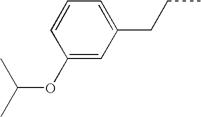 | Me |
| 123 | 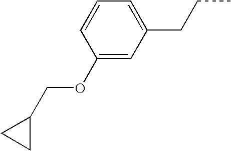 | 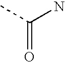 |
| 124 | 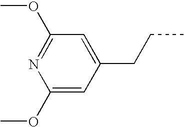 | Me |
| 125 | 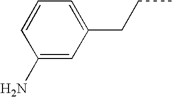 | Me |
| 126 | 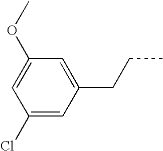 | 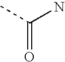 |
| 127 | 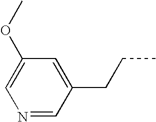 | 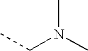 |
| 128 | 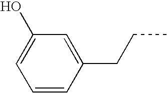 | 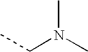 |
| 132 | 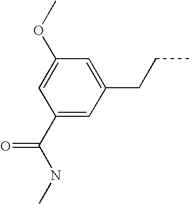 | Me |
| 133 | 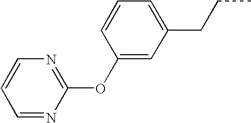 | Me |
| 134 | 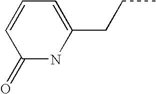 | Me |
| 136 | 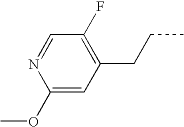 | 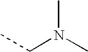 |
| 138 | 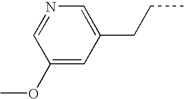 | Me |
| 139 | 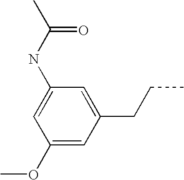 | Me |
| 140 | 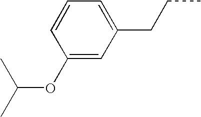 | 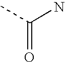 |
| 141 | 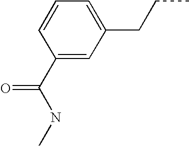 | Me |
| 142 | 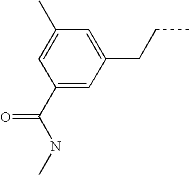 | Me |
| 143 | 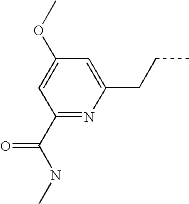 | Me |

### Example 24

### N′-[5-[2-(4-methoxyphenyl)ethyl]-2H-pyrazol-3-yl]-N-[(3-methylisoxazol-5-yl)methyl]pyrimidine-2,4-diamine (also known as N′-[5-[2-(4-methoxyphenyl)ethyl]-2H-pyrazol-3-yl]-N-[(3-methyl-1,2-oxazol-5-yl)methyl]pyrimidine-2,4-diamine)

A mixture of 2-chloro-N-[5-[2-(4-methoxyphenyl)ethyl]-1H-pyrazol-3-yl]pyrimidin-4-amine (0.10 g, 0.3 mmol), (3-methylisoxazol-5-yl)methanamine hydrochloride (also known as (3-methyl-i1,2-oxazol-5-yl)methanamine hydrochloride; 0.068 g, 0.45 mmol) and N,N-diisopropylethylamine (0. 159 ml, 0.91 mmol) in 2-methoxyethanol (3 ml) was heated at 170° C. in a Emrys Optimiser microwave for 3 hours. The mixture was concentrated in vacuo and the residue was dissolved in a mixture of dimethylformamide and acetonitrile (1 :3.8) and purified directly by preparative hplc eluting with a gradient of acetonitrile in water containing 1% ammonia. The fractions containing product were combined and evaporated to leave compound 18 in table 3 (0.039 g, 32% yield).

¹H NMR (300 MHz, DMSO): 2.16 (3H, s), 2.71-2.88 (4H, m), 3.71 (3H, s), 4.53 (2H, d), 6.10 (1H, s), 6.23 (2H, s), 6.84 (2H, d), 7.14 (2H, d), 7.22 (1H, s), 7.83 (1H, d), 9.40 (1H, s), 11.93 (1H, s).

MS: m/z 406 (MH⁺).

2-chloro-N-[5-[2-(4-methoxyphenyl)ethyl]-1H-pyrazol-3-yl]pyrimidin-4-amine, used as starting material, was prepared as follows:
a) To a solution of methyl 3-(4-methoxyphenyl)propanoate (7.77 g, 40 mmol) and acetonitrile (2.09 ml, 40 mmol) in toluene (30 ml) cooled to 0° C. was added sodium hydride (60% dispersion in oil, 1.92 g, 48 mmol). The mixture was stirred at 0° C. for 15 minutes and then heated to reflux for 2 hours. The mixture was evaporated and the residue was dissolved in water and then extracted with dichloromethane. The aqueous layer was acidified using 2M hydrochloric acid and then extracted with dichloromethane (2×). The organic extracts were combined, washed with 2M hydrochloric acid, water and finally with brine and then dried over magnesium sulfate. The solution was evaporated under reduced pressure to leave a yellow oil which solidified on standing. The solid was refluxed in ethanol (25 ml) and hydrazine hydrate (0.549 ml, 11.3 mmol) for 3.5 hours. The mixture was evaporated and the residue dissolved in ethyl acetate and the solution was washed twice with water and then with brine. The organic layer was separated, dried with magnesium sulfate and then evaporated under reduced pressure to leave 5-[2-(4-methoxyphenyl)ethyl]-1H-pyrazol-3-amine (2.13 g, 25% yield over 2 steps).

¹H NMR (300 MHz, DMSO): 2.62-2.81 (4H, m), 3.72 (3H, s), 4.39 (1H, s), 5.17 (1H, s), 6.83 (2H, d), 7.12 (2H, d), 11.15 (1H, s).

MS: m/z 218 (MH⁺).
b) To a solution of 5-[2-(4-methoxyphenyl)ethyl]-1H-pyrazol-3-amine (2.02 g, 9.30 mmol) in ethanol (40 ml) was added di-iso-propylethylamine (2.7 ml, 15.5 mmol) followed by 2,4-dichloropyrimidine (1.155 g, 7.75 mmol). The mixture was heated at 50° C. for 70 hours. The mixture was allowed to cool to room temperature and then water was added to yield an oily emulsion. The mixture was concentrated to remove the bulk of the ethanol and the mixture was then extracted with ethyl acetate. The organic layer was separated and then washed with water and brine before drying over magnesium sulfate. The mixture was evaporated and the residue triturated with dichloromethane. The resulting solid was filtered and washed with a mixture of 50% diethyl ether in hexane and then dried in a vacuum dessicator overnight to give 2-chloro-N-[5-[2-(4-methoxyphenyl)ethyl]-1H-pyrazol-3-yl]pyrimidin-4-amine (1.50 g, 59% yield).

¹H NMR (300 MHz, DMSO): 2.85 (4H, s), 3.72 (3H, s), 5.75 (1H, s), 6.09 (1H, s), 6.85 (2H, d), 7.15 (2H, d), 8.16 (1H, d), 10.26 (1H, s), 12.19 (1H, s).

MS: m/z 330 (MH⁺).

(3-methyl-1,2-oxazol-5-yl)methanamine hydrochloride, used as starting material, was prepared as outlined in Example 1.

### Example 25

### N-[(3-cyclopropylisoxazol-5-yl)methyl]-N ′-[5-[2-(4-methoxyphenyl)ethyl]-2H-pyrazol-3- yl]pyrimidine-2,4-diamine (also known as N-[(3-cyclopropyl-1,2-oxazol-5-yl)methyl]-N′-[5-[2-(4-methoxyphenyl)ethyl]-2H-pyrazol-3-yl]pyrimidine-2,4-diamine)

Prepared in an analogous way to example 24 but starting with (3-cyclopropylisoxazol-5-yl)methanamine hydrochloride (also known as (3-cyclopropyl-1,2-oxazol-5-yl)methanamine hydrochloride; 0.080 g, 0.45 mmol) to give example 25 in table 3 (0.027 g, 21% yield).

¹H NMR (300 MHz, DMSO): 0.65-0.73 (2H, m), 0.90-0.99 (2H, m), 1.94 (1H, ddd), 2.74-2.87 (4H, m), 3.72 (3H, s), 4.51 (2H, m), 5.99 (1H, s), 6.28 (2H, m), 6.84 (2H, d), 7.10-7.19 (3H, m), 7.82 (1H, d), 9.34 (1H, s), 11.89 (1H, s).

MS: m/z 432 (MH⁺).

(3-cyclopropyl-1,2-oxazol-5-yl)methanamine hydrochloride, used as starting material, was prepared as in Example 3.

### Example 26

### 5-[[[4-[[5-[2-(4-methoxyphenyl)ethyl]-2H-pyrazol-3-yl]amino]pyrimidin-2-yl]amino]methyl]isoxazole-3-carboxamide (also known as 5-[[[4-[[5-[2-(4-methoxyphenyl)ethyl]-2H-pyrazol-3-yl]amino]pyrimidin-2-yl]amino]methyl]-1,2-oxazole-3-carboxamide)

Prepared in an analogous way to example 24 but starting with 5-(aminomethyl)isoxazole-3-carboxamide trifluoroacetate (also known as 5-(aminomethyl)-1,2-oxazole-3-carboxamide trifluoroacetate; 0.1 17 g, 0.45 mmol) to give example 26 in table 3 (0.030 g, 23% yield).

¹H NMR (300 MHz, DMSO): 2.77-2.86 (4H, m), 3.71 (3H, s), 4.62 (2H, d), 6.27 (2H, m), 6.52 (1H, s), 6.84 (2H, d), 7.15 (2H, s), 7.30 (1H, s), 7.74 (1H, s), 7.84 (1H, d), 8.02 (1H, s), 9.38 (1H, s), 11.92 (1H, s).

MS: m/z 435 (MH⁺).

5-(aminomethyl)-1,2-oxazole-3-carboxamide, used as starting material, can be prepared as described in Example 4.

### Example 27

### N′-[5-[2-(3-methoxyphenyl)ethyl]-2H-pyrazol-3-yl]-N-[(3-methylisoxazol-5-yl)methyl]pyrimidine-2,4-diamine (also known as N′-[5-[2-(3-methoxyphenyl)ethyl]-2H-pyrazol-3-yl]-N-[(3-methyl-1,2-oxazol-5-yl)methyl]pyrimidine-2,4-diamine)

A mixture of 2-chloro-N-[5-[2-(3-methoxyphenyl)ethyl]-1H-pyrazol-3-yl]pyrimidin-4-amine (0.10 g, 0.3 mmol), (3-methylisoxazol-5-yl)methanamine hydrochloride (also known as (3-methyl-1,2-oxazol-5-yl)methanamine hydrochloride; 0.091 g, 0.6 mmol) and N,N-diisopropylethylamine (0.212 ml, 1.2 mmol) in 2-methoxyethanol (3 ml) was heated at 200° C. in a Emrys Optimiser microwave for 2 hours. The mixture was concentrated in vacuo and the residue was dissolved in a mixture of dimethylformamide and acetonitrile (1:3.8) and purified directly by preparative hplc eluting with a gradient of acetonitrile in water containing 1% ammonia. The fractions containing product were combined and concentrated. The resultant precipitate was filtered and the residue was washed with water and then dried under vacuum to leave compound 21 in table 3 (0.041 g, 34% yield).

H NMR (300 MHz, DMSO): 2.16 (3H, s), 2.76-2.95 (4H, m), 3.73 (3H, s), 4.53 (2H, d), 6.10 (1H, s), 6.19-6.37 (2H, m), 6.72-6.85 (3H, m), 7.13-7.25 (2H, m), 7.83 (1H, s), 9.34 (1H, s), 11.90 (1H, s).

MS: m/z 406 (MH⁺).

2-chloro-N-[5-[2-(3-methoxyphenyl)ethyl]-1H-pyrazol-3-yl]pyrimidin-4-amine, used as starting material, was prepared as follows:
a) In an analogous reaction to that described for example 24a but starting with ethyl 3-(3-methoxyphenyl)propanoate (10.4 g, 53.5 mmol) gave 5-[2-(3-methoxyphenyl)ethyl]-1H-pyrazol-3-amine (5.48 g, 47% yield over 2 steps).

¹H NMR (300 MHz, DMSO): 2.64-2.87 (4H, m), 3.73 (3H, s), 4.40 (1H, s), 5.19 (1H, s), 6.71-6.82 (3H, m), 7.18 (1H, t), 11.07 (1H, s).

MS: m/z 218 (MH⁺).
b) In an analogous reaction to that described for example 24b but starting with 5-[2-(3-methoxyphenyl)ethyl]-1H-pyrazol-3-amine (2.08 g, 9.55 mmol) gave 2-chloro-N-[5-[2-(3-methoxyphenyl)ethyl]-1H-pyrazol-3-yl]pyrimidin-4-amine (1.29 g, 49% yield).

¹H NMR (300 MHz, DMSO): 2.89 (4H, s), 3.73 (3H, s), 6.11 (1H, s), 6.73-6.84 (3H, m), 7.20 (2H, t), 8.16 (1H, d), 10.27 (1H, s), 12.20 (1H, s).

MS: m/z 330 (MH⁺).

(3-methyl-1,2-oxazol-5-yl)methanamine hydrochloride, used as starting material, was prepared as outlined in Example 1.

### Example 28

### N-[(3-cyclopropyl-1,2-oxazol-5-yl)methyl]-N′-[5-[2-(3-methoxyphenyl)ethyl]-2H-pyrazol-3-yl]pyrimidine-2,4-diamine

Prepared in an analogous way to example 27 but using (3-cyclopropylisoxazol-5-yl)methanamine hydrochloride (also known as (3-cyclopropyl-1,2-oxazol-5-yl)methanamine hydrochloride; 0.080 g, 0.45 mmol). After initial purification by preparative hplc a second purification step by preparative hplc, eluting with a gradient of acetonitrile (containing 0.2% trifluroracetic acid) in water (containing 0.2% trifluroracetic acid) was applied. The fractions containing product were combined and concentrated to leave compound 22 in table 3 (0.030 g, 23% yield).

¹H NMR (300 MHz, DMSO): 0.65-0.74 (2H, m), 0.95 (2H, dd), 1.94 (1H, ddd), 2.78-2.92 (4H, m), 3.73 (3H, s), 4.50 (2H, d), 5.99 (1H, s), 6.13-6.39 (2H, m), 6.72-6.84 (3H, m), 7.16 (1H, m), 7.19 (1H, t), 7.82 (1H, d), 9.34 (1H, s), 11.90 (1H, s).

MS: m/z 432 (MH⁺)

(3-cyclopropyl-1,2-oxazol-5-yl)methanamine hydrochloride, used as starting material, was prepared as in Example 3.

### Example 29

### 5-[[[4-[[5-[2-(3-methoxyphenyl)ethyl]-2H-pyrazol-3-yl]amino]pyrimidin-2-yl]amino]methyl]isoxazole-3-carboxamide (also known as 5-[[[4-[[5-[2-(3-methoxyphenyl)ethyl]-2H-pyrazol-3-yl]amino]pyrimidin-2-yl]amino]methyl]-1,2-oxazole-3-carboxamide)

Prepared in an analogous way to example 27 but using 5-(aminomethyl)isoxazole-3-carboxamide trifluoroacetate (also known as 5-(aminomethyl)-1,2-oxazole-3-carboxamide trifluoroacetate; 0.1 17 g, 0.45 mmol) to give example 29 in table 3 (0.026 g, 20% yield).

¹H NMR (300 MHz, DMSO): 2.78-2.93 (4H, m), 3.73 (3H, s), 4.61 (2H, d), 6.13-6.42 (2H, m), 6.52 (1H, s), 6.72-6.84 (3H, m), 7.19 (1H, t), 7.22-7.30 (1H, m), 7.73 (1H, s), 7.83 (1H, d), 8.01 (1H, s), 9.37 (1H, s), 11.92 (1H, s).

MS: m/z 435 (MH⁺).

5-(aminomethyl)-1,2-oxazole-3-carboxamide trifluoroacetate, used as starting material, can be prepared as described in Example 4.

### Example 30

### N-[(3-methylisoxazol-5-yl)methyl]-N′-(5-phenethyl-1H-pyrazol-3-yl)pyrimidine-2,4-diamine (also known as N-[(3-methyl-1,2-oxazol-5-yl)methyl]-N′-(5-phenethyl-1H-pyrazol-3-yl)pyrimidine-2,4-diamine)

A mixture of 2-chloro-N-(5-phenethyl-1H-pyrazol-3-yl)pyrimidin-4-amine (0.10 g, 0.33 mmol), (3-methylisoxazol-5-yl)methanamine hydrochloride (also known as (3-methyl-1,2-oxazol-5-yl)methanamine hydrochloride; 0.06 g, 0.4 mmol) and N,N-diisopropylethylamine (0. 175 ml, 1.0 mmol) in 2-methoxyethanol (2 ml) was heated at 170° C. in a Emrys Optimiser microwave for 2 hours. A further portion of (3-methylisoxazol-5-yl)methanamine hydrochloride (also known as (3-methyl-1,2-oxazol-5-yl)methanamine hydrochloride; 0.015 g, 0.1 mmol) was added and the mixture heated at 200° C. in the microwave for 1 hour. The mixture was evaporated in vacuo and the residue was partitioned between ethyl acetate and water. The organic phase was separated and then washed with brine. The organic phase was dried over magnesium sulfate and then evaporated. The residue was dissolved in a mixture of dimethylformamide and acetonitrile (1:3.8) and purified directly by preparative hplc eluting with a gradient of acetonitrile in water (containing 1% ammonia). The fractions containing product were evaporated to leave compound 24 in table 3 (0.051 g, 41% yield).

¹H NMR (300 MHz, DMSO): 2.17 (3H, s), 2.86 (4H, m), 4.53 (2H, d), 6.11 (1H, s), 6.24 (2H, s), 7.13-7.33 (6H, m), 7.83 (1H, d), 9.37 (1H, s), 11.93 (1H, s).

MS: m/z 376 (MH⁺).

2-chloro-N-(5-phenethyl-1H-pyrazol-3-yl)pyrimidin-4-amine, used as starting material, was prepared as follows:
a) In an analogous reaction to that described for example 24a but starting with ethyl 3-phenylpropanoate (17.83 g, 100 mmol) gave 5-phenethyl-1H-pyrazol-3-amine (6.47 g, 35% yield over 2 steps).

¹H NMR (300 MHz, DMSO): 2.65-2.90 (4H, m), 4.33 (2H, s), 7.15-7.30 (5H, m), 11.08 (1H, s).

MS: m/z 188 (MH⁺).
b) In an analogous reaction to that described for example 24b but starting with 5-phenethyl-1H-pyrazol-3-amine (2.25 g, 12.0 mmol) gave 2-chloro-N-(5-phenethyl-1H-pyrazol-3-yl)pyrimidin-4-amine (2.05 g, 68% yield).

¹H NMR (300 MHz, DMSO): 2.90 (4H, m), 6.08 (1H, s), 7.16-7.32 (6H, m), 8.16 (1H, d), 10.27 (0.5H, s), 12.21 (0.5H, s).

MS: m/z 300 (MH⁺).

(3-methyl-1,2-oxazol-5-yl)methanamine hydrochloride, used as starting material, was prepared as outlined in Example 1.

### Example 31

### N′-[5-[2-(2-methoxyphenyl)ethyl]-1H-pyrazol-3-yl]-N-[(3-methyl1,2-oxazol-5-yl)methyl]pyrimidine-2,4-diamine hydrochloride

Prepared using an analogous method to Example 3, but starting with 5-[2-(2-methoxyphenyl)ethyl]-1H-pyrazol-3-amine (78mg, 0.36 mmol) to give the title compound (51 mg, 32% yield)

1H NMR (300.132 MHz, DMSO) δ 2.17 (s, 3H), 2.84 (s, 4H), 3.78 (s, 3H), 4.69 (s, 2H), 6.18-6.44 (m, 3H), 6.84 (t, 1H), 6.95 (d, 1H), 7.09-7.12 (m, 1H), 7.15-7.21 (m, 1H), 7.87 (d, 1H). MS: m/z 406 (MH⁺)

5-[2-(2-methoxyphenyl)ethyl]-1H-pyrazol-3-amine, used as starting material, was prepared using an analogous method to Example 24a, but starting with methyl 3-(2-methoxyphenyl)propanoate (5 g, 25.7 mmol) to give 5-[2-(2-methoxyphenyl)ethyl]-1H-pyrazol-3-amine (3.6 g, 64%) as a golden oil.

1H NMR (300.132 MHz, CDCl3) 6 2.70-2.77 (m, 2H), 2.80-2.85 (m, 2H), 3.74 (s, 3H), 5.37 (s, 1H), 6.79 (t, 2H), 7.01 (d, 1H), 7.12 (t, 1H). MS: m/z 218 (MH⁺)

Methyl 3-(2-methoxyphenyl)propanoate, used as a starting material for the above intermediate, was prepared as follows:
a) 3-(2-Methoxyphenyl)propanoic acid (15 g, 83.3 mmol, 1 eq) was dissolved in methanol (100 ml) and conc sulphuric acid (0.5 ml) added. The solution was stirred at RT for 18 hours, then concentrated under reduced pressure. The residue was partitioned between water (150 ml) and EtOAc (200 ml) and the two phases separated. The organic phase was washed with water (2×100 ml), satd NaHCO₃ solution (2×50 ml), brine (1×50 ml) then dried over MgSO₄. This was then concentrated to give Methyl 3-(2-methoxyphenyl)propanoate as a colourless oil (14.2 g, 88%).

1H NMR (300.132 MHz, CDCl3) δ 2.53 (t, 2H), 2.86 (t, 2H), 3.58 (s, 3H), 3.74 (s, 3H), 6.75-6.82 (m, 2H), 7.05-7.14 (m, 2H).

### Example 32

### N′-[5-[2-(4-methoxyphenyl)ethyl]-2H-pyrazol-3-yl]-N-[(3-pyrimidin-2-yl-1,2-oxazol-5-yl)methyl]pyrimidine-2,4-diamine

2-chloro-N-[5-[2-(4-methoxyphenyl)ethyl]-2H-pyrazol-3-yl]pyrimidin-4-amine (100 mg, 0.30 mmol, 1 eq) and (3-pyrimidin-2-yl-1,2-oxazol-5-yl)methanamine. trifluoroacetic acid salt (68mg, 0.45 mmol, 1.5eq) were combined in 2-methoxyethanol (3 ml) containing diisopropylethylamine (159[l, 0.91 mmol, 3eq). The reaction was heated in the microwave at 170° C. for 1 h. The reaction was heated again at 170° C. for a further 2 h before the solvent was evaporated under reduced pressure. The crude product was dissolved in 1 ml of DMF and 3.8 ml of acetonitrile, filtered and then purified by basic reverse phase prep. eluting with a gradient of acetonitrile in water (both containing 1% ammonium hydroxide). The desired fractions were evaporated to give the title compound (0.0169 g, 12%).

¹H NMR (300.132 MHz, DMSO) δ 2.73-2.87 (4H, m),3.71 (3H, s),4.68 (2H, d), 6.30 (2H, m),6.80 (1H, s), 6.82 (2H, d), 7.12 (2H, d), 7.36 (1H, s), 7.59 (1H, t), 7.85 (1H, d), 8.93 (2H, d),9.43 (1H, s), 11.90 (1H, s) MS: M/Z 470 (MH+)

2-chloro-N-[5-[2-(4-methoxyphenyl)ethyl]-2H-pyrazol-3-yl]pyrimidin-4-amine, used as starting material was prepared as follows:—
a) Acetonitrile (2.09 ml, 40 mmol, 1.2 eq) was added to a slurry of sodium hydride (1.92 g, 48 mmol, 1.2 eq, 60% dispersion in mineral oil) in anhydrous toluene (30 ml) at 0° C. containing 3-(4-methoxyphenyl)-propionic acid methyl ester (7.77 g, 40 mmol, 1 eq). The reaction was stirred for 15 mins at 0° C. before heating to reflux for 2 h. After allowing to cool, the solvent was evaporated under reduced pressure. The residue was dissolved into water and acidified with 2M HCl and extracted with DCM. The DCM extracts were combined, washed with 2M HCl, followed by water and brine, dried with magnesium sulphate, filtered and evaporated under reduced pressure to yield a yellow oil which solidified on standing. 5-(4-Methoxyphenyl)-3-oxo-pentanenitrile was obtained as a crude off-white solid (2.09 g, 26%).

¹H NMR (300.132 MHz, DMSO) δ 2.77 (2H, m), 3.29 (4H, m), 3.72 (3H, s), 6.81-6.88 (2H, m), 7.08-7.16 (2H, m) MS: M/Z 202,(MH−)
b) 5-(4-Methoxyphenyl)-3-oxo-pentanenitrile (2.09 g, 10.30 mmol, 1 eq) and hydrazine hydrate (549 μl, 11.3 mmol, 1.1 eq) were combined in ethanol (25 ml) and refluxed for 3.5 h. The ethanol was evaporated and the residue crystallised on standing. This was extracted into ethyl acetate, washed with water and brine. The organic layer was dried with magnesium sulphate, filtered and evaporated under reduced pressure to afford 5-[2-(4-methoxyphenyl)ethyl]-2H-pyrazol-3-amine as an oil which solidified on standing (2.04 g, 97%)

¹H NMR (300.132 MHz, DMSO) δ 2.62-2.81 (4H, m), 3.72 (3H, s), 4.39 (1H, s), 5.17 (1H, s), 6.83 (2H, d), 7.12 (2H, d), 11.15 (1H, s) MS: M/Z 218,(MH⁺)
c) To 5-[2-(4-methoxyphenyl)ethyl]-2H-pyrazol-3-amine (2.02 g, 9.30 mmol, 1.2 eq) in ethanol (40 ml) was added N,N-diisopropylethylamine (2.7 ml, 15.5 mmol, 2 eq) followed by 2,4-dichloropyrimidine (1.155 g, 7.75 mmol, 1 eq). The reaction was heated at 50° C. for 70 h. The reaction was cooled then water added to yield an oily emulsion. The reaction was concentrated under reduced pressure to yield a precipitate. This was extracted into ethyl acetate and organic layer was washed with water and brine and dried over magnesium sulphate. The solvent was evaporated under reduced pressure to yield an oil which was triturated with DCM and a white solid was precipitated. This was filtered, washed with 50% ether/hexane and dried overnight to afford 2-chloro-N-[5-[2-(4-methoxyphenyl)ethyl]-2H-pyrazol-3-yl]pyrimidin-4-amine (1.50 g, 59%)

¹H NMR (300.132 MHz, DMSO) δ2.85 (4H, s), 3.72 (3H, s), 5.75 (1H, s), 6.09 (1H, s), 6.85 (2H, d), 7.15 (2H, d), 8.16 (1H, d), 10.26 (1H, s), 12.19 (1H, s) MS: M/Z 330,(MH+)

(3-Pyrimidin-2-yl-1,2-oxazol-5-yl)methanamine.TFA salt used as starting material was prepared as follows:—

To a solution of tert-butyl N-[(3-pyrimidin-2-yl-1,2-oxazol-5-yl)methyl]carbamate (9.27 g, 33.55 mmol) in DCM (220 ml) was added trifluoroacetic acid (24.9 ml, 335.5 mmol). The reaction mixture was stirred at room temperature overnight. The mixture was evaporated to dryness to give a clear brown oil. This was triturated with diethyl ether, resulting in a light-brown solid which was collected, washed with diethyl ether and dried in a desiccator at r.t. under high vacuum to constant weight. (3-Pyrimidin-2-yl-1,2-oxazol-5-yl)methanamine TFA salt was obtained as a light-brown solid (9.91 g). MS: m/z 176.86 (MH⁺).

tert-Butyl N-[(3-pyrimidin-2-yl-1,2-oxazol-5-yl)methyl]carbamate was prepared as follows:—

(NE)-N-(pyrimidin-2-ylmethylidene)hydroxylamine (15.4 g, 125.08 mmol) was suspended and stirred in the DCM (850 ml), tert-butyl N-prop-2-ynylcarbamate (38.81 g, 250.06 mmol) was added and the mixture cooled to 10° C. under nitrogen in an ice/water bath. 13% Aq. sodium hypochlorite solution (1 19.4 ml, 250.12 mmol) was added dropwise over 10 mins with vigorous stirring, the reaction mixture temperature never rising above 15° C. It was then allowed to warm to r.t. and stirred vigorously overnight. The reaction mixture was filtered through a bed of celite and the filtrate separated. The organic phase was washed with saturated brine, dried over MgSO₄ and evaporated to dryness giving a brown oil. The oil was dissolved in DCM and purified by column chromatography using EtOAc/isohexane 2:1. The appropriate fractions were combined and evaporated to yield tert-butyl N-[(3-pyrimidin-2-yl-1,2-oxazol-5-yl)methyl]carbamate (9.27 g, 13.4%). MS: m/z 277 (MH⁺)

(NE)-N-(pyrimidin-2-ylmethylidene)hydroxylamine was prepared as follows:—

2-(Diethoxymethyl)pyrimidine (53.46 g, 293.3 mmol) and hydroxylamine hydrochloride (24.46 g, 352.1 mmol) were dissolved in ethanol (500 ml) (containing water (50 ml)). The reaction mixture was stirred o/n at 60° C. The reaction mixture was neutralised with solid NaHCO₃ to pH 6 and then evaporated to dryness a brown solid. This was extracted on a sintered funnel and washed with 1:1 MeOH/DCM until all the product had been dissolved. All extracts were combined and evaporated to dryness affording a brown solid. The crude product was purified by column chromatography eluting with a gradient of 10-30% MeOH/DCM. The desired fractions were combined and evaporated giving a brown solid. This solid was triturated with diethyl ether, collected and dried in a desiccator at room temperature under high vacuum to constant weight. (NE)-N-(Pyrimidin-2-ylmethylidene)hydroxylamine was obtained as a brown solid (30.79 g, 85.2%). MS: m/z 124 (MH⁺)

2-(Diethoxymethyl)pyrimidine was prepared as follows:—

2,2-Diethoxy-acetamidinehydrochloride (71.43 g, 391.08 mmol) and 3-dimethylaminoacrolein (37.51 ml, 337.13 mmol) were dissolved in dry ethanol (440 ml).

The reaction mixture was brought to reflux in an oil bath and 25% wt. sodium methoxide solution (120.26 ml, 525.92 mmol) was then added dropwise over 50 mins and the resulting suspension stirred at reflux overnight. The reaction mixture was cooled to room temperature, filtered and the filtrate evaporated to dryness giving a thick brown cloudy oil. Purified by column chromatography using 50% EtOAc in isohexane as eluant. The appropriate fractions were combined and evaporated to give the desired product (53.46 g, 87%).

### Example 33

### N′-[5-[2-(3-methoxyphenyl)ethyl]-2H-pyrazol-3-yl]-N-[(3-pyrimidin-2-yl1,2-oxazol-5-yl)methyl]pyrimidine-2,4-diamine

Prepared in an analogous way to Example 27, but using (3-pyrimidin-2-yl-1,2-oxazol-5-yl)methanamine trifluoroacetic acid salt (176mg, 0.61 mmol, 2 eq) and N,N-diisopropylethylamine (212 μl, 1.21 mmol, 4 eq) to give the title compound (0.0245 g, 16% yield).

1H NMR (300.132 MHz, DMSO): δ 2.77-2.92 (m, 4H), 3.72 (s, 3H), 4.68 (d, 2H), 6.27 (s, 2H), 6.70-6.86 (m, 4H), 7.17 (t, 1H), 7.35 (s, 1H), 7.59 (t, 1H), 7.86 (d, 1H), 8.93 (d, 2H), 9.42 (s, 1H), 11.93 (s, 1H). MS: m/z 470 (MH+).

2-chloro-N-[5-[2-(3-methoxyphenyl)ethyl]-1H-pyrazol-3-yl]pyrimidin-4-amine, used as starting material, was prepared as outlined in Example 27.

(3-Pyrimidin-2-yl-1,2-oxazol-5-yl)methanamine.TFA salt was synthesized as outlined in Example 32.

### Example 34

### N-[(3-methyl-1,2-oxazol-5-yl)methyl]-N′-[5-(phenoxymethyl)-2H-pyrazol-3-yl]pyrimidine-2,4-diamine

2-Chloro-N-[5-(phenoxymethyl)-2H-pyrazol-3-yl]pyrimidin-4-amine (80 mg, 0.25 mmol, 1.0 eq) and (3-methyl1,2-oxazol-5-yl)methanamine (53mg, 0.35 mmol, 1.4 eq) and N,N-diisopropylethylamine (110 μl, 0.63 mmol, 2.5 eq) were combined in 2-methoxyethanol (4 ml) and heated in a microwave to 200° C. for 1 h. The mixture was concentrated and the residue purified by preparative HPLC (basic system, gradient 15-45% acetonitrile in water containing 1% ammonium hydroxide). Concentration of the product-containing fractions gave the title compound (13mg, 14%) as a white solid.

¹H NMR (499.803 MHz, DMSO) δ 2.15 (s, 3H), 4.58 (s, 2H), 5.03 (s, 2H), 6.08 (s, 1H), 6.24-6.26 (m, 2H), 6.91 (t, 1H), 6.99 (d, 2H), 7.25 (t, 2H), 7.85 (d, 1H), 8.06 (s, 1H) MS: m/z 378 (MH+).

(3-methyl-1,2-oxazol-5-yl)methanamine was synthesized as outlined in Example 1.

2-Chloro-N-[5-(phenoxymethyl)-2H-pyrazol-3-yl]pyrimidin-4-amine used as a starting material was prepared as follows:
a) To a stirred slurry of 60% NaH in mineral oil (2.89 g, 72.2 mmol, 1.2 eq) in dry 1,4-dioxane (100 ml) containing acetonitrile (3.78 ml, 72.2 mmol, 1.2 eq), at room temperature under N₂, was added methyl 2-phenoxyacetate (10 g, 60.2 mmol, 1 eq). The reaction mixture was refluxed for 24 h. Water was added (3 drops) and the mixture concentrated to dryness, dissolved in water (120 ml) and washed with DCM (3×120 ml). The aqueous layer was carefully acidified to approx pH1-3 using conc HCl and extracted with DCM (4×120 ml). The combined organic extracts were dried over MgSO₄ and concentrated to dryness. The residue was dissolved in ethanol (80 ml) and hydrazine hydrate (5.85 ml, 120.4 mmol, 2 eq) added. The mixture was heated to reflux for 18 h. After this time the solution was concentrated to dryness, washed onto a pre-equilibrated SCX-2 column using methanol. 2% Ammonium hydroxide in methanol was used to liberate the product and the product containing fractions combined and concentrated to give 5-(phenoxymethyl)-1H-pyrazol-3-amine as a white solid (2.7 g, 24%).

¹H NMR (300.132 MHz, DMSO) δ 5.13 (s, 2H), 6.12 (s, 1H), 6.95-7.04 (m, 3H), 7.28-7.34 (m, 2H). MS: m/z 190 (MH+)
b) 5-(phenoxymethyl)-1H-pyrazol-3-amine (1 g, 4.44 mmol, 1 eq), 2,4-dichloropyrimidine (663 mg, 4.44 mmol, 1 eq) and N,N-diisopropylethylamine (1.94 ml, 4.44 mmol, 2.5 eq) were combined in ethanol (25 ml) at room temperature. The mixture was warmed to 40° C. and stirred at this temp for 8 d. The mixture was poured into cold water (100 ml) and the precipitate filtered, washed thoroughly with water and dried under vacuum to give 2-chloro-N-[5-(phenoxymethyl)-2H-pyrazol-3-yl]pyrimidin-4-amine as a brown solid (490mg, 37%).

¹H NMR (300.132 MHz, DMSO) δ 5.09 (s, 2H), 6.45 (s, 1H), 6.92-7.06 (m, 4H), 7.32 (t, 2H), 8.18 (d, 1H), 10.40 (s, 1H), 12.70 (s, 1H). MS: m/z 302 (MH+)

### Example 35

### N-[(3-cyclopropyl1,2-oxazol-5-yl)methyl]-N′-[5-(phenoxymethyl)-2H-pyrazol-3-yl]pyrimidine-2,4-diamine

Prepared in an analogous way to Example 34, using (3-cyclopropylisoxazol-5-yl)methanamine (also known as (3-cyclopropyl-1,2-oxazol-5-yl)methanamine; 62 mg, 0.35 mmol) and 2-chloro-N-[5-(phenoxymethyl)-2H-pyrazol-3-yl]pyrimidin-4-amine (80 mg, 0.25 mmol) to give the title compound (12 mg, 12%) as a white solid.

1H NMR (499.803 MHz, DMSO) δ 0.67-0.70 (m, 2H), 0.90-0.94 (m, 2H), 1.88-1.92 (m, 1H), 4.55 (s, 2H), 5.03 (s, 2H), 5.99 (s, 1H), 6.24-6.27 (m, 2H), 6.91 (t, 1H), 6.99 (d, 2H), 7.25 (t, 2H), 7.85 (d, 1H). MS: m/z 404 (MH+).

(3-Cyclopropylisoxazol-5-yl)methanamine (also known as (3-cyclopropyl-1,2-oxazol-5-yl)methanamine), used as starting material, can be prepared as outlined in Example 3.

### Example 36

### 5-[[[4-[[5-(phenoxymethyl)-2H-pyrazol-3-yl]amino]pyrimidin-2-yl]amino]methyl]1,2-oxazole-3-carboxamide

Prepared in an analogous way to Example 35, using 5-(aminomethyl)1,2-oxazole-3-carboxamide (63 mg, 0.35 mmol, 1.4 eq) and chloro-N-[5-(phenoxymethyl)-2H-pyrazol-3-yl]pyrimidin-4-amine (80 mg, 0.35 mmol, 1 eq) to give the title compound (32 mg, 32%) as a white solid.

¹H NMR (499.803 MHz, DMSO) δ 4.66 (s, 2H), 5.03 (s, 2H), 6.25-6.29 (m, 2H), 6.53 (s, 1H), 6.91 (t, 1H), 6.99 (d, 2H), 7.25 (t, 2H), 7.86 (d, 1H) MS: m/z 407 (MH+).

5-(Aminomethyl)-1,2-oxazole-3-carboxamide, used as starting material, can be prepared as outlined in Example 4.

### Example 37

### N-[(3-methyl1,2-oxazol-5-yl)methyl]-N′-[5-[2-(4-phenylmethoxyphenyl)ethyl]-2H-pyrazol-3-yl]pyrimidine-2,4-diamine

A mixture of 5-[2-(4-phenylmethoxyphenyl)ethyl]-2H-pyrazol-3-amine (114 mg, 0.39 mmol, 1.3 eq) and 4-chloro-N-[(3-methyl-1,2-oxazol-5-yl)methyl]pyrimidin-2-amine (67 mg, 0.30 mmol, 1 eq) in ethanol (10 ml) (containing a few drops of 4M HCl in dioxane) was refluxed for 18 h to yield a pale yellow solution. The solvent was evaporated under reduced pressure. The crude product was purified on acidic reverse phase prep. HPLC using a 35-45% gradient of acetonitrile in water containing 0.2% TFA. The desired fractions were poured onto a SCX-2 column which had been pre-wet with methanol. After washing several times with methanol the product was finally eluted with 10% ammonium hydroxide solution in methanol. Evaporation under reduced pressure afforded the title compound as a white solid (34.7 mg, 19% yield).

1H NMR (300.132 MHz, DMSO): 6 2.16 (s, 3H), 3.22-3.37 (m, 4H), 4.57 (d, 2H), 5.06 (s, 2H), 6.15 (s, 1H), 6.15-6.40 (m, 1H), 6.92 (d, 2H), 7.14 (d, 2H), 7.30-7.55 (m, 6H), 7.57-7.73 (m, 1H), 7.84 (d, 1H), 9.86 (s, 1H), 12.03 (s, 1H). MS: m/z 482 (MH+).

5-[2-(4-Phenylmethoxyphenyl)ethyl]-2H-pyrazol-3-amine, used as starting material was prepared in a similar way to 5-[2-(3-methoxyphenyl)ethyl]-2H-pyrazol-3-amine in Example 27a, but using ethyl 3-(4-phenylmethoxyphenyl)propanoate as a starting material. The desired compound was obtained as a yellow solid (1.08 g, 25% yield).

MS: m/z 482 (MH+) 294.

4-chloro-N-[(3-methyl-1,2-oxazol-5-yl)methyl]pyrimidin-2-amine was synthesized as outlined in Example 13.

### Example 38

### N-[(3-methyl1,2-oxazol-5-yl)methyl]-N′-[5-[2-(3-phenylmethoxyphenyl)ethyl]-2H-pyrazol-3-yl]pyrimidine-2,4-diamine

A mixture of 5-[2-(3-phenylmethoxyphenyl)ethyl]-2H-pyrazol-3-amine (152 mg, 0.52 mmol, 1 eq) and 4-chloro-N-[(3-methyl-1,2-oxazol-5-yl)methyl]pyrimidin-2-amine (117 mg, 0.52 mmol, 1 eq) in ethanol (8 ml) (containing a few drops of 4M HCl in dioxane) was heated at 80° C. in a glass tube for 18 h. The precipitated product was filtered, washed with ethanol and dried. The product was suspended in water and basified by the addition of ammonium hydroxide solution. The product was extracted into ethyl acetate and the organic layer separated. The organic layer was washed with saturated sodium hydrogen carbonate, washed with brine, dried with magnesium sulphate, filtered and evaporated under reduced pressure to give the title compound as a solid. (129.7 mg, 52% yield)

¹H NMR (300.132 MHz, DMSO): δ 2.16 (s, 3H), 2.81-2.90 (m, 4H), 4.53 (d, 2H), 5.06 (s, 2H), 6.09 (s, 1H), 6.28 (s, 2H), 6.80-6.86 (m, 2H), 6.90 (s, 1H), 7.20 (t, 2H), 7.28-7.46 (m, 5H), 7.82 (d, 1H), 9.34 (s, 1H), 11.91 (s, 1H). MS: m/z 482 (MH+).

5-[2-(3-Phenylmethoxyphenyl)ethyl]-2H-pyrazol-3-amine, used as starting material was prepared in a similar way to 5-[2-(3-methoxyphenyl)ethyl]-2H-pyrazol-3-amine in Example 27, but using benzyl 3-(3-phenylmethoxyphenyl)propanoate as a starting material. The desired compound was obtained as a yellow oil (2.45 g, 40% yield).

¹H NMR (300.132 MHz, DMSO): δ 2.68-2.84 (m, 4H), 4.42 (s, 2H), 5.07 (s, 2H), 5.19 (s, 1H), 6.77-6.90 (m, 3H), 7.18 (t, 1H), 7.29-7.48 (m, 5H). MS: m/z 294 (MH+).

4-chloro-N-[(3-methyl-1,2-oxazol-5-yl)methyl]pyrimidin-2-amine was synthesized as outlined in Example 13.

### Example 39

### N-[(3-methyl1,2-oxazol-5-yl)methyl]-N′-[5-[2-(2-phenylmethoxyphenyl)ethyl]-1H-pyrazol-3-yl]pyrimidine-2,4-diamine hydrochloride

Prepared using an analogous method to Example 37, but using 5-[2-(2-phenylmethoxyphenyl)ethyl]-1H-pyrazol-3-amine (105 mg, 0.36 mmol) as a starting material, to give the title compound (118 mg, 63% yield)

1H NMR (300.132 MHz, DMSO) δ 2.13 (s, 3H), 2.84-2.95 (m, 4H), 4.65 (s, 2H), 5.13 (s, 2H), 6.17-6.31 (m, 2H), 6.38 (s, 1H), 6.85 (t, 1H), 7.03 (d, 1H), 7.10-7.19 (m, 2H), 7.28-7.40 (m, 3H), 7.46 (d, 2H), 7.88 (d, 1H). MS: m/z 482 (MH+)

5-[2-(2-phenylmethoxyphenyl)ethyl]-1H-pyrazol-3-amine, used as a starting material, was prepared in a similar way to Example 34a, but using methyl 3-(2-phenylmethoxyphenyl)propanoate (3.9 g, 14.4 mmol) as a starting material to give 5-[2-(2-phenylmethoxyphenyl)ethyl]-1H-pyrazol-3-amine (1.6 g, 38%) as a brown gum. MS: m/z 294 ((M−H)⁻)

Methyl 3-(2-phenylmethoxyphenyl)propanoate was prepared using a method analogous to Example 31, using 3-(2-benzyloxyphenyl)propionic acid (7 g, 27.3 mmol) to give methyl 3-(2-phenylmethoxyphenyl)propanoate (6.66 g, 90%) as a colourless oil.

1H NMR (300.132 MHz, CDCl3) δ 2.65 (t, 2H), 3.01 (t, 2H), 3.64 (s, 3H), 5.08 (s, 2H), 6.86-6.90 (m, 2H), 7.13-7.18 (m, 2H), 7.28-7.43 (m, 5H).

### Example 40

### N′-[5-[2-[3-(2-methoxyethoxy)phenyl]ethyl]-2H-pyrazol-3-yl]-N-[(3-methyl1,2-oxazol-5-yl)methyl]pyrimidine-2,4-diamine

Prepared in an analogous way to example 38, but starting with 5-[2-[3-(2-methoxyethoxy)phenyl]ethyl]-2H-pyrazol-3-amine (136 mg, 0.52 mmol, 1 eq). Isolated as a solid (124.8 mg, 53% yield).

1H NMR (300.132 MHz, DMSO): δ 2.00 (s, 3H), 2.64-2.73 (m, 4H), 3.13 (s, 3H), 3.47 (t, 2H), 3.89 (t, 2H), 4.36 (d, 2H), 5.94 (s, 1H), 6.09 (s, 2H), 6.55-6.67 (m, 3H), 7.01 (t, 2H), 7.66 (d, 1H), 9.17 (s, 1H), 11.73 (s, 1H). MS: m/z 450 (MH+).

5-[2-[3-(2-methoxyethoxy)phenyl]ethyl]-2H-pyrazol-3-amine used as starting material was prepared from 2-methoxyethyl 3-[3-(2-methoxyethoxy)phenyl]propanoate in a similar manner to 5-[2-(3-methoxyphenyl)ethyl]-2H-pyrazol-3-amine in example 27a. Isolated as yellow oil (2.78 g, 81% yield).

1H NMR (300.132 MHz, DMSO): δ 2.68-2.84 (m, 4H), 3.31 (s, 3H), 3.65 (dd, 2H), 4.06 (dd, 2H), 4.40 (s, 2H), 5.19 (s, 1H), 6.71-6.81 (m, 3H), 7.17 (t, 1H), 11.08 (s, 1H). MS: m/z 262 (MH+).

### Example 41

### 3-[2-[5-[[2-[(3-methyl1,2-oxazol-5-yl)methylamino]pyrimidin-4-yl]amino]-1H-pyrazol-3-yl]ethyl]phenol

To a stirred solution of N′-[5-[2-(3-methoxyphenyl)ethyl]-2H-pyrazol-3-yl]-N-[(3-methylisoxazol-5-yl)methyl]pyrimidine-2,4-diamine (also known as N′-[5-[2-(3-methoxyphenyl)ethyl]-2H-pyrazol-3-yl]-N-[(3-methyl-1,2-oxazol-5-yl)methyl]pyrimidine-2,4-diamine; 100 mg, 0.25 mmol, 1 eq) in DCM (10 ml) at 0° C. under N₂ was slowly added a 1M solution of boron tribromide (1.52 ml, 1.52 mmol, 5 eq). The reaction was allowed to warm to r.t. overnight. The reaction mixture was cooled in ice and methanol was slowly added (5 ml) to yield a pale yellow solution. The solution was evaporated under reduced pressure. After basifying, the product was purified on the basic reverse phase prep. HPLC using a 20-40% gradient of acetonitrile in water containing 1% ammonia in the aqueous eluent. The clean fractions were taken and evaporated under reduced pressure to low volume. The precipitate that formed was filtered, washed with water and dried in a vacuum dessicator overnight at 60° C. to afford the title compound as a pale pink solid (59 mg, 60% yield).

1H NMR (300.132 MHz, DMSO): δ 2.17 (s, 3H), 2.80 (s, 4H), 4.53 (d, 2H), 6.10 (s, 1H), 6.17-6.36 (m, 2H), 6.55-6.68 (m, 3H), 7.07 (t, 1H), 7.18 (t, 1H), 7.82 (d, 1H), 9.23 (s, 1H), 9.34 (s, 1H), 11.91 (s, 1H). MS: m/z 392 (MH+)

N′-[5-[2-(3-methoxyphenyl)ethyl]-2H-pyrazol-3-yl]-N-[(3-methylisoxazol-5-yl)methyl]pyrimidine-2,4-diamine (also known as N′-[5-[2-(3-methoxyphenyl)ethyl]-2H-pyrazol-3-yl]-N-[(3-methyl-1,2-oxazol-5-yl)methyl]pyrimidine-2,4-diamine), used as starting material was prepared by method outlined in Example 27 (678 mg, 47% yield).

1H NMR (300.132 MHz, DMSO): δ 2.16 (s, 3H), 2.81-2.90 (m, 4H), 3.73 (s, 3H), 4.53 (d, 2H), 6.10 (s, 1H), 6.17-6.44 (m, 2H), 6.72-6.84 (m, 3H), 7.19 (t, 1H), 7.19 (s, 1H), 7.82 (d, 1H), 9.34 (s, 1H), 11.90 (s, 1H). MS: m/z 392 (MH+)

### Example 42

### N′-[5-[2-(3,5-dimethoxyphenyl)ethyl]-2H-pyrazol-3-yl]-N-[(3-methyl1,2-oxazol-5-yl)methyl]pyrimidine-2,4-diamine

Prepared in an analogous way to example 37, but using 5-[2-(3,5-dimethoxyphenyl)ethyl]-2H-pyrazol-3-amine (124 mg, 0.42 mmol, 1.3 eq) in ethanol (5 ml). After purification (using a 25-45% gradient of acetonitrile in water containing 1% ammonium hydroxide), the fractions were evaporated to low volume. A white solid precipitated which was filtered, washed with water and dried overnight to give the title compound as a white solid (67 mg, 49% yield).

1H NMR (300.132 MHz, DMSO): δ 2.16 (s, 3H), 2.83 (s, 4H), 3.71 (s, 6H), 4.52 (d, 2H), 6.09 (s, 1H), 6.17-6.36 (m, 3H), 6.41 (m, 2H), 7.13-7.23 (m, 1H), 7.82 (d, 1H), 9.34 (s, 1H), 11.89 (s, 1H). MS: m/z 436 (MH+).

5-[2-(3,5-dimethoxy)ethyl]-2H-pyrazol-3-amine, used as starting material was prepared as follows:
a) Acetonitrile (2.29 ml, 43.61 mmol, 1.2 eq) was added to a slurry of sodium hydride (1.75 g dispersion in mineral oil, 43.61 mmol, 1.2 eq) in anhydrous toluene (70 ml) and the mixture stirred at room temperature for 30 minutes. Ethyl 3-(3,5-dimethoxyphenyl)propanoate (8.66 g, 36.34 mmol, 1 eq) in toluene (60 ml) was added and the reaction was refluxed for 18 h. After cooling and quenching with a small amount of water, the solvent was evaporated under reduced pressure. The residue was dissolved in 2M HCl (50 ml). The acidic solution was then extracted twice with ethyl acetate. The organic extracts were combined, washed with water, followed by brine and finally dried over magnesium sulphate. After filtering, the solvent was evaporated under reduced pressure to yield the crude product as a yellow oil. The oil was purified by column chromatography and the product eluted with DCM. Fractions containing clean product were combined and evaporated to yield a cream solid. (3.76 g, 44% yield). To the solid (3.72 g, 15.96 mmol, 1 eq) in ethanol (55 ml) was added hydrazine hydrate (852 μl, 17.56 mmol, 1.1 eq). The reaction was refluxed for 24 h before allowing to cool. After evaporating under reduced pressure, the residue was dissolved in dichloromethane, washed with water, followed by brine, dried with magnesium sulphate, filtered and evaporated under reduced pressure to afford 5-[2-(3,5-dimethoxyphenyl)ethyl]-2H-pyrazol-3-amine as a pale yellow solid (3.76 g. 42% over 2 steps).

¹H NMR (300.132 MHz, DMSO) δ 2.64-2.82 (4H, m),3.71 (6H, s),4.07-4.72 (2H, m), 5.20 (1H, s), 6.31 (1H, t), 6.38 (2H, d). MS: m/z 248 (MH+)

### Example 43

### 5-[2-[5-[[2-[(3-methyl1,2-oxazol-5-yl)methylamino]pyrimidin-4-yl]amino]-1H-pyrazol-3-yl]ethyl]benzene-1,3-diol

To a stirred solution of N′-[5-[2-(3,5-dimethoxyphenyl)ethyl]-2H-pyrazol-3-yl]-N-[(3-methyl1,2-oxazol-5-yl)methyl]pyrimidine-2,4-diamine (0.488 g, 1. 12 mmol) in dichloromethane (30 ml) at 0° C. under nitrogen, boron tribromide solution (1M in DCM, 5.6 ml, 5.6 mmol) was added slowly. The reaction was allowed to warm to r.t. overnight. After this time, a pale pink precipitate had formed. The reaction mixture was cooled in ice and methanol was slowly added to yield a pale yellow solution. The solution was evaporated under reduced pressure to yield a grey solid. The residue was dissolved into water and basified to pH 8 by the addition of ammonium hydroxide solution. The aqueous layer was extracted with ethyl acetate, washed with 20% aqueous ammonia, water and finally brine. It was then dried with magnesium sulphate, filtered, and evaporated under high vacuum to yield a cream solid (0.1927 g, 42%)

¹H NMR (500.13 MHz, DMSO-d₆) δ 2.19 (3H, s), 2.74-2.82 (4H, m), 4.59 (2H, d), 6.08-6.09 (2H, m), 6.09 (1H, s), 6.10-6.12 (2H, d), 6.14 (1H, d), 6.8 (1H, s), 7.86 (1H, d), 8.62 (2H, s), 8.90 (1H, s), 11.20 (1H, s); MS: m/z 408.53 (MH⁺)

N′-[5-[2-(3,5-dimethoxyphenyl)ethyl]-2H-pyrazol-3-yl]-N-[(3-methyl1,2-oxazol-5-yl)methyl]pyrimidine-2,4-diamine, used as starting material was prepared as follows:

A mixture of 5-[2-(3,5-dimethoxyphenyl)ethyl]-2H-pyrazol-3-amine (619 mg, 2.5 mmol), 4-chloro-N-[(3-methyl1,2-oxazol-5-yl)methyl]pyrimidin-2-amine (562 mg, 2.5 mmol), and ethanol (15 ml) were stirred and heated at 80° C. for 18 hours. The precipitate was filtered and washed with ice cold ethanol and then washed with ether to give the product (0.9898 g, 91%).

5-[2-(3,5-Dimethoxyphenyl)ethyl]-2H-pyrazol-3-amine was prepared as outlined in Example 42.

### Example 44

### N′-[5-[(3,5-Dimethoxyphenoxy)methyl]-2H-pyrazol-3-yl]-N-[(3-methyl1,2-oxazol-5-yl)methyl]pyrimidine-2,4-diamine

4-Chloro-N-[(3-methyl1,2-oxazol-5-yl)methyl]pyrimidin-2-amine (80 mg, 0.36 mmol, 1 eq) and 5-[(3,5-dimethoxyphenoxy)methyl]-1H-pyrazol-3-amine (127 mg, 0.51 mmol, 1.4 eq) were combined in ethanol (5 ml) and heated to 80° C. for 18 hours. After this time the solution was basified using ammonium hydroxide and purifed by prep HPLC (basic system, gradient 20-40% acetonitrile in water containing 1% ammonium hydroxide). The desired fractions were combined and concentrated to give N′-[5-[(3,5-Dimethoxyphenoxy)methyl]-2H-pyrazol-3-yl]-N-[(3-methyl1,2-oxazol-5-yl)methyl]pyrimidine-2,4-diamine (73 mg, 46%) as a white solid.

¹H NMR (300.132 MHz, DMSO) δ 2.16 (s, 3H), 3.71 (s, 6H), 4.54 (s, 2H), 4.98 (s, 2H), 6.11 (t, 1H), 6.13 (s, 1H), 6.20-6.20 (m, 3H), 6.31 (s, 1H), 7.87 (d, 1H). MS m/z 438 (MH+)

5-[(3,5-Dimethoxyphenoxy)methyl]-1H-pyrazol-3-amine used as a starting material above was made in an analogous way to example 42 using methyl 2-(3,5-dimethoxyphenoxy)acetate. as a starting material (1.7 g, 30%).

¹H NMR (300.132 MHz, DMSO) δ 3.70 (s, 6H), 5.08 (s, 2H), 6.13 (t, 2H), 6.18 (s, 1H), 6.19 (s, 1H). MS: m/z 250 (MH+)
a) Methyl 2-(3,5-dimethoxyphenoxy)acetate, used as starting material above, was made as follows:

3,5-Dimethoxyphenol (5 g, 32.4 mmol, 1 eq), N,N-diisopropylamine (6.78 ml, 38.9 mmol, 1.2 eq) and methyl bromoacetate (5.46 g, 35.7 mmol, 1.1 eq) were combined in DCM (100 ml) and the mixture heated to reflux (T=50° C.) for 18 hours. After this time the solution was cooled and washed with 2M HCl (3×40 ml), saturated aqueous NaHCO₃ solution (3×40 ml), then brine (2×40 ml), dried (MgSO₄) and concentrated to methyl 2-(3,5-dimethoxyphenoxy)acetate (5.19 g, 71%) as a colourless oil.

¹H NMR (300.132 MHz, DMSO) δ 3.70 (s, 3H), 3.71 (s, 6H), 4.75 (s, 2H), 6.08-6.14 (m, 3H).

Chloro-N-[(3-methyl1,2-oxazol-5-yl)methyl]pyrimidin-2-amine, used as starting material, was prepared as follows:—

(3-Methyl-1,2-oxazol-5-yl)methanamine (9.3 g, 83 mmol) and 2-methylsulfonylpyrimidin-4-ol (9.8 g, 69 mmol) were heated together at 160° C. for 4 h. The mixture was allowed to cool then dissolved in dichloromethane and purified by column chromatography eluting with 5-15% methanol in dichloromethane to give the desired product as a brown gum (8.88 g, 62%).

1H NMR (DMSO) δ 2.19 (s, 3H), 4.57 (s, 2H), 5.6 (d, 1H), 6.19 (s, 1H), 7.03 (bs, 1H), 7.61 (d, 1H), 11 (bs, 1H); MS: m/z 207 (MH+)

(3-methyl-1,2-oxazol-5-yl)methanamine, used as starting material, was prepared as outlined in Example 1.

### Example 45

### N′-[5-[2-(2,5-dimethoxyphenyl)ethyl]-2H-pyrazol-3-yl]-N-[(3-methyl1,2-oxazol-5-yl)methyl]pyrimidine-2,4-diamine

A mixture of 5-[2-(2,5-dimethoxyphenyl)ethyl]-2H-pyrazol-3-amine (0.248 g, 1 mmol), 4-chloro-N-[(3-methyl1,2-oxazol-5-yl)methyl]pyrimidin-2-amine (0.225 g, 1 mmol), and ethanol (5 ml) was stirred and heated at 80° C. o/n under an inert atmosphere. A yellow precipitate formed. The suspension was allowed to cool to room temperature, filtered and washed with ice-cold ethanol (30 ml) and ether (20 ml) to give a pale yellow precipitate (0.354 g, 81%).

¹H NMR (399.9 MHz, DMSO-d₆) δ2.19 (3H, s), 2.82 (4H, s), 3.67 (3H, s), 3.74 (3H, s), 4.72 (2H, d), 6.28-6.38 (2H, d), 6.75 (2H, q), 6.87-6.90 (1H, m), 7.90 (1H, s), 8.88 (1H,s), 11.25 (1H, s), 12.45-12.75 (2H, d)

MS: m/z 436 (MH⁺)

5-[2-(2,5-Dimethoxyphenyl)ethyl]-2H-pyrazol-3-amine, used as starting material, was prepared as follows:

Sodium hydride (60%, 0.240 g, 6 mmol) was added to a stirred solution of methyl 3-(2,5-dimethoxyphenyl)propanoate (1.125 g, 5 mmol) in 1,4 dioxane (25 ml) in dry acetonitrile (0.314 ml, 6 mmol) under nitrogen. The mixture was stirred at r.t for 10 mins then heated at reflux under nitrogen for 18 h. After this time, the mixture was cooled to r.t. upon which a precipitate formed. Ethanol (2 ml) was added, followed by hydrazine monohydrochloride (0.686 g, 10 mmol). The mixture was heated to reflux for 4 h. In this time, the precipitate went into solution and a solid appeared. After filtration, the reaction mixture was concentrated in vacuo and partitioned between 2N HCl and ethyl acetate (25 ml each). The aqueous layer was basified with ammonium hydroxide solution to pH 8, extracted with ethyl acetate and dried with MgSO4. This was filtered, and the solvents were evaporated in vacuo to give an orange oil (0.690 g, 56%).

Chloro-N-[(3-methyl1,2-oxazol-5-yl)methyl]pyrimidin-2-amine, used as starting material, was prepared as outlined in Example 44.

(3-methyl-1,2-oxazol-5-yl)methanamine, used as starting material, was prepared as outlined in Example 1.

### Example 46

### N′-[5-[2-(3,4-dimethoxyphenyl)ethyl]-1H-pyrazol-3-yl]-N-[(3-methyl1,2-oxazol-5-yl)methyl]pyrimidine-2,4-diamine hydrochloride

4-Chloro-N-[(3-methyl1,2-oxazol-5-yl)methyl]pyrimidin-2-amine (80 mg, 0.36 mmol, 1 eq) and 5-[2-(3,4-dimethoxyphenyl)ethyl]-1H-pyrazol-3-amine (89 mg, 0.36 mmol, 1 eq) were combined in ethanol (5 ml) and heated to 80° C. for 24 h. The mixture was cooled to r.t. and the precipitate collected by filtration, washed with ice-cold ethanol, ether and dried under vacuum to give N′-[5-[2-(3,4-dimethoxyphenyl)ethyl]-1H-pyrazol-3-yl]-N-[(3-methyl1,2-oxazol-5-yl)methyl]pyrimidine-2,4-diamine hydrochloride (82 mg, 48%) as an off-white solid.

1H NMR (300.132 MHz, DMSO) δ 2.18 (s, 3H), 2.83 (s, 4H), 3.71 (s, 3H), 3.72 (s, 3H), 4.68 (s, 2H), 6.20 (s, 1H), 6.26 (s, 1H), 6.38 (s, 1H), 6.69-6.72 (m, 1H), 6.80-6.84 (m, 2H), 7.87 (d, 1H). MS: m/z 436 (MH+)

Chloro-N-[(3-methyl1,2-oxazol-5-yl)methyl]pyrimidin-2-amine, used as starting material, was prepared as outlined in Example 44.

5-[2-(3,4-dimethoxyphenyl)ethyl]-1H-pyrazol-3-amine, used as starting material, was prepared in a method analogous to that in example 42 using methyl 3-(3′,4′-dimethoxyphenyl)propanoate (5 g, 22.3 mmol) as starting material to give 5-[2-(3,4-dimethoxyphenyl)ethyl]-1H-pyrazol-3-amine (2.2 g, 40% yield) as a golden oil. MS: m/z 248 (MH+).

### Example 47

### N′-[5-[2-(4-methoxy-2-methyl-phenyl)ethyl]-2H-pyrazol-3-yl]-N-[(3-methyl1,2-oxazol-5-yl)methyl]pyrimidine-2,4-diamine

A mixture of 5-[2-(4-methoxy-2-methyl-phenyl)ethyl]-2H-pyrazol-3-amine (0.232 g, 1 mmol), 4-chloro-N-[(3-methyl1,2-oxazol-5-yl)methyl]pyrimidin-2-amine (0.225 g, 1 mmol), and ethanol (5 ml) was stirred and heated at 80° C. for 6 h. The yellow needle-like crystals were filtered and washed with ice-cold ethanol and then washed with ether to give the final product (0.215 g, 51%).

¹H NMR (399.9 MHz, DMSO-d₆) δ2.19 (3H, s), 2.25 (3H, s), 2.79 (4H, s), 3.71 (3H, s), 4.70-4.72 (2, m), 6.28 (2H, s), 6.67-6.70 (1H, m), 6.74 (1H, d), 7.05 (1H, d), 7.89-7.91 (1H, m), 8.76-8.9 (1H, s), 11.18-11.32 (1H, s), 12.39-12.50 (1H, s), 12.57-12.75 (1H, s)

MS: m/z 420.49 (MH⁺)

4-Chloro-N-[(3-methyl1,2-oxazol-5-yl)methyl]pyrimidin-2-amine, used as starting material, was prepared as outlined in Example 44.

5-[2-(4-methoxy-2-methyl-phenyl)ethyl]-2H-pyrazol-3-amine, used as starting material, was prepared in a method analogous to that in example 42 using methyl 3-(4-methoxy-2-methyl-phenyl)propanoate as starting material to give 5-[2-(4-methoxy-2-methyl-phenyl)ethyl]-2H-pyrazol-3-amine as a red solid. MS: m/z 232 (MH+).

### Example 48

### 3-[2-[5-[[2-[(3-methyl-1,2-oxazol-5-yl)methylamino]pyrimidin-4-yl]amino]-2H-pyrazol-3-yl]ethyl]benzonitrile

A mixture of 3-[2-(5-amino-2H-pyrazol-3-yl)ethyl]benzonitrile (128 mg, 0.6 mmol), 4-chloro-N-[(3-methylisoxazol-5-yl)methyl]pyrimidin-2-amine (also known as 4-chloro-N-[(3-methyl-1,2-oxazol-5-yl)methyl]pyrimidin-2-amine; 135 mg, 0.6 mmol) and ethanol (5 ml) was heated at reflux for 18 h. The reaction mixture was cooled and the crystallized solid was filtered off, washed with ethanol and diethyl ether to afford the title compound as a solid (179 mg, 74.5%). ¹H NMR (399.9 MHz, DMSO-d₆) δ2.19 (3H, s), 2.86-3.02 (4H, m), 4.70-4.71 (2H, m), 6.29 (1H, s), 6.38 (1H, s), 7.50 (1H, t), 7.56 (1H, d), 7.66-7.70 (2H, m), 7.91 (1H, s), 8.86 (1H, s), 11.24 (1H, s), 12.42 (1H, s), 12.74 (1H, s). MS: m/z 401 (MH+).

4-Chloro-N-[(3-methyl1,2-oxazol-5-yl)methyl]pyrimidin-2-amine, used as starting material, was prepared as outlined in Example 44.

3-[2-(5-amino-2H-pyrazol-3-yl)ethyl]benzonitrile_used as starting material was prepared as outlined in Example 42 for 5-[2-(3,5-dimethoxy) ethyl]-2H-pyrazol-3-amine, starting from methyl-3-(3-cyanophenyl)propanoate (880 mg, 4.66 mmol) as starting material. 3-[2-(5-amino-2H-pyrazol-3-yl)ethyl]benzonitrile was obtained as an oil (256 mg, 26%). MS: m/z 213 (MH+).

Methyl-(3-cyanophenyl)propanoate was prepared as follows: 3-(3-cyanophenyl)propanoic acid (993 mg, 4.0 mmol) in methanol (15 ml) was heated at reflux for 18 h. After evaporating under reduced pressure, the crude product was dissolved in dichloromethane, washed with saturated aqueous sodium hydrogen carbonate, brine and finally dried over magnesium sulphate. Filtration and evaporation under reduced pressure gave yield to methyl 3-(3-cyanophenyl)propanoate as an oil (1.09 g, 96%). ¹H NMR (399.9 MHz, DMSO-d₆) δ2.69 (2H, t), 2.94 (2H, t), 3.59 (3H, s), 7.50 (1H, t), 7.59-7.62 (1H, m), 7.66-7.68 (1H, m), 7.72-7.73 (1H, m).

### Example 49

### N′-[5-[2-(3-fluoro-5-methyl-phenyl)ethyl]-1H-pyrazol-3-yl]-N-[(3-methyl1,2-oxazol-5-yl)methyl]pyrimidine-2,4-diamine

Prepared in an analogous way to example 45, but starting with 5-[2-(3-fluoro-5-methyl-phenyl)ethyl]-1H-pyrazol-3-amine (143 mg, 0.52 mmol) and 4-chloro-N-[(3-methylisoxazol-5-yl)methyl]pyrimidin-2-amine (also known as 4-chloro-N-[(3-methyl-1,2-oxazol-5-yl)methyl]pyrimidin-2-amine; 117 mg, 0.52 mmol) as starting materials to afford the title compound as a white solid (85 mg, 35%). ¹H NMR (499.8 MHz, DMSO-d₆) δ2.21 (3H, s), 2.96 (1H, s), 2.98-2.99 (2H, m), 3.08 (2H, t), 4.72 (2H, s), 6.24 (2H, d), 6.55 (1H, d), 7.37 (2H, d), 7.42 (1H, s), 7.89 (1H, d), 10.69 (1H, s). MS: m/z 462 (MH+).

5-[2-(3-fluoro-5-methyl-phenyl)ethyl]-1H-pyrazol-3-amine used as starting material was prepared as outlined in Example 42 for 5-[2-(3,5-dimethoxy) ethyl]-2H-pyrazol-3-amine, starting from methyl 3-[3-fluoro-5-(trifluoromethyl)phenyl]propanoate (651 mg, 2.6 mmol as starting material. 5-[2-(3-fluoro-5-methyl-phenyl)ethyl]-1H-pyrazol-3-amine was obtained as a white solid (150 mg, 21%). ¹H NMR (399.9 MHz, DMSO-d₆) δ2.93 (2H, t), 3.05 (2H, t), 5.61 (1H, s), 7.45-7.50 (3H, m). MS: m/z 274 (MH+).

Methyl 3-[3-fluoro-5-(trifluoromethyl)phenyl]propanoate amine was prepared by reduction of methyl (E)-3-[3-fluoro-5-(trifluoromethyl)phenyl]prop-2-enoate (993 mg, 4.0 mmol) with 10% Pd/C (100 mg) in ethanol (15 ml) under a hydrogen atmosphere. After filtration through celite and evaporation methyl 3-[3-fluoro-5-(trifluoromethyl)phenyl]propanoate was obtained as an oil (650 mg, 65%). ¹H NMR (399.9 MHz, DMSO-d₆) δ2.73 (2H, t), 2.97 (2H, t), 3.60 (3H, s), 7.47-7.50 (3H, m)

Methyl (E)-3-[3-fluoro-5-(trifluoromethyl)phenyl]prop-2-enoate was prepared as follows:

3-fluoro-5-trifluromethylbenzaldehyde (0.999 g, 5.2 mmol) and methyl(triphenyl-phosphoranylidene)acetate (2.62 g, 7.8 mmol) in dichloromethane (25 ml) were stirred at ambient temperature for 4 h. After evaporating under reduced pressure the crude product was purified by column chromatography on silica using a gradient 2-10% of ethyl acetate in hexanes. The desired fractions were taken and evaporated to afford methyl (E)-3-[3-fluoro-5-(trifluoromethyl)phenyl]prop-2-enoate as an oil (1.08 g 84%). ¹H NMR (399.9 MHz, DMSO-d₆) δ3.76 (3H, s), 6.92 (1H, d), 7.68-7.74 (3H, m), 8.01-8.02 (2H, m).

### Example 50

### 5-[[[4-[(5-phenethyl-2H-pyrazol-3-yl)amino]pyrimidin-2-yl]amino]methyl]-1,2-oxazole-3-carboxamide

2-chloro-N-(5-phenethyl-2H-pyrazol-3-yl)pyrimidin-4-amine (100 mg, 0.33 mmol, 1 eq) and 5-(aminomethyl)-1,2-oxazole-3-carboxamide trifluoroacetic acid salt (86 mg, 0.33 mmol, 1.2 eq) were combined in 2-methoxyethanol (2 ml) containing diisopropylethylamine (175 μl, 1.00 mmol, 3 eq). The reaction was heated at 170° C. in the microwave for 3 h. An additional 0.3 eq of amine (25 mg, 0.1 mmol) was added and the reaction was heated for 60 mins at 175° C. then for 60 mins at 200° C. The solvent was evaporated under reduced pressure. The residue was extracted into ethyl acetate and washed with water and brine. Dried with magnesium sulphate, filtered and evaporated. The compound was then purified by basic reverse phase prep. HPLC. The desired fractions were taken, evaporated to afford the title compound as a solid (25.8 mg, 19%)

¹H NMR (300.132 MHz, DMSO) δ2.76-2.96 (4H, m), 4.61 (2H, d), 6.31 (2H, s), 6.52 (1H, s), 7.14-7.33 (6H, m), 7.73 (1H, s), 7.83 (1H, d), 8.02 (1H, s), 9.36 (1H, s), 11.93 (1H, s) MS: M/Z 405,(MH+)

2-chloro-N-(5-phenethyl-2H-pyrazol-3-yl)pyrimidin-4-amine, used as starting material was prepared as outlined in Example 30a).

5-(Aminomethyl)-1,2-oxazole-3-carboxamide was synthesized as outlined in Example 4.

### Example 51

### N-[(3-methyl1,2-oxazol-5-yl)methyl]-N′-[5-[2-[3-(trifluoromethoxy)phenyl]ethyl]-1H-pyrazol-3-yl]pyrimidine-2,4-diamine

Prepared in an analogous way to example 38 but starting with 5-[2-[3-(trifluoromethoxy)phenyl]ethyl]-1H-pyrazol-3-amine (112 mg, 0.50 mmol, 1 eq). The title compound was isolated as a solid (88.6 mg, 39% yield).

1H NMR (300.132 MHz, DMSO): δ 2.16 (s, 3H), 2.81-3.01 (m, 4H), 4.53 (d, 2H), 6.11 (s, 1H), 6.18-6.36 (m, 2H), 7.15-7.30 (m, 4H), 7.42 (t, 1H), 7.83 (d, 1H), 9.36 (s, 1H), 11.91 (s, 1H). MS: m/z 460 (MH+).

5-[2-[3-(trifluoromethoxy)phenyl]ethyl]-1H-pyrazol-3-amine used as starting material, was prepared as outlined in Example 42 for 5-[2-(3,5-dimethoxy)ethyl]-2H-pyrazol-3-amine, but using ethyl 3-[3-(trifluoromethoxy)phenyl]propanoate to afford a brown oil (620 mg, 10% yield)

1H NMR (300.132 MHz, CDCl3): δ 2.86 (t, 2H), 2.93 (t, 2H), 5.44 (s, 1H), 7.03-7.10 (m, 3H), 7.30 (t, 2H). MS: m/z 272 (MH+).

Ethyl 3-[3-(trifluoromethoxy)phenyl]propanoate was prepared as follows:
a) 3-Trifluoromethoxybenzaldehyde (4.945 g, 26 mmol) and ethyl 2-(triphenylphosphoranylidine)acetate (9.995 g, 28.6 mmol) were dissolved in THF and stirred at r.t. for 6 h. The crude product was dissolved in 5% (Ethyl Acetate : Isohexane) and filtered through a plug of silica. The first eluant was collected and afforded upon evaporation ethyl-3-[3-(trifluoromethoxy)phenyl]prop-2-enoate as a colourless oil. (5.75 g, 90%, as a 20:1 mixture of trans:cis alkene isomers)

Trans Isomer: 95%

1H NMR (300.132 MHz, CDCl3): δ 1.34 (t, 3H), 4.28 (q, 2H), 6.45 (d, 1H), 7.16-7.29 (m, 1H), 7.31-7.51 (m, 3H), 7.65 (d, 1H).

Cis Isomer: 5%

1H NMR (300.132 MHz, CDCl3): δ 1.23 (t, 3H), 4.17 (q, 2H), 6.01 (d, 1H), 6.90 (d, 1H), 7.17-7.51 (m, 4H).
b) To ethyl (E/Z)-3-[3-(trifluoromethoxy)phenyl]prop-2-enoate (5.75 g, 22.1 mmol) dissolved in ethyl acetate (50 mL) (under nitrogen) was added 10% palladium on carbon (20 mg). The reaction mixture was stirred under hydrogen for 2d. The mixture was filtered through celite and evaporated to afford ethyl 3-[3-(trifluoromethoxy)phenyl]propanoate as a colourless oil.

(Yield 5.75 g, 99%)

1H NMR (300.132 MHz, CDCl3): δ 1.23 (t, 3H), 2.62 (t, 2H), 2.97 (t, 2H), 4.13 (q, 2H), 7.00-7.16 (m, 3H), 7.23-7.35 (m, 1H).

### Example 52

### N-[(3-methyl1,2-oxazol-5-yl)methyl]-N′-[5-[2-(3-methylphenyl)ethyl]-1H-pyrazol-3-yl]pyrimidine-2,4-diamine hydrochloride

Prepared using an analogous method to example 46, but starting with 5-[2-(3-methylphenyl)ethyl]-1H-pyrazol-3-amine (77 mg, 0.36 mmol) to give the title compound (51 mg, 32% yield)

1H NMR (300.132 MHz, DMSO) δ 2.18 (s, 3H), 3.85 (s, 3H), 4.72 (s, 2H), 5.06 (s, 2H), 6.27 (s, 1H), 6.37 (s, 1H), 6.97-7.03 (m, 1H), 7.16-7.26 (m, 2H), 7.91 (d, 1H). MS: m/z 390 (MH+)

5-[2-(3-Methylphenyl)ethyl]-1H-pyrazol-3-amine, used as a starting material, was prepared using an analogous method to example 34a), but starting with methyl 3-(3-methylphenyl)propanoate (4 g, 22.4 mmol) to give 5-[2-(3-Methylphenyl)ethyl]-1H-pyrazol-3-amine (3.1 g, 69%) as a brown gum. MS: m/z 202 (MH+)

Methyl 3-(3-methylphenyl)propanoate was prepared using a method analogous to example 31a), using 3-(3-methylphenyl)propanoic acid (7 g, 42.6 mmol) to give methyl 3-(3-methylphenyl)propanoate (7 g, 92%) as a colourless oil.

1H NMR (300.132 MHz, CDCl3) δ 2.25 (s, 3H), 2.54 (t, 2H), 2.84 (t, 2H), 3.59 (s, 3H), 6.91-6.95 (m, 3H), 7.07-7.12 (m, 1H). MS: N/A

### Example 53

### N′-[5-[2-(3-bromophenyl)ethyl]-1H-pyrazol-3-yl]-N-[(3-methyl1,2-oxazol-5-yl)methyl]pyrimidine-2,4-diamine hydrochloride

Prepared using an analogous method to example 46, but starting with 5-[2-(3-bromophenyl)ethyl]-1H-pyrazol-3-amine (95 mg, 0.36 mmol) to give the title compound (82 mg, 46% yield)

1H NMR (300.132 MHz, DMSO) δ 2.18 (s, 3H), 2.89 (s, 4H), 4.70 (s, 2H), 6.16-6.33 (m, 2H), 6.38 (s, 1H), 7.19-7.26 (m, 2H), 7.35-7.40 (m, 1H), 7.43 (s, 1H), 7.86 (d, 1H). MS: m/z 456 (MH+)

5-[2-(3-bromophenyl)ethyl]-1H-pyrazol-3-amine, used as a starting material, was prepared using an analogous method to example 34a), but starting with methyl 3-(3-bromophenyl)propanoate (7 g, 28.8 mmol) to give 5-[2-(3-bromophenyl)ethyl]-1H-pyrazol-3-amine (4.9 g, 60%) as a brown gum. MS: m/z 280 ((M−H)⁻)

Methyl 3-(3-bromophenyl)propanoate was prepared using a method analogous to example 31a), using 3-(3-bromophenyl)propanoic acid (10 g, 43.6 mmol) to give methyl 3-(3-bromophenyl)propanoate (10 g, 94%) as a colourless oil. 7.03-7.10 (m, 2H), 7.25-7.26 (m, 2H).

### Example 54

### N′-[5-(2-benzo [1,3]dioxol-5-ylethyl)-2H-pyrazol-3-yl]-N-[(3-methyl1,2-oxazol-5-yl)methyl]pyrimidine-2,4-diamine

Prepared in an analogous way to example 38, but starting with (5-(2-benzo[1,3]dioxol-5-ylethyl)-2H-pyrazol-3-amine (128 mg, 0.55 mmol, 1 eq). The HCl salt precipitated out of the reaction mixture on cooling and was filtered and dried. The product was suspended in water and basified by the addition of ammonium hydroxide solution before extraction into ethyl acetate. The organic layer was separated, washed with saturated sodium hydrogen carbonate and then brine. Dried with magnesium sulphate, filtered and evaporated to afford the title compound as a solid. (132.1 mg, 57% yield).

1H NMR (300.132 MHz, DMSO): δ 2.17 (s, 3H), 2.76-2.84 (m, 4H), 4.53 (d, 2H), 5.96 (s, 2H), 6.10 (s, 1H), 6.26 (s, 2H), 6.68 (dd, 1H), 6.78-6.82 (m, 2H), 7.19 (s, 1H), 7.83 (d, 1H), 9.34 (s, 1H), 11.88 (s, 1H). MS: m/z 420 (MH+).

(5-(2-Benzo[1,3]dioxol-5-ylethyl)-2H-pyrazol-3-amine used as starting material was prepared in a similar manner to 5-[2-(3-methoxyphenyl)ethyl]-2H-pyrazol-3-amine in example 27a). Product was obtained as yellow oil. (3.04 g, 44% yield).

1H NMR (300.132 MHz, DMSO): δ 2.63-2.79 (m, 4H), 4.40 (s, 2H), 5.18 (s, 1H), 5.95 (s, 2H), 6.66 (dd, 1H), 6.77-6.81 (m, 2H). MS: m/z 232 (MH+).

### Example 55

### N-[(3-methyl1,2-oxazol-5-yl)methyl]-N′-[5-[2-(3-morpholin-4-ylphenyl)ethyl]-1H-pyrazol-3-yl]pyrimidine-2,4-diamine

Prepared in an analogous way to example 38, but starting with 5-[2-(3-morpholin-4-ylphenyl)ethyl]-1H-pyrazol-3-amine (112 mg, 0.50 mmol, 1 eq). The title compound was isolated as a white solid (105.7 mg, 53% yield).

1H NMR (300.132 MHz, DMSO): δ 2.16 (s, 3H), 2.83 (s, 4H), 3.07 (t, 4H), 3.71 (t, 4H), 4.53 (d, 2H), 6.10 (s, 1H), 6.21-6.36 (m, 2H), 6.69 (d, 1H), 6.76 (dd, 1H), 6.81 (s, 1H), 7.13 (t, 1H), 7.14 (m, 1H), 7.82 (d, 1H), 9.34 (s, 1H), 11.89 (s, 1H).

MS: m/z 461 (MH+).

5-[2-(3-morpholin-4-ylphenyl)ethyl]-1H-pyrazol-3-amine (470 mg, 85% yield) used as starting material was prepared in a similar manner to 5-[2-(3-methoxyphenyl)ethyl]-2H-pyrazol-3-amine in example 27a) using ethyl 3-(3-morpholin-4-ylphenyl)propanoate as starting material.

1H NMR (300.132 MHz, DMSO): δ 2.64-2.83 (m, 4H), 3.08 (t, 4H), 3.73 (t, 4H), 4.40 (s, 2H), 5.20 (s, 1H), 6.67 (d, 1H), 6.75 (dd, 1H), 6.79 (s, 1H), 7.12 (t, 1H), 11.06 (s, 1H). MS: m/z 273 (MH+).

Ethyl 3-(3-morpholin-4-ylphenyl)propanoate was prepared as follows:
a) 3-morpholin-4-yl benzoic acid (5.185 g, 25 mmol, 1 eq), 2-chloro-4,6-dimethoxy-1,3-5-triazine (5.22 g, 29.75 mmol, 1.19 eq) and N-methylmorpholine (7.588 g, 75 mmol, 3 eq) were stirred in anhydrous tetrahydrofuran (50 ml) at room temperature for an hour. A precipitate was observed. N,O-Dimethylhydroxylamine hydrochloride (2.44 g, 25 mmol, 1 eq) was then added and the reaction was stirred overnight at room temperature for 16 hours. The reaction mixture was diluted with ether and the organic layer washed with water then saturated sodium carbonate and finally brine. The organic layer were dried and evaporated under reduced pressure to yield 7.73 g as a pink oil. This was loaded onto a 120 g silica column in dichloromethane and eluted with 50-100% ethyl acetate in hexane. The clean fractions were combined and evaporated to yield N-methoxy-N-methyl-3-morpholin-4-yl-benzamide as a yellow oil. (2.77 g, 44% yield)

1H NMR (300.132 MHz, DMSO): δ 3.13 (t, 4H), 3.23 (s, 3H), 3.56 (s, 3H), 3.74 (t, 4H), 6.98 (d, 1H), 7.06 (m, 2H), 7.29 (m, 1H). MS: m/z 251 (MH+).
b)Bis(cyclopentadienyl)zirconium chloride hydride (4.28 g, 16.60 mmol, 1.5 eq) was added portionwise to a solution of N-methoxy-N-methyl-3-morpholin-4-yl-benzamide (2.77 g, 11.07 mmol, 1 eq) in tetrahydrofuran (50 ml). The reaction was stirred at room temperature for 15 mins after the initial evolution of gas. The reaction was evaporated to low volume and then dry loaded onto silica. The product was purified on a 40 g silica column eluting with 0-40% ethyl acetate in hexane over 20 mins. The clean fractions were combined to yield 3-morpholin-4-ylbenzaldehyde as a yellow oil. (1.34 g, 63%)

1H NMR (300.132 MHz, DMSO): δ 3.19 (t, 4H), 3.76 (t, 4H), 7.29-7.35 (m, 2H), 7.42-7.49 (m, 2H), 9.95 (s, 1H). MS: m/z 192 (MH+).
c)Ethyl 2-(triphenylphosphoranylidene)acetate (3.485 g, 10 mmol, 1 eq) was added to 3-morpholin-4-ylbenzaldehyde (1.33 g, 6.95 mmol,1 eq) in anhydrous tetrahydrofuran (30 ml). The reaction was stirred at room temperature overnight. The solvent was evaporated under reduced pressure and the residue dry loaded onto silica in dichloromethane. The product was purified on a 40 g silica column eluting with 0-25% ethyl acetate in hexane. The clean fractions were taken and evaporated to yield ethyl-3-(3-morpholin-4-ylphenyl)prop-2-enoate (mainly trans) as a yellow/green oil. (1.71 g, 94%)

1H NMR (300.132 MHz, DMSO): δ 1.26 (t, 3H), 3.16 (t, 4H), 3.74 (t, 4H), 4.19 (q, 2H), 6.64 (d, 1H), 7.01 (dd, 1H), 7.13 (d, 1H), 7.24-7.30 (m, 2H), 7.60 (d, 1H).

MS: m/z 262 (MH+).
d)To ethyl-3-(3-morpholin-4-ylphenyl)prop-2-enoate (1.658 g, 6.34 mmol, 1 eq) in ethanol (35 ml) was added 10% palladium on charcoal (166 mg). The reaction was stirred under a hydrogen balloon for 18 hours. The palladium residues were filtered and the filtrate evaporated under reduced pressure to yield ethyl 3-(3-morpholin-4-ylphenyl)propanoate as a clear oil. (1.636 g, 98%) as a clear oil.

1HNMR (300.132 MHz, CDCl3): δ 1.24 (t, 3H), 2.61 (t, 2H), 2.91 (t, 2H), 3.15 (t, 4H), 3.85 (t, 4H), 4.13 (q, 2H), 6.70-6.79 (m, 3H), 7.16-7.22 (m, 1H). MS: m/z 264 (MH+).

### Example 56

### 3-[2-[5-[[2-[(3-cyclopropyl-1,2-oxazol-5-yl)methylamino]pyrimidin-4-yl]amino]-1H-pyrazol-3-yl]ethyl]phenol

N-[(3-Cyclopropyl-1,2-oxazol-5-yl)methyl]-N′-[5-[2-(3-methoxyphenyl)ethyl]-2H-pyrazol-3-yl]pyrimidine-2,4-diamine (191 mg) was dissolved in DCM (20 ml) and cooled to 0° C. under nitrogen. Boron tribromide solution was added dropwise and the reaction was allowed to warm to room temperature and stirred overnight. The reaction was quenched carefully with methanol (10 ml) and the solution was evaporated to dryness. The crude product was loaded onto a SCX-2 column, washed with methanol and then eluted with 2N ammonia in methanol to give the product as a yellow gum. Trituration with ether gave a white solid, which was then filtered and dried in a vacuum oven at 45° C. overnight (130 mg, 71%). 1H NMR (DMSO 400.13 MHz) δ 0.69 (m, 2H), 0.95 (m, 2H), 1.93 (m, 1H), 2.79 (s, 4H), 4.51 (d, 2H), 6.0 (s, 1H), 6.28 (bs, 1H), 6.57 (m, 1H), 6.65 (m, 2H), 7.05 (t, 1H), 7.15 (bs, 1H), 7.83 (d, 1H), 9.21 (s, 1H), 9.35 (bs, 1H), 11.92 (s, 1H)

MS: m/z 418 (MH+).

N-[(3-Cyclopropyl-1,2-oxazol-5-yl)methyl]-N′-[5-[2-(3-methoxyphenyl)ethyl]-2H-pyrazol-3-yl]pyrimidine-2,4-diamine used as starting material was prepared as in Example 28.

### Example 57

### N′-[5-[2-(3-chloro-5-fluoro-phenyl)ethyl]-2H-pyrazol-3-yl]-N-[(3-methyl-1,2-oxazol-5-yl)methyl]pyrimidine-2,4-diamine

A mixture of 5-[2-(3-chloro-5-fluoro-phenyl)ethyl]-2H-pyrazol-3-amine (0.096 g, 0.4 mmol), 4-chloro-N-[(3-methyl-1,2-oxazol-5-yl)methyl]pyrimidin-2-amine (0.090 g, 0.4 mmol) and ethanol (5 ml) was stirred and heated in a microwave at 120° C. for 30 mins. On cooling the product precipitated out. This was filtered, washed with ice cold ethanol (5 ml) and ether (2 ml) to give a pale yellow solid. The crude product was purified by reverse-phase prep. HPLC (basic) using a 31-51% gradient of acetonitrile in water containing 1% ammonium hydroxide solution, and a white solid was obtained (0.054 g, 32%).

¹H NMR (399.9 MHz, DMSO-d₆) δ 2.17 (3H, s), 2.88 (4H, m), 4.54 (2H, s), 6.10- 6.40 (2H, d), 7.10 (1H, d), 7.20-7.30 (2H, m), 7.80 (1H, d), 9.35-9.50 (1H, s), 11.90-12.00 (1H, s) MS: m/z 428.38 (MH⁺)

5-[2-(3-Chloro-5-fluoro-phenyl)ethyl]-2H-pyrazol-3-amine, used as starting material was prepared as follows:—

Sodium hydride (60%, 0.288 g, 7.20 mmol) was added to a stirred solution of methyl 3-(3-chloro-5-fluoro-phenyl)propanoate (1.3 g, 6.0 mmol) in 1,4 dioxane (30 ml) and dry acetonitrile (0.377 ml, 7.20 mmol) under nitrogen. The mixture was stirred at r.t. for 10 mins and then refluxed (under nitrogen) overnight. After this time, the mixture was cooled to r.t. and ethanol (3 ml) was added, followed by hydrazine monohydrochloride (0.823 g, 12.0 mmol). The mixture was then refluxed overnight. The reaction mixture was allowed to cool to room temperature and filtered. The solution was concentrated in vacuo and then partitioned between 2N HCl and ethyl acetate (25 ml each). The aqueous layer was extracted with ethyl acetate and basified with ammonium hydroxide solution to pH 8. This was then extracted using ethyl acetate, washed with water and brine, dried (MgSO₄), filtered and evaporated to dryness to give a dark orange gum. This was purified by reverse phase prep. HPLC (basic) using a 28-38% gradient of acetonitrile in water containing 1% ammonium hydroxide solution, and a white solid was obtained (0.1 15 g, 8%).

Methyl 3-(3-chloro-5-fluoro-phenyl)propanoate, used as starting material in the synthesis of 5-[2-(3-chloro-5-fluoro-phenyl)ethyl]-2H-pyrazol-3-amine was prepared as follows:—

A solution of 3-(3-chloro-5-fluorophenyl)propionic acid (1.015 g, 5 mmol) in a mixture of toluene: methanol (10 ml:5 ml) was treated dropwise at room temperature with 2M (Trimethylsilane)diazomethane (3 ml). The reaction mixture was stirred under nitrogen for 1 h and the solution was evaporated to dryness to give the crude product. The crude product was dissolved in DCM and washed with sodium bicarbonate, water, brine and dried with MgSO4. After filtration the solvent was evaporated off to give methyl 3-(3-chloro-5-fluoro-phenyl)propanoate (0.794 g product, 73%).

4-Chloro-N-[(3-methyl-1,2-oxazol-5-yl)methyl]pyrimidin-2-amine was prepared as outlined in Example 13.

### Example 58

### N′-[5-[2-[3-(aminomethyl)phenyl]ethyl]-1H-pyrazol-3-yl]-N-[(3-methyl-1,2-oxazol-5-yl)methyl]pyrimidine-2,4-diamine

Lithium aluminium hydride (72 mg, 1.88 mmol) was added to a suspension of 3-[2-[5-[[2-[(3-methyl-1,2-oxazol-5-yl)methylamino]pyrimidin-4-yl]amino]-2H-pyrazol-3-yl]ethyl]benzonitrile (301 mg, 0.75 mmol) in anhydrous tetrahydrofuran (30 ml). The reaction mixture was stirred at room temperature for 2 h. The reaction was quenched by neutralisation to pH 6-7 at 0° C. with 1M hydrochloric acid, evaporated to dryness and purified on an SCX 2 column. Product was eluted using 3.5N ammonia in methanol. After evaporation under reduced pressure the crude product was purified by reverse phase prep. HPLC (acidic) using a 5-95% gradient of acetonitrile in water containing 1% formic acid, followed by reverse phase prep HPLC (basic) using a gradient 0-95% of acetonitrile in water containing 1% ammonia The clean fractions were taken and evaporated to afford N′-[5-[2-[3-(aminomethyl)phenyl]ethyl]-1H-pyrazol-3-yl]-N-[(3-methyl-i ,2-oxazol-5-yl)methyl]pyrimidine-2,4-diamine as a white solid (23. lmg, 7.6%).

1H NMR (500.13 MHz, DMSO-d₆) δ 2.17 (3H, s), 2.85 (2H, d), 2.90 (1H, d), 2.91 (1H, s), 3.83 (2H, s), 4.54 (2H, d), 6.12 (1H, s), 7.16 (1H, d), 7.21 (2H, d), 7.26 (1H, s), 7.28 (2H, t), 7.84 (1H, d). MS: m/z 405 (MH+).

3-[2-[5-[[2-[(3-Methyl-1,2-oxazol-5-yl)methylamino]pyrimidin-4-yl]amino]-2H-pyrazol-3-yl]ethyl]benzonitrile was prepared as described in Example 48.

### Example 59

### N,N-dimethyl-3-[2-[5-[[2-[(3-methyl-1,2-oxazol-5-yl)methylamino]pyrimidin-4-yl]amino]-2H-pyrazol-3-yl]ethyl]benzamide

Prepared in an analogous way to example 108, starting from of 3-[2-(5-amino-2H-pyrazol-3-yl)ethyl]-N,N-dimethyl-benzamide (130 mg, 0.45 mmol) and 4-chloro-N-[(3-methylisoxazol-5-yl)methyl]pyrimidin-2-amine (also known as 4-chloro-N-[(3-methyl-1,2-oxazol-5-yl)methyl]pyrimidin-2-amine; 113 mg, 0.5 mmol). Purified by reverse phase prep. HPLC (acidic) using a 0-95% gradient of acetonitrile in water containing 1% formic acid. The clean fractions were taken and evaporated to afford the title compound as a white solid (60 mg, 27%).

1H NMR (500.13 MHz, DMSO-d₆) δ 2.16 (3H, s), 2.86-2.92 (2H, m), 2.90 (6H, s), 2.93-2.99 (2H, m), 4.54 (2H, d), 6.03 (1H, s), 6.08 (1H, s), 6.26 (1H, d), 6.76 (1H, s), 7.17 (1H, d), 7.20 (1H, s), 7.75-7.83 (2H, m), 7.85 (1H, d), 8.89 (1H, s). MS: m/z 447 (MH+).

4-Chloro-N-[(3-methyl-1,2-oxazol-5-yl)methyl]pyrimidin-2-amine was prepared as outlined in Example 13.

3-[2-(5-amino-2H-pyrazol-3-yl)ethyl]-N,N-dimethyl-benzamide used as starting material was prepared using an analogous procedure to that for 5-[2-(3,5-dimethoxy)ethyl]-2H-pyrazol-3-amine) in Example 42, starting from methyl 3-[3-(dimethylcarbamoyl)phenyl]propanoate (1.3 g, 6.85 mmol), sodium hydride (329 mg dispersion in mineral oil, 8.22 mmol), acetonitrile (430 μL, 8.22 mmol) and hydrazine monohydrochloride (939 mg, 13.7 mmol). The crude product was purified by normal phase chromatography on silica gel using a 50-100% gradient of ethyl acetate in hexanes. The clean fractions were taken and evaporated to afford 3-[2-(5-amino-2H-pyrazol-3-yl)ethyl]-N,N-dimethyl-benzamide as a yellow gum (485 mg, 27%).

¹H NMR (399.9 MHz, DMSO-d₆) δ 2.72-2.76 (2H, m), 2.84-2.89 (6H, m), 2.90 (2H, m), 4.40 (2H, s), 5.18 (1H, s), 7.19-7.22 (1H, m), 7.27-7.30 (1H, m), 7.32 (1H, s), 7.35 (1H, d), 11.0 (1H, s). MS: m/z 259 (MH+).

Methyl 3-[3-(dimethylcarbamoyl)phenyl]propanoate was prepared from the reduction of methyl (E)-3-[3-(dimethylcarbamoyl)phenyl]prop-2-enoate (2.335 g, 10.0 mmol) with 10% Pd/C (234 mg) in ethanol (50 ml) under a hydrogen atmosphere. Filtered through celite, evaporated to afford to afford methyl 3-[3-(dimethylcarbamoyl)phenyl]propanoate as an oil (1.35 g, 55%). ¹H NMR (399.9 MHz, DMSO-d₆) δ2.67 (2H, t), 2.90 (6H, t), 2.98 (2H, s), 3.59 (3H, s), 7.20-7.40 (4H, m) ). MS: m/z 236 (MH+).

Methyl (E)-3-[3-(dimethylcarbamoyl)phenyl]prop-2-enoate was prepared using an 25 analogous procedure to that for methyl (E)-3-[3-fluoro-5-(trifluoromethyl)phenyl]prop-2-enoate in Example 49, starting from 3-formyl-N,N-dimethyl-benzamide (3.015 g, 17 mmol) and methyl(triphenyl-phosphoranylidene)acetate (8.53 g, 25.5 mmol) in dichloromethane (35 ml). The crude product was purified by normal phase chromatography on silica gel using a 0-2.5% gradient of methanol in dichloromethane, followed by a silica gel column using a 50-75% gradient of ethyl acetate in hexanes. The clean fractions were taken and evaporated to afford methyl (E)-3-[3-(dimethylcarbamoyl)phenyl]prop-2-enoate as a gum (2.4 g 64%).

¹H NMR (399.9 MHz, DMSO-d₆) δ 2.90-2.95 (3H, s), 2.95-3.05 (3H, s), 3.75 (3H, s), 6.70-6.75 (1H, d), 7.40-7.50 (2H, m), 7.65-7.75 (1H, d), 7.75 (1H, t), 7.80 (1H, d).

### Example 60

### N′-[5-[2-(2,4-dimethoxypyrimidin-5-yl)ethyl]-1H-pyrazol-3-yl]-N-[(3-methyl-1,2-oxazol-5-yl)methyl]pyrimidine-2,4-diamine

Prepared using an analogous procedure to that in Example 57, starting from 5-[2-(2,4-dimethoxypyrimidin-5-yl)ethyl]-1H-pyrazol-3-amine (100 mg, 0.40 mmol) and 4-chloro-N-[(3-methyl-1,2-oxazol-5-yl)methyl]pyrimidin-2-amine (90 mg, 0.40 mmol). Purified by reverse phase prep. HPLC (basic) using a 2.5-97.5% gradient of acetonitrile in water containing 1% ammonia. The clean fractions were taken and evaporated to affordthe title compound as a white solid (68 mg, 39%).

¹H NMR (399.9 MHz, DMSO-d₆) δ2.18 (3H, d), 2.76-2.79 (4H, m), 3.87 (3H, s), 3.94 (3H, s), 4.52 (2H, d), 6.10 (1H, s), 6.29 (2H, s), 7.19 (1H, s), 7.83 (1H, d), 8.03 (1H, s), 9.34 (1H, s), 11.89 (1H, s). MS: m/z 438 (MH+).

4-Chloro-N-[(3-methyl-1,2-oxazol-5-yl)methyl]pyrimidin-2-amine was prepared as outlined in Example 13.

5-[2-(2,4-dimethoxypyrimidin-5-yl)ethyl]-1H-pyrazol-3-amine used as starting material was prepared using an analogous procedure to that for 5-[2-(3,5-dimethoxy)ethyl]-2H-pyrazol-3-amine) in Example 42, starting from methyl 3-(2,4-dimethoxypyrimidin-5-yl)propanoate (611 mg, 2.7 mmol), sodium hydride (130 mg dispersion in mineral oil, 3.24 mmol), acetonitrile (430 uL, 8.22 mmol) and hydrazine monohydrochloride (370 mg, 5.4 mmol). The crude product was purified by normal phase chromatography on silica gel using a 0-20% gradient of methanol in dichloromethane. The clean fractions were taken and evaporated to afford 5-[2-(2,4-dimethoxypyrimidin-5-yl)ethyl]-1H-pyrazol-3-amine as an oil (139 mg, 19%). ¹H NMR (399.9 MHz, DMSO-d₆) δ2.65-2.71 (4H, m), 3.87 (3H, s), 3.93 (3H, s), 4.44 (2H, s), 5.17 (1H, s), 8.03 (1H, s), 10.91 (1H, s).

MS: m/z 250 (MH+).

Methyl 3-(2,4-dimethoxypyrimidin-5-yl)propanoate used as starting material was prepared using an analogous procedure to that for methyl 3-[3-(dimethylcarbamoyl)phenyl]propanoate in Example 59 starting from methyl (E)-3-(2,4-dimethoxypyrimidin-5-yl)prop-2-enoate (774 mg, 3.45 mmol) with 5% Pt/C (80 mg) in N,N-dimethylformamide (10 ml) under a hydrogen atmosphere. Filtered through celite, evaporated to afford to afford methyl 3-(2,4-dimethoxypyrimidin-5-yl)propanoate as an oil (611 m g, 78%). ¹H NMR (399.9 MHz, DMSO-d₆) δ2.55-2.59 (1H, m), 2.57-2.58 (1H, m), 2.68-2.72 (2H, m), 3.59 (3H, s), 3.87 (3H, s), 3.93 (3H, s), 8.13 (1H, s)

Methyl (E)-3-(2,4-dimethoxypyrimidin-5-yl)prop-2-enoate was prepared as follows:

A suspension (E)-3-(2,4-dimethoxypyrimidin-5-yl)prop-2-enoic acid (1.05 g, 5.0 mmol) in a mixture of methanol (5 ml) and toluene (10 ml), was treated at ambient temperature with a solution of trimethylsilyl diazomethane (2M in hexanes, 3.0 ml, 6.0 mmol). Stirred for 1 hour and evaporated to afford methyl (E)-3-(2,4-dimethoxypyrimidin-5-yl)prop-2-enoate as a solid (0.77 g, 69%). ¹H NMR (399.9 MHz, DMSO-d₆) δ3.73 (3H, s), 3.96 (3H, s), 3.99-4.09 (3H, m), 6.69 (1H, d), 7.55 (1H, d), 8.73 (1H, s).

### Example 61

### [5-[[[4-[[5-[2-(3-methoxyphenyl)ethyl]-2H-pyrazol-3-yl]amino]pyrimidin-2-yl]amino]methyl]-1,2-oxazol-3-yl]methanol

To 2-chloro-N-[5-[2-(3-methoxyphenyl)ethyl]-2H-pyrazol-3-yl]pyrimidin-4-amine (250 mg, 0.76 mmoles) was added [5-(aminomethyl)-1,2-oxazol-3-yl]methanol (146 mg) followed by 2-methoxyethanol (4 ml) and diisopropylethylamine (265 ml). The reaction mixture was heated in the microwave at 200° C. for 60 mins. The solvent was evaporated under reduced pressure. The crude product was purified by flash chromatogephy using a silica column, eluting with 5-10% methanol in dichloromethane. Desired fractions were combined and evaporated to give product as a yellow foam 287 mg (90%).

1H NMR (DMSO 400.13 MHz) δ 2.85 (m, 4H), 3.72 (s, 3H), 4.44 (d, 2H), 4.56 (d, 2H), 5.36 (t, 1H), 6.21 (s, 1H), 6.29 (bs, 1H), 6.75 (m, 1H), 6.81 (m, 2H), 7.19 (t, 1H), 7.81 (d, 1H), 9.32 (bs, 1H), 11.9 (s, 1H)

MS: m/z 422 (MH+)

2-chloro-N-[5-[2-(3-methoxyphenyl)ethyl]-2H-pyrazol-3-yl]pyrimidin-4-amine, used as starting material was prepared as in Example 27.

[5-(aminomethyl)-1,2-oxazol-3-yl]methanol, used as starting material was prepared as follows:

tert-butyl N-[[3-(hydroxymethyl)-1,2-oxazol-5-yl]methyl]carbamate (3.36 g, 14.72 mmoles) was dissolved in dichloromethane (67 ml) and trifluoroacetic acid (5.47 ml) was added. The reaction was stirred at room temperature for 2d. The mixture was evaporated to dryness, loaded onto a SCX-2 column and washed with methanol. The product was eluted with 3.5N ammonia in methanol to give product as a white solid (after trituration with diethyl ether) (1.24 g, 66%).

tert-butyl N-[[3-(hydroxymethyl)-1,2-oxazol-5-yl]methyl]carbamate was prepared as follows:—

Ethyl 5-[[(2-methylpropan-2-yl)oxycarbonylamino]methyl]-1,2-oxazole-3-carboxylate (5 g, 18.50 mmoles) was dissolved in ethanol (50 ml) and cooled to 0° C. Sodium borohydride (1.89 g, 49.95 mmoles) was added portionwise and the reaction was stirred at room temperature overnight. The mixture was quenched with aqueous sodium bicarbonate solution. The mixture was then extracted with ethyl acetate, washed with brine, dried (MgSO₄) and evaporated to give the product as a colouress oil (4.22 g, 100%).

Ethyl 5-[[(2-methylpropan-2-yl)oxycarbonylamino]methyl]-1,2-oxazole-3-carboxylate, used as starting material, was prepared as follows:—

Tert-butyl N-prop-2-ynylcarbamate (40.97 g, 0.26mol, 1 eq) was dissolved in anhydrous THF (150 mL) and N,N-diethylethanamine (22 mL, 0.16 mol, 1.2 eq) added. A solution of ethyl-2-chloro-2-hydroxyimino-acetate (20 g, 0. 13mol, 1 eq) in anhydrous THF (350mL) was added dropwise over 7 h. The reaction was stirred at room temperature overnight then evaporated to dryness. The residue was dissolved in DCM and washed with water, brine and dried (MgSO₄). After filtration, the solution was evaporated to give the crude product as a yellow oil. This was purified by silica column chromatography, eluting with 20% -60% ether in iso-hexane to give ethyl 5-[[(2-methylpropan-2-yl)oxycarbonylamino]methyl]-1,2-oxazole-3-carboxylate as a white solid (20.12 g, 56%).

1H NMR (CDCl3 400.13 MHz) δ 1.39-1.47 (12H, m), 4.40-4.49 (5H, m), 5.0 (1H, s), 6.58 (1H, s). MS m/z 269 (M−H).

### Example 62

### N′-[5-[2-(5-fluoro-2-methoxy-pyridin-4-yl)ethyl]-1H-pyrazol-3-yl]-N-[(3-methyl-1,2-oxazol-5-yl)methyl]pyrimidine-2,4-diamine hydrochloride

5-[2-(5-fluoro-2-methoxy-pyridin-4-yl)ethyl]-1H-pyrazol-3-amine (60 mg, 0.254 mmol) was heated with 4-chloro-N-[(3-methyl-1,2-oxazol-5-yl)methyl]pyrimidin-2-amine (58 mg, 0.254 mmol) in ethanol (1.5 ml) at 80° C. for 24 h. The mixture was allowed to cool to room temperature and the precipitated solid was collected by filtration, washed with ethanol and dried under vacuum to afford the title compound as an off-white solid (58 mg, 50% yield).

¹H NMR (399.902 MHz, DMSO) δ 2.19 (s, 3H), 2.93 (s, 4H), 3.80 (s, 3H), 4.70 (d, 2H), 6.20-6.45 (bm, 3H), 6.74 (d, 1H), 7.89 (bs, 1H), 8.06 (d, 1H), 8.78 (bs, 1H), 11.21 (bs, 1H), 12.47 (bs, 1H), 12.56 (bs, 1H). MS: m/z 425 (MH+)

4-Chloro-N-[(3-methyl-1,2-oxazol-5-yl)methyl]pyrimidin-2-amine was prepared as outlined in Example 13.

5-[2-(5-fluoro-2-methoxy-pyridin-4-yl)ethyl]-1H-pyrazol-3-amine, used as starting material was prepared as follows:

Methyl 3-(5-fluoro-2-methoxy-pyridin-4-yl)propanoate (260 mg, 1.22 mmol) and acetonitrile (78 μl, 1.46 mmol) were stirred in anhydrous 1,4-dioxane (6 ml) under nitrogen. Sodium hydride (60% dispersion on mineral oil, 59 mg, 1.46 mmol) was added and the mixture was stirred at room temperature for 10 mins, then heated at reflux for 16 h. After cooling to room temperature, ethanol (1 ml) was added followed by hydrazine monohydrochloride (168 mg, 2.44 mmol) and the mixture was heated again at reflux for 24 h. The mixture was evaporated to dryness and the residue was purified on a silica isolute column, eluting with 0-3% methanol in DCM, to afford 5-[2-(5-fluoro-2-methoxy-pyridin-4-yl)ethyl]-1H-pyrazol-3-amine as a yellow solid (128 mg, 40% yield).

¹H NMR (399.902 MHz, CDCl3) δ 2.84-2.94 (m, 4H), 3.87 (s, 3H), 5.46 (s, 1H), 6.53 (d, 1H), 7.92 (d, 1H). MS: m/z 237 (MH+).

Methyl 3-(5-fluoro-2-methoxy-pyridin-4-yl)propanoate, used as starting material was prepared as follows:

10% Pd/C (25 mg) was added to a solution of methyl 3-(5-fluoro-2-methoxy-pyridin-4-yl)prop-2-enoate (315 mg, 1.49 mmol) in ethanol (25 ml) and the mixture was stirred at room temperature under a balloon of hydrogen for 1 h. The mixture was filtered, washed through with ethanol and the filtrate evaporated under vacuum to afford methyl 3-(5-fluoro-2-methoxy-pyridin-4-yl)propanoate as a colourless oil (296 mg, 93% yield).

¹H NMR (399.902 MHz, CDCl3) δ 2.65 (t, 2H), 2.94 (t, 2H), 3.69 (s, 3H), 3.88 (s, 3H), 6.58 (d, 1H), 7.91 (d, 1H). MS: m/z 214 (MH+)

Methyl 3-(5-fluoro-2-methoxy-pyridin-4-yl)prop-2-enoate, used as starting material was prepared as follows:

Methyl 2-triphenylphosphoranylideneacetate (1.52 g, 4.54 mmol) was added portionwise to a stirred solution of 5-fluoro-2-methoxy-pyridine-4-carbaldehyde (470 mg, 3.03 mmol) in DCM (10 ml) under nitrogen. Stirring was continued at room temperature for 16 h. The solution was evaporated and the crude product was adsorbed onto silica, then purified on a silica isolute column, eluting with 2-4% ethyl acetate in hexane, to afford methyl 3-(5-fluoro-2-methoxy-pyridin-4-yl)prop-2-enoate as a white solid (330 mg, 52% yield).

¹H NMR (399.902 MHz, DMSO) δ 3.77 (s, 3H), 3.86 (s, 3H), 6.91 (d, 1H), 7.32 (d, 1H), 7.60 (d, 1H), 8.26 (d, 1H). MS: m/z 212 (MH+)

5-Fluoro-2-methoxy-pyridine-4-carbaldehyde, used as starting material was prepared as follows:

(5-Fluoro-2-methoxy-pyridin-4-yl)methanol (1.40 g, 8.91 mmol) was stirred in DCM (50 ml). Dess-Martin periodinane (4.535 g, 10.69 mmol) in DCM (70 ml) was added slowly and stirring continued at room temperature for 1.5 h. The solution was then washed with 1M NaOH(aq) (2×75 ml), water (75 ml), brine, dried over MgSO₄, filtered and evaporated to afford 5-fluoro-2-methoxy-pyridine-4-carbaldehyde as a yellow oil (0.481 g, 35% yield).

1H NMR (399.902 MHz, CDCl3) δ 3.94 (s, 3H), 7.08-7.11 (m, 1H), 8.20-8.22 (m, 1H), 10.32 (s, 1H).

(5-Fluoro-2-methoxy-pyridin-4-yl)methanol, used as starting material was prepared as follows:—

Borane-tetrahydrofuran complex (1M solution in THF, 52.6 ml, 52.6 mmol) was added slowly to a solution of 5-fluoro-2-methoxy-pyridine-4-carboxylic acid (2 g, 11.7 mmol) in THF (100 ml) under nitrogen. The reaction mixture was stirred at room temperature for 2.5 h. The solvent was then evaporated and the residue was stirred in methanol (40 ml) for 16 h. The solvent was evaporated and the residue was purified on a silica isolute column, eluting with 0-1% MeOH in DCM to afford (5-fluoro-2-methoxy-pyridin-4-yl)methanol as a white solid (1.42 g, 77% yield).

¹H NMR (399.902 MHz, CDCl3) δ 3.90 (s, 3H), 4.76 (s, 2H), 6.84-6.87 (m, 1H), 7.92 (d, 1H). MS: m/z 158 (MH+)

### Example 63

### 3-[2-[5-[[2-[[3-(dimethylaminomethyl)-1,2-oxazol-5-yl]methylamino]pyrimidin-4-yl]amino]-1H-pyrazol-3-yl]ethyl]phenol

3-[2-[5-[[2-[[3-(hydroxymethyl)-1,2-oxazol-5-yl]methylamino]pyrimidin-4-yl]amino]-1H-pyrazol-3-yl]ethyl]phenol (97 mg, 0.24 mmoles) was suspended in DCM (5 ml) and thionyl chloride (87 μL, 1.19 mmoles) was added. The reaction was stirred at room temperature overnight. A further amount of thionyl chloride (87 [L, 1.19 mmoles) was added and the reaction was stirred for 2 h. The mixture was evaporated to dryness and then 2M dimethylamine solution in THF (5 ml) was added. The mixture was heated at 75° C. for 3 h. The mixture was evaporated to dryness. Purification by silica column chromatography, eluting with 5-10% methanol (containing 10% 7N ammonia in methanol) in dichloromethane, gave the crude product. The crude product was purified by reverse-phase prep. HPLC (basic) using a 5-98% gradient of acetonitrile in water containing 1% ammonium hydroxide solution, and a solid was obtained (26 mg 25%).

1H NMR (DMSO 400.13 MHz) δ 2.16 (s, 6H), 2.84 (s, 4H), 3.45 (s, 2H), 4.61 (d, 2H), 6.21 (s, 1H), 6.31 (bs, 1H), 6.63 (m, 1H), 6.70 (m, 2H), 7.11 (t, 1H), 7.25 (bs, 1H), 7.38 (d, 1H), 9.40 (bs, 1H), 11.96 (bs, 1H)

MS: m/z 435 (MH+).

3-[2-[5-[[2-[[3-(hydroxymethyl)-1,2-oxazol-5-yl]methylamino]pyrimidin-4-yl]amino]-1H-pyrazol-3-yl]ethyl]phenol, used as starting material was prepared as follows:—

[5-[[[4-[[5-[2-(3-methoxyphenyl)ethyl]-2H-pyrazol-3-yl]amino]pyrimidin-2-yl]amino]methyl]-1,2-oxazol-3-yl]methanol (120 mg, 0.28 mmoles) was dissolved in DCM (6 ml) and cooled to 0° C. under nitrogen. Boron tribromide solution (1M in DCM, 1.42 ml, 1.42 mmoles) was added dropwise and the reaction was allowed to warm to room temperature and stirred overnight. A further amount of boron tribromide (0.3 ml) was subsequently added. After 5 h, the reaction mixture was quenched with methanol (10 ml). The yellow solution was stirred for 1 h then evaporated to dryness. The crude product was loaded onto a SCX-2 column, washed with methanol. The product was eluted with 3.5N ammonia in methanol to give the desired crude product as a yellow foam after evaporation (97 mg, 85%). The product was used further without any purification.

MS: m/z 408 (MH+).

[5-[[[4-[[5-[2-(3-methoxyphenyl)ethyl]-2H-pyrazol-3-yl]amino]pyrimidin-2-yl]amino]methyl]-1,2-oxazol-3-yl]methanol was prepared as in Example 61.

### Example 64

### 5-[[[4-[[5-[2-(3-methoxyphenyl)ethyl]-2H-pyrazol-3-yl]amino]pyrimidin-2-yl]amino]methyl]-N-methyl-1,2-oxazole-3-carboxamide

To 2-chloro-N-[5-[2-(3-methoxyphenyl)ethyl]-2H-pyrazol-3-yl]pyrimidin-4-amine(100 mg, 0.30 mmoles) was added 5-(aminomethyl)-N-methyl-1,2-oxazole-3-carboxamide (88 mg, 0.45 mmoles) followed by 2-methoxyethanol (3 ml) and diisopropylethylamine (159 μL). The reaction was heated in the microwave at 200° C. for 60 mins. The solvent was evaporated under reduced pressure. The crude product was purified by silica column chromatography, eluting with 5-10% methanol in dichloromethane. Desired fractions were combined and evaporated to give the product as a yellow foam. Trituration with diethyl ether and filtration gave a pale yellow solid (80 mg (60%)

1H NMR (DMSO 400.13 MHz) δ 2.64 (d, 3H), 2.75 (m, 4H), 3.64 (s, 3H), 4.52 (d, 2H), 6.21 (bs, 1H), 6.43 (s, 1H), 6.64 (m, 1H), 6.7 (m, 2H), 7.08 (t, 1H), 7.15 (s, 1H), 7.73 (d, 1H), 8.48 (d, 1H), 9.25 (s, 1H), 11.82 (s, 1H)

MS: m/z 449 (MH+).

2-chloro-N-[5-[2-(3-methoxyphenyl)ethyl]-2H-pyrazol-3-yl]pyrimidin-4-amine, used as starting material was prepared as in Example 27.

5-(aminomethyl)-N-methyl-1,2-oxazole-3-carboxamide, used as starting material was prepared as follows:—

tert-Butyl N-[[3-(methylcarbamoyl)-1,2-oxazol-5-yl]methyl]carbamate (928 mg, 3.63 mmol, 1 eq) was dissolved in dichloromethane (10 mL). 6M HCl in propanol (1 mL) was added and the reaction was stirred at room temperature for 6 h. The mixture was evaporated to dryness, triturated with DCM, filtered and washed with diethyl ether to give 5-(aminomethyl)-N-methyl-1,2-oxazole-3-carboxamide. HCl salt as a white solid (532 mg, 77%).

1H NMR (400.13 MHz DMSO) δ 2.78 (3H, d), 4.32 (3H, s), 6.93 (1H, s), 8.77 (4H, m). MS m/z 156 (MH+).

tert-Butyl N-[[3-(methylcarbamoyl)-1,2-oxazol-5-yl]methyl]carbamate, used as starting material was prepared as follows:—

Ethyl 5-[[(2-methylpropan-2-yl)oxycarbonylamino]methyl]-1,2-oxazole-3-carboxylate (1 g, 3.7 mmol, 1 eq) was dissolved in 2M methylamine in THF (5mL) and stirred at room temperature overnight. The mixture was evaporated to dryness, triturated with diethyl ether and dried to give the product as a white solid (929 mg, 98%).

1H NMR (CDCl3 400.13 MHz) δ 1.43 (9H, s), 2.99 (3H, d), 4.45 (2H, d), 4.98 (1H, s), 6.6 (1H, s), 6.75 (1H, s). MS m/z 254 (M−H).

Ethyl 5-[[(2-methylpropan-2-yl)oxycarbonylamino]methyl]-1,2-oxazole-3-carboxylate used as starting material was prepared as shown in Example 61.

### Example 65

### 5-[[[4-[[5-[2-(3-hydroxyphenyl)ethyl]-2H-pyrazol-3-yl]amino]pyrimidin-2-yl]amino]methyl]-N-methyl-1,2-oxazole-3-carboxamide

5-[[[4-[[5-[2-(3-methoxyphenyl)ethyl]-2H-pyrazol-3-yl]amino]pyrimidin-2-yl]amino]methyl]-N-methyl-1,2-oxazole-3-carboxamide (70 mg, 0.16 mmoles) was dissolved in DCM (7 ml) and cooled to 0° C. under nitrogen. Boron tribromide (0.8 ml, 0.78 mmoles) solution was added dropwise and the reaction was allowed to warm to room temperature and stirred for 3 h. The reaction mixture was quenched carefully with methanol (5 ml) and the solution was evaporated to dryness. The crude product was loaded onto a SCX-2 column, washed with methanol and eluted with 2N ammonia in methanol to give the product as a yellow gum. Trituration with ether gave a cream solid which was filtered and dried in a vacuum oven at 45° C. (52 mg (75%).

1H NMR (DMSO 500.13 MHz @373K) d 2.7 (d, 3H), 2.79 (s, 4H), 4.6 (d, 2H), 6.28 (bs, 1H), 6.51 (s, 1H), 6.55 (m, 1H), 6.62 (m, 2H), 7.04 (t, 1H), 7.28 (bs, 1H), 7.81 (d, 1H), 8.56 (d, 1H), 9.2 (s, 1H), 9.38 (bs, 1H), 11.9 (bs, 1H)

MS: m/z 435 (MH+).

5-[[[4-[[5-[2-(3-methoxyphenyl)ethyl]-2H-pyrazol-3-yl]amino]pyrimidin-2-yl]amino]methyl]-N-methyl-1,2-oxazole-3-carboxamide used as starting material, was prepared as in Example 64.

### Example 122

### N-[(3-methyl-1,2-oxazol-5-yl)methyl]-N′-[5-[2-(3-propan-2-yloxyphenyl)ethyl]-1H-pyrazol-3-yl]pyrimidine-2,4-diamine

2-chloro-N-[5-[2-(3-propan-2-yloxyphenyl)ethyl]-1H-pyrazol-3-yl]pyrimidin-4-amine (60 mg, 0. 17 mmol, 1 eq) was dissolved in 2-methoxyethanol (5 ml) and (3-methyl-1,2-oxazol-5-yl)methanamine hydrochloride (50 mg, 0.34 mmol, 2 eq) and N-ethyl-N-propan-2-yl-propan-2-amine (103 μl, 0.59 mmol, 3.5 eq) were added. The mixture was heated to 180° C. for 90 mins in the microwave reactor. The solvent was evaporated under reduced pressure and the residue purified by basic reverse-phase prep HPLC (gradient 25-75% MeCN in 1% aq NH₃). Clean fractions were evaporated to afford N-[(3-methyl-1,2-oxazol-5-yl)methyl]-N′-[5-[2-(3-propan-2-yloxyphenyl)ethyl]-1H-pyrazol-3-yl]pyrimidine-2,4-diamine (25.4 mg, 35%) as a beige solid.

¹H NMR (399.902 MHz, DMSO) δ 1.17 (d, J=6.0 Hz, 6H), 2.10 (s, 3H), 2.78 (m, 4H), 3.21 (s, 1H), 4.48 (m, 3H), 6.03 (s, 1H), 6.21 (s, 1H), 6.68 (m, 3H), 7.10 (m, 2H), 7.75 (d, J=5.8 Hz, 1H), 9.27 (s, 1H), 11.83 (s, 1H). MS: m/z=434 (MH+)

(3-methyl-1,2-oxazol-5-yl)methanamine hydrochloride, used as starting material, was prepared as outlined in Example 1.

2-chloro-N-[5-[2-(3-propan-2-yloxyphenyl)ethyl]-1H-pyrazol-3-yl]pyrimidin-4-amine used a starting material was prepared as follows:—

2,4-dichloropyrimidine (177 mg, 1.2 mmol, 1 eq) was dissolved in ethanol (5 ml) and N-ethyl-N-propan-2-yl-propan-2-amine (0.25 ml, 1.4 mmol, 1.2 eq) and 5-[2-(3-propan-2-yloxyphenyl)ethyl]-1H-pyrazol-3-amine (290 mg, 1.3 mmol, 1.1 eq) were added. The mixture was stirred at 50° C. for 3 days. The reaction mixture was added slowly to water (10 ml), sonicated and left to stand overnight. The red-brown precipitate was collected by filtration, washed with water and dried in vacuo. The precipitate was dissolved in a minimum amount of methanol, water was added dropwise and the colourless precipitate was filtered and washed with water and dried in vacuo to give 2-chloro-N-[5-[2-(3-propan-2-yloxyphenyl)ethyl]-1H-pyrazol-3-yl]pyrimidin-4-amine (121.6 mg, 29%) as a colourless solid.

¹H NMR (399.902 MHz, DMSO) δ 1.17 (d, J=6.0 Hz, 6H), 2.81 (s, 4H), 4.49 (septet, J=6.0 Hz, 1H), 6.02 (s, 1H), 6.69 (m, 4H), 7.10 (t,J=8.1 Hz, 1H), 8.09 (d, J=5.8 Hz, 1H), 10.22 (s, 1H). MS: m/z=358 (MH+).

5-[2-(3-propan-2-yloxyphenyl)ethyl]-1H-pyrazol-3-amine was prepared as follows:—

Methyl 3-(3-propan-2-yloxyphenyl)propanoate (680 mg, 3.1 mmol, 1 eq) was dissolved in 1,4-dioxane (20 ml). Sodium hydride (60% suspension) (147 mg, 3.7 mmol, 1.2 eq) and dry acetonitrile (0. 19 ml, 3.7 mmol, 1.2 eq) were added. The solution was stirred at room temperature for 10 mins and then at 100° C. overnight. The mixture was cooled to room temperature and dry ethanol (2 ml) and hydrazine hydrochloride (420 mg, 6.1 mmol, 2 eq) were added. The mixture was refluxed overnight, cooled, evaporated and then partitioned between 1M HCl and EtOAc. The aqueous layer was basified with conc. ammonia then extracted with EtOAc. The organic extracts were combined and washed with water then brine, dried and evaporated. The crude product was purified by silica column chromatography, eluting with 0.5-7% MeOH in DCM. The clean fractions were evaporated to yield 5-[2-(3-propan-2-yloxyphenyl)ethyl]-1H-pyrazol-3-amine (296 mg, 39%) as a brown oil.

¹H NMR (399.902 MHz, DMSO) δ 1.18 (d, J=5.7 Hz, 6H), 2.63 (m, 2H), 2.73 (m, 2H), 4.33 (bs, 1H), 4.50 (septet, J=6.0 Hz, 1H), 5.12 (s, 1H), 6.66 (m, 3H), 7.08 (t, J=8.1 Hz, 1H), 11.03 (bs, 1H). MS: m/z=246 (MH+).

Methyl 3-(3-propan-2-yloxyphenyl)propanoate was prepared as follows:—

Methyl 3-(3-hydroxyphenyl)propanoate (1 g, 5.5 mmol, 1 eq) was dissolved in dry acetone (20 ml) and anhydrous potassium carbonate (921 mg, 6.7 mmol, 1.2 eq) and 2-iodopropane (0.67 ml, 6.7 mmol, 1.2 eq) were added. The mixture was heated to 55° C. under nitrogen for 24 h. Further potassium carbonate (844 mg, 5.6 mmol, 1 eq) and 2-iodopropane (0.4 ml, 4.0 mmol, 0.8 eq) were then added and stirring at 56° C. was continued for 24 h. The solvent was evaporated and the residue dissolved in water (25 ml). The solution was extracted with diethyl ether (3×10 ml) and the extracts were combined, dried and evaporated. The crude product was purified by silica column chromatography, eluting with 0-10% MeOH in DCM. The pure fractions were combined, evaporated and dried to give methyl 3-(3-propan-2-yloxyphenyl)propanoate (686 mg, 56%) as a yellow oil.

¹H NMR (399.902 MHz, DMSO) δ 1.18 (d, J=5.9 Hz, 6H), 2.55 (t, J=7.6 Hz, 2H), 2.74 (t, J=7.6 Hz, 2H), 3.52 (s, 3H), 4.51 (septet, J=6.0 Hz, 1H), 6.67 (m, 3H), 7.09 (t, J=8.0 Hz, 1H).

Methyl 3-(3-hydroxyphenyl)propanoate was prepared as follows:—

3-(3-hydroxyphenyl)propanoic acid (3 g, 18.0 mmol, 1 eq) was dissolved in dry DMF (50 ml) and to this was added potassium hydrogen carbonate (2.17 g, 21.7 mmol, 1.2 eq). The reaction mixture was stirred at room temperature under nitrogen for 10 mins. Methyl iodide (1.24 ml, 19.9 mmol, 1.1 eq) was then added and the mixture was heated at 40° C. overnight. The solvent was evaporated and the residue dissolved in diethyl ether and washed with water followed, by ammonium chloride solution, dried and evaporated to give methyl 3-(3-hydroxyphenyl)propanoate (3.205 g, 98%) as a brown oil.

¹H NMR (399.902 MHz, DMSO) δ 2.59 (t, J=7.9 Hz, 2H), 2.77 (t, J=7.7 Hz, 2H), 3.59 (s, 3H), 6.60 (m, 3H), 7.06 (m, 1H), 9.24 (s, 1H). MS: m/z=179 (M−H+)

### Example 123

### 5-[[[4-[[5-[2-[3-(cyclopropylmethoxy)phenyl]ethyl]-1H-pyrazol-3-yl]amino]pyrimidin-2-yl]amino]methyl]-1,2-oxazole-3-carboxamide

2-chloro-N-[5-[2-[3-(cyclopropylmethoxy)phenyl]ethyl]-1H-pyrazol-3-yl]pyrimidin-4-amine (100 mg, 0.27 mmol, 1 eq) was dissolved in 2-methoxyethanol and 5-(aminomethyl)-1,2-oxazole-3-carboxamide hydrochloride (97 mg, 54 mmol, 2 eq) and N-ethyl-N-propan-2-yl-propan-2-amine (165 μl, 0.95 mmol, 3.5 eq) were added. The mixture was heated to 180° C. for 105 mins in the microwave reactor. The solvent was evaporated under reduced pressure and the residue purified on basic reverse phase prep HPLC (gradient 25-85% MeCN in 1% aq NH₃). The clean fractions were evaporated to give 5-[[[4-[[5-[2-[3-(cyclopropylmethoxy)phenyl]ethyl]-1H-pyrazol-3-yl]amino]pyrimidin-2-yl]amino]methyl]-1,2-oxazole-3-carboxamide (14.8 mg, 12%) as a beige solid.

¹H NMR (399.902 MHz, DMSO) δ 0.22 (m, 2H), 0.47 (m, 2H), 1.13 (m, 1H), 2.78 (m, 4H), 3.70 (d, J=7.1 Hz, 2H), 4.54 (d, J=5.8 Hz, 2H), 6.24 (s, 1H), 6.45 (s, 1H), 7.10 (t, J=8.0 Hz, 1H), 7.19 (s, 1H), 7.66 (s, 1H), 7.76 (d, J=5.7 Hz, 1H), 9.30 (s, 1H), 11.84 (s, 1H). MS: m/z=475 (MH+).

5-(Aminomethyl)-1,2-oxazole-3-carboxamide hydrochloride used a starting material was prepared as follows:—

Tert-butyl N-[(3-carbamoyl-1,2-oxazol-5-yl)methyl]carbamate (1.6 g, 6.63 mmol, 1 eq) was dissolved in dichloromethane (32 mL). 6M HCl in propanol (1.6 mL) was added and the reaction was stirred at room temperature for 6 h. The mixture was evaporated to dryness, triturated with DCM, filtered and washed with diethyl ether to give 5-(aminomethyl)-1,2-oxazole-3-carboxamide hydrochloride salt as white solid (1. 17 g, 100%).

1H NMR (400.13 MHz DMSO) δ 4.38 (2H, s), 6.40 (1H, s), 7.85 (1H, s), 8.15 (1H, s), 8.76 (3H, s)

tert-Butyl N-[(3-carbamoyl-1,2-oxazol-5-yl)methyl]carbamate used as starting material was prepared as follows:—

Ethyl 5-[[(2-methylpropan-2-yl)oxycarbonylamino]methyl]-1,2-oxazole-3-carboxylate (2 g, 7.4 mmol, 1 eq) was dissolved in 3.5N ammonia in methanol (10 mL) and stirred at room temperature overnight. The mixture was evaporated to dryness, triturated with diethyl ether and dried on the filter to give product as a white solid (1.6 g, 90%).

1H NMR (CDCl3 400.13 MHz) δ 1.44 (9H, s), 4.45 (2H, d), 4.96 (1H, s), 5.58 (1H, s), 6.61 (1H, s), 6.65 (1H, s). MS m/z 240 (M−H).

Ethyl 5-[[(2-methylpropan-2-yl)oxycarbonylamino]methyl]-1,2-oxazole-3-carboxylate used as starting material was prepared as follows:—

tert-butyl N-prop-2-ynylcarbamate (40.97 g, 0.26 mol, 1 eq) was dissolved in anhydrous THF (150 mL) and N,N-diethylethanamine (22 mL, 0. 16 mol, 1.2 eq) added. A solution of ethylchlorooximidoacetate (20 g, 0.13 mol, 1 eq) in anhydrous THF (350 mL) was added dropwise over 7 h. The reaction was stirred at room temperature overnight then evaporated to dryness. The residue was dissolved in DCM and washed with water, brine and dried (MgSO₄). After filtration, the solution was evaporated to give the crude product as a yellow oil. This was purified by silica column chromatography, eluting with 20% -60% ether in iso-hexane to give ethyl 5-[[(2-methylpropan-2-yl)oxycarbonylamino]methyl]-1,2-oxazole-3-carboxylate as a white solid (20.12 g, 56%).

1H NMR (CDCl3 400.13 MHz) δ 1.39-1.47 (12H, m), 4.40-4.49 (5H, m), 5.0 (1H, s), 6.58 (1H, s). MS m/z 269 (M−H).

tert-butyl N-prop-2-ynylcarbamate, used as starting material was prepared as follows:—

(2-Methylpropan-2-yl)oxycarbonyl tert-butyl carbonate (99.3 g, 455 mmol) was added portion wise over 30 mins to a stirred solution of prop-2-yn-1-amine (25 g, 455 mmol) in anhydrous diethyl ether (500 mL) at 0-10° C. The mixture was allowed to reach room temperature and stirred under an atmosphere of nitrogen for 72 h. The reaction mixture was evaporated to dryness, triturated at −10° C. with hexanes (400 ml), filtered to give a solid, washed with hexane and dried to afford of tert-butyl N-prop-2-ynylcarbamate as white crystalline solid (62.5 g, 88.5%). ¹H NMR (399.9 MHz, CDCl₃) 6 1.41-1.51 (9H, m), 2.22 (1H, t), 3.92 (2H, d), 4.75 (1H, s)

2-Chloro-N-[5-[2-[3-(cyclopropylmethoxy)phenyl]ethyl]-1H-pyrazol-3-yl]pyrimidin-4-amine was prepared as follows:—

5-[2-[3-(cyclopropylmethoxy)phenyl]ethyl]-1H-pyrazol-3-amine (560 mg, 2.4 mmol, 1. 1 eq) was dissolved in ethanol (10 ml) and N-ethyl-N-propan-2-yl-propan-2-amine (0.46 ml, 2.6 mmol, 1.2 eq) and 2,4-dichloropyrimidine (325 mg, 2.2 mmol, 1.0 eq) were added. The mixture was stirred at 40° C. for 3 days. The reaction mixture was, added slowly to water (30 ml), sonicated and the precipitate was collected by filtration, washed (2:1 mixture of water and MeOH) and dried in vacuo to yield 2-chloro-N-[5-[2-[3-(cyclopropylmethoxy)phenyl]ethyl]-1H-pyrazol-3-yl]pyrimidin-4-amine (380 mg, 47%) as a beige solid.

¹H NMR (399.902 MHz, DMSO) δ 0.23 (m, 2H), 0.48 (m, 2H), 1.12 (m, 1H), 2.81 (m, 4H), 3.71 (d, J=7.0 Hz, 2H), 6.01 (bs, 1H), 6.69 (m, 3H), 7.10 (m, 1H), 8.09 (d, J=5.7 Hz, 1H), 10.20 (s, 1H), 12.12 (s, 1H). MS: m/z=370 (MH+).

5-[2-[3-(cyclopropylmethoxy)phenyl]ethyl]-1H-pyrazol-3-amine was prepared as follows:—

LDA (3.61 ml, 7.2 mmol, 2.0 eq) was added to dry THF (15 ml) and the solution was cooled to −78° C. Acetonitrile (377 μl, 7.2 mmol, 2.0 eq) was added dropwise and the mixture was stirred for 10 mins. Methyl 3-[3-(cyclopropylmethoxy)phenyl]propanoate (845 mg, 3.6 mmol, 1.0 eq) in THF (5 ml) was added quickly and after 10 mins the mixture was allowed to warm up to room temperature. The mixture was quenched with 1 N HCl (20 ml), extracted with diethyl ether (3×20 ml), dried and evaporated. The residue was dissolved in ethanol (20 ml), hydrazine (350 μl, 7.2 mmol, 2.0 eq) was added and the solution was refluxed for 24 h. The reaction mixture was cooled, evaporated to dryness, dissolved in water (30 ml) and extracted with diethyl ether (3×20 ml). The extracts were combined, dried and evaporated to dryness. The residue was purified by silica column chromatography, eluting with 3-8% MeOH in DCM. The desired fractions were combined and evaporated to yield 5-[2-[3-(cyclopropylmethoxy)phenyl]ethyl]-1H-pyrazol-3-amine (568 mg, 61%) as a brown oil.

1H NMR (399.902 MHz, DMSO) δ 0.24 (m, 2H), 0.49 (m, 2H), 1.13 (m, 1H), 2.64 (m, 2H), 2.73 (m, 2H), 3.71 (d, J=7.0 Hz, 2H), 4.25 (bs, 2H), 5.13 (bs, 1H), 6.67 (m, 3H), 7.09 (t, J=8.1 Hz, 1H), 11.00 (bs, 1H). MS: m/z=258 (MH+).

Methyl 3-[3-(cyclopropylmethoxy)phenyl]propanoate was prepared as follows:—

Methyl 3-(3-hydroxyphenyl)propanoate (1 g, 5.5 mmol, 1.0 eq) was dissolved in dry acetone (20 ml) and anhydrous potassium carbonate (1.54 g, 11.1 mmol, 2.0 eq), potassium iodide (185 mg 1.1 mmol, 0.2 eq) and (bromomethyl)cyclopropane (1.08 ml, 11.1 mmol, 2.0 eq) were added. The mixture was stirred at 55° C. under nitrogen for 2 days. The reaction mixture was cooled to room temperature, evaporated to dryness and the residue was dissolved in water (25 ml) and extracted with diethyl ether (3×10 ml). The extracts were combined, dried (MgSO₄) and evaporated to dryness. The residue was dissolved in a small amount of DCM and purified by silica column chromatography, eluting with DCM. The pure fractions were combined and evaporated to give methyl 3-[3-(cyclopropylmethoxy)phenyl]propanoate (856 mg, 66%) as a colourless oil.

¹H NMR (399.902 MHz, DMSO) δ 0.24 (m, 2H), 0.49 (m, 2H), 1.13 (m, 1H), 2.55 (t, J=7.7 Hz, 2H), 2.74 (t, J=7.6 Hz, 2H), 3.52 (s, 3H), 3.72 (d, J=7.0 Hz, 2H), 6.66 (m, 1H), 6.68 (m, 1H), 6.71 (m, 1H), 7.09 (t, J=7.8 Hz, 1H). MS: m/z=235 (MH+).

Methyl 3-(3-hydroxyphenyl)propanoate was prepared as follows:—

3-(3-hydroxyphenyl)propanoic acid (3 g, 18.0 mmol, 1 eq) was dissolved in dry DMF (50 ml), potassium hydrogen carbonate (2.17 g, 21.7 mmol, 1.2 eq) was added and the mixture was stirred at room temperature under nitrogen for 10 mins. Methyl iodide (1.24 ml, 19.9 mmol, 1.1 eq) was added and the mixture was heated at 40° C. overnight. The solvent was evaporated and the residue dissolved in diethyl ether and washed with water followed, by ammonium chloride solution, dried and evaporated to give methyl 3-(3-hydroxyphenyl)propanoate (3.205 g, 98%) as a brown oil.

¹H NMR (399.902 MHz, DMSO) 6 2.59 (t, J=7.9 Hz, 2H), 2.77 (t, J=7.7 Hz, 2H), 3.59 (s, 3H), 6.60 (m, 3H), 7.06 (m, 1H), 9.24 (s, 1H). MS: m/z=179 (M−H+)

### Example 124

### N′-[5-[2-(2,6-dimethoxypyridin-4-yl)ethyl]-1H-pyrazol-3-yl]-N-[(3-methyl-1,2-oxazol-5-yl)methyl]pyrimidine-2,4-diamine

4-chloro-N-[(3-methyl-1,2-oxazol-5-yl)methyl]pyrimidin-2-amine (72.4 mg, 0.32 mmol, 1 eq) was added to a stirred solution of 5-(2-(2,6-dimethoxypyridin-4-yl)ethyl)-1H-pyrazol-3-amine (80 mg, 0.32 mmol, 1 eq) in ethanol (5 ml) at room temperature. The resulting solution was stirred at 80° C. for 45 h. The reaction mixture was cooled and a precipitate formed. The mixture was filtered and the solid washed with ethanol to afford N′-[5-[2-(2,6-dimethoxypyridin-4-yl)ethyl]-1H-pyrazol-3-yl]-N-[(3-methyl-1,2-oxazol-5-yl)methyl]pyrimidine-2,4-diamine (59.3 mg) as a white solid. The filtrate was concentrated and further product (32.0 mg) precipitated and was collected by filtration.

1H NMR (399.902 MHz, DMSO) δ 2.24 (s, 3H), 2.90 (m, 4H), 3.87 (s, 6H), 4.75 (d, J=5.7 Hz, 2H), 6.29 (s, 2H), 6.32 (s, 1H), 6.43 (s, 1H), 7.95 (s, 1H), 8.86 (s, 1H), 11.26 (s, 1H), 12.47 (s, 1H), 12.70 (s, 1H). MS: m/z=437 (MH+)

5-(2-(2,6-dimethoxypyridin-4-yl)ethyl)-1H-pyrazol-3-amine used as starting material was prepared as follows:—

Acetonitrile (0.209 mL, 4.00 mmol, 2 eq) was added dropwise to a stirred solution of lithium diisopropylamide (2.220 mL, 4.00 mmol, 2 eq) in THF (15 mL) cooled to −78° C., over a period of 1 minute under nitrogen. The resulting solution was stirred for 10 mins. A solution of methyl 3-(2,6-dimethoxypyridin-4-yl)propanoate (450 mg, 2.00 mmol, 1 eq) in THF (15 mL) was added. The resulting solution was stirred at −78° C. for 30 mins, then allowed to warm to room temperature. Ethanol (20 mL) and hydrazine hydrochloride (301 mg, 4.40 mmol, 2.2 eq) were added and the solution was refluxed for 18 h. The reaction mixture was evaporated to dryness, redissolved in Et2O (20 mL) and washed with water (3×10 mL). The organic layer was dried over MgSO4, filtered and evaporated to afford crude product. The crude product was purified by silica column chromatography, eluting with a gradient of 2-8% MeOH in DCM. Pure fractions were evaporated to dryness to afford 5-(2-(2,6-dimethoxypyridin-4-yl)ethyl)-1H-pyrazol-3-amine (385 mg, 1.55 mmol, 78%) as a colourless oil which crystallised upon standing.

1H NMR (399.902 MHz, DMSO) δ 2.74 (m, 4H), 3.82 (s, 6H), 4.41 (bs, 2H), 5.18 (bs, 1H), 6.24 (s, 2H), 11.06 (bs, 1H) MS: m/z=249 (MH+)

Methyl 3-(2,6-dimethoxypyridin-4-yl)propanoate prepared as follows:—

(E)-methyl 3-(2,6-dimethoxypyridin-4-yl)acrylate (400 mg, 1.79 mmol) and Pd/C 10% (50 mg) in ethanol (50 mL) were stirred under an atmosphere of hydrogen at room temperature for 18 h. The reaction mixture was filtered to remove the catalyst and the fitrate evaporated under reduced pressure to give methyl 3-(2,6-dimethoxypyridin-4-yl)propanoate (400 mg, 99%).

1H NMR (399.902 MHz, DMSO) δ 2.69 (t, J=7.7 Hz, 2H), 2.83 (t, J=7.5 Hz, 2H), 3.64 (s, 3H), 3.87 (s, 6H), 6.30 (s, 2H) Plus ethanol. MS: m/z=226 (MH+)

(E)-methyl 3-(2,6-dimethoxypyridin-4-yl)acrylate was prepared as follows:—

2,6-dimethoxypyridine-4-carbaldehyde (580 mg, 3.5 mmol, 1 eq) was dissolved in DCM (12 ml) under nitrogen and methyl (triphenylphosphoranylidene)acetate (1.745 g, 5.2 mmol, 1.5 eq) was added portionwise. The mixture was stirred at room temperature overnight and then evaporated to dryness. The crude product was purified by silica column chromatography, eluting with 3-10% EtOAc in isohexane. The desired fractions were combined and evaporated to give (E)-methyl 3-(2,6-dimethoxypyridin-4-yl)acrylate (464 mg, 60%) as a pale yellow solid.

¹H NMR (399.902 MHz, DMSO) δ 3.68 (s, 3H), 3.80 (s, 6H), 6.65 (s, 2H), 6.76 (d, J=16.2 Hz, 1H), 7.47 (d, J=16.2 Hz, 1H). MS: m/z=224 (MH+)

2,6-dimethoxypyridine-4-carbaldehyde was prepared as follows:—

(2,6-dimethoxypyridin-4-yl)methanol (620 mg, 3.7 mmol, 1 eq) was stirred in dry DCM (30 ml) under nitrogen. Dess Martin periodinane (1.87 g, 4.4 mmol, 1.2 eq) in DCM (30 ml) was slowly added and the mixture was stirred for 30 mins. The solution was washed with NaOH (aq) followed by water, dried (MgSO₄) and evaporated to give 2,6-dimethoxypyridine-4-carbaldehyde (587 mg, 96%) as a purple solid.

¹H NMR (399.902 MHz, DMSO) δ 3.98 (s, 6H), 6.86 (s, 2H), 10.03 (s, 1H). MS: m/z=168 (MH+).

(2,6-Dimethoxypyridin-4-yl)methanol was prepared as follows:—

Crude 2,6-dimethoxypyridine-4-carboxylic acid (65 mol% by NMR) (1.5 g, 8.2 mmol, 1 eq) was dissolved in dry THF (100 ml) under nitrogen and BH₃.THF adduct (1M in THF; 36.8 ml, 36.8 mmol, 4.5 eq) was added dropwise. The reaction was stirred at room temperature for 2.5 h. The solvent was evaporated and methanol (30 ml) was then added. The solution was stirred at room temperature for 30 mins then evaporated to dryness. The resulting oil was purified by silica column chromatography, eluting with 0-1% MeOH in DCM. Desired fractions were combined and evaporated to give (2,6-dimethoxypyridin-4-yl)methanol (536 mg, 39%) as a colourless solid.

¹H NMR (399.902 MHz, DMSO) δ 3.81 (s, 6H), 4.42 (d, J=5.9 Hz, 2H), 5.29 (t, J=5.9 Hz, 1H), 6.29 (s, 2H). MS: m/z=170 (MH+).

2,6-Dimethoxypyridine-4-carboxylic acid was prepared as follows:—

2,6-Dichloropyridine-4-carboxylic acid (3 g, 15.6 mmol, 1 eq)) was dissolved in dry DMF (40 ml) and sodium methoxide (2.96 g, 54.7 mmol, 3.5 eq) added under nitrogen. The mixture was heated under reflux for 7.5 h, then cooled. A further 1.4 g sodium methoxide was added and the reaction mixture was refluxed overnight. A further 1.7 g sodium methoxide was added and the reaction mixture was refluxed for a further 4.5 h. The reaction mixture was cooled, added to an equal volume of ice-water and acidified. The precipitate was collected by filtration, washed with water to give crude 2,6-dimethoxypyridine-4-carboxylic acid (2.7 g, 98% but only 65 mol%) as a yellow solid.

4-Chloro-N-[(3-methyl-1,2-oxazol-5-yl)methyl]pyrimidin-2-amine was prepared as outlined in Example 13.

### Example 125

### N′-[5-[2-(3-aminophenyl)ethyl]-1H-pyrazol-3-yl]-N-[(3-methyl-1,2-oxazol-5-yl)methyl]pyrimidine-2,4-diamine

tert-butyl N-[3-[2-(5-amino-2H-pyrazol-3-yl)ethyl]phenyl]carbamate (100 mg, 0.3 mmol, 1 eq) was dissolved in ethanol and 4-chloro-N-[(3-methyl-1,2-oxazol-5-yl)methyl]pyrimidin-2-amine (75 mg, 0.3 mmol, 1 eq) was added. The mixture was stirred at 80° C. for 40 h. The reaction mixture was evaporated and the residue purified by basic prep. HPLC, eluting with acetonitrile in water with 1% ammonia. 10 ml HCl (4 M) in dioxane was added and the solution was stirred at room temperature for 1 h The solvent was evaporated and the residue was dissolved in dichloromethane (20 ml), washed with saturated NaHCO₃ solution (20 ml), dried (MgSO₄), evaporated and dried in vacuo to give N′-[5-[2-(3-aminophenyl)ethyl]-1H-pyrazol-3-yl]-N-[(3-methyl-1,2-oxazol-5-yl)methyl]pyrimidine-2,4-diamine (81.6 mg, 63%) as a yellow solid.

¹H NMR (399.902 MHz, DMSO) δ 2.22 (s, 3H), 2.81 (m, 4H), 4.59 (d, J=6.2 Hz, 2H), 4.99 (bs, 1H), 6.17 (s, 1H), 6.31 (bs, 1H), 6.47 (m, 3H), 6.97 (t, J=7.8 Hz, 1H), 7.28 (bs, 1H), 7.88 (d, J=5.7 Hz, 1H), 9.44 (bs, 1H), 11.97 (bs, 1H). MS: m/z=391 (MH+)

tert-butyl N-[3-[2-(5-amino-2H-pyrazol-3-yl)ethyl]phenyl]carbamate used as starting material was prepared as follows:—

LDA (3.58 ml, 7.2 mmol, 4.0 eq) was added to THF (20 ml) and the mixture cooled to −78° C. Acetonitrile (374 μl, 7.2 mmol, 4.0 eq) was slowly added and the solution stirred for 10 mins. Methyl 3-[3-[(2-methylpropan-2-yl)oxycarbonylamino]phenyl]propanoate (500 mg, 1.8 mmol, 1.0 eq) was rapidly added. The reaction was stirred for 30 mins, then allowed to warm to room temperature. The mixture was quenched with 1 N HCl (30 ml) at 0° C., quickly extracted with diethyl ether (3×20 ml), dried over MgSO₄ and evaporated. The residue was dissolved in ethanol and hydrazine monohydrate (174 μl, 3.6 mmol, 2.0 eq) was added. The solution was refluxed for 24 h. The reaction mixture was cooled, evaporated to dryness, dissolved in water and extracted with diethyl ether. The extracts were combined, dried (MgSO₄), evaporated and dried in vacuo to give tert-butyl N-[3-[2-(5-amino-2H-pyrazol-3-yl)ethyl]phenyl]carbamate (500 mg, 92%) as a yellow solid.

¹H NMR (399.902 MHz, DMSO) δ 1.53 (s, 10H), 2.74 (m, 2H), 2.83 (m, 2H), 4.37 (bs, 1H), 5.26 (bs, 1H), 6.88 (d, J=7.7 Hz, 1H), 7.19 (t, J=7.8 Hz, 1H), 7.29 (d, J=7.7 Hz, 1H), 7.44 (s, 1H), 9.28 (s, 1H), 11.15 (bs, 1H). MS: m/z=303 (MH+).

Methyl 3-[3-[(2-methylpropan-2-yl)oxycarbonylamino]phenyl]propanoate was prepared as follows:—

3-[3-[(2-Methylpropan-2-yl)oxycarbonylamino]phenyl]propanoic acid (3 g, 11.3 mmol, 1.0 eq) was dissolved in dry DMF (50 ml) and potassium hydrogen carbonate (2.17 g, 13.6 mmol, 1.2 eq) was added. The mixture was stirred at room temperature under nitrogen for 10 mins. Methyl iodide (0.78 ml, 12.44 mmol, 1.1 eq) was added and the mixture was heated at 40° C. overnight. The solvent was evaporated and the residue dissolved in diethyl ether (30 ml), washed with water (20 ml), washed with saturated ammonium chloride solution (20 ml), dried (MgSO₄) and evaporated to give methyl 3-[3-[(2-methylpropan-2-yl)oxycarbonylamino]phenyl]propanoate (3.08 g, 97%) as a pale yellow solid.

¹H NMR (399.902 MHz, DMSO) δ 1.53 (s, 9H), 2.64 (t, J=7.6 Hz, 3H), 2.85 (t, J=7.6 Hz, 2H), 3.64 (s, 3H), 6.87 (d, J=7.5 Hz, 1H), 7.20 (t, J=7.8 Hz, 1H), 7.30 (d, J=8.4 Hz, 1H), 7.39 (s, 1H), 9.29 (s, 1H). MS: m/z=224 (MH+ minus t-butyl group).

4-Chloro-N-[(3-methyl-1,2-oxazol-5-yl)methyl]pyrimidin-2-amine was prepared as outlined in Example 13.

### Example 126

### 5-[[[4-[[5-[2-(3-chloro-5-methoxy-phenyl)ethyl]-2H-pyrazol-3-yl]amino]pyrimidin-2-yl]amino]methyl]-1,2-oxazole-3-carboxamide

To 2-chloro-N-[5-[2-(3-chloro-5-methoxy-phenyl)ethyl]-2H-pyrazol-3-yl]pyrimidin-4-amine (60 mg, 0. 16 mmol, 1 eq) was added 5-(aminomethyl)-1,2-oxazole-3-carboxamide hydrochloride (44 mg, 0.25 mmol, 1.5 eq) followed by 2-methoxyethanol (3 ml) and N-ethyl-N-propan-2-yl-propan-2-amine (87[L, 0.49 mmol, 3 eq). The reaction was heated in the microwave at 190° C. for 60 mins. The solvent was evaporated under reduced pressure and the crude product was purified by preparative HPLC using decreasingly polar mixtures of water (containing 1% ammonium hydroxide) and MeCN as eluents to give title compound as a white solid (56 mg, 76%).

1H NMR (DMSO 400.13 MHz) δ 2.87 (4H, m), 3.75 (3H, s), 4.60 (2H, d), 6.31 (1H, s), 6.52 (1H, s), 6.78 (1H, s), 6.83 (1H, s), 6.89 (1H, s), 7.34 (1H, s), 7.73 (1H, s), 7.83 (1H, s), 8.00 (1H, s), 9.36 (1H, s), 11.91 (1H, s). MS m/z 469 (MH+).

2-Chloro-N- {5-[2-(3-chloro-5-methoxyphenyl)ethyl]-1H-pyrazol-3-yl}pyrimidin-4-amine, used as starting material was prepared as follows:—

5-[2-(3-Chloro-5-methoxyphenyl)ethyl]-1H-pyrazol-3-amine (193 mg, 0.765 mmol) was stirred with N-ethyl-N-propan-2-yl-propan-2-amine (267[l, 1.53 mmol) and 2,4-dichloropyrimidine (1 14 mg, 0.765 mmol) in ethanol (5 ml) under nitrogen. The solution was heated at 50° C. for 4 days. The solution was concentrated under vacuum and water added to the residue. The mixture was then evaporated to dryness. The residue was then triturated with DCM (one drop methanol) and filtered to afford the product, 2-chloro-N- {5-[2-(3-chloro-5-methoxyphenyl)ethyl]-1H-pyrazol-3-yl}pyrimidin-4-amine, as a white solid (27 mg, 11%). The filtrate was evaporated and purified by silica column chromatography, eluting with 1-3% MeOH in DCM to afford a further crop of the product as a white solid (125 mg, 51% yield).

¹H NMR (399.902 MHz, DMSO) δ 2.90 (s, 4H), 3.76 (s, 3H), 6.11 (bs, 1H), 6.78-6.81 (m, 1H), 6.84-6.87 (m, 1H), 6.89-6.92 (m, 1H), 7.21 (bs, 1H), 8.16 (d, 1H), 10.28 (s, 1H), 12.20 (s, 1H); m/z (ES+) [M+H]+=364.

5-[2-(3-Chloro-5-methoxyphenyl)ethyl]-1H-pyrazol-3-amine, used as starting material was prepared as follows:—

Methyl 3-(3-chloro-5-methoxyphenyl)propanoate (880 mg, 3.85 mmol) and acetonitrile (242 μl, 4.62 mmol) were stirred in 1,4-dioxane (16 ml) under nitrogen. Sodium hydride (111 mg, 60% dispersion on mineral oil, 2.78 mmol) was added and the mixture was stirred at room temperature for 10 mins, then refluxed under nitrogen for 18 h. The mixture was allowed to cool to room temperature, ethanol (2 ml) was then added followed by hydrazine monohydrochloride (528 mg, 7.70 mmol) and the mixture was refluxed for 22 h. The mixture was concentrated under vacuum and the residue was partitioned between ethyl acetate (10 ml) and 2M HCl(aq) (15 ml). The organic phase was then washed with sat. aq. NaHCO₃, dried over MgSO₄, filtered, evaporated and purified by silica column chromatography, eluting with 0-3.5% MeOH in DCM to afford 5-[2-(3-chloro-5-methoxyphenyl)ethyl]-1H-pyrazol-3-amine as a light brown gum (414 mg, 43%).

¹H NMR (399.902 MHz, DMSO) δ 2.65-2.86 (m, 4H), 3.75 (s, 3H), 4.42 (bs, 2H), 5.19 (s, 1H), 6.75-6.78 (m, 1H), 6.82-6.85 (m, 1H), 6.86 (s, 1H), 11.03 (bs, 1H); m/z (ES+) [M+H]+=252.

Methyl 3-(3-chloro-5-methoxyphenyl)propanoate, used as starting material was prepared as follows:—

Platinum(IV) oxide (36 mg, 0.155 mmol) was added to a solution of methyl 3-(3-chloro-5-methoxy-phenyl)prop-2-enoate (880 mg, 3.88 mmol) in ethyl acetate (45 ml) and the mixture was stirred at room temperature under a hydrogen balloon for 20 h. The catalyst was removed by filtration, washed with ethyl acetate and the filtrate was evaporated to afford methyl 3-(3-chloro-5-methoxyphenyl)propanoate as a colourless oil (0.89 g, quant. yield).

¹H NMR (399.902 MHz, CDCl₃) δ 2.61 (t, 2H), 2.89 (t, 2H), 3.68 (s, 3H), 3.77 (s, 3H), 6.62-6.64 (m, 1H), 6.73-6.75 (m, 1H), 6.77-6.79 (m, 1H); m/z (ES+) [M+Na]+=251.

Methyl 3-(3-chloro-5-methoxy-phenyl)prop-2-enoate, used as starting material was prepared as follows:—

Methyl (triphenylphosphoranylidene)acetate (2.95 g, 8.79 mmol) was added portionwise to a stirred solution of 3-chloro-5-methoxybenzaldehyde (1 g, 5.86 mmol) in DCM (25 ml) under nitrogen. The reaction mixture was stirred at room temperature for 18 h. The solution was then evaporated to dryness. The residue was purified by silica column chromatography, eluting with 2-3% ethyl acetate in hexane. Product fractions were combined and evaporated to afford methyl 3-(3-chloro-5-methoxy-phenyl)prop-2-enoate as a white solid (1.13 g, 85% yield).

¹H NMR (399.902 MHz, CDCl₃) δ 3.81 (s, 3H), 3.82 (s, 3H), 6.41 (d, 1H), 6.91 (d, 2H), 7.10 (t, 1H), 7.57 (d, 1H).

5-(Aminomethyl)-1,2-oxazole-3-carboxamide hydrochloride used as starting material was prepared as in Example 123.

### Example 127

### N-[[3-(dimethylaminomethyl)-1,2-oxazol-5-yl]methyl]-N′-[5-[2-(5-methoxypyridin-3-yl)ethyl]-1H-pyrazol-3-yl]pyrimidine-2,4-diamine

5-(2-(5-methoxypyridin-3-yl)ethyl)-1H-pyrazol-3-amine (113 mg, 0.52 mmol, 1 eq), 4-chloro-N-[[3-(chloromethyl)-1,2-oxazol-5-yl]methyl]pyrimidin-2-amine (134 mg, 0.52 mmol, 1 eq) and 4M HCl in dioxane (0.065 ml, 0.26 mmol, 0.5 eq) were dissolved in 2-propanol (3 ml) and sealed into a microwave tube. The reaction was heated to 120° C. for 30 mins in the microwave reactor and cooled to room temperature. N-Methylmethanamine (1.782 ml, 10.35 mmol, 20 eq, 33% solution in ethanol) was added and the reaction was refluxed for 30 mins. The resulting mixture was evaporated to dryness and the residue was purified by preparative HPLC using decreasingly polar mixtures of water (containing 1% ammonium hydroxide) and MeCN as eluents. Fractions containing the desired compound were evaporated to dryness to afford N-[[3-(dimethylaminomethyl)-1,2-oxazol-5-yl]methyl]-N′-[5-[2-(5-methoxypyridin-3-yl)ethyl]-1H-pyrazol-3-yl]pyrimidine-2,4-diamine (9 mg, 3.95%) as an orange gum.

1H NMR (700.034 MHz, DMSO) δ 2.10-2.12 (6H, m), 2.82-2.93 (4H, m), 3.40 (2H, s), 3.80 (3H, s), 4.55 (2H, d), 6.13-6.18 (2H, m), 7.21-7.23 (2H, m), 7.83 (1H, d), 8.03 (1H, d), 8.10-8.12 (1H, m), 9.41 (1H, s), 11.97 (1H, s). MS. m/z 450 (MH+).

5-[2-(5-Methoxypyridin-3-yl)ethyl]-1H-pyrazol-3-amine, used as starting material was prepared as follows:—

Methyl 3-(5-methoxypyridin-3-yl)propanoate (840 mg, 4.30 mmol) and acetonitrile (270 □l, 5.16 mmol) were stirred in 1,4-dioxane (18 ml) under nitrogen. Sodium hydride (206 mg, 60% dispersion on mineral oil, 5.16 mmol) was added and the mixture was stirred at room temperature for 10 mins and then refluxed under nitrogen for 18 h. The reaction mixture was allowed to cool to room temperature. Ethanol (3 ml) was added, followed by hydrazine monohydrochloride (590, 8.61 mmol). The mixture was refluxed for a further 22 h and then left stand at room temperature for 3 days. The mixture was evaporated to dryness and the residue partitioned between water (20 ml) and ethyl acetate (15 ml). The layers were separated and the aqueous phase extracted with ethyl acetate (2×15 ml). Sat. aq. NaHCO₃ and NaCl were added to the aqueous phase, which was then re-extracted with ethyl acetate (3×10 ml). The combined organic extracts were dried over MgSO₄, filtered and evaporated to dryness. The crude product was purified by silica column chromatography, eluting with 0-10% MeOH in DCM to afford 5-[2-(5-methoxypyridin-3-yl)ethyl]-1H-pyrazol-3-amine as a yellow gummy oil (444 mg, 47% yield).

¹H NMR (399.902 MHz, DMSO) δ 2.71-2.79 (m, 2H), 2.82-2.90 (m, 2H), 3.81 (s, 3H), 4.44 (bs, 2H), 5.19 (s, 1H), 7.20-7.23 (m, 1H), 8.03 (d, 1H), 8.11 (d, 1H), 11.08 (bs, 1H), m/z (ES+) [M+H]+=219.

Methyl 3-(5-methoxypyridin-3-yl)propanoate, used as starting material was prepared as follows:—

10% Pd/C (65 mg) was added to a solution of methyl 3-(5-methoxypyridin-3-yl)prop-2-enoate (850 mg, 4.40 mmol) in ethanol (65 ml) and the mixture was stirred at room temperature under a balloon of hydrogen for 18 h. A further portion of catalyst was added and the mixture was stirred under hydrogen for a further 24 h. The mixture was filtered, washed through with ethanol and the filtrate was evaporated under vacuum to afford methyl 3-(5-methoxypyridin-3-yl)propanoate as a colourless oil (849 mg, 99%).

¹H NMR (399.902 MHz, CDCl₃) δ 2.64 (t, 2H), 2.95 (t, 2H), 3.68 (s, 3H), 3.85 (s, 3H), 7.03-7.06 (m, 1H), 8.09 (d, 1H), 8.17 (d, 1H); m/z (ES+) [M+H]+=196.

Methyl 3-(5-methoxypyridin-3-yl)prop-2-enoate, used as starting material was prepared as follows:—

5-Bromo-3-methoxypyridine (1 g, 5.32 mmol) was stirred with tris(2-methylphenyl)phosphane (162 mg, 0.53 mmol), N,N-diethylethanamine (2.97 ml, 21.27 mmol) and palladium (II) acetate (120 mg, 0.53 mmol) in acetonitrile (100 ml) and the mixture was purged with nitrogen. Methyl prop-2-enoate (1.44 ml, 15.96 mmol) was added and the mixture was refluxed for 18 h. The solvent was evaporated and the residue was purified by silica column chromatography, eluting with 0-1% MeOH in DCM, to afford methyl 3-(5-methoxypyridin-3-yl)prop-2-enoate as a pale yellow solid (1.02 g, 99% yield).

¹H NMR (399.902 MHz, DMSO) δ 3.69 (s, 3H), 3.81 (s, 3H), 6.80 (d, 1H), 7.63 (d, 1H), 7.71-7.74 (m, 1H), 8.25 (d, 1H), 8.40 (d, 1H); m/z (ES+) [M+H]+=194.

4-chloro-N-[[3-(chloromethyl)-1,2-oxazol-5-yl]methyl]pyrimidin-2-amine used as starting material was prepared as follows:—

To a stirred solution of 2-[[3-(hydroxymethyl)-1,2-oxazol-5-yl]methylamino]pyrimidin-4-ol (1.24 g, 5.58 mmol, 1 eq) and N-ethyl-N-propan-2-yl-propan-2-amine (2.2 mL, 12.83 mmol, 2.3 eq) in toluene (24 mL) was added phosphorous oxychloride (1.15 mL, 12.28 mmol, 2.2 eq). The reaction was heated at 80° C. for 2 h, allowed to cool to room temperature and then poured into a saturated sodium bicarbonate solution. The product was extracted with ethyl acetate (x2), washed with brine, dried (MgSO₄), filtered and evaporated to give an orange gum. The crude product was dissolved in DCM and purified by silica column chromatography, eluting with 20-50% ethyl acetate in iso-hexane, to give product as a white solid (751 mg, 52%). 1H NMR (CDCl3 400.13 MHz) δ 4.55 (2H, s), 4.75 (2H, d), 5.64 (1H, s), 6.29 (1H, s), 6.67 (1H, d), 8.18 (1H, d). MS m/z 259 (MH+).

2-[[3-(Hydroxymethyl)-1,2-oxazol-5-yl]methylamino]pyrimidin-4-ol used as starting material was prepared as follows:—

[5-(aminomethyl)-1,2-oxazol-3-yl]methanol (1.35 g, 10 mmol, 1.2 eq) and 2-methylsulfonylpyrimidin-4-ol (1.24 g, 8.7 mmol, 1 eq) were heated together at 160° C. for 4 h. The mixture was allowed to cool to room temperature and suspended in methanol and filtered. The filtrate was evaporated to dryness and purified by silica column chromatography, eluting with 5-15% methanol in dichloromethane to give product as a cream solid (1.27 g, 66%).

1H NMR (DMSO 400.13 MHz) δ 4.45 (2H, d), 4.60 (2H, d), 5.39 (1H, t), 5.60 (1H, d), 6.28 (1H, s), 7.04 (1H, s), 7.6 (1H, d), 11.04 (1H, s)

2-Methylsulfonylpyrimidin-4-ol used as starting material was prepared as follows:—

2-Thiouracil (84 g, 0.66 mol, 1 eq) was dissolved in aqueous sodium hydroxide (26 g, 0.68 mol, 1.05 eq in 80 mL water). The solution was diluted with MeOH (160 mL). Iodomethane (47 mL, 0.75 mol, 1.15 eq) was added dropwise. The temperature was kept between 35-40° C. A precipitate formed and the mixture was heated at 40° C. for 1 h. The mixture was stirred at room temperature overnight, filtered and the solid was washed with water, methanol and dried at 45° C. in a vacuum oven to give 2-methylsulfonylpyrimidin-4-ol (53 g, 57%).

1H NMR (DMSO 400.13 MHz) δ 2.37 (3H, s), 5.97 (1H, d), 7.74 (1H, d)

[5-(Aminomethyl)-1,2-oxazol-3-yl]methanol used as starting material was prepared as follows:—

tert-butyl N-[[3-(hydroxymethyl)-1,2-oxazol-5-yl]methyl]carbamate (4.45 g, 19.5 mmol, 1 eq) was dissolved in dichloromethane (89 mL) and trifluoroacetic acid (7.24 mL, 97 mmol, 5 eq) was added. The reaction was stirred at room temperature for 5 h. The mixture was evaporated to dryness, dissolved in methanol and loaded onto a SCX-2 column. This was then further washed with methanol. The product was eluted with 3.5N ammonia in methanol. The desired fractions were collected and evaporated to dryness. The residue was then triturated with diethyl ether to give the product as a purple solid (1.35 g, 54%).

1H NMR (DMSO 400.13 MHz) δ 2.1 (2H, s), 3.78 (2H, s), 4.45 (2H, s), 5.39 (1H, s), 6.29 (1H, s).

tert-Butyl N-[[3-(hydroxymethyl)-1,2-oxazol-5-yl]methyl]carbamate used as starting material was prepared as follows:—

Ethyl 5-[[(2-methylpropan-2-yl)oxycarbonylamino]methyl]-1,2-oxazole-3-carboxylate (5 g, 18.5 mmol, 1 eq) was dissolved in ethanol (50 mL) and cooled to 0° C. Sodium borohydride (1.89 g, 49.95 mmol, 5 eq) was added portionwise and the reaction was stirred at room temperature overnight. The mixture was quenched with aqueous sodium bicarbonate solution, extracted with ethyl acetate (x3), washed with brine, dried (MgSO₄) and evaporated to give product as a colouress oil (4.45 g, >100%).

1H NMR (CDCl3 400.13 MHz) δ 1.43 (9H, s), 4.4 (2H, d), 4.72 (2H, s), 5.0 (1H, s), 6.22 (1H, s). MS m/z 173 (MH+−56).

Ethyl 5-[[(2-methylpropan-2-yl)oxycarbonylamino]methyl]-1,2-oxazole-3-carboxylate used as starting material was prepared as in Example 64.

### Example 128

### 3-[2-[5-[[2-[[3-(dimethylaminomethyl)-1,2-oxazol-5-yl]methylamino]pyrimidin-4-yl]amino]-1H-pyrazol-3-yl]ethyl]phenol

3-[2-[5-[[2-[[3-(hydroxymethyl)-1,2-oxazol-5-yl]methylamino]pyrimidin-4-yl]amino]-1H-pyrazol-3-yl]ethyl]phenol (97 mg, 0.24 mmol, 1 eq) was suspended in DCM (5 mL) and thionyl chloride (87 uL, 1. 19 mmol, 5 eq) was added. The reaction was stirred at room temperature overnight. 2M N-Methylmethanamine solution in THF (5 mL) was added and the mixture was heated at 75° C. for 3 h. The mixture was evaporated to dryness and purified by silica column chromatography, eluting with a gradient of 5-10% methanol (containing 10% 7N ammonia in methanol) in dichloromethane to give the crude product. The crude product was purified by preparative HPLC using decreasingly polar mixtures of water (containing 1% ammonium hydroxide) and MeCN as eluents. Fractions containing the desired compound were evaporated to dryness to afford 3-[2-[5-[[2-[[3-(dimethylaminomethyl)-1,2-oxazol-5-yl]methylamino]pyrimidin-4-yl]amino]-1H-pyrazol-3-yl]ethyl]phenol as a white solid (26 mg, 25%).

1HNMR (DMSO 400.13 MHz) δ 2.16 (6H, s), 2.84 (4H, s), 3.45 (2H, s), 4.61 (2H, d), 6.21 (1H, s), 6.31 (1H, s), 6.63 (1H, m), 6.70 (2H, m), 7.11 (1H, t), 7.25 (1H, s), 7.38 (1H, d), 9.40 (1H, s), 11.96 (1H, s). MS m/z 435 (MH+).

3-[2-[5-[[2-[[3-(Hydroxymethyl)-1,2-oxazol-5-yl]methylamino]pyrimidin-4-yl]amino]-1H-pyrazol-3-yl]ethyl]phenol used as starting material was prepared as follows:—

[5-[[[4-[[5-[2-(3-Methoxyphenyl)ethyl]-2H-pyrazol-3-yl]amino]pyrimidin-2-yl]amino]methyl]-1,2-oxazol-3-yl]methanol (120 mg, 0.28 mmol, 1 eq) was dissolved in DCM (6 mL) and cooled to 0° C. under nitrogen. Boron tribromide (1.42 mL, 1.42 mmol, 5 eq, 1M in DCM) solution was added dropwise and the reaction was allowed to warm to room temperature and stirred overnight. The reaction was quenched with methanol (10 mL), stirred for 1 h and then evaporated to dryness. The crude product was dissolved in methanol and loaded onto a SCX-2 column. This was washed with methanol and then the product was eluted with 3.5N ammonia in methanol. After evaporation, the product was obtained as a yellow foam (97 mg, 85%).

MS m/z 408 (MH+)

[5-[[[4-[[5-[2-(3-Methoxyphenyl)ethyl]-2H-pyrazol-3-yl]amino]pyrimidin-2-yl]amino]methyl]-1,2-oxazol-3-yl]methanol used as starting material was prepared as follows:—

To 2-chloro-N-[5-[2-(3-methoxyphenyl)ethyl]-2H-pyrazol-3-yl]pyrimidin-4-amine (250 mg, 0.76 mmol, 1 eq) was added [5-(aminomethyl)-1,2-oxazol-3-yl]methanol (146 mg, 1.14 mmol, 1.5 eq) followed by 2-methoxyethanol (4 ml) and N-ethyl-N-propan-2-yl-propan-2-amine (265 μL, 1.52 mmol, 2 eq). The reaction was heated in the microwave at 200° C. for 60 mins, allowed to cool and evaporated under reduced pressure. The crude product was purified by silica column chromatography, eluting with 5-10% methanol in dichloromethane. Clean fractions were combined and evaporated to give product as a yellow foam (287 mg, 90%).

[5-(Aminomethyl)-1,2-oxazol-3-yl]methanol, used as starting material, was prepared as in Example 127.

2-Chloro-N-[5-[2-(3-methoxyphenyl)ethyl]-2H-pyrazol-3-yl]pyrimidin-4-amine, used as starting material, was prepared as in Example 27.

### Example 132

### 3-Methoxy-N-methyl-5-[2-[5-[[2-[(3-methyl-1,2-oxazol-5-yl)methylamino]pyrimidin-4-yl]amino]-1H-pyrazol-3-yl]ethyl]benzamide

A mixture of 3-[2-(5-amino-1H-pyrazol-3-yl)ethyl]-5-methoxy-N-methylbenzamide (138 mg, 0.5 mmol, 1.0 eq), 4-chloro-N-[(3-methyl-1,2-oxazol-5-yl)methyl]pyrimidin-2-amine (113 mg, 0.5 mmol, 1.0 eq) and ethanol (2.5 ml) were stirred and heated at 80° C. overnight under an atmosphere of nitrogen. The resulting suspension was allowed to cool to room temperature and filtered to give the crude product as a white solid. This material was purified by reverse-phase preparative HPLC (basic) using a 20-40% gradient of acetonitrile in water containing 1% ammonium hydroxide solution. The clean fractions were taken and evaporated to afford the title compound as a white solid, (107 mg, 46% yield).

¹H NMR (500.13 MHz, DMSO-d₆, CD₃CO₂D) δ 2.18 (3H, s), 2.80-2.81 (3H, m), 2.88-2.93 (2H, m), 2.94-2.99 (2H, m), 3.79 (3H, s), 4.58 (2H, s), 6.08-6.10 (2H, m), 6.29 (1H, d), 6.92 (1H, t), 7.21 (1H, t), 7.31 (1H, t), 7.86 (1H, d)

MS: m/z 463 (MH+)

4-Chloro-N-[(3-methyl-1,2-oxazol-5-yl)methyl]pyrimidin-2-amine was prepared as outlined in Example 13.

3-[2-(5-Amino-1H-pyrazol-3-yl)ethyl]-5-methoxy-N-methylbenzamide, used as starting material was prepared as follows:—

Lithium diisopropylamide solution (1.8M in tetrahydrofuran/heptane/ethylbenzene, 11.11 mL, 20.0 mmol, 4.0 eq) was added to anhydrous tetrahydrofuran (35 ml) at −78° C. and the mixture stirred at this temperature under an atmosphere of nitrogen. Acetonitrile (1.05 ml, 20.0 mmol, 4.0 eq) was added dropwise and the solution maintained at −78° C. for 10 mins. A solution of methyl 3-[3-methoxy-5-(methylcarbamoyl)phenyl]propanoate (1.26 g, 5.0 mmol, 1.0 eq) in tetrahydrofuran (10 mL) was added rapidly and the mixture stirred at −78° C. for 10 mins and then allowed to warm to 5° C. over 20 mins. Hydrazine hydrochloride (1.38 g, 20.0 mmol, 4.0 eq) and ethanol (35 ml) were then added and the mixture heated at 78° C. for 18 h. The mixture was evaporated, dissolved in methanol (50 ml) and applied to a SCX-2 cation exchange cartridge. The cartridge was eluted with methanol (8×50 ml) and then with methanol containing ammonia (2M anhydrous). The clean fractions were taken and evaporated to afford the title compound as a clear oil, (990 mg, 72% yield). MS: m/z 275 (MH+)

Methyl 3-[3-methoxy-5-(methylcarbamoyl)phenyl]propanoate, used as starting material was prepared as follows:—

To a mixture of methyl 3-[3-methoxy-5-(methylcarbamoyl)phenyl]prop-2-enoate (5.7 g, 23.0 mmol, 1.0 eq) in ethyl acetate (120 mL) was added 5% palladium on charcoal catalyst (750 mg) and the reaction mixture was stirred in an atmosphere of hydrogen for 18 h at room temperature. The mixture absorbed 620 mL of hydrogen. The suspension was then flushed with nitrogen, filtered and evaporated. This gave methyl 3-[3-methoxy-5-(methylcarbamoyl)phenyl]propanoate as an oil, 5.7 g.

MS: m/z 252 (MH+)

Methyl 3-[3-methoxy-5-(methylcarbamoyl)phenyl]prop-2-enoate, used as starting material was prepared as follows:

A mixture of 3-formyl-5-methoxy-N-methylbenzamide (4.9 1 g, 25.4 mmol, 1.0 eq) and methyl (triphosphoranylidene) acetate (12.74 g, 38.10 mmol, 1.5 eq) dissolved in anhydrous tetrahydrofuran (240 mL) was stirred at room temperature in an atmosphere of nitrogen for 18 h. After evaporation of the solvent, the crude product was purified by silica column chromatography, eluting with a 0-20% gradient of ethyl acetate in dichloromethane. The clean fractions were taken and evaporated to give Methyl 3-[3-methoxy-5-(methylcarbamoyl)phenyl]prop-2-enoate as a white solid, 5.7 g.

MS: m/z 250 (MH+)

3-Formyl-5-methoxy-N-methylbenzamide, used as starting material was prepared as follows:

A stirred solution of methyl 3-formyl-5-methoxybenzoate (6.22 g, 32.0 mmol, 1.0 eq) and methylamine solution (2.0M in tetrahydrofuran, 86.4 mL, 172.8 mmol, 5.4 eq) in anhydrous tetrahydrofuran (120 mL) was cooled to -50° C. under nitrogen. Trimethylaluminium solution (2.0M in toluene, 43.2 mL, 86.40 mmol, 2.7 eq) was added slowly over 10 mins and the mixture was allowed to warm slowly to room temperature and then allowed to stand for 96 h. The mixture was cooled in an ice/methanol bath and a solution of potassium sodium tartrate (20% in water, 40 mL) was added dropwise. Water (300 mL) and ethyl acetate (400 mL) were added and the mixture transferred to a separating funnel. Hydrochloric acid (2M aqueous, 300 mL) was added to give a clear solution. The layers were separated and the aqueous was extracted with more ethyl acetate. The combined ethyl acetate extracts were washed with 0.5M aqueous HCl solution, water, sodium bicarbonate solution, brine, then dried over magnesium sulphate, filtered and evaporated to give the product as a white solid, 4.9 g, (79% yield).

¹H NMR (399.9 MHz, CDCl₃) δ 3.03-3.04 (3H, m), 3.90 (3H, s), 6.39 (1H, s), 7.49-7.50 (1H, m), 7.62-7.63 (1H, m), 7.79 (1H, t), 9.99 (1H, s)

MS: m/z 194 (MH+)

The preparation of methyl 3-formyl-5-methoxybenzoate, used as starting material is described by Zhao, He; Thurkauf, Andrew in Synthetic Communications (2001), 31(12), 1921-1926.

### Example 133

### N-[(3-methyl-1,2-oxazol-5-yl)methyl]-N′-[5-[2-(3-pyrimidin-2-yloxyphenyl)ethyl]-1H-pyrazol-3-yl]pyrimidine-2,4-diamine hydrochloride

5- {2-[3-(Pyrimidin-2-yloxy)phenyl]ethyl} -1H-pyrazol-3-amine (40 mg, 0.142 mmol) was heated with 4-chloro-N-[(3-methyl-1,2-oxazol-5-yl)methyl]pyrimidin-2-amine (32 mg, 0.142 mmol) in ethanol (1.5 ml) at 80° C. for 18 h. The mixture was allowed to cool to room temperature and the precipitated product was collected by filtration and washed with a little ethanol, then dried under vacuum to afford the title compound as a pale yellow solid (29 mg, 40% yield).

¹H NMR (399.902 MHz, DMSO) δ 2.17 (s, 3H), 2.86-2.98 (m, 4H), 4.70 (d, 2H), 6.28 (bs, 2H), 6.38 (bs, 1H), 7.00-7.05 (m, 1H), 7.05-7.08 (m, 1H), 7.13 (d, 1H), 7.26 (t, 1H), 7.35 (t, 1H), 7.89 (bd, 1H), 8.64 (d, 2H), 8.78 (bs, 1H), 11.22 (bs, 1H), 12.42 (bs, 1H), 12.56 (bs, 1H)

MS: m/z 470 (MH+)

4-Chloro-N-[(3-methyl-1,2-oxazol-5-yl)methyl]pyrimidin-2-amine was prepared as outlined in Example 13.

5-{2-[3-(Pyrimidin-2-yloxy)phenyl]ethyl}-1H-pyrazol-3-amine, used as starting material, was prepared as follows:—

Dry acetonitrile (138 μl, 2.63 mmol) was added dropwise to a stirred solution of LDA (1.46 ml, 1.8M solution in THF, 2.63 mmol) in THF (4 ml) at −78° C. under nitrogen and the mixture was stirred at −78° C. for 10 mins. A solution of methyl 3-(3-pyrimidin-2-yloxyphenyl)propanoate (340 mg, 1.32 mmol) in THF (6 ml) was added rapidly and stirring was continued at −78° C. for 20 mins, before the reaction mixture was allowed to warm to room temperature. The mixture was poured into aq. NH₄Cl (40 ml) and the aqueous phase was extracted with ether (3×20 ml). The combined extracts were dried over MgSO₄, filtered and evaporated. The residue was dissolved in ethanol (8 ml), hydrazine monohydrate (128 μl, 2.63 mmol) was added and the mixture was refluxed for 18 h. The mixture was allowed to cool and evaporated to dryness. The residue was partitioned between DCM (15 ml) and water (20 ml), the layers were separated and the aqueous extracted with a further portion of DCM (15 ml). The combined DCM extracts were washed with brine, dried over MgSO₄, filtered and evaporated. The crude product was purified by silica column chromatography, eluting with 0-5% MeOH in DCM, to afford the product, 5-[2-(3-pyrimidin-2-yloxyphenyl)ethyl]-1H-pyrazol-3-amine, as a colourless gum (40 mg, 11% yield).

¹H NMR (399.902 MHz, CDCl₃) δ 2.69-2.92 (m, 4H), 4.31 (bs, 2H), 5.22 (bs, 1H), 6.98-7.03 (m, 1H), 7.05-7.07 (m, 1H), 7.12 (d, 1H), 7.27 (t, 1H), 7.34 (t, 1H), 8.65 (d, 2H), 11.09 (bs, 1H), MS: m/z 282 (MH+)

Methyl 3-(3-pyrimidin-2-yloxyphenyl)propanoate, used as starting material, was prepared as follows:—

10% Pd/C (100 mg) was added to a solution of methyl 3-(3-pyrimidin-2-yloxyphenyl)prop-2-enoate (0.96 g, 3.75 mmol) in ethanol (100 ml) and the mixture was stirred at room temperature under a balloon of hydrogen for 18 h. The solution was filtered and the filtrate was evaporated to dryness under vacuum. The residue was purified by silica column chromatography, eluting with 15-45% ethyl acetate in hexane, to afford the product, methyl 3-[3-(pyrimidin-2-yloxy)phenyl]propanoate, as a white solid (540 mg, 56% yield).

¹H NMR (399.902 MHz, CDCl3) δ 2.66 (t, 2H), 2.89 (t, 2H), 3.59 (s, 3H), 7.00-7.05 (m, 1H), 7.05-7.08 (m, 1H), 7.10-7.14 (m, 1H), 7.27 (t, 1H), 7.35 (t, 1H), 8.65 (d, 2H); MS: m/z 259 (MH+)

Methyl 3-(3-pyrimidin-2-yloxyphenyl)prop-2-enoate, used as starting material, was prepared as follows:—

Methyl (triphenylphosphoranylidene)acetate (2.25 g, 6.74 mmol) was added portionwise to a stirred suspension of 3-(pyrimidin-2-yloxy)benzaldehyde (900 mg, 4.50 mmol) in DCM (20 ml) under nitrogen. The reaction mixture was stirred at room temperature for 18 h. The solution was then concentrated under vacuum, adsorbed onto silica and purified by silica column chromatography, eluting with 15-30% ethyl acetate in hexane to afford the product, methyl 3-(3-pyrimidin-2-yloxyphenyl)prop-2-enoate, as a white solid (0.97 g, 84% yield).

¹H NMR (399.902 MHz, CDCl₃) δ 3.80 (s, 3H), 6.43 (d, 1H), 7.06 (t, 1H), 7.21-7.25 (m, 1H), 7.36-7.38 (m, 1H), 7.39-7.46 (m, 2H), 7.69 (d, 1H), 8.57 (d, 2H); MS: m/z 257 (MH+)

3-(Pyrimidin-2-yloxy)benzaldehyde, used as starting material, was prepared as follows:—

(3-Pyrimidin-2-yloxyphenyl)methanol (1 g, 4.95 mmol) was suspended in DCM (40 ml) and stirred under nitrogen. Dess-Martin periodinane (2.52 g, 5.93 mmol) in DCM (40 ml) was added slowly and the mixture was stirred at room temperature for a further 30 min. The mixture was washed with 1N NaOH(aq) (2×35 ml), water/brine (30 ml), dried over MgSO₄, filtered and evaporated to afford the product, 3-(pyrimidin-2-yloxy)benzaldehyde, as a white solid (1.17 g, quant. yield).

¹H NMR (399.902 MHz, CDCl₃) δ 7.37 (t, 1H), 7.61-7.67 (m, 1H), 7.74 (t, 1H), 7.77-7.80 (m, 1H), 7.88 (d, 1H), 8.73 (d, 2H), 10.08 (s, 1H); MS: m/z 201 (MH+)

### Example 134

### 6-[2-[5-[[2-[(3-methyl-1,2-oxazol-5-yl)methylamino]pyrimidin-4-yl]amino]-2H-pyrazol-3-yl]ethyl]-1H-pyridin-2-one dihydrochloride

N′-[5-[2-(6-methoxypyridin-2-yl)ethyl]-1H-pyrazol-3-yl]-N-[(3-methyl-1,2-oxazol-5-yl)methyl]pyrimidine-2,4-diamine hydrochloride (85 mg, 0.226 mmol) was stirred in ethanol (15 ml) and conc. aqueous HCl (1.5 ml) at 80° C. for 2 days. The mixture was allowed to cool and poured into ice-water, then allowed to warm to room temperature over 1 h. The precipitated product was collected by filtration, washed with water and dried under vacuum to afford the title compound as a cream solid (70 mg, 67%).

¹H NMR (399.902 MHz, DMSO) δ 2.19 (3H, s), 2.71-2.83 (2H, m), 2.86-2.95 (2H, m), 4.70 (2H, d), 5.98 (1H, d), 6.16 (1H, d), 6.22-6.45 (3H, bm), 7.29-7.37 (1H, m), 7.87 (1H, bs), 8.74 (1H, bs), 11.22 (1H, bs), 11.60 (1H, bs), 12.46 (1H, bs); MS: m/z 393 (MH+)

N′-[5-[2-(6-methoxypyridin-2-yl)ethyl]-1H-pyrazol-3-yl]-N-[(3-methyl-1,2-oxazol-5-yl)methyl]pyrimidine-2,4-diamine hydrochloride, used as starting material, was prepared as follows:—

5-[2-(6-Methoxypyridin-2-yl)ethyl]-1H-pyrazol-3-amine (80 mg, 0.367 mmol) was heated with 4-chloro-N-[(3-methyl-1,2-oxazol-5-yl)methyl]pyrimidin-2-amine (83 mg, 0.367 mmol) in ethanol (2 ml) in a microwave reactor at 120° C. for 1 h. The precipitated solid was collected by filtration, washed with ethanol and dried under vacuum to afford N′-[5-[2-(6-methoxypyridin-2-yl)ethyl]-1H-pyrazol-3-yl]-N-[(3-methyl-1,2-oxazol-5-yl)methyl]pyrimidine-2,4hydrochloride as an off-white solid (106 mg, 65%).

¹H NMR (399.902 MHz, DMSO) δ 2.19 (s, 3H), 2.92-3.06 (m, 4H), 3.84 (s, 3H), 4.70 (d, 2H), 6.19-6.46 (bm, 3H), 6.63 (d, 1H), 6.82 (d, 1H), 7.60 (t, 1H), 7.89 (bs, 1H), 8.78 (bs, 1H), 11.20 (bs, 1H), 12.44 (bs, 1H), 12.56 (bs, 1H); MS: m/z 407 (MH+)

4-Chloro-N-[(3-methyl-1,2-oxazol-5-yl)methyl]pyrimidin-2-amine was prepared as outlined in Example 13.

5-[2-(6-Methoxypyridin-2-yl)ethyl]-1H-pyrazol-3-amine, used as starting material, was prepared as follows:—

Dry acetonitrile (268 μl, 5.122 mmol) was added dropwise to a stirred solution of LDA (1.46 ml, 1.8M solution in THF, 5.122 mmol) in THF (20 ml) at −78° C. (under nitrogen) and the mixture was stirred at −78° C. for 10 mins. Methyl 3-(6-methoxypyridin-2-yl)propanoate (500 mg, 2.561 mmol) was added rapidly and the reaction mixture was stirred at −78° C. for 20 mins, then allowed to warm to room temperature. Ethanol (20 ml) was added followed by hydrazine monohydrochloride (439 mg, 6.403 mmol) and the solution was refluxed for 18 h. The solvent was evaporated under vacuum, the residue was purified by silica column chromatography, eluting with 0-4% MeOH in DCM. Fractions containing product were evaporated to afford 5-[2-(6-methoxypyridin-2-yl)ethyl]-1H-pyrazol-3-amine as a yellow gum (450 mg, 80% yield).

¹H NMR (399.902 MHz, DMSO) δ 2.77-2.97 (m, 4H), 3.85 (s, 3H), 4.30 (bs, 2H), 5.18 (bs, 1H), 6.62 (d, 1H), 6.83 (d, 1H), 7.59 (t, 1H), 11.10 (bs, 1H); MS: m/z (MH+) 219.

Methyl 3-(6-methoxypyridin-2-yl)propanoate, used as starting material, was prepared as follows:—

10% Pd/C (140 mg) was added to a solution of methyl 3-(6-methoxypyridin-2-yl)prop-2-enoate (1.43 g, 7.40 mmol) in ethanol (150 ml) and the mixture was stirred at room temperature under a balloon of hydrogen for 18 h. The catalyst was removed by filtration and washed with ethanol. The filtrate was evaporated under vacuum to give the product, methyl 3-(6-methoxypyridin-2-yl)propanoate, as a colourless oil (1.45 g, quant. yield).

¹H NMR (399.902 MHz, DMSO) δ 2.73 (t, 2H), 2.96 (t, 2H), 3.60 (s, 3H), 3.82 (s, 3H), 6.62 (d, 1H), 6.85 (d, 1H), 7.60 (t, 1H); MS: m/z (MH+) 196.

Methyl 3-(6-methoxypyridin-2-yl)prop-2-enoate, used as starting material, was prepared as follows:—

2-Bromo-6-methoxypyridine (2 g, 10.64 mmol) was added to a mixture of bis(tri-tbutylphosphine)palladium(0) (327 mg, 0.64 mmol) and cesium carbonate (3.82 g, 11.70 mmol) in dioxane (20 ml). The reaction mixture was stirred under nitrogen. Methyl acrylate (1.92 ml, 21.27 mmol) was added and the mixture was heated at 90° C. for 18 h. The reaction mixture was allowed to cool to room temperature, diluted with ether, filtered and washed through with ether. The filtrate was evaporated to dryness and purified by silica column chromatogrpahy, eluting with 0-5% ethyl acetate in hexane) to afford methyl 3-(6-methoxypyridin-2-yl)prop-2-enoate as a white solid (1.81 g, 88% yield).

¹H NMR (399.902 MHz, DMSO) δ 3.76 (s, 3H), 3.91 (s, 3H), 6.88 (d, 1H), 6.90 (d, 1H), 7.31 (d, 1H), 7.62 (d, 1H), 7.77 (t, 1H); MS: m/z 194 (MH+).

### Example 136

### N-[[3-(dimethylaminomethyl)-1,2-oxazol-5-yl]methyl]-N′-[5-[2-(5-fluoro-2-methoxy-pyridin-4-yl)ethyl]-1H-pyrazol-3-yl]pyrimidine-2,4-diamine

5-[2-(5-Fluoro-2-methoxy-pyridin-4-yl)ethyl]-1H-pyrazol-3-amine (65 mg, 0.275 mmol) was heated with 4-chloro-N-[[3-(chloromethyl)-1,2-oxazol-5-yl]methyl]pyrimidin-2-amine (72 mg, 0.275 mmol) in ethanol (2 ml) at 80° C. for 18 h. The mixture was allowed to cool and the precipitated solid was collected by filtration and washed with ethanol. The solid was then stirred again in ethanol (2 ml) and N-methylmethanamine (2M solution in ethanol, 1 ml) was added. The mixture was heated at 80° C. for 30 min. The solution was allowed to cool and evaporated to dryness and then diluted with water (8 ml). The aqueous phase was extracted with ethyl acetate (3×8 ml), dried over MgSO₄, filtered and evaporated to afford N-[[3-(dimethylaminomethyl)-1,2-oxazol-5-yl]methyl]-N′-[5-[2-(5-fluoro-2-methoxy-pyridin-4-yl)ethyl]-1H-pyrazol-3-yl]pyrimidine-2,4-diamine as an off-white glassy solid (40 mg, 32% yield).

¹H NMR (399.902 MHz, DMSO) δ 2.17 (s, 6H), 2.87-3.04 (m, 4H), 3.45 (s, 2H), 3.85 (s, 3H), 4.61 (d, 2H), 6.22 (s, 1H), 6.14-6.40 (bs, 2H), 6.81 (d, 1H), 7.29 (bs, 1H), 7.90 (d, 1H), 8.10 (s, 1H), 9.45 (bs, 1H), 12.01 (bs, 1H); m/z (ES+) [M+H]+=468.

5-[2-(5-Fluoro-2-methoxy-pyridin-4-yl)ethyl]-1H-pyrazol-3-amine, used as starting material was prepared as follows:—

3-Amino-5-hydroxypyrazole (0.56 g, 5.65 mmol) and triphenylphosphine (1.78 g, 6.78 mmol) were stirred in DCM (16 ml) under nitrogen and the reaction mixture was cooled in an ice-bath. Diisopropylazodicarboxylate (1.34 ml, 6.78 mmol) was added dropwise over a period of 10 min. The reaction mixture was then stirred in the ice-bath for 1 h. (5-Fluoro-2-methoxy-pyridin-4-yl)methanol (1.07 g, 6.78 mmol) in THF (15 ml) was added slowly over 5-10 min. The reaction mixture was stirred and allowed to warm to room temperature over 1 h. This was then stirred for a further 18 h. The mixture was filtered and washed through with DCM (10 ml). The filtrate was extracted with 2M HCl(aq) (3×8 ml) and the combined extracts were basified with 6N NaOH(aq). The basified aqueous phase was extracted with DCM (3×20 ml). The combined extracts were filtered, dried over MgSO₄, filtered and evaporated. The crude product was purified by silica column chromatography, eluting with 0-3% MeOH in DCM, to afford 5-[(5-fluoro-2-methoxy-pyridin-4-yl)methoxy]-1H-pyrazol-3-amine as a white solid (354 mg, 26% yield).

¹H NMR (399.902 MHz, DMSO) δ 3.75 (s, 3H), 4.70 (s, 1H), 4.91 (s, 2H), 5.06 (s, 2H), 6.76 (d, 1H), 8.04 (d, 1H), 10.37 (s, 1H); m/z (ES+) [M+H]+=239.

(5-Fluoro-2-methoxy-pyridin-4-yl)methanol, used as starting material, was prepared as follows:—

Borane-tetrahydrofuran complex (1M solution in THF, 52.6 ml, 52.6 mmol) was added slowly to a solution of 5-fluoro-2-methoxy-pyridine-4-carboxylic acid (2 g, 11.7 mmol) in THF (100 ml) under nitrogen. The reaction mixture was stirred at room temperature for 2.5 h. The solvent was evaporated and the residue was stirred in methanol (40 ml) for 18 h. The solvent was evaporated and the crude product was purified by silica column chromatography, eluting with 0-1% MeOH in DCM. Pure product fractions were combined and evaporated to afford (5-fluoro-2-methoxypyridin-4-yl)methanol as a white solid (1.42 g, 77%).

¹H NMR (399.902 MHz, CDCl₃) δ 3.90 (s, 3H), 4.76 (s, 2H), 6.84-6.87 (m, 1H), 7.92 (d, 1H); m/z (ES+) [M+H]+=158.

4-Chloro-N-[[3-(chloromethyl)-1,2-oxazol-5-yl]methyl]pyrimidin-2-amine, used as a starting material, was prepared as follows:—

2-[[3-(Hydroxymethyl)-1,2-oxazol-5-yl]methylamino]pyrimidin-4-ol (1.24 g, 5.58 mmol, 1 eq) and N-ethyl-N-propan-2-yl-propan-2-amine (2.2 mL, 12.83 mmol, 2.3 eq) were stirred in toluene (24 mL) and phosphorous oxychloride (1.15 mL, 12.28 mmol, 2.2 eq) was added dropwise. The reaction was heated at 80° C. for 2 h, then allowed to cool and poured into saturated sodium bicarbonate solution. The product was extracted with ethyl acetate (×2), washed with brine, dried (MgSO₄), filtered and evaporated to give an orange gum. The crude product was dissolved in DCM and purified by silica column chromatography, eluting with 20-50% ethyl acetate in iso-hexane to give the product as a white solid (75 lmg, 52%).

1H NMR (CDCl3 400.13 MHz) δ 4.55 (2H, s), 4.75 (2H, d), 5.64 (1H, s), 6.29 (1H, s), 6.67 (1H, d), 8.18 (1H, d). MS m/z 259 (MH+).

2-[[3-(Hydroxymethyl)-1,2-oxazol-5-yl]methylamino]pyrimidin-4-ol was prepared as follows:—

[5-(Aminomethyl)-1,2-oxazol-3-yl]methanol (1.35 g, 10 mmol, 1.2 eq) and 2-methylsulfonylpyrimidin-4-ol (1.24 g, 8.7 mmol, 1 eq) were heated together at 160° C. for 4 h. The mixture was allowed to cool, then suspended in methanol and filtered. The filtrate was evaporated to dryness and purified by silica column chromatography, eluting with 5-15% methanol in dichloromethane to give product as a cream solid (1.27 g, 66%).

1H NMR (DMSO 400.13 MHz) δ 4.45 (2H, d), 4.60 (2H, d), 5.39 (1H, t), 5.60 (1H, d), 6.28 (1H, s), 7.04 (1H, s), 7.6 (1H, d), 11.04 (1H, s)

2-Methylsulfanylpyrimidin-4-ol was prepared as follows:—

2-Thiouracil (84 g, 0.66 mol, 1 eq) was dissolved in aqueous sodium hydroxide (26 g, 0.68 mol, 1.05 eq in 80 mL water). The solution was diluted with MeOH (160 mL). Iodomethane (47 mL, 0.75 mol, 1.15 eq) was added dropwise with ice bath cooling to keep temp between 35-40° C. A precipitate formed and the mixture was heated at 40° C. for 1 h. The mixture was stirred at room temperature overnight, filtered and the solid washed with water, methanol and dried (vacuum oven at 45° C.) to give 2-methylsulfanylpyrimidin-4-ol (53 g, 57%).

1H NMR (DMSO 400.13 MHz) δ 2.37 (3H, s), 5.97 (1H, d), 7.74 (1H, d)

[5-(Aminomethyl)-1,2-oxazol-3-yl]methanol was prepared as follows:—

tert-Butyl N-[[3-(hydroxymethyl)-1,2-oxazol-5-yl]methyl]carbamate (4.45 g, 19.5 mmol, 1 eq) was dissolved in dichloromethane (89 mL) and trifluoroacetic acid (7.24 mL, 97 mmol, 5 eq) was added. The reaction was stirred at room temperature for 5 h. The mixture was evaporated to dryness, dissolved in methanol and loaded onto a SCX-2 column. After washing with methanol, the product was eluted with 3.5N ammonia in methanol. After trituration with diethyl ether, the product was obtained as a purple solid (1.35 g, 54%) after.

1H NMR (DMSO 400.13 MHz) δ 2.1 (2H, s), 3.78 (2H, s), 4.45 (2H, s), 5.39 (1H, s), 6.29 (1H, s).

tert-Butyl N-[[3-(hydroxymethyl)-1,2-oxazol-5-yl]methyl]carbamate was prepared as follows:—

Ethyl 5-[[(2-methylpropan-2-yl)oxycarbonylamino]methyl]-1,2-oxazole-3-carboxylate (5 g, 18.5 mmol, 1 eq) was dissolved in ethanol (50 mL) and cooled to 0° C. Sodium borohydride (1.89 g, 49.95 mmol, 5 eq) was added portionwise and the reaction was stirred at room temperature overnight. The mixture was quenched with aqueous sodium bicarbonate solution, extracted with ethyl acetate (×3), washed with brine, dried (MgSO₄) and evaporated to give product as a colouress oil (4.45 g, >100%).

1H NMR (CDCl3 400.13 MHz) δ 1.43 (9H, s), 4.4 (2H, d), 4.72 (2H, s), 5.0 (1H, s), 6.22 (1H, s). MS m/z 173 (MH+−56).

Ethyl 5-[[(2-methylpropan-2-yl)oxycarbonylamino]methyl]-1,2-oxazole-3-carboxylate was prepared as shown in Example 61.

### Example 138

### N′-[5-[2-(5-methoxypyridin-3-yl)ethyl]-1H-pyrazol-3-yl]-N-[(3-methyl-1,2-oxazol-5-yl)methyl]pyrimidine-2,4-diamine

5-[2-(5-Methoxypyridin-3-yl)ethyl]-1H-pyrazol-3-amine (102 mg, 0.467 mmol) and 4-chloro-N-[(3-methyl-1,2-oxazol-5-yl)methyl]pyrimidin-2-amine (106 mg, 0.467 mmol) were heated with HCl (37 [2l, 4M solution in dioxane, 0.148 mmol) in ethanol (1 ml) in a microwave reactor at 120° C. for 30 min. The solution was allowed to stand at 5° C. for 24 h and the precipitated solid was collected by filtration. The solid were combined with the filtrate, evaporated to dryness and purified by preparative HPLC using decreasingly polar mixtures of water (containing 0.1% NH₃) and MeCN as eluents. Fractions containing the desired compound were evaporated to dryness to afford N′-[5-[2-(5-methoxypyridin-3-yl)ethyl]-1H-pyrazol-3-yl]-N-[(3-methyl-1,2-oxazol-5-yl)methyl]pyrimidine-2,4-diamine as a brown glassy solid (15 mg, 8% yield).

1H NMR (399.902 MHz, DMSO) δ 2.22 (3H, s), 2.87-3.02 (4H, m), 3.85 (3H, s), 4.58 (2H, d), 6.01-6.44 (2H, bs), 6.15 (1H, s), 7.19-7.28 (1H, bd), 7.29 (1H, s), 7.88 (1H, d), 8.09 (1H, d), 8.17 (1H, d), 9.40 (1H, bs), 11.96 (1H, bs); m/z (ES+) [M+H]+=407.

4-Chloro-N-[(3-methyl-1,2-oxazol-5-yl)methyl]pyrimidin-2-amine was prepared as outlined in Example 13.

5-[2-(5-Methoxypyridin-3-yl)ethyl]-1H-pyrazol-3-amine, used as starting material, was prepared as described for Example 127.

### Example 139

### N-[3-methoxy-5-[2-[5-[[2-[(3-methyl-1,2-oxazol-5-yl)methylamino]pyrimidin-4-yl]amino]-2H-pyrazol-3-yl]ethyl]phenyl]acetamide

A mixture of N-{3-[2-(3-amino-1H-pyrazol-5-yl)ethyl]-5-methoxyphenyl}acetamide (138 mg, 0.5 mmol, 1.0 eq), 4-chloro-N-[(3-methyl-1,2-oxazol-5-yl)methyl]pyrimidin-2-amine (113 mg, 0.5 mmol, 1.0 eq), and ethanol (2.5 ml) was stirred and heated at 85° C. for 4 h under an atmosphere of nitrogen. The resulting suspension was allowed to cool to room temperature and then filtered to give N-[3-methoxy-5-[2-[5-[[2-[(3-methyl-1,2-oxazol-5-yl)methylamino]pyrimidin-4-yl]amino]-1H-pyrazol-3-yl]ethyl]phenyl]acetamide as a white solid, (142 mg, 61% yield).

¹H NMR (500.13 MHz, DMSO-d₆, CD₃CO₂D ) δ 2.03 (3H, s), 2.20 (3H, s), 2.85-2.90 (4H, m), 3.72 (3H, s), 4.66 (2H, s), 6.17 (2H, s), 6.45 (1H, d), 6.50 (1H, t), 7.04 (1H, s), 7.08 (1H, s), 7.86 (1H, d)

MS: m/z 463 (MH+)

4-Chloro-N-[(3-methyl-1,2-oxazol-5-yl)methyl]pyrimidin-2-amine was prepared as outlined in Example 13.

N-{3-[2-(3-amino-1H-pyrazol-5-yl)ethyl]-5-methoxyphenyl}acetamide, used as starting material was prepared as follows:—

Lithium diisopropylamide solution (1.8M in tetrahydrofuran/heptane/ethylbenzene, 17.8 mL, 32.0 mmol, 4.0 eq) was added to anhydrous tetrahydrofuran (52 ml) at −78° C. and the mixture stirred at this temperature under an atmosphere of nitrogen. Acetonitrile (1.7 ml, 32.0 mmol, 4.0 eq) was added dropwise and the solution maintained at −78° C. for 5 mins. A solution of methyl 3-(3-acetamido-5-methoxyphenyl)propanoate (2.02 g, 8.0 mmol, 1.0 eq) in tetrahydrofuran (20 mL) was added rapidly and the mixture stirred at −78° C. for 5 mins and then allowed to warm to 5° C. over 30 mins. Hydrazine hydrochloride (2.20 g, 32.0 mmol, 4.0 eq) and ethanol (56 ml) were then added and the mixture heated at 68° C. for 4 h. The mixture was evaporated, water (100 mL) was added and the mixture acidified with hydrochloric acid (2.0M, 50 ml) and then extracted with ethyl acetate (2×100 ml). The aqueous layer was basified with concentrated sodium hydroxide solution and then extracted with ethyl acetate. The organic layer was separated, washed with brine, dried over magnesium sulphate and evaporated to give a foam. The crude product was purified by silica column chromatography, eluting with a 3-10% gradient of methanol containing ammonia (2.0M) in dichloromethane. The clean fractions were taken and evaporated to afford the desired compound as a clear gum, 417 mg (19%).

MS: m/z 275 (MH+)

Methyl 3-(3-acetamido-5-methoxyphenyl)propanoate, used as starting material was prepared as follows:

A mixture of methyl 3-(3-amino-5-methoxyphenyl)propanoate (2.0 g, 9.55 mmol, 1.0 eq) and acetic anhydride (2.71 mL, 28.65 mmol, 3.0 eq) was heated at 120° C. for 20 mins. Water (20 ml) was added and the mixture was heated for a further 20 mins. After cooling, the mixture was partitioned between ethyl acetate and aq. sodium bicarbonate solution. The organic layer was washed with brine, dried over magnesium sulphate and evaporated to give the desired compound as an oil, (2.4 g, 100% yield).

MS: m/z 252 (MH+)

Methyl 3-(3-amino-5-methoxyphenyl)propanoate, used as starting material was prepared as follows:

A mixture of methyl 3-{3-[(tert-butoxycarbonyl)amino]-5-methoxyphenyl}propanoate (3.05 g, 9.85 mmol, 1.0 eq) and trifluoroacetic acid (15.2 mL, 197 mmol, 20.0 eq) was stirred at room temperature overnight. The trifluoroacetic acid was evaporated and the residue partitioned between ethyl acetate (150 ml) and aq. sodium bicarbonate solution (100 ml). The ethyl acetate extracts were combined and washed with brine, dried over magnesium sulphate and evaporated to give the desired compound as a clear oil, (2.0 g, 97% yield).

1H NMR (399.9 MHz, CDCl3) δ 2.57-2.61 (2H, m), 2.80-2.84 (2H, m), 3.29 (2H, s), 3.67 (3H, s), 3.74 (3H, s), 6.09 (1H, t), 6.14 (1H, q), 6.17 (1H, t)

MS: m/z 210 (MH+)

Methyl 3-{3-[(tert-butoxycarbonyl)amino]-5-methoxyphenyl}propanoate, used as starting material was prepared as follows:—

A mixture of methyl 3-[3-methoxy-5-[(2-methylpropan-2-yl)oxycarbonylamino]phenyl]prop-2-enoate (3.26 g, 10.6 mmol, 1.0 eq) dissolved in ethyl acetate (100 mL) and 5% palladium on charcoal catalyst (750 mg) was stirred at room temperature under an atmosphere of hydrogen for 2 h. The mixture absorbed 320 mL of hydrogen. The suspension was then flushed with nitrogen, filtered and evaporated. This gave methyl 3- {3-[(tert-butoxycarbonyl)amino]-5-methoxyphenyl}propanoate as an oil, (3.16 g, 96% yield).

MS: m/z 310 (MH+)

Methyl 3-[3-methoxy-5-[(2-methylpropan-2-yl)oxycarbonylamino]phenyl]prop-2-enoate, used as starting material was prepared as follows:

A mixture of tert-butyl (3-formyl-5-methoxyphenyl)carbamate (4.78 g, 19.0 mmol, 1.0 eq) and methyl (triphosphoranylidene) acetate (6.99 g, 20.9 mmol, 1. eq) dissolved in anhydrous tetrahydrofuran (200 mL) was stirred at room temperature under an atmosphere of nitrogen for 48 h. After evaporation of the solvent, the crude product was purified by silica column chromatography, eluting with dichloromethane. The clean fractions were taken and evaporated to give methyl 3-[3-methoxy-5-[(2-methylpropan-2-yl)oxycarbonylamino]phenyl]prop-2-enoate as a white solid, (3.35 g, 57%).

¹H NMR (399.9 MHz, CDCl₃) δ 1.52 (9H, s), 3.80 (3H, s), 3.81 (3H, s), 6.40 (1H, d), 6.51 (1H, s), 6.73 (1H, t), 7.08 (2H, s), 7.59 (1H, d)

MS: m/z 308 (MH+)

tert-Butyl (3-formyl-5-methoxyphenyl)carbamate, used as starting material was prepared as follows:—

A suspension of tert-butyl [3-(hydroxymethyl)-5-methoxyphenyl]carbamate (5.32 g, 21.0 mmol, 1.0 eq) and manganese (IV) dioxide (activated 5um, 7.3 g, 84 mmol, 4.0 eq) in ethyl acetate (230 mL) was stirred for 18 h at room temperature under nitrogen. The reaction mixture was then refluxed for 2 h. The mixture was filtered and evaporated to give tert-butyl (3-formyl-5-methoxyphenyl)carbamate as a white solid, (5.0 g, 95% yield).

MS: m/z 252 (MH+)

tert-Butyl [3-(hydroxymethyl)-5-methoxyphenyl]carbamate, used as starting material was prepared as follows:—

Sodium borohydride (4.77 g, 126.0 mmol, 6.0 eq) was added to a stirred solution of methyl 3-[(tert-butoxycarbonyl)amino]-5-methoxybenzoate (5.91 g; 21. 0 mmol, 1.0 eq) in methanol (51 mL) and tetrahydrofuran (50 mL) at room temperature. The mixture was stirred for 30 mins and then allowed to stand for 72 h. A further amount of sodium borohydride (4.77 g, 126 mmol, 6.0 eq) was added. The mixture was stirred for 18 h. The resulting solution was neutralised by the addition of hydrochloric acid (0.5M aqueous) and then extracted with ethyl acetate (400 mL). The ethyl acetate extract was washed with water, brine, dried over magnesium sulphate, filtered and then evaporated to give crude tert-butyl [3-(hydroxymethyl)-5-methoxyphenyl]carbamate as a clear gum, (6.0 g, 113%). This material was used without further purification.

MS: m/z 254 (MH+)

Methyl 3-[(tert-butoxycarbonyl)amino]-5-methoxybenzoate, used as starting material was prepared as follows:—

3-Methoxy-5-(methoxycarbonyl)benzoic acid (6.3 1 g, 30.0 mmol, 1.0 eq) was dissolved in warm tert-butanol (50 mL). N,N-diethylethanamine (4.19 mL, 30.0 mmol, 1.0 eq) was added followed by diphenyl phosphoryl azide (6.47 mL, 30.0 mmol, 1.0 eq) and the mixture was refluxed for 3.5 hours. The solvent was evaporated and the residue partitioned between ethyl acetate (400 mL) and water (200 mL). The organic layer was separated, washed with brine, dried over magnesium sulphate and evaporated to give the crude product. The crude product was purified by silica column chromatography, eluting with a 1-5% gradient of ethyl acetate in dichloromethane. The clean fractions were taken and evaporated to give methyl 3-[(tert-butoxycarbonyl)amino]-5-methoxybenzoate as a white solid, (6.60 g, 78%).

¹H NMR (399.9 MHz, CDCl₃) δ1.52 (9H, s), 3.83 (3H, s), 3.90 (3H, s), 6.60 (1H, s), 7.24-7.25 (1H, m), 7.37 (1H, s), 7.49-7.50 (1H, m)

The preparation of 3-methoxy-5-(methoxycarbonyl)benzoic acid, used as starting material is described by Zhao, He; Thurkauf, Andrew in Synthetic Communications (2001), 31(12), 1921-1926.

### Example 140

### 5-[[[4-[[5-[2-(3-propan-2-yloxyphenyl)ethyl]-1H-pyrazol-3-yl]amino]pyrimidin-2- yl]amino]methyl]-1,2-oxazole-3-carboxamide

2-Chloro-N-[5-[2-(3-propan-2-yloxyphenyl)ethyl]-1H-pyrazol-3-yl]pyrimidin-4-amine (60 mg, 0. 17 mmol, 1.0 eq)) was dissolved in 2-methoxyethanol (4 ml) and 5-(aminomethyl)-1,2-oxazole-3-carboxamide (60 mg, 0.34 mmol, 2.0 eq) and N-ethyl-N-propan-2-yl-propan-2-amine (117 μl, 0.59 mmol, 3.5 eq) were added. The mixture was heated to 180° C. for a total of 90 mins in the microwave reactor. The solvent was evaporated under reduced pressure and the crude product purified by reverse-phase prep. HPLC (basic) using a gradient of 29-49% acetonitrile in water containing 1% ammonium hydroxide solution. The clean fractions were taken and evaporated to afford as a beige solid. (39 mg, 50% yield).

¹H NMR (399.902 MHz, DMSO) δ 1.16 (d, J=6.1 Hz, 6H), 2.78 (m, 4H), 4.48 (m, 1H), 4.54 (d, J=5.6 Hz, 2H), 6.24 (s, 1H), 6.45 (s, 1H), 6.69 (m, 3H), 7.09 (t, J=7.8 Hz, 1H), 7.21 (s, 1H), 7.66 (s, 1H), 7.77 (d, J=5.4 Hz, 1H), 7.94 (s, 1H), 9.33 (s, 1H), 11.86 (s, 1H). MS: m/z=463 (MH+)

2-chloro-N-[5-[2-(3-propan-2-yloxyphenyl)ethyl]-1H-pyrazol-3-yl]pyrimidin-4-amine, used as starting material, was prepared as follows:—

2,4-Dichloropyrimidine (177 mg, 1. 18 mmol, 1.0 eq) was dissolved in ethanol (5 ml) and N-ethyl-N-propan-2-yl-propan-2-amine (0.25 ml, 1.42 mmol, 1.2 eq) and 5-[2-(3-propan-2-yloxyphenyl)ethyl]-1H-pyrazol-3-amine (290 mg, 1.30 mmol, 1.1 eq) were added. The mixture was stirred at 50° C. for 3 days. The reaction mixture was added slowly to water (10 ml), sonicated and the precipitate collected by filtration, washed with water and dried in vacuo to give 2-chloro-N-[5-[2-(3-propan-2-yloxyphenyl)ethyl]-1H-pyrazol-3-yl]pyrimidin-4-amine (122 mg, 29%) as a white solid.

¹H NMR (399.902 MHz, DMSO) δ 1.17 (d, J=6.0 Hz, 6H), 2.81 (s, 4H), 4.49 (septet, J=6.0 Hz, 1H), 6.02 (s, 1H), 6.69 (m, 4H), 7.10 (t,J=8.1 Hz, 1H), 8.09 (d, J=5.8 Hz, 1H), 10.22 (s, 1H). MS: m/z=358 (MH+)

5-[2-(3-Propan-2-yloxyphenyl)ethyl]-1H-pyrazol-3-amine was prepared as follows:—

Methyl 3-(3-propan-2-yloxyphenyl)propanoate (680 mg, 3.06 mmol, 1.0 eq) was dissolved in 1,4-dioxan (20 ml) under nitrogen and sodium hydride 60% suspension (147 mg, 3.67 mmol, 1.2 eq) and dry acetonitrile (0.19 ml, 3.67 mmol, 1.2 eq) were added. The solution was stirred at room temperature for 10 mins and then heated to 100° C. for 18 h. The mixture was then cooled to room temperature and ethanol (2 ml) and hydrazine hydrochloride (420 mg, 6.12 mmol, 2.0 eq) were added. The mixture was heated to 100° C. for 18 h. The solvent was evaporated and the residue partitioned between 1M HCl and ethyl acetate. The aqueous layer was basified with concentrated ammonia solution and extracted with ethyl acetate. The organic extracts were washed with water then brine, dried over MgSO4 and evaporated. The residue was purified by silica column chromatography, eluting with a gradient of 0.5-7% methanol in DCM. The clean fractions were evaporated to give 5-[2-(3-propan-2-yloxyphenyl)ethyl]-1H-pyrazol-3-amine (296 mg, 39%) as a brown oil.

¹H NMR (399.902 MHz, DMSO) δ 1.18 (d, J=5.7 Hz, 6H), 2.63 (m, 2H), 2.73 (m, 2H), 4.33 (bs, 1H), 4.50 (septet, J=6.0 Hz, 1H), 5.12 (s, 1H), 6.66 (m, 3H), 7.08 (t, J=8.1 Hz, 1H), 11.03 (bs, 1H). MS: m/z=246 (MH+)

Methyl 3-(3-propan-2-yloxyphenyl)propanoate was prepared as follows:—

Methyl 3-(3-hydroxyphenyl)propanoate (1.0 g, 5.55 mmol, 1.0 eq) was dissolved in dry acetone (20 ml) and anhydrous potassium carbonate (921 mg, 6.66 mmol, 1.2 eq) and 2-iodopropane (0.67 ml, 6.66 mmol, 1.2 eq) were added. The mixture was refluxed at 55° C. under nitrogen for 24 h. A further equivalent of potassium carbonate (844 mg, 5.55 mmol, 1.0 eq) and 2-iodopropane (0.4 ml, 5.55 mmol, 1.0 eq) were then added and stirring at 55° C. was continued for 24 h. The solvent was then evaporated and the residue dissolved in water (25 ml). The solution was extracted with diethyl ether (3×10 ml) and the extracts were combined, dried and evaporated. The crude product was purified by silica column chromatography, eluting with 0-10% MeOH in DCM. The product-containing fractions were combined, evaporated and dried to give methyl 3-(3-propan-2-yloxyphenyl)propanoate (686 mg, 56%) as a pale yellow oil.

¹H NMR (399.902 MHz, DMSO) δ 1.18 (d, J=5.9 Hz, 6H), 2.55 (t, J=7.6 Hz, 2H), 2.74 (t, J=7.6 Hz, 2H), 3.52 (s, 3H), 4.51 (septet, J=6.0 Hz, 1H), 6.67 (m, 3H), 7.09 (t, J=8.0 Hz, 1H).

Methyl 3-(3-hydroxyphenyl)propanoate was prepared as follows:—

3-(3-Hydroxyphenyl)propanoic acid (3.0 g, 18.1 mmol, 1.0 eq) was dissolved in dry DMF (50 ml), potassium hydrogen carbonate (2.17 g, 21.7 mmol, 1.2 eq) was added and the mixture was stirred at room temperature under nitrogen for 10 mins. Methyl iodide (1.24 ml, 19.9 mmol, 1.1 eq) was then added and the mixture was heated at 40° C. overnight. The solvent was evaporated and the residue dissolved in diethyl ether (50 ml), washed with water (20 ml) then ammonium chloride solution (20 ml), dried over MgSO4 and evaporated to give methyl 3-(3-hydroxyphenyl)propanoate (3.21 g, 98%) as a brown oil.

¹H NMR (399.902 MHz, DMSO) δ 2.59 (t, J=7.9 Hz, 2H), 2.77 (t, J=7.7 Hz, 2H), 3.59 (s, 3H), 6.60 (m, 3H), 7.06 (m, 1H), 9.24 (s, 1H). MS: m/z=179 M−(H+) [ES−]

5-(Aminomethyl)-1,2-oxazole-3-carboxamide was prepared as in Example 123.

### Example 141

### N-methyl-3-[2-[5-[[2-[(3-methyl-1,2-oxazol-5-yl)methylamino]pyrimidin-4-yl]amino]-1H-pyrazol-3-yl]ethyl]benzamide

3-[2-(5-Amino-1H-pyrazol-3-yl)ethyl]-N-methyl-benzamide (98 mg, 0.6 mmol) and 4-chloro-N-[(3-methyl-1,2-oxazol-5-yl)methyl]pyrimidin-2-amine (90 mg, 0.4 mmol) in ethanol (3 ml) were heated at 180° C. in a microwave reactor for 30 mins. The reaction mixture was cooled and concentrated. The crude product was purified by reverse phase prep. HPLC (basic) using a 15-40% gradient of acetonitrile in water containing 1% ammonia. The clean fractions were taken and evaporated to afford the title compound as a white solid (59 mg, 34%).

1H NMR (500.13 MHz, DMSO-d₆) δ 2.19 (3H, s), 2.78-2.82 (3H, m), 2.89-2.92 (2H, m), 2.94-3.01 (2H, m), 4.59 (2H, d), 6.11 (2H, s), 6.27 (1H, s), 7.35 (2H, q), 7.64 (1H, s), 7.65 (1H, d), 7.73 (1H, s), 7.87 (1H, d), 7.94 (1H, s), 8.80 (1H, s), 11.69 (1H, s)

MS m/z: 433 (MH+)

4-Chloro-N-[(3-methyl-1,2-oxazol-5-yl)methyl]pyrimidin-2-amine was prepared as outlined in Example 13.

3-[2-(5-amino-1H-pyrazol-3-yl)ethyl]-N-methyl-benzamide, used as starting material was prepared as follows:—

To a stirred suspension of 3-[2-(5-amino-1H-pyrazol-3-yl)ethyl]benzoic acid (1.620 g, 7.0 mmol) and 2M N-methylmethanamine in THF (5.25 mL, 10.5 mmol) in dry DMF (50 mL), dry N-ethyl-N-propan-2-yl-propan-2-amine (4.63 mL, 4 eq, 28.0 mmol) was added. O-(7-Azabenzotriazol-1-Y1)-N,N,N′,N′-Tetramethyluronium Hexafluoro-Phosphate (2.93 g, 7.7 mmol) was then added and the mixture left to stir for 18 h. The reaction mixture was evaporated to dryness, dissolved in ethyl acetate and then partitioned between water (30 ml) and ethyl acetate (30 ml). The aqueous layer was washed with ethyl acetate (3×30 ml). The organic layers were combined, washed sequentially with brine (1×30 ml), 0.5N citric acid (1×30 ml) and NaHCO₃ solution (1×30 ml) and evaporated to dryness to afford crude 3-[2-(5-amino-1H-pyrazol-3-yl)ethyl]-N-methyl-benzamide as an orange gum (1.3594 g). The crude product was purified by silica column chromatography, eluting with a gradient of 0-10% MeOH in DCM. Pure fractions were evaporated to dryness to afford pure 3-[2-(5-amino-1H-pyrazol-3-yl)ethyl]-N-methyl-benzamide (0.330 g, 28%).

1H NMR (399.9 MHz, DMSO-d₆) δ 2.74-2.79 (2H, m), 2.76-2.78 (3H, m), 2.89 (2H, d), 3.20-3.45 (2H, s), 5.21 (1H, s), 7.35-7.36 (2H, m), 7.63-7.66 (1H, m), 7.72 (1H, s), 8.36-8.37 (1H, m)

MS: m/z 245.41 (MH⁺)

3-[2-(5-Amino-1H-pyrazol-3-yl)ethyl]benzoic acid used as starting material, was prepared as follows:—

A suspension of 3-[2-(5-amino-2H-pyrazol-3-yl)ethyl]benzonitrile (4.000 g, 19.0 mmol) in an aqueous solution of sodium hydroxide (1OM, 40 ml) was heated at 95-100° C. for 5 h. The reaction mixture was cooled to 5-10° C. in an ice/water bath and acidified to pH3 by the dropwise addition of conc. HCl (approx. 50 ml). The resultant cream solid was removed by filtration, washed with water and then dried in a vacuum oven over the weekend to leave pure 3-[2-(5-amino-1H-pyrazol-3-yl)ethyl]benzoic acid (4.4208 g, 101% yield).

¹H NMR (399.9 MHz, DMSO-d₆) δ2.79 (2H, d), 2.95 (2H, d), 5.29 (1H, s), 7.41 (1H, t), 7.48 (1H, d), 7.77 (1H, s), 7.79 (1H, s), 7.82 (1H, d)

MS: m/z 232.39 (MH⁺)

3-[2-(5-amino-2H-pyrazol-3-yl)ethyl]benzonitrile, used as starting material, was prepared as follows:—

Sodium hydride (60%, 3.0 g, 75.6 mmol) was added to a stirred solution of methyl 3-(3-cyanophenyl)propanoate (1 1.9 g, 63.0 mmol) in dry 1,4 dioxane (350 ml) and dry acetonitrile (3.95 ml, 75.6 mmol) under nitrogen to give a cloudy grey mixture. This was stirred at room temperature for 10 mins and then refluxed under nitrogen overnight to give a dark orange solution. The reaction mixture was cooled and ethanol (25 ml) was added followed by hydrazine monohydrochloride (8.635 g, 126 mmol). The reaction mixture was refluxed overnight. The reaction mixture was cooled, filtered, and evaporated to dryness to afford crude 3-[2-(5-amino-2H-pyrazol-3-yl)ethyl]benzonitrile (16 g). The crude product was purified by silica column chromatography, eluting isocratically with 8% MeOH in DCM. Pure fractions were evaporated to dryness to afford 3-[2-(5-amino-2H-pyrazol-3-yl)ethyl]benzonitrile as an orange gum, (5.1 g, 38%).

1H NMR (399.9 MHz, DMSO-d₆) δ 2.73-2.76 (2H, m), 2.88-2.92 (2H, m), 4.07-4.08 (1H, m), 4.50 (2H, s), 5.17 (1H, s), 7.47-7.51 (1H, m), 7.55-7.58 (1H, m), 7.64-7.66 (2H, m)

MS: m/z 213.41 (MH⁺)

Methyl 3-(3-cyanophenyl)propanoate, used as starting material, was prepared as follows:—

To a solution of methyl (E)-3-(3-cyanophenyl)prop-2-enoate (12.36 g, 66.00 mmol) dissolved in DMF (250 ml), was added platinum catalyst (1.24 g) and the reaction mixture was stirred under hydrogen overnight. The mixture was filtered through celite, washed with DMF, then evaporated to dryness to give a grey-brown liquid. The solid was dissolved in DCM (150 ml) and washed sequentially with water (3×80 ml) and brine (1×80 ml), then dried with MgSO₄, and evaporated to dryness to afford methyl 3-(3-cyanophenyl)propanoate as a brown liquid (11.949 g, 96%).

¹H NMR (399.9 MHz, DMSO-d₆) δ2.69 (2H, t), 2.90-2.94 (2H, m), 3.59 (3H, s), 7.50 (1H, t), 7.60-7.62 (1H, m), 7.66-7.69 (1H, m), 7.73 (1H, d)

Methyl (E)-3-(3-cyanophenyl)prop-2-enoate, used as starting material, was prepared as follows:—

Methyl (triphenyphosphoranylidene)acetate (38.12 g, 114 mmol) was added to a mixture of 3-cyanobenzaldehyde (9.97 g, 76 mmol) in DCM (150 ml) and the reaction mixture was stirred for 6 h at room temperature. The reaction mixture was evaporated to dryness to afford crude methyl (E)-3-(3-cyanophenyl)prop-2-enoate. The crude product was purified by silica column chromatography, eluting isocratically with 50% ethyl acetate in isohexanes. Pure fractions were evaporated to dryness to afford pure methyl (E)-3-(3-cyanophenyl)prop-2-enoate (12.36 g, 87%).

¹H NMR (399.9 MHz, DMSO-d₆) δ 3.76 (3H, s), 6.84 (1H, s), 7.64 (1H, t), 7.68 (1H, s), 7.87-7.89 (1H, m), 8.06-8.09 (1H, m), 8.27 (1H, t)

### Example 142

### N,3-dimethyl-5-[2-[5-[[2-[(3-methyl-1,2-oxazol-5-yl)methylamino]pyrimidin-yl]amino]-1H-pyrazol-3-yl]ethyl]benzamide

3-[2-(5-Amino-1H-pyrazol-3-yl)ethyl]-N,5-dimethyl-benzamide (142 mg, 0.6 mmol) and 4-chloro-N-[(3-methyl-1,2-oxazol-5-yl)methyl]pyrimidin-2-amine (135 mg, 0.25 mmol) in ethanol (4 ml) were heated at 180° C. in a microwave reactor for 30 mins. The reaction mixture was cooled and the suspension was filtered. The crude product was washed with cold ethanol (5 ml) and diethyl ether (3×10 ml). The residue was air-dried to give N,3-dimethyl-5-[2-[5-[[2-[(3-methyl-1,2-oxazol-5-yl)methylamino]pyrimidin-4-yl]amino]-1H-pyrazol-3-yl]ethyl]benzamide as a cream solid (133 mg, 49.6%). 1H NMR (399.9 MHz, DMSO-d₆) δ 2.19 (3H, s), 2.33 (3H, s), 2.77 (3H, d), 2.90 (4H, s), 4.70-4.71 (2H, m), 6.28 (2H, s), 6.38 (1H, s), 7.20 (1H, s), 7.49-7.52 (2H, m), 7.89 (1H, s), 8.33-8.34 (1H, m), 8.79 (1H, s), 11.23 (1H, s), 12.45 (1H, s). MS m/z: 447 (MH+)

4-Chloro-N-[(3-methyl-1,2-oxazol-5-yl)methyl]pyrimidin-2-amine was prepared as outlined in Example 13.

3-[2-(5-Amino-1H-pyrazol-3-yl)ethyl]-N,5-dimethyl-benzamide, used as starting material, was prepared as follows:—

Anhydrous acetonitrile (653 μl, 12.5 mmol) was added to anhydrous THF (50 ml), containing a solution of 1.8 M lithium diisopropylamide (in THF; 6.97 ml) at −78° C. The solution was stirred at −78° C. for 10 mins. A solution of methyl 3-[3-methyl-5-(methylcarbamoyl)phenyl]propanoate (1.475 g, 6.25 mmol) in anhydrous THF (10 ml) was added rapidly and the reaction mixture stirred at −78° C. for 30 mins. The reaction mixture was stirred at 20° C. for 1 h. Two additional equivalents of the acetonitrile anion were added (prepared at −78° C.) and the mixture stirred for 1 h. The reaction mixture was quenched with 1N HCl solution and extracted with diethyl ether (3×40 ml). The extracts were dried (MgSO4), filtered and evaporated. The residue was dissolved in ethanol (25 ml) and refluxed with hydrazine monohydrate (1 ml) for 18 h. The reaction mixture was cooled and evaporated to dryness. The residue was dissolved and partitioned between water and DCM (20 ml:40 ml). The aqueous layer was extracted with DCM (4×25 ml). The extracts were washed with saturated brine solution (25 ml), filtered and evaporated to give 3-[2-(5-amino-1H-pyrazol-3-yl)ethyl]-N,5-dimethyl-benzamide, as a yellow foam (0.685 g, 42%). 1H NMR (399.9 MHz, DMSO-d₆) δ 2.32 (3H, s), 2.69-2.79 (2H, m), 2.80 (3H, d), 2.83-2.90 (2H, m), 5.20 (1H, s), 7.19 (1H, s), 7.48 92H, d), 8.31 (1H, s). MS m/z: 259 (MH+).

Methyl 3-[3-methyl-5-(methylcarbamoyl)phenyl]propanoate, used as starting material, was prepared as follows:—

Methyl (E)-3-[3-methyl-5-(methylcarbamoyl)phenyl]prop-2-enoate (3.27 g, 14 mmol) was dissolved in a mixture of ethanol (50 ml) and DMF (10 ml). To this was added 10% Pd/C (300 mg) and the reaction mixture was stirred under a hydrogen atmosphere overnight. The reaction mixture was filtered through celite and evaporated to afford to give methyl 3-[3-methyl-5-(methylcarbamoyl)phenyl]propanoate as an oil 2.78 g, ( 84.5%). 1H NMR (399.9 MHz, DMSO-d₆) δ 2.32 (3H, s), 2.65 (2H, t), 2.77 (3H, d), 2.85 (2H, d), 3.60 (3H, s), 7.19-7.19 (1H, m), 7.48 (2H, s), 8.31 (1H, d). MS m/z: 258 (M+Na+).

Methyl (E)-3-[3-methyl-5-(methylcarbamoyl)phenyl]prop-2-enoate was prepared as follows:

Methyl(triphenyl-phosphoranylidene)acetate (10.02 g, 30 mmol) was added under nitrogen to a stirred solution of 3-formyl-N,5-dimethyl-benzamide (3.55 g, 20 mmol) in dry DCM (50 ml) at 0° C. The reaction mixture was stirred at 20° C. for 18 h. The solvent was evaporated and the crude product was purified by silica column chromatography, eluting with a 25-50% gradient of ethyl acetate in hexanes. The pure fractions were combined and evaporated to give methyl (E)-3-[3-methyl-5-(methylcarbamoyl)phenyl]prop-2-enoate a white solid (3.25 g, 70%). 1H NMR (399.9 MHz, DMSO-d₆) 6 2.38 (3H, s), 2.76-2.86 (3H, m), 3.70-3.80 (3H, m), 6.69 (2H, d), 7.61-7.71 (3H, m), 7.96 (1H, s), 8.38-8.47 (1H, m). MS m/z: 234 (MH+).

3-formyl-N,5-dimethyl-benzamide used as starting material was prepared using an analogous method to that outlined in Example 139 for tert-Butyl (3-formyl-5-methoxyphenyl)carbamate except using 3-(hydroxymethyl)-N,5-dimethyl-benzamide (3.59 g, 20 mmol) and manganese (IV) dioxide (activated 5 um, 6.960 mol), to give 3-formyl-N,5-dimethyl-benzamide as a white solid (3.54 g, 100%). 1H NMR (399.9 MHz, DMSO-d₆) δ 2.46 (3H, s), 2.81-2.82 (3H, m), 7.86 (1H, d), 7.98 (1H, t), 8.17 (1H, s), 8.60-8.61 (1H, m), 10.04 (1H, s).

3-(hydroxymethyl)-N,5-dimethyl-benzamide was prepared from:—

A solution of trimethylaluminium (2M in toluene, 25 ml, 12.5 mmol) was added dropwise at −50° C. to a stirred solution of methyl 3-(hydroxymethyl)-5-methyl-benzoate (3.5 g, 20 mmol) and methylamine (2.0M solution in THF, 50 ml, 100 mmol) in dry THF (100 ml). The reaction mixture was stirred for 15 mins at −50° C., then at 20° C. for 18 h. The reaction was cooled to −50° C. and quenched with saturated potassium sodium tartrate solution and stirred for 1 h. The reaction mixture was extracted with ethyl acetate (2×50 ml) and washed with saturated brine solution (25 ml). The extracts were dried (MgSO4), filtered and evaporated. The crude product was purified by silica column chromatography, eluting with a gradient of 0-5% methanol in dichloromethane. The pure fractions were combined and evaporated to dryness to give 3-(hydroxymethyl)-N,5-dimethyl-benzamide as an oil (3.7 g, ˜100%). ¹H NMR (399.9 MHz, DMSO-d₆) δ2.35 (3H, s), 2.78 (3H, d), 4.52 (2H, d), 5.22 (1H, t), 7.27-7.28 (1H, m), 7.52 (1H, s), 7.60 (1H, s), 8.34 (1H, d). MS m/z: 180 (MH+)

Methyl 3-(hydroxymethyl)-5-methyl-benzoate was prepared as follows:

A solution of borane-DMS complex (2M in THF, 30 ml, 60 mmol) was added dropwise at 0° C. to a stirred solution of 3-methoxycarbonyl-5-methylbenzoic acid (9.72 g, 50 mmol) in anhydrous THF (50 ml), under nitrogen. The reaction mixture was stirred at 20° C. for 30 mins and then heated at 60° C. for 18 h. The reaction mixture was cooled and quenched with a mixture of 1:2 water/glacial acetic acid (7.2 ml). The reaction mixture was concentrated and partitioned between ethyl acetate (50 ml) and potassium carbonate solution (2M, 25 ml). The organic phase was washed with hydrochloric acid (1M, 25 ml), saturated sodium bicarbonate and saturated brine solution. The organic extracts were dried over magnesium sulphate, filtered and evaporated to give methyl 3-(hydroxymethyl)-5-methyl-benzoate as a clear oil, (8.16 g, 91%). ¹H NMR (399.9 MHz, DMSO-d₆) δ2.37 (3H, s), 3.86 (3H, s), 4.54 (2H, d), 5.28 (1H, t), 7.40-7.41 (1H, m), 7.66 (1H, d), 7.75 (1H, d)

### Example 143

### 4-Methoxy-N-methyl-6-[2-[5-[[2-[(3-methyl-1,2-oxazol-5-yl)methylamino]pyrimidin-4-yl]amino]-1H-pyrazol-3-yl]ethyl]pyridine-2-carboxamide

6-[2-(5-amino-1H-pyrazol-3- yl)ethyl]-4-methoxy-N-methyl-pyridine-2-carboxamide (138 mg, 0.5 mmol) and 4-chloro-N-[(3-methyl-1,2-oxazol-5-yl)methyl]pyrimidin-2-amine (103 mg, 0.5 mmol) in ethanol (4 ml) were heated at 120° C. in a microwave reactor for 1 h. The reaction mixture was cooled and filtered to give the crude product. The crude product was washed with cold methanol (10 ml) and diethyl ether (2×10 ml) and air-dried. The crude product was purified by reverse phase prep. HPLC (Basic) using a 20-40% gradient of acetonitrile in water containing 1% ammonia. The clean fractions were taken and evaporated to afford the title compound as a white solid (69 mg, 30%).

1H NMR (500.13 MHz, DMSO-d₆) δ 2.19 (3H, s), 2.87 (3H, d), 3.00-3.05 (2H, m), 3.06-3.11 (2H, m), 3.89 (3H, s), 4.58 (2H, d), 6.07 (1H, s), 6.12 (1H, s), 6.30 (1H, s), 6.70 (1H, s), 6.97 (1H, d), 7.40 (1H, d), 7.87 (1H, d), 8.27 (1H, s), 8.85 (1H, s), 11.70 (1H, s). MS m/z: 464 (MH+)

4-Chloro-N-[(3-methyl-1,2-oxazol-5-yl)methyl]pyrimidin-2-amine was prepared as outlined in Example 13.

6-[2-(5-amino-1H-pyrazol-3-yl)ethyl]-4-methoxy-N-methyl-pyridine-2-carboxamide used as starting material was prepared following the procedure for 3-[2-(5-amino-1H-pyrazol-3-yl)ethyl]-N,5-dimethyl-benzamide in Example 142, but starting from methyl 3-[4-methoxy-6-(methylcarbamoyl)pyridin-2-yl]propanoate (581 mg, 2.3 mmol), acetonitrile ( 481 ul, 9.2 mmol), 1.8 M LDA in THF (5 ml, 9.2 mmol) and hydrazine hydrochloride (631 mg, 9.20 mmol). The crude product was purified by silica column chromatography, eluting with a gradient of 0-10% methanol in dichloromethane. Pure fractions were combined and evaporated to give 6-[2-(5-amino-1H-pyrazol-3-yl)ethyl]-4-methoxy-N-methyl-pyridine-2-carboxamide as a gum (454 mg, 71%).

1H NMR (399.9 MHz, DMSO-d₆) δ 2.84 (3H, d), 2.89-2.94 (2H, m), 2.99-3.03 (2h, m), 3.87 (3H, s), 5.17 (1H, m), 6.99 (1H, d), 7.37 (1H, m), 8.42 (1H, s), 8.55 (1H, d). MS m/z: 276 (MH+).

Methyl 3-[4-methoxy-6-(methylcarbamoyl)pyridin-2-yl]propanoate was prepared following the procedure for methyl 3-[3-methyl-5-(methylcarbamoyl)phenyl]propanoate in Example 142, but starting from methyl (E)-3-[4-methoxy-6-(methylcarbamoyl)pyridin-2-yl]prop-2-enoate (676 mg, 2.7 mmol) to afford methyl 3-[4-methoxy-6-(methylcarbamoyl)pyridin-2-yl]propanoate as an oil (595 mg, 87%).

1H NMR (399.9 MHz, DMSO-d₆) δ 2.84 (3H, d), 2.88 (2H, d), 3.03 (2H, t), 3.62 (3H, s), 3.88 (3H, s), 7.05 (1H, d), 7.38 (1H, d), 8.51-8.52 (1H, m).

Methyl (E)-3-[4-methoxy-6-(methylcarbamoyl)pyridin-2-yl]prop-2-enoate used as starting material was prepared following the procedure for methyl-5-(methylcarbamoyl)phenyl]prop-2-enoate in Example 142, but starting from 6-formyl-4-methoxy-N-methyl-pyridine-2-carboxamide (1.27 g, 6.5 mmol) and methyl(triphenyl-phosphoranylidene)acetate (3.26 g, 9.75 mmol). The crude product was purified by silica column chromatography, eluting with a gradient of 25-40% ethyl acetate in hexanes. Pure fractions were combined and evaporated to give methyl (E)-3-[4-methoxy-6-(methylcarbamoyl)pyridin-2-yl]prop-2-enoate as a white solid (680 mg, 42%). 1H NMR (399.9 MHz, DMSO-d₆) δ 2.85-2.89 (3H, m), 3.78 (3H, s), 3.93 (3H, s), 7.34-7.38 (1H, m), 7.49-7.53 (2H, m), 7.67 (1H, s), 8.92 (1H, d). MS m/z: 251 (MH+).

6-formyl-4-methoxy-N-methyl-pyridine-2-carboxamide used as starting material was prepared using an analogous method to that used for tert-butyl (3-formyl-5-methoxyphenyl)carbamate in Example 139,, but starting from 6-(hydroxymethyl)-4-methoxy-N-methyl-pyridine-2-carboxamide (1.34 g, 6.80 mmol) and manganese (IV) dioxide (activated 5 um, 2.37 g, 27.2 mmol). The crude product was purified by silica column chromatography, eluting with a gradient of 2-5% methanol in dichloromethane. Pure fractions were combined and evaporated to give to give the title compound as a white solid (1.27 g, 96%). 1H NMR (399.9 MHz, DMSO-d₆) δ 2.84-2.88 (3H, m), 2.90 (1H, s), 4.00 (3H, s), 7.57 (1H, d), 7.75 (1H, d), 8.80 (1H, s), 10.00 (1H, d). MS m/z: 195 (MH+).

6-(Hydroxymethyl)-4-methoxy-N-methyl-pyridine-2-carboxamide used as starting material was prepared following the procedure for 3-(hydroxymethyl)-N,5-dimethyl-benzamide in Example 142, but starting from methyl 6-(hydroxymethyl)-4-methoxy-pyridine-2-carboxylate (1.5 g, 7.6 mmol), trimethylaluminium (2M in toluene, 19 ml, 9.5 mmol) and methylamine (2.0M solution in THF, 19 ml, 38 mmol). The crude product was purified by silica column chromatography, eluting with a gradient of 0-5% methanol in dichloromethane. Pure fractions were combined and evaporated to give to give the title compound as a white solid (1.36 g, 91%).

1H NMR (399.9 MHz, DMSO-d₆) δ 2.82-2.83 (3H, m), 3.90 (3H, s), 4.59 (2H, d), 5.41-5.48 (1H, m), 7.14 (1H, d), 7.40 (1H, d), 8.67-8.69 (1H, m). MS m/z: 197 (MH+)

Methyl 6-(hydroxymethyl)-4-methoxy-pyridine-2-carboxylate used as starting material, was prepared following the procedure described by Atsushi Kittaka, Yuichi Sugano, Masami Otsuka and Masaji Ohno, Tetrahedron, Vol 44, No 10, p 2821 (1988)—example 4, Man-designed bleomycins. synthesis of dioxygen activating molecules and a DNA cleaving molecule based on bleomycin-Fe(II)-0₂ complex.

TABLE 4

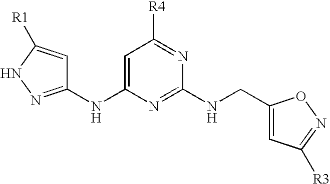

| Ex- | | | |
|---|---|---|---|
| ample | R1 | R4 | R3 |
| 66 | 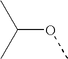 | H | Me |
| 67 | 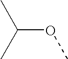 | H | 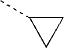 |
| 68 | 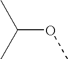 | Me | Me |
| 69 | 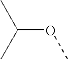 | Me | 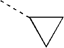 |
| 70 | 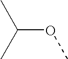 | OMe | Me |
| 71 | 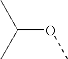 | OMe | 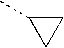 |
| 72 | 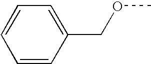 | H | Me |
| 73 | 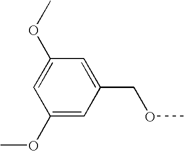 | H | Me |
| 74 | 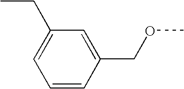 | H | Me |
| 75 | 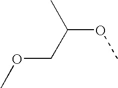 | H | Me |
| 76 | 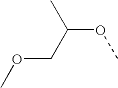 | H | 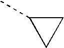 |
| 77 | 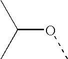 | H | 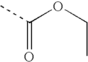 |
| 78 | 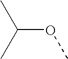 | H | 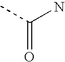 |
| 79 | 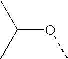 | H | 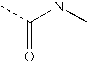 |
| 80 | 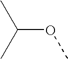 | H | 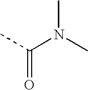 |
| 81 | 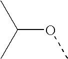 | H | 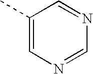 |
| 82 | 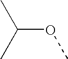 | H | 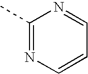 |
| 83 | 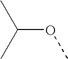 | H | 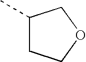 |
| 84 | 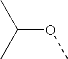 | H | 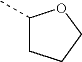 |
| 85 | 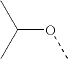 | H | 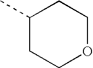 |
| 86 | 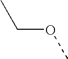 | H | Me |
| 87 | 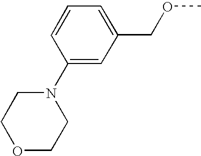 | H | Me |
| 88 | 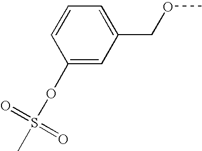 | H | Me |
| 89 | 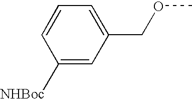 | H | Me |
| 90 | 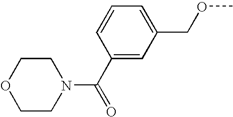 | H | Me |
| 91 | 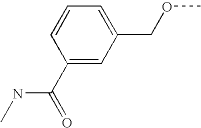 | H | Me |
| 92 | 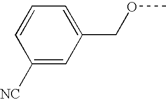 | H | Me |
| 93 | 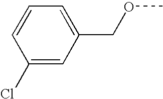 | H | Me |
| 94 | 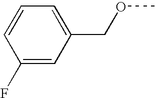 | H | Me |
| 95 | 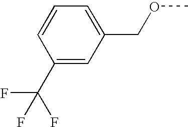 | H | Me |
| 96 | 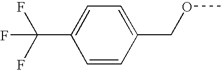 | H | Me |
| 97 | 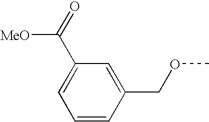 | H | Me |
| 98 | 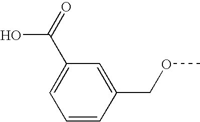 | H | Me |
| 99 | 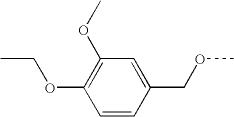 | H | Me |
| 100 | 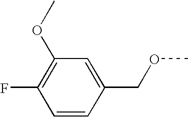 | H | Me |
| 101 | 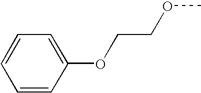 | H | Me |
| 102 | 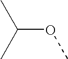 | H | 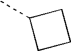 |
| 103 | 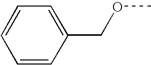 | H | 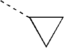 |
| 131 | 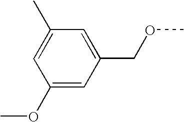 | H | Me |
| 135 | 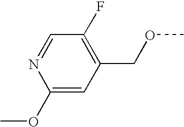 | H | Me |
| 137 | 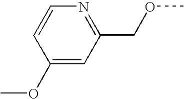 | H | Me |
| 144 | 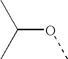 | H | 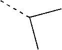 |
| 145 | 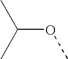 | H | 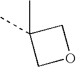 |
| 146 | 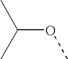 | H | 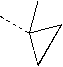 |
| 147 | 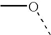 | H | Me |

### Example 66

### N′-(5-isopropoxy-2H-pyrazol-3-yl)-N-[(3-methylisoxazol-5-yl)methyl]pyrimidine-2,4-diamine (also known as N-[(3-methyl-1,2-oxazol-5-yl)methyl]-N′-(5-propan-2-yloxy-2H-pyrazol-3-yl)pyrimidine-2,4-diamine)

To a stirred degassed solution of 5-bromo-N′-(5-isopropoxy-1H-pyrazol-3-yl)-N-[(3-methylisoxazol-5-yl)methyl]pyrimidine-2,4-diamine (also known as 5-bromo-N-[(3-methyl-1,2-oxazol-5-yl)methyl]-N′-(5-propan-2-yloxy-1H-pyrazol-3-yl)pyrimidine-2,4-diamine; 0.12 g, 0.29 mmol) in ethanol (15 ml) was added 10% palladium on carbon (12 mg). The mixture was stirred at room temperature for 24 hours under an atmosphere of hydrogen. The mixture was filtered through Celite and the residue washed with ethanol and then with a mixture of dichloromethane/dimethylformamide and finally with methanolic ammonia solution. The filtrate was evaporated and the residue dissolved in methanol and then purified using an Isolute SCX-3 column eluting with methanolic ammonia solution. Fractions containing product were combined and evaporated to leave example 66 in table 4 (0.045 g, 46% yield).

¹H NMR (300 MHz, DMSO): 1.27 (6H, d), 2.20 (3H, s), 4.52-4.71 (3H, m), 5.21 (1H, s), 6.02 (1H, d), 6.17 (1H, s), 7.71 (1H, s), 7.91 (1H, d), 9.98 (1H, s), 11.81 (1H, s).

MS: m/z 330 (MH⁺).

5-bromo-N′-(5-isopropoxy-1H-pyrazol-3-yl)-N-[(3-methylisoxazol-5-yl)methyl]pyrimidine-2,4-diamine (also known as 5-bromo-N-[(3-methyl-1,2-oxazol-5-yl)methyl]-N′-(5-propan-2-yloxy-1H-pyrazol-3-yl)pyrimidine-2,4-diamine), used as starting material, was prepared as follows:
a) To a solution of 5-isopropoxy-1H-pyrazol-3-amine (2.0 g, 14.2 mmol) in dry tetrahydrofuran (60 ml) under a nitrogen atmosphere was added triethylamine and the mixture cooled to 0° C. A solution of 5-bromo-2,4-dichloropyrimidine (3.23 g, 14.2 mmol) in dry tetrahydrofuran (30 ml) was added dropwise and the mixture was allowed to stir at room temperature for 18 hours. The mixture was evaporated and the residue crystallised with ethyl acetate. The mixture was filtered and the residue triturated thoroughly with water. The resultant solid was filtered and then left to dry overnight to give 5-bromo-2-chloro-N-(5-isopropoxy-1H-pyrazol-3-yl)pyrimidin-4-amine (1.645 g, 35% yield).

MS: m/z 332 (MH⁺).
b) A mixture of 5-bromo-2-chloro-N-(5-isopropoxy- I H-pyrazol-3-yl)pyrimidin-4-amine (0.20 g, 0.6 mmol), N-[(3-methylisoxazol-5-yl)methyl]methanamine hydrochloride (also known as N-methyl-1-(3-methyl-1,2-oxazol-5-yl)methanamine hydrochloride; 0.116 g, 0.78 mmol) and di-iso-propylethylamine (0.419 ml, 2.4 mmol) in 2-methoxyethanol (3 ml) was heated in a microwave at 200° C. for 30 minutes. The mixture was concentrated and the residue purified by flash chromatography on silica eluting with a mixture of 50% iso-hexane in ethylacetate. The fractions containing product were combined and evaporated to leave 5-bromo-N′-(5-isopropoxy-1H-pyrazol-3-yl)-N-[(3-methylisoxazol-5-yl)methyl]pyrimidine-2,4-diamine (also known as 5-bromo-N-[(3-methyl-1,2-oxazol-5-yl)methyl]-N′-(5-propan-2-yloxy-1H-pyrazol-3-yl)pyrimidine-2,4-diamine) (0. 125 g, 51% yield).

MS: m/z 408 (MH⁺).

5-isopropoxy-1H-pyrazol-3-amine, used as starting material, can be prepared according to the literature (Sato, Tadahisa; Mizukawa, Hiroki; Kawagishi, Toshio. Preparation of 3-alkoxy-5-amino-1H-pyrazoles as intermediates for photographic magenta couplers JP01013072).

### Example 67

### N-[(3-cyclopropylisoxazol-5-yl)methyl]-N ′-(5-isopropoxy-2H-pyrazol-3-yl)pyrimidine-2,4-diamine (also known as N-[(3-cyclopropyl-1,2-oxazol-5-yl)methyl]-N′-(5-propan-2-yloxy-2H-pyrazol-3-yl)pyrimidine-2,4-diamine)

To a stirred degassed solution of 5-bromo-N-[(3-cyclopropylisoxazol-5-yl)methyl]-N′-(5-isopropoxy-1H-pyrazol-3-yl)pyrimidine-2,4-diamine (also known as 5-bromo-N-[(3-cyclopropyl-1,2-oxazol-5-yl)methyl]-N′-(5-propan-2-yloxy-1H-pyrazol-3-yl)pyrimidine-2,4-diamine; 0.152 g, 0.37 mmol) in ethanol (15 ml) was added 10% palladium on carbon (15 mg). The mixture was stirred at room temperature for 24 hours under an atmosphere of hydrogen. The mixture was filtered through Celite and the residue washed with ethanol and then with methanolic ammonia solution. The filtrate was evaporated and the residue dissolved in methanol and purified using an Isolute SCX-3 column eluting with methanolic ammonia solution. Fractions containing product were combined and evaporated to leave a residue. The solid was then purified again by preparative hplc using a gradient of acetonitrile in water containing 1% ammonia solution. The fractions containing product were combined and then evaporated to leave example 67 in table 4 (0.041 g, 31% yield).

¹H NMR (300 MHz, DMSO): 0.69-0.74 (2H, m), 0.94-1.00 (2H, m), 1.27 (6H, d), 1.90-2.01 (1H, m), 4.49-4.71 (3H, m), 5.28 (1H, s), 5.96-6.10 (2H, m), 7.68 (1H, s), 7.93 (1H, s), 10.00 (1H, s), 11.92 (1H, s).

MS: m/z 356 (MH⁺).

5-bromo-N-[(3-cyclopropylisoxazol-5-yl)methyl]-N′-(5-isopropoxy-1H-pyrazol-3-yl)pyrimidine-2,4-diamine (also known as 5-bromo-N-[(3-cyclopropyl-1,2-oxazol-5-yl)methyl]-N′-(5-propan-2-yloxy-1H-pyrazol-3-yl)pyrimidine-2,4-diamine), used as starting material, was prepared as follows:
a) In an analogous reaction to that described in example 66b, 5-bromo-2-chloro-N-(5-isopropoxy-1H-pyrazol-3-yl)pyrimidin-4-amine (0.30 g, 0.9 mmol) was reacted with (3-cyclopropylisoxazol-5-yl)methanamine hydrochloride (also known as (3-cyclopropyl-1,2-oxazol-5-yl)methanamine hydrochloride; 0.205 g, 1. 17 mmol) to give 5-bromo-N-[(3-cyclopropylisoxazol-5-yl)methyl]-N′-(5-isopropoxy-1H-pyrazol-3-yl)pyrimidine-2,4-diamine (also known as 5-bromo-N-[(3-cyclopropyl-1,2-oxazol-5-yl)methyl]-N′-(5-propan-2-yloxy-1H-pyrazol-3-yl)pyrimidine-2,4-diamine; 0.176 g, 45% yield).

¹H NMR (300 MHz, DMSO): 0.77 (2H, m), 1.05 (2H, m), 1.32 (6H, d), 2.01 (1H, m), 4.59 (2H, s), 4.71 (1H, m), 5.69 (1H, s), 6.12 (1H, s), 8.02 (1H, s), 8.17 (1H, s), 9.40 (1H, bs), 11.82 (1H, bs).

MS: m/z 436 (MH⁺).

(3-cyclopropyl-1,2-oxazol-5-yl)methanamine hydrochloride, used as starting material, was prepared as in Example 3.

### Example 68

### N′-(5-isopropoxy-1H-pyrazol-3-yl)-6-methyl-N-[(3-methylisoxazol-5-yl)methyl]pyrimidine-2,4-diamine (also known as 6-methyl-N-[(3-methyl-1,2-oxazol-5-yl)methyl]-N′-(5-propan-2-yloxy-1H-pyrazol-3-yl)pyrimidine-2,4-diamine)

A mixture of 4-chloro-6-methyl-N-[(3-methylisoxazol-5-yl)methyl]pyrimidin-2-amine (also known as 4-chloro-6-methyl-N-[(3-methyl-1,2-oxazol-5-yl)methyl]pyrimidin-2-amine; 0.20 g, 0.84 mmol) and 5-isopropoxy-1H-pyrazol-3-amine (0.178 g, 1.26 mmol) in anhydrous 1-methylpyrrolidinone (2 mL) and 4M hydrogen chloride solution in dioxane (0.42 mL) was heated at 110° C. for 4 hours. The mixture was left to stand at room temperature overnight and was then diluted with saturated sodium bicarbonate solution and extracted with ethyl acetate (×2). The organic extracts were washed with brine, dried over magnesium sulfate, filtered and then evaporated to leave an orange oil. The oil was purified by chromatography on silica eluting with a mixture of 2-4% methanol in dichloromethane. Fractions containing product were combined and then evaporated to leave a solid which was triturated with diethyl ether to leave example 68 in table 4 (0.039 g, 12% yield).

¹H NMR (500 MHz, DMSO at 373K): 1.28 (d, 6H), 2.15 (s, 3H), 2.19 (s, 3H), 4.58 (d, 2H), 4.64 (bs, 1H), 5.25 (bs, 1H), 5.41 (bs, 1H), 6.12 (s, 1H), 7.2 (bs, 1H), 9.33 (bs, 1H), 11.39 (bs, 1H).

MS: m/z 344 (MH⁺).

4-chloro-6-methyl-N-[(3-methylisoxazol-5-yl)methyl]pyrimidin-2-amine (also known as 4-chloro-6-methyl-N-[(3-methyl-1,2-oxazol-5-yl)methyl]pyrimidin-2-amine), used as starting material, was prepared as follows:
a) (3-methylisoxazol-5-yl)methanamine hydrochloride (also known as (3-methyl-1,2-oxazol-5-yl)methanamine hydrochloride; 2.09 g, 14.0 mmol) was dissolved in diglyme (8 ml) and di-iso-propylethylamine (2.43 ml) added. After a few minutes 6-methyl-2-methylsulfanyl-3H-pyrimidin-4-one (2.0 g, 12.8 mmol) was added in a single portion and the solution was then heated at 160° C. for 3 hours. The orange solution was allowed to cool to room temperature and then dissolved in dichloromethane and purified directly by chromatography on silica eluting with a mixture of 2.5-20% methanol in dichloromethane. Fractions containing product were combined and evaporated to leave a solid which was triturated with diethyl ether to give 6-methyl-2-[(3-methylisoxazol-5-yl)methylamino]-3H-pyrimidin-4-one (0.914 g, 32% yield).

¹H NMR (400 MHz, DMSO): 2.02 (s, 3H), 2.2 (s, 3H), 4.56 (s, 2H), 5.5 (s, 1H), 6.19 (s, 1H), 6.94 (bs, 1H), 10.8 (bs, 1H).
b) A mixture of 6-methyl-2-[(3-methylisoxazol-5-yl)methylamino]-3H-pyrimidin-4-one (also known as 6-methyl-2-[(3-methyl-1,2-oxazol-5-yl)methylamino]-3H-pyrimidin-4-one; 0.914 g, 4.15 mmol) and di-iso-propylethylamine (0.938 ml, 5.4 mmol) was stirred in toluene (5 ml) and then phosphorous oxychloride (0.465 ml, 4.98 mmol) was added dropwise. The mixture was stirred at room temperature for 30 minutes then heated at 80° C. for 2 hours. The mixture was allowed to cool to room temperature and then poured into a saturated sodium bicarbonate solution. The product was extracted with ethyl acetate (×2) and the combined extracts were washed with brine, dried over magnesium sulfate, filtered and then evaporated to leave an orange gum. The gum was triturated with diethyl ether to give 4-chloro-6-methyl-N-[(3-methylisoxazol-5-yl)methyl]pyrimidin-2-amine (also known as 4-chloro-6-methyl-N-[(3-methyl-1,2-oxazol-5-yl)methyl]pyrimidin-2-amine; 0.728 g, 73% yield).

¹H NMR (400 MHz, DMSO): 2.19 (s, 3H), 2.27 (s, 3H), 4.55 (d, 2H), 6.15 (s, 1H), 6.68 (s, 1H), 8.09 (t, 1H).

MS: m/z 239 (MH⁺).

5-Isopropoxy-1H-pyrazol-3-amine was synthesized as outlined in Example 66.

### Example 69

### N-[(3-cyclopropylisoxazol-5-yl)methyl]-N ′-(5-isopropoxy-2H-pyrazol-3-yl)-6-methyl-pyrimidine-2,4-diamine (also known as N-[(3-cyclopropyl-1,2-oxazol-5-yl)methyl]-6-methyl-N′-(5-propan-2-yloxy-2H-pyrazol-3-yl)pyrimidine-2,4-diamine)

A mixture of 2-chloro-N-(5-isopropoxy- I H-pyrazol-3-yl)-6-methyl-pyrimidin-4-amine (0.214 g, 0.80 mmol), (3-cyclopropylisoxazol-5-yl)methanamine hydrochloride (also known as (3-cyclopropyl-1,2-oxazol-5-yl)methanamine hydrochloride; 0.168 g, 0.96 mmol) and di-iso-propylethylamine (0.18 ml, 1.04 mmol) in 1-butanol (5 ml) was heated at 120° C. for 2 days. The mixture was diluted with ethyl acetate and washed with water, brine, dried over magnesium sulfate and then evaporated to leave an orange gum. The gum was purified by chromatography on silica eluting with a mixture of 0-5% methanol in dichloromethane. Fractions containing product were combined and evaporated to leave a solid which was triturated with diethyl ether to give example 69 in table 4 (0.118 g, 40% yield).

¹H NMR (500 MHz, DMSO 373K): 0.73 (m, 2H), 0.95 (m, 2H), 1.29 (d, 6H), 1.92 (m, 1H), 2.15 (s, 3H), 4.56 (d, 2H), 4.6 (s, 1H), 5.33 (bs, 1H), 5.96 (bs, 1H), 6.02 (s, 1H), 7.08 (bs, 1H), 9.2 (bs, 1H), 11.39 (bs, 1H).

MS: m/z 370 (MH⁺).

2-chloro-N-(5-isopropoxy-1H-pyrazol-3-yl)-6-methyl-pyrimidin-4-amine, used as starting material, was prepared as follows:
a) A mixture of 2,4-dichloro-6-methyl pyrimidine (1. 16 g, 7.08 mmol), 5-isopropoxy-1H-pyrazol-3-amine (1.0 g, 7.08 mmol) and sodium carbonate (0.826 g, 7.79 mmol) in ethanol (50 ml) was heated at 50° C. for 7 days. The mixture was evaporated and the residue taken up in ethyl acetate and then washed with saturated sodium bicarbonate solution followed by water and then brine. The organic phase was dried over magnesium sulfate, filtered and then evaporated to leave a brown oil. The oil was purified by chromatography on silica eluting with a mixture of 25-60% ethyl acetate in iso-hexane. Fractions containing product were combined evaporated to leave 2-chloro-N-(5-isopropoxy-1H-pyrazol-3-yl)-6-methyl-pyrimidin-4-amine (0.214 g, 11% yield).

¹H NMR (400 MHz, DMSO): 1.28 (d, 6H), 2.29 (s, 3H), 4.52 (bs, 1H), 5.6 (bs, 1H), 6.5-7.5 (bs, 1H), 10.08 (bs, 1H), 11.9 (bs, 1H).

MS: m/z 268 (MH⁺).

(3-cyclopropyl-1,2-oxazol-5-yl)methanamine hydrochloride, used as starting material, was prepared as in Example 3.

### Example 70

### N′-(5-isopropoxy-2H-pyrazol-3-yl)-6-methoxy-N-[(3-methylisoxazol-5-yl)methyl]pyrimidine-2,4-diamine (also known as 6-methoxy-N-[(3-methyl-1,2-oxazol-5-yl)methyl]-N′-(5-propan-2-yloxy-2H-pyrazol-3-yl)pyrimidine-2,4-diamine)

6-chloro-N′-(5-isopropoxy-1H-pyrazol-3-yl)-N-[(3-methylisoxazol-5-yl)methyl]pyrimidine-2,4-diamine (also known as 6-chloro-N-[(3-methyl-1,2-oxazol-5-yl)methyl]-N′-(5-propan-2-yloxy-1H-pyrazol-3-yl)pyrimidine-2,4-diamine; 0.140 g, 0.38 mmol) was dissolved in methanol (3 ml) and sodium methoxide (0. 104 g, 1.92 mmol) was added. The mixture was heated at 140° C. for 1 hour in a Emrys Optimiser microwave. The reaction was diluted with saturated ammonium chloride solution and then extracted with ethyl acetate (×2). The organic extracts were washed with water and then with brine, dried over magnesium sulfate, filtered and then evaporated to leave a yellow oil. The oil was purified by chromatography on silica eluting with a mixture of 0-5% methanol in dichloromethane. Fractions containing product were combined and evaporated to leave a solid which was triturated with diethyl ether to give example 70 in table 4 (0.045 g, 32% yield).

¹H NMR (500 MHz, DMSO 373K): 1.28 (d, 6H), 2.19 (s, 3H), 3.78 (s, 3H), 4.57 (d, 2H), 4.6 (bs, 1H), 5.21 (bs, 1H), 5.39 (bs, 1H), 6.12 (s, 1H), 7.35 (bs, 1H), 9.23 (bs, 1H), 11.35 (bs, 1H).

MS: m/z 360 (MH⁺).

6-chloro-N′-(5-isopropoxy-1H-pyrazol-3-yl)-N-[(3-methylisoxazol-5-yl)methyl]pyrimidine-2,4-diamine (also known as 6-chloro-N-[(3-methyl-1,2-oxazol-5-yl)methyl]-N′-(5-propan-2-yloxy-1H-pyrazol-3-yl)pyrimidine-2,4-diamine), used as starting material, was prepared as follows:
a) A solution of 2,4,6-trichloropyrimidine (1.3 g, 7.08 mmol) and sodium carbonate (0.751 g, 7.08 mmol) in ethanol (20 ml) was cooled to 0° C. and then 5-isopropoxy-1H-pyrazol-3-amine (1.0 g, 7.08 mmol) was added. The mixture was stirred at room temperature overnight and then evaporated. The residue was taken up in ethyl acetate (50 ml) and washed with water (50 ml) and then with brine (25 ml). The organic extracts were dried over magnesium sulfate, filtered and then evaporated to leave a yellow oil. The oil was purified by chromatography on silica eluting with a mixture of 25-60% ethyl acetate in iso-hexane. The fractions containing product were combined and evaporated to leave a solid that was triturated with diethyl ether to give 2,6-dichloro-N-(5-isopropoxy-1H-pyrazol-3-yl)pyrimidin-4-amine (1.06 g, 52% yield).

¹HNMR(400 MHz, DMSO 373K): 1.31 (d, 6H), 4.5 (bs, 1H), 5.62 (s, 1H), 7.19 (bs, 1H), 10.16 (bs, 1H), 11.72 (bs, 1H).

MS: m/z 288 (MH⁺).
b) A mixture of 2,6-dichloro-N-(5-isopropoxy-1H-pyrazol-3-yl)pyrimidin-4-amine (0.350 g, 1.21 mmol), (3-methylisoxazol-5-yl)methanamine hydrochloride (also known as (3-methyl-1,2-oxazol-5-yl)methanamine hydrochloride; 0.361 g, 2.43 mmol) and di-iso-propylethylamine (0.634 ml, 3.64 mmol) was heated in 1-hexanol (5 ml) at 120° C. for 3 hours. The mixture was evaporated and the residue was dissolved in ethyl acetate (20 ml) and then washed with water (20 ml) followed by brine (20 ml). The organic extract was dried over magnesium sulfate, filtered and then evaporated to leave a yellow oil. The oil was purified by chromatography on silica eluting with a mixture of 0-5% methanol in dichloromethane. Fractions containing product were combined and evaporated to leave a solid that was triturated with diethyl ether to give 6-chloro-N′-(5-isopropoxy-1H-pyrazol-3-yl)-N-[(3-methylisoxazol-5-yl)methyl]pyrimidine-2,4-diamine (also known as 6-chloro-N-[(3-methyl-1,2-oxazol-5-yl)methyl]-N′-(5-propan-2-yloxy-1H-pyrazol-3-yl)pyrimidine-2,4-diamine; 0.140 g, 32% yield).

¹H NMR (500 MHz, DMSO 373K): 1.26 (d, 6H), 2.18 (s, 3H), 4.55 (m, 3H), 5.47 (bs, 1H), 6.1-6.25 (m, 2H), 7.55 (bs, 1H), 9.5 (bs, 1H), 11.45 (bs, 1H). MS: m/z 364 (MH⁺).

### Example 71

### N-[(3-cyclopropylisoxazol-5-yl)methyl]-N ′-(5-isopropoxy-2H-pyrazol-3-yl)-6-methoxy-pyrimidine-2,4-diamine (also known as N-[(3-cyclopropyl-1,2-oxazol-5-yl)methyl]-6-methoxy-N′-(5-propan-2-yloxy-2H-pyrazol-3-yl)pyrimidine-2,4-diamine)

Prepared in an analogous way to example 70 but starting with 6-chloro-N-[(3-cyclopropylisoxazol-5-yl)methyl]-N′-(5-isopropoxy-1H-pyrazol-3-yl)pyrimidine-2,4-diamine (also known as 6-chloro-N-[(3-cyclopropyl-1,2-oxazol-5-yl)methyl]-N′-(5-propan-2-yloxy-1H-pyrazol-3-yl)pyrimidine-2,4-diamine; 0.14 g, 0.35 mmol) to give example 71 in table 4 (0.067 g, 49% yield).

¹H NMR (500 MHz, DMSO 373K): 0.72 (m, 2H), 0.95 (m, 2H), 1.28 (d, 6H), 1.94 (m, 1H), 3.77 (s, 1H), 4.55 (d, 2H), 4.62 (bs, 1H), 5.21 (bs, 1H), 5.39 (bs, 1H), 6.04 (s, 1H), 7.33 (bs, 1H), 9.34 (bs, 1H), 11.34 (bs, 1H).

MS: m/z 386 (MH⁺).

6-chloro-N-[(3-cyclopropylisoxazol-5-yl)methyl]-N′-(5-isopropoxy-1H-pyrazol-3-yl)pyrimidine-2,4-diamine (also known as 6-chloro-N-[(3-cyclopropyl-1,2-oxazol-5-yl)methyl]-N′-(5-propan-2-yloxy-1H-pyrazol-3-yl)pyrimidine-2,4-diamine), used as starting material, was prepared as follows:
a) In an analogous reaction to that described for example 70b, 2,6-dichloro-N-(5-isopropoxy-1H-pyrazol-3-yl)pyrimidin-4-amine was reacted with (3-cyclopropylisoxazol-5-yl)methanamine hydrochloride (also known as (3-cyclopropyl-1,2-oxazol-5-yl)methanamine hydrochloride) to give 6-chloro-N-[(3-cyclopropylisoxazol-5-yl)methyl]-N′-(5-isopropoxy-1H-pyrazol-3-yl)pyrimidine-2,4-diamine (also known as 6-chloro-N-[(3-cyclopropyl-1,2-oxazol-5-yl)methyl]-N′-(5-propan-2-yloxy-1H-pyrazol-3-yl)pyrimidine-2,4-diamine; 0.14 g, 30% yield).

¹H NMR (500 MHz, DMSO 373K): 0.72 (m, 2H), 0.95 (m, 2H), 1.29 (d, 6H), 1.94 (m, 1H), 4.55 (m, 3H), 5.4 (bs, 1H), 6.04-6.2 (m, 2H), 7.5 (bs, 1H), 9.6 (bs, 1H), 11.42 (bs, 1H).

MS: m/z 390 (MH⁺).

(3-cyclopropyl-1,2-oxazol-5-yl)methanamine hydrochloride, used as starting material, was prepared as in Example 3.

### Example 72

### N′-(5-benzyloxy-1H-pyrazol-3-yl)-N-[(3-methylisoxazol-5-yl)methyl]pyrimidine-2,4-diamine (also known as N-[(3-methyl-1,2-oxazol-5-yl)methyl]-N′-(5-phenylmethoxy-1H-pyrazol-3-yl)pyrimidine-2,4-diamine)

A mixture of N-(5-benzyloxy-1H-pyrazol-3-yl)-2-chloro-pyrimidin-4-amine (0.045 g, 0.15 mmol), (3-methylisoxazol-5-yl)methanamine hydrochloride (also known as (3-methyl-1,2-oxazol-5-yl)methanamine hydrochloride; 0.045 g, 0.3 mmol) and di-iso-propylethylamine (0.078 ml, 0.45 mmol) in 2-methoxyethanol (2 ml) was heated at 160° C. for 1 hour in an Emrys Optimiser microwave. The mixture was evaporated and the residue purified by preparative hplc eluting with a gradient of acetonitrile in water both containing 1% formic acid to give example 72 in table 4 as the formate salt (0.008 g, 13% yield).

MS: m/z 378 (MH⁺).

(3-methyl-1,2-oxazol-5-yl)methanamine hydrochloride, used as starting material, was prepared as outlined in Example 1.

N-(5-benzyloxy-1H-pyrazol-3-yl)-2-chloro-pyrimidin-4-amine, used as starting material, was prepared a s follows:
a) A solution of 2,4-dichloropyrimdine (0.294 g, 2.0 mmol) and 5-benzyloxy-1H-pyrazol-3-amine (0.34 g, 1.8 mmol) and triethylamine (0.326 ml, 2.34 mmol) in ethanol (25 ml) was heated at 60° C. for 6 days. The mixture was evaporated and the residue partitioned between ethyl acetate (25 ml) and water (20 ml). The layers were separated and the aqueous layer was extracted with further portions of ethyl acetate (2×20 ml). The combined organic extracts were washed with brine, dried over magnesium sulfate, filtered and then evaporated. The residual oil was purified by chromatography on silica eluting with a mixture of 0-3% methanol in dichloromethane. Fractions containing product were combined and evaporated to leave N-(5-benzyloxy-1H-pyrazol-3-yl)-2-chloro-pyrimidin-4-amine (0.090 g, 17% yield).

MS: m/z 302 (MH⁺).

5-benzyloxy-1H-pyrazol-3-amine, used as starting material, was obtained as follows:
i) A solution of 5-amino-2H-pyrazol-3-ol (6.0 g, 60.6 mmol) was stirred in dichloromethane (75 ml). Triphenylphosphine (19.06 g, 72.7 mmol) was added and the mixture was then cooled to 5-10mC. Di-iso-propylazodicarboxylate (14.31 ml, 72.7 mmol) was added dropwise over a period of 20 minutes, maintaining the internal temperature <15° C. The mixture was then held at 10° C. for a further 20 minutes. Benzyl alcohol (7.52 ml, 72.7 mmol) was added dropwise and the mixture stirred at 5-10C for 1 hour and then allowed to warm to room temperature and stirred under nitrogen for 60 hours. The mixture was filtered and the filtrate was then extracted with 1 M hydrochloric acid (3×) and the combined extracts washed with dichloromethane (1 5 ml). The aqueous phase was basified with sodium bicarbonate (6.7 g) and the mixture was then extracted with dichloromethane (2×40 ml). The combined organic extracts were evaporated to leave a brown oil which was purified by chromatography on silica eluting with a mixture of 0-3% methanol in dichloromethane. The fractions containing product were combined and then evaporated to leave 5-benzyloxy-1H-pyrazol-3-amine (0.67 g, 6% yield).

¹H NMR (300 MHz, CDCl₃): 5.05 (s, 1H), 5.12 (s, 2H), 7.25-7.45 (m, 5H).

MS: m/z 190 (MH⁺).

### Example 73

### N′-[5-[(3,5-dimethoxyphenyl)methoxy]-1H-pyrazol-3-yl]-N-[(3-methylisoxazol-5-yl)methyl]pyrimidine-2,4-diamine (also known as N′-[5-[(3,5-dimethoxyphenyl)methoxy]-1H-pyrazol-3-yl]-N-[(3-methyl-1,2-oxazol-5-yl)methyl]pyrimidine-2,4-diamine)

Prepared in an analogous way to example 72 by reacting 2-chloro-N-[5-[(3,5-dimethoxyphenyl)methoxy]-1H-pyrazol-3-yl]pyrimidin-4-amine (0.052, 0.144 mmol) with (3-methylisoxazol-5-yl)methanamine hydrochloride (also known as (3-methyl-1,2-oxazol-5-yl)methanamine hydrochloride; 0.043 g, 0.29 mmol). After the reaction was complete the mixture was purified by preparative hplc eluting with a gradient of 25-45% acetonitrile in water containing 1% ammonia. The fractions containing product were combined and evaporated to leave example 73 in table 4 (0.022 g, 35% yield).

¹H NMR (300 MHz, DMSO): 2.18 (s, 3H), 3.73 (s, 6H), 4.58 (d, J=5.6 Hz, 2H), 5.07 (s, 2H), 5.30 (s, 1H), 6.02 (d, J=5.5 Hz, 1H), 6.17 (s, 1H), 6.43 (t, J=2.0 Hz, 1H), 6.59 (d, J=2.0 Hz, 2H), 7.69 (s, 1H), 7.92 (d, J=5.5 Hz, 1H), 10.00 (s, 1H), 11.90 (s, 1H).

MS: m/z 438 (MH⁺). (3-methyl-1,2-oxazol-5-yl)methanamine hydrochloride, used as starting material, was prepared as outlined in Example 1.

2-chloro-N-[5-[(3,5-dimethoxyphenyl)methoxy]-1H-pyrazol-3-yl]pyrimidin-4-amine, used as starting material, was prepared as follows:
a) A solution of 2,4-dichloropyrimdine (0.131 g, 0.88 mmol) and 5-[(3,5-dimethoxyphenyl)methoxy]-1H-pyrazol-3-amine (0.20 g, 0.80 mmol) and triethylamine (0.224 ml, 1.6 mmol) in ethanol (15 ml) was heated at 60° C. for 6 days. A further portion of 5-[(3,5-dimethoxyphenyl)methoxy]-1H-pyrazol-3-amine (0.060 g, 0.24 mmol) was added and the mixture heated at 60° C. for a further 18 hours. The mixture was evaporated and the residue partitioned between ethyl acetate (20 ml) and water (15 ml). The layers were separated and the aqueous phase was then further extracted with ethyl acetate (2×15 ml). The combined organic extracts were washed with brine, dried over magnesium sulfate, filtered and then evaporated. The residual oil was purified by chromatography on silica, eluting with a mixture of 0-3% methanol in dichloromethane to give 2-chloro-N-[5-[(3,5-dimethoxyphenyl)methoxy]-1H-pyrazol-3-yl]pyrimidin-4-amine (0.053 g, 18% yield).

MS: m/z 360 (MH⁺).

5-[(3,5-dimethoxyphenyl)methoxy]-1H-pyrazol-3-amine, used as starting material, was prepared as follows:
i) In an analogous reaction to that described for example 72i, 5-amino-2H-pyrazol-3-ol (3.0 g, 30.3 mmol) was reacted with 3,5-dimethoxybenzyl alcohol (6.12 g, 36.3 mmol) to give 5-[(3,5-dimethoxyphenyl)methoxy]-1H-pyrazol-3-amine (0.615 g, 8% yield).

¹H NMR (300 MHz, DMSO): 3.74 (s, 6H), 5.17 (s, 2H), 5.26 (s, 1H), 6.48 (s, 1H), 6.59 (s, 2H).

MS: m/z 250 (MH⁺).

### Example 74

### N′-[5-[(3-ethylphenyl)methoxy]-2H-pyrazol-3-yl]-N-[(3-methyl1,2-oxazol-5-yl)methyl]pyrimidine-2,4-diamine

Prepared in an analogous way to example 38, but starting with 5-[(3-ethylphenyl)methoxy]-2H-pyrazol-3-amine (153.5 mg, 0.71 mmol, 1 eq) and using a 35-55% gradient of acetonitrile in water containing 1% ammonia to purify. The title compound was obtained as a solid (47.7 mg, 17% yield).

1H NMR (300.132 MHz, DMSO): δ 1.19 (t, 3H), 2.19 (s, 3H), 2.62 (q, 2H), 4.58 (d, 2H), 5.10 (s, 2H), 5.29 (s, 1H), 6.02 (s, 1H), 6.17 (s, 1H), 7.13-7.31 (m, 4H), 7.69 (s, 1H), 7.91 (d, 1H), 10.00 (s, 1H), 11.91 (s, 1H). MS: m/z 406 (MH+).

5-[(3-ethylphenyl)methoxy]-2H-pyrazol-3-amine, used as starting material was prepared as follows:
a) 1M Borane. THF complex (60 ml, 60 mmol, 3 eq) was added to a solution of anhydrous tetrahydrofuran (50 ml) containing m-ethylbenzoic acid (3 g, 19.98 mmol, 1 eq) and was stirred at room temperature for 3days. The reaction was quenched by the dropwise addition of methanol until the evolution of gas had ceased. Some water was also added. The solvent was evaporated under reduced pressure to yield a white residue. The residue was extracted into ethyl acetate and washed with water then brine. Dried with magnesium sulphate, filtered and evaporated to afford (3-ethylphenyl)methanol as a yellow oil. (2.67 g, 98% yield).

1H NMR (300.132 MHz, DMSO): δ 1.18 (t, 3H), 2.60 (q, 2H), 4.47 (d, 2H), 5.09 (t, 1H), 7.05-7.16 (m, 3H), 7.23 (t, 1H).
b) 3-amino-5-hydroxypyrazole (1.62 g, 16.30 mmol, 1 eq) in Dichloromethane (20 ml) was cooled to 0 degc. Triphenylphosphine was then added to the reaction mixture (5.145 g, 19.60 mmol, 1.2 eq). Diisopropyl azodicarboxylate (3.86 ml, 19.60 mmol, 1.20) was then added dropwise over 15 mins. The reaction was held at 0 deg for 60 mins (a beige ppt came out of solution) before (3-ethylphenyl)methanol (2.67 g, 19.60 mmol, 1.2 eq) in dichloromethane (20 ml) was added dropwise. The reaction was held at 0 degc for a further 60 mins before warming to room temperature overnight. The reaction mixture was filtered and the filtrate partioned three times with 2M aqueous HCl. The washings were combined and extracted with ethyl acetate. After separation the acidic layer was basified by the addition of ammonia and re-extracted twice with ethyl acetate. The ethyl acetate extracts were combined, washed with brine, and dried with magnesium sulphate. The solvent was evaporated under reduced pressure to afford 5-[(3-ethylphenyl)methoxy]-2H-pyrazol-3-amine crude as a yellow oil (540 mg), which was used further without purification.

### Example 75

### N⁴-[5-(2-methoxy-1-methylethoxy)-1H-pyrazol-3-yl]-N²-[(3-methylisoxazol-5-yl)methyl]pyrimidine-2,4-diamine (also known as N′-[5-(1-methoxypropan-2-yloxy)-1H-pyrazol-3-yl]-N-[(3-methyl-1,2-oxazol-5-yl)methyl]pyrimidine-2,4-diamine)

2-Chloro-N-[5-(2-methoxy-1-methylethoxy)-1H-pyrazol-3-yl]pyrimidin-4-amine (55 mg, 0.194 mmol) and [(3-methylisoxazol-5-yl)methyl]amine. HCl (also known as (3-methyl-1,2-oxazol-5-yl)methanamine hydrochloride; 58 mg, 0.388 mmol) were heated with DIPEA (102ul, 0.582 mmol) in 2-methoxyethanol (2 ml) in a microwave reactor at 160° C. for an initial period of 30 min, then for a further 20 min. The solution was evaporated to dryness and the residue was purified by reverse phase acidic prep hplc, using a gradient of 5-50% MeCN in H₂O+0.2% TFA. The product fractions were neutralised with aqueous NaHCO₃, concentrated under vacuum to remove organic solvents and extracted with ethyl acetate (3×15 ml). The combined extracts were dried over MgSO₄, filtered and evaporated. The gummy residue was triturated with a mixture of ether and hexane to crystallize the product, the solvent was evaporated and the product was dried under vacuum to afford the title compound as a white solid (30 mg, 43% yield).

¹H NMR (300.132 MHz, DMSO) δ 1.24 (d, 3H), 2.19 (s, 3H), 3.30 (s, 3H—obscured by water peak), 3.36-3.54 (m, 2H), 4.58 (d, 2H), 4.62-4.76 (m, 1H), 5.23 (bs, 1H), 6.04 (bs, 1H), 6.16 (s, 1H), 7.67 (bs, 1H), 7.90 (d, 1H), 9.97 (bs, 1H), 11.86 (bs, 1H); MS: m/z 360 (MH⁺)

(3-methyl-1,2-oxazol-5-yl)methanamine hydrochloride, used as starting material, was prepared as outlined in Example 1.

2-Chloro-N-[5-(2-methoxy-1-methylethoxy)-1H-pyrazol-3-yl]pyrimidin-4-amine, used as starting material was prepared as follows:
a) 3-Amino-5-hydroxypyrazole (1 g, 10.09 mmol) was stirred in dichloromethane (15 ml) under nitrogen. Triphenylphosphine (3.18 g, 12.11 mmol) was then added and the reaction mixture was cooled in an ice-bath. Diisopropylazodicarboxylate (2.38 ml, 12.11 mmol) was added dropwise over a period of 15 min (temp <15° C.). The reaction mixture was then stirred in the ice-bath for 1 h. 1-Methoxy-2-propanol (1.19 ml, 12.1lmmol) was added dropwise over 10 min, the reaction mixture was allowed to warm to room temperature over 1 h and stirred under nitrogen for 3 days.

The reaction mixture was filtered to remove some undissolved solid and washed through with dichloromethane. The filtrate was extracted with 2M HCl (aq) (2×10 ml) and the combined extracts were washed with dichloromethane (10 ml). The aqueous phase was basified with solid NaHCO₃, and re-extracted with dichloromethane (3×10 ml). The basic aqueous phase was then evaporated to dryness and washed with ethyl acetate, filtered to remove inorganics and washed through with ethyl acetate. The solid filtered from the aqueous phase was re-dissolved in aqueous Na₂CO₃, then re-extracted with ethyl acetate; the pH of the aqueous was then adjusted to pH7-8 and re-extracted with ethyl acetate. The ethyl acetate extracts and washes were combined, dried over MgSO₄, filtered and evaporated to give the product, 5-(2-methoxy-1-methylethoxy)-1H-pyrazole-3-amine, as an orange/brown oil (0.60 g, 35%).

¹H NMR (300.132 MHz, DMSO) δ 1.18 (d, 3H), 3.26 (s, 3H), 3.31-3.48 (m, 2H), 4.52-4.64 (m, 1H), 4.67 (s, 1H), 4.86 (bs, 2H), 10.34 (bs, 1H); MS: m/z 172 (MH⁺).
b) 5-(2-Methoxy-1-methylethoxy)-1H-pyrazole-3-amine (0.41 g, 2.39 mmol) was stirred in ethanol (30 ml) under nitrogen. Triethylamine (0.668 ml, 4.79 mmol) was added, followed by 2,4-dichloropyrimidine (357 mg, 2.39 mmol). The solution was heated at 65° C. for 3 days. The solution was allowed to cool and the solvent was removed under vacuum. The residue was purified on a 20 g silica isolute column, eluting with 0-3% methanol in dichloromethane, to afford the product, 2-chloro-N-[5-(2-methoxy-1-methylethoxy)-1H-pyrazol-3-yl]pyrimidin-4-amine, as a pale yellow solid (1 14 mg, 17% yield).

MS: m/z 282 (M−H).

### Example 76

### N²-[(3-cyclopropylisoxazol-5-yl)methyl]-N4-[5-(2-methoxy-1-methylethoxy)-1H-pyrazol-3-yl]pyrimidine-2,4-diamine (also known as N-[(3-cyclopropyl-1,2-oxazol-5-yl)methyl]-N′-[5-(1-methoxypropan-2-yloxy)-1H-pyrazol-3-yl]pyrimidine-2,4-diamine)

2-Chloro-N-[5-(2-methoxy-1-methylethoxy)-1H-pyrazol-3-yl]pyrimidin-4-amine (55 mg, 0.194 mmol) and 1-(3-cyclopropylisoxazol-5-yl)methanamine.HCl (also known as (3-cyclopropyl-1,2-oxazol-5-yl)methanamine hydrochloride; 51 mg, 0.291 mmol) were heated with DIPEA (102 ul, 0.582 mmol) in 2-methoxyethanol (2 ml) in a microwave reactor at 160° C. for and initial period of 40 min, then for a further 1 h. The solvent was removed under vacuum and the residue was purified by reverse phase basic prep hplc, using a gradient of 20-40% MeCN in H₂O+1% NH₄OH (aq). The combined product fractions were evaporated to give a gum, which was then triturated with ether and hexane to crystallize the product. The solvent was evaporated and the solid dried under vacuum to afford the title compound as a white solid (27 mg, 36%).

¹H NMR (300.132 MHz, DMSO) δ 0.64-0.77 (m, 2H), 0.91-1.03 (m, 2H), 1.24 (d, 3H), 1.89-2.02 (m, 1H), 3.30 (s, 3H - obscured by water peak), 3.38-3.55 (m, 2H), 4.56 (d, 2H), 4.64-4.77 (m, 1H), 5.22 (bs, 1H), 6.02 (d, 1H), 6.06 (s, 1H), 7.65 (bs, 1H), 7.91 (d, 1H), 9.98 (bs, 1H), 11.87 (bs, 1H); MS: m/z 386 (MH⁺).

2-Chloro-N-[5-(2-methoxy-1-methylethoxy)-1H-pyrazol-3-yl]pyrimidin-4-amine, used as starting material was prepared as per example 75a).

3-Cyclopropyl-1,2-oxazol-5-yl)methanamine hydrochloride was synthesized as outlined in Example 3.

### Example 77

### Ethyl 5-[[[4-[(5-propan-2-yloxy-2H-pyrazol-3-yl)amino]pyrimidin-2-yl]amino]methyl]1,2-oxazole-3-carboxylate

To a solution of 2-chloro-N-(5-propan-2-yloxy-2H-pyrazol-3-yl)pyrimidin-4-amine (0.741 g, 2.92 mmol,1.00 eq) in 2-methoxy ethanol (15 ml) in a microwave tube was added the ethyl 5-(aminomethyl)1,2-oxazole-3-carboxylate.TFA salt (1.005 g, 3.52 mmol, 1.2 eq) followed by DIPEA (1.27 ml, 7.30 mmol, 2.5 eq. The mixture was then heated to 200° for 45 mins in the microwave. The solvent was removed under vacuum and the residue was dissolved in dichloromethane and washed with water followed by brine. The organic layer was then dried over MgSO4 and reduced under vacuum to give 0.939 g brown gum. The residue was purified by column chromatography, eluting with isohexane/ethyl acetate (50/50). The appropriate fractions were collected and reduced under vacuum to give the title compound as a yellow solid (311 mg, 28% yield).

¹H NMR (500.133 MHz, d₄ acetic acid): δ 1.25-1.32 (9H, m), 4.35 (2H, q), 4.55-4.60 (1H, m), 4.70 (2H, s), 5.38 (1H, s), 6.13 (1H, d), 6.59 (1H, s), 7.88 (1H, d); MS: m/z 388 (MH⁺).

2-chloro-N-(5-propan-2-yloxy-2H-pyrazol-3-yl)pyrimidin-4-amine, used as starting material, was prepared as follows:

2,4-Dichloropyrimidine (10.051 g, 67.0 mmol .1 eq) and 3-isopropoxy-1H-pyrazol-5-amine (10.0 g, 70.0 mmol, 1.05 eq) were mixed together in ethanol (100 ml) and stirred at 60° C. under nitrogen atmosphere for 5 days. The reaction mixture was reduced in vacuo and the residue was dissolved in ethyl acetate (200 ml) and washed with water twice (200 ml) followed by brine (100 ml). The ethyl acetate layer was dried over MgSO4 and filtered, reduced under vacuum to leave a crude, pale yellow oil, yield 17.1 g. Purification by flash column chromatography using silica, eluting with a mixture of dichloromethane 95% and methanol 5% to dichloromethane 90% and methanol 10%, gave yield to an oily solid (13.7 g). The oily solid was dissolved in hot diethyl ether (100 ml). Upon standing a white solid crystallised out which was filtered, washed with ether (10 ml) and dried to give a white crystalline solid, which was an impurity. The filtrate was reduced in vacuo and then dissolved in a mixture of 50% hot methanol in diethyl ether. Again a solid slowly crystallised out which was filtered off, washed with a mixture of 50% methanol in diethyl ether (100 ml), and dried to give the title compound as a white solid (5.003 g, 29% yield).

¹H NMR (500.133 MHz, d₄ acetic acid) δ 1.31 (6H, d), 4.47-4.54 (1H, m), 5.61 (1H, s), 6.97 (1H, d), 8.10 (1H, d); MS: m/z 254 (MH⁺).

5-(Aminomethyl)1,2-oxazole-3-carboxylate, used as starting material can be prepared by the method described in the literature (Barlaam, Bernard; Pape, Andrew; Thomas, Andrew. Preparation of pyrimidine derivatives as modulators of insulin-like growth factor-1 receptor (IGF-1). WO2003048133).

### Example 78

### 5-[[[4-[(5-propan-2-yloxy-2H-pyrazol-3-yl)amino]pyrimidin-2-yl]amino]methyl]-1,2-oxazole-3-carboxamide

To a stirred degassed solution of 5-[[[5-bromo-4-[(5-propan-2-yloxy-2H-pyrazol-3-yl)amino]pyrimidin-2-yl]amino]methyl]-1,2-oxazole-3-carboxamide (140 mg, 0.32 mmol) in ethanol (15 mL) was added Pd/C catalyst (14 mg). Hydrogen gas was introduced by balloon and the mixture was stirred at room temperature for 30 h. The reaction mixture was then filtered and washed with ethanol followed by methanolic ammonia. The filtrate was then evaporated in vacuo and put onto a SCX column and the free base washed off with methanolic ammonia solution. This solution then evaporated in vacuo to give the title compound as an off-white solid (110 mg, 99%).

¹H NMR (300.132 MHz, DMSO) 8 1.26 (6H, d), 4.67 (3H, s), 5.22 (1H, s), 6.04 (1H, d), 6.56 (1H, s), 7.75 (1H, s), 7.91 (1H, d), 8.04 (1H, s), 9.98 (1H, s), 11.8 (1H, s); MS: m/z 359.5 (MH⁺).

5-[[[5-bromo-4-[(5-propan-2-yloxy-2H-pyrazol-3-yl)amino]pyrimidin-2-yl]amino]methyl]-1,2-oxazole-3-carboxamide used as starting material was prepared as follows:—

5-bromo-2-chloro-N-(5-propan-2-yloxy-2H-pyrazol-3-yl)pyrimidin-4-amine (0.30 g, 0.90 mmol), 5-(aminomethyl)1,2-oxazole-3-carboxamide TFA salt (0.299 g, 1.17 mmol), DIPEA (628[L, 3.6 mmol) and 2-methoxyethanol (4 mL) were added and reacted in a microwave at 200° for 30 mins. The mixture was evaporated in vacuo and purified by flash column chromatography. The appropriate fractions were collected and evaporated in vacuo to give a pale yellow solid (0.166 g, 42%).

¹H NMR (300.132 MHz, DMSO) δ 1.32 (6H, d), 4.65-4.75 (3H, m), 5.70 (1H, s), 6.63 (1H, bs), 7.81 (1H, s), 8.10 (2H, bs), 8.18 (1H, s), 9.43 (1H, bs),11.80 (1H, bs); MS: m/z 439 (MH⁺).

5-bromo-2-chloro-N-(5-propan-2-yloxy-2H-pyrazol-3-yl)pyrimidin-4-amine used as starting material was prepared as follows:—

To a solution of 3-isopropoxy-1 H-pyrazol-5-amine (also known as 5-isopropoxy-1H-pyrazol-3-amine; 2.005 g, 14.2 mmol), in dry THF (60 ml) under nitrogen was added triethylamine (2.37 mL, 17 mmol). This mixture was cooled to 0° C. and a solution of 2,4-dichloro-5-bromopyrimidine (3.23 g, 14.2 mmol) in dry THF (30 ml) was added dropwise. The mixture was then allowed to stir at room temp for 18 h. After this time the mixture was evaporated in vacuo to give a yellow solid, which was crystallised with ethyl acetate, filtered and dried under high vaccuum to give pale yellow solid. The solid was washed thoroughly with water and filtered off. Product was left to dry overnight (1.645 g, 35%) MS: m/z 332 (MH⁺).

5-Isopropoxy-1H-pyrazol-3-amine was synthesized as outlined in Example 66.

5-(Aminomethyl)1,2-oxazole-3-carboxamide, used as starting material was prepared in an analogous method to that described for (3-pyrimidin-2-yl-1,2-oxazol-5-yl)methanamine in Example 32, except using 2-oxoacetamide as starting material.

### Example 79

### N-methyl-5-[[[4-[(5-propan-2-yloxy-2H-pyrazol-3-yl)amino]pyrimidin-2-yl]amino]methyl]1,2-oxazole-3-carboxamide

To a test tube was added ethyl 5-[[[4-[(5-propan-2-yloxy-2H-pyrazol-3-yl)amino]pyrimidin-2-yl]amino]methyl]1,2-oxazole-3-carboxylate (100 mg, 0.26 mmol) followed by the 2M methylamine in methanol (4.00 ml). The mixture was shaken for 3 hours at room temperature for 3 hours. After this time the mixture was concentrated to give a yellow gum. This gum was dissolved in DMF (4 ml) and purified by basic prep HPLC using a gradient of 15-35% MeCN in H2O+1% NH4OH. The appropriate fractions were collected and concentrated to give the title compound as a white solid (57 mg 59% yield).

¹H NMR (500.133 MHz, DMSO): δ 1.27 (6H, d), 2.78 (3H, s), 4.68 (3H, m), 5.28 (1H, s), 6.08 (1H, s), 6.51 (1H, s), 7.34 (1H, s), 7.88 (1H, d), 8.15 (1H, s), 9.43 (1H, s), 11.41 (1H, s);

MS: m/z 373 (MH⁺)

5-[[[4-[(5-Propan-2-yloxy-2H-pyrazol-3-yl)amino]pyrimidin-2-yl]amino]methyl]1,2-oxazole-3-carboxylate was synthesized as outlined in Example 77.

### Example 80

### N,N-dimethyl-5-[[[4-[(5-propan-2-yloxy-2H-pyrazol-3-yl)amino]pyrimidin-2-yl]amino]methyl]1,2-oxazole-3-carboxamide

To a test tube was added ethyl 5-[[[4-[(5-propan-2-yloxy-2H-pyrazol-3-yl)amino]pyrimidin-2-yl]amino]methyl]1,2-oxazole-3-carboxylate (62 mg, 0.16 mmol) followed by dimethylamine in 33% absolute ethanol (4 mL). The mixture was shaken and heated to 75° C. for 3 h. After this time the mixture was reduced under vacuum to give a yellow gum. This gum was dissolved in DMF (4 ml) and purified by basic prep. HPLC using a gradient of 15-35% MeCN in H₂O+1% NH₄OH. The appropriate fractions were collected and reduced under vacuum to give the title compound as a white solid (13 mg 21% yield).

¹H NMR (300.132 MHz, DMSO): δ 1.27 (6H, d), 2.99 (3H, s), 3.05 (3H, s), 4.68 (3H, d), 5.28 (1H, s), 6.05 (1H, s), 6.48 (1H, s), 7.73 (1H, s), 7.91 (1H, d), 10.09 (1H, s), 11.85 (1H, s)

MS: m/z 387 (MH⁺)

5-[[[4-[(5-Propan-2-yloxy-2H-pyrazol-3-yl)amino]pyrimidin-2-yl]amino]methyl]1,2-oxazole-3-carboxylate was synthesized as outlined in Example 77.

### Example 81

### N′-(5-propan-2-yloxy-2H-pyrazol-3-yl)-N-[(3-pyrimidin-5-yl1,2-oxazol-5-yl)methyl]pyrimidine-2,4-diamine

To a solution of 2-chloro-N-(5-propan-2-yloxy-2H-pyrazol-3-yl)pyrimidin-4-amine (100 mg, 0.39 mmol, 1 eq) in 2-methoxy ethanol (3 ml)in a microwave tube was added (3-pyrimidin-5-yl1,2-oxazol-5-yl)methanamine.TFA salt (117 mg, 0.40 mmol, 1.02 eq). The mixture was then heated to 200° C. for 30 mins in the microwave (Smith Synthesiser). The solvent was removed in vacuo. The residue was dissolved in methanol and put onto a 5 g Isolute SCX-3 column. The compound was then washed off with methanolic ammonia and reduced under vacuum to give a brown gum. The gum was dissolved in 4 ml DMF and purified by basic prep HPLC using a gradient 15-30% MeCN in H2O+1% NH4OH. The appropriate fractions were collected and reduced under vacuum to give the title compound as an off-white solid (50 mg, 33% yield).

¹H NMR (500.133 MHz, DMSO): δ 1.27 (6H, d), 4.60-4.75 (3H, m), 5.40 (1H, bs), 6.16 (1H, bs), 6.97 (1H, s), 7.48 (1H, bs), 7.96 (1H, s), 9.17 (2H, s), 9.24 (1H, s), 9.49 (1H, bs), 11.45 (1H, bs); MS: m/z 394 (MH⁺).

2-Chloro-N-(5-propan-2-yloxy-2H-pyrazol-3-yl)pyrimidin-4-amine, used as starting material was prepared as in Example 77.

(3-Pyrimidin-2-yl-1,2-oxazol-5-yl)methanamine.TFA salt was synthesized as outlined in Example 32.

### Example 82

### N′-(5-propan-2-yloxy-2H-pyrazol-3-yl)-N-[(³-pyrimidin-2-yl-1,2-oxazol-5-yl)methyl]pyrimidine-2,4-diamine

To a solution of 2-chloro-N-(5-propan-2-yloxy-2H-pyrazol-3-yl)pyrimidin-4-amine (0.1 g, 0.39 mmol) in 2-methoxy ethanol (3 mL) in a microwave tube was added (3-pyrimidin-2-yl-1,²-oxazol-5-yl)methanamine.TFA salt (0.137 g, 0.47 mmol). The mixture was then heated to 200° for 30 mins in the microwave. After this time the solvent was removed in vacuo. The residue was dissolved in methanol and purified by chromatography using a SCX-3 column. The compound was washed off with methanolic ammonia to give a brown tar, which was subsequently purified by flash column chromatography, eluting with DCM/MeOH (95%/5%). The desired fractions were collected and reduced in vacuo to give a brown gum. The gum was dissolved in 4 ml DMF and purified by basic prep. HPLC using a gradient 15-35% MeCN in H₂O+1% NH₄OH. The appropriate fractions were collected and reduced in vacuo to give the title product (0.034 g, 22%).

¹H NMR (300.132 MHz, DMSO) δ 1.26 (6H, d), 4.57-4.77 (3H, m), 5.23 (1H, s), 6.06 (1H, s), 6.84 (1H, s), 7.61 (1H, t), 7.79 (1H, s), 7.92 (1H, d), 8.96 (2H, d), 9.94 (1H, s), 11.87 (1H, s); MS: m/z 394 (MH⁺).

2-Chloro-N-(5-propan-2-yloxy-2H-pyrazol-3-yl)pyrimidin-4-amine, used as starting material was prepared as in Example 77.

(3-pyrimidin-2-yl-1,2-oxazol-5-yl)methanamine.TFA salt used as starting material was prepared as outlined in Example 81.

### Example 83

### N-[[3-(oxolan-3-yl)1,2-oxazol-5-yl]methyl]-N′-(5-propan-2-yloxy-2H-pyrazol-3-yl)pyrimidine-2,4-diamine

To a solution of 2-chloro-N-(5-propan-2-yloxy-2H-pyrazol-3-yl)pyrimidin-4-amine (100 mg, 0.39 mmol, 1 eq) in 2-methoxy ethanol (3 ml)in a microwave tube was added [3-(oxolan-3-yl)1,2-oxazol-5-yl]methanamine (150 mg, 0.89 mmol, 2.3 eq). The mixture was then heated to 200° C. for 45 mins in the microwave (Smith Synthesiser). The solvent was removed in vacuo. The residue was dissolved in methanol and put onto a 5 g Isolute SCX-3 column. The compound was then washed off with methanolic ammonia and reduced under vacuum to give a gum. The gum was dissolved in 4 mL DMF and purified by basic prep HPLC using a gradient 20-40% MeCN in H₂O+1% NH₄OH. The appropriate fractions were collected and reduced under vacuum to give the title compound as a pale orange solid (42 mg, 28% yield).

¹H NMR (300.132 MHz, DMSO): 8 1.27 (6H, d), 1.93-2.01 (1H, m), 2.22-2.31 (1H, m), 3.35-4.01 (5H, m), 4.51-4.73 (3H, m), 5.19 (1H, s), 6.04 (1H, s), 6.29 (1H, s), 7.70 (1H, s), 7.93 (1H, s), 9.97 (1H, s), 11.87 (1H, s); MS: m/z 386 (MH⁺).

2-Chloro-N-(5-propan-2-yloxy-2H-pyrazol-3-yl)pyrimidin-4-amine, used as starting material was prepared as in Example 77.

[3-(Oxolan-3-yl)1,2-oxazol-5-yl]methanamine, used as starting material was prepared in an analogous method to that described for (3-pyrimidin-2-yl-1,2-oxazol-5-yl)methanamine in Example 32, except using oxolane-3-carbaldehyde as starting material. Final yield was 86%.

### Example 84

### N-[[3-(oxolan-2-yl)1,2-oxazol-5-yl]methyl]-N′-(5-propan-2-yloxy-2H-pyrazol-3-yl)pyrimidine-2,4-diamine

To a solution of 2-chloro-N-(5-propan-2-yloxy-2H-pyrazol-3-yl)pyrimidin-4-amine (100 mg, 0.39 mmol, 1 eq) in 2-methoxy ethanol (3 ml)in a microwave tube was added [3-(oxolan-2-yl)1,2-oxazol-5-yl]methanamine (150 mg, 0.89 mmol, 2.3 eq). The mixture was then heated to 200° C. for 45 mins in the microwave (Smith Synthesiser). The solvent was removed in vacuo. The residue was dissolved in methanol and put onto a 5 g Isolute SCX-3 column. The compound was washed off with methanolic ammonia and reduced under vacuum to give a gum. The gum was dissolved in 4 mL DMF and purified by basic prep. HPLC using a gradient 20-40% MeCN in H₂O+1% NH₄OH. The appropriate fractions were collected and reduced under vacuum to give the title compound as an off-white solid (18 mg, 12% yield).

¹H NMR (500.133 MHz, d₄ acetic acid): δ 1.27 (6H, d), 1.90-1.93 (3H, m), 2.15-2.23 (1H, m), 3.72-3.78 (1H, m), 3.80-3.86 (1H, m), 4.52-4.57 (1H, m), 4.61 (2H, s), 4.84-4.88 (1H, m), 5.42 (1H, s), 6.14 (1H, d), 6.21 (1H, s), 7.86 (1H, d); MS: m/z 386 (MH⁺).

2-Chloro-N-(5-propan-2-yloxy-2H-pyrazol-3-yl)pyrimidin-4-amine, used as starting material was prepared as in Example 77.

[3-(Oxolan-2-yl)1,2-oxazol-5-yl]methanamine, used as starting material was prepared in an analogous method to that described for (3-pyrimidin-2-yl-1,2-oxazol-5-yl)methanamine in Example 32, except using oxolane-2-carbaldehyde as starting material.

### Example 85

### N-[[3-(oxan-4-yl)1,2-oxazol-5-yl]methyl]-N′-(5-propan-2-yloxy-2H-pyrazol-3-yl)pyrimidine-2,4-diamine

To a solution of 2-chloro-N-(5-propan-2-yloxy-2H-pyrazol-3-yl)pyrimidin-4-amine (100 mg, 0.39 mmol, 1 eq) in 2-methoxy ethanol (3 ml)in a microwave tube was added [3-(oxan-4-yl)1,2-oxazol-5-yl]methanamine ( (11 3 mg, 0.62 mmol, 1.6 eq). The mixture was then heated to 200° C. for 45 mins in the microwave (Smith Synthesiser). The solvent was removed in vacuo. The residue was dissolved in methanol and put onto a 5 g Isolute SCX-3 column. The compound was then washed off with methanolic ammonia and reduced under vacuo to give a gum. The gum was dissolved in 4 ml DMF and purified by basic prep HPLC using a gradient 20-40% MeCN in H₂O+1% NH₄O H. The appropriate fractions were collected and reduced under vacuum to give the title compound as a pale cream solid (35 mg, 22% yield).

¹H NMR (500.133 MHz, d₄ acetic acid): δ 1.27 (6H, d), 1.62-1.71 (2H, m), 1.77-1.83 (2H, m), 2.88-2.97 (1H, m), 3.39-3.46 (2H, m), 3.84-3.89 (2H, m), 4.55-4.62 (3H, m), 5.39 (1H, s), 6.11 (1H, d), 6.21 (1H, s), 7.88 (1H, d); MS: m/z 400 (MH⁺).

2-Chloro-N-(5-propan-2-yloxy-2H-pyrazol-3-yl)pyrimidin-4-amine, used as starting material was prepared as in Example 77.

[3-(Oxan-4-yl)1,2-oxazol-5-yl]methanamine, used as starting material was prepared in an analogous method to that described for (3-pyrimidin-2-yl-1,2-oxazol-5-yl)methanamine in Example 32, except using oxane-4-carbaldehyde as starting material.

### Example 86

### N′-(5-ethoxy-1H-pyrazol-3-yl)-N-[(3-methyl1,2-oxazol-5-yl)methyl]pyrimidine-2,4-diamine

A mixture of 3-ethoxy-5-aminopyrazole (also known as 5-ethoxypyrazol-3-amine; 0.21 g, 1.65 mmol) and 4-chloro-2-(5-aminomethyl-3-methylisoxazole)pyrimidine (also known as 4-chloro-N-[(3-methyl-1,2-oxazol-5-yl)methyl]pyrimidin-2-amine; 0.371 g, 1.65 mmol) in ethanol (5 mL) was heated at 80° C. overnight. The mixture was allowed to cool, diluted with ethanol and then filtered. The filtered solid was dissolved in a mixture of acetonitrile, dimethylformaide and aqueous ammonia solution and purified by reverse phase preparative chromatography eluting with a gradient of acetonitrile in water (containing 1% ammonia). Fractions containing product were combined and concentrated in vacuo. The resultant precipitate was collected by filtration and dried under vacuum at room temperature to yiled the title compound (0.118 g, 23% yield).

¹H NMR (300 MHz, DMSO+acetic acid): δ 7.89 (d, 1H), 6.15 (s, 1H), 6.06 (d, 1H), 5.32 (br s, 1H), 4.57 (s, 2H), 4.08 (q, 2H), 2.18 (s, 3H), 1.29 (t, 3H).

MS: m/z 316 (MH⁺).

4-Chloro-N-[(3-methyl-1,2-oxazol-5-yl)methyl]pyrimidin-2-amine was prepared as outlined in Example 13.

3-Ethoxy-5-aminopyrazole (also known as 5-ethoxypyrazol-3-amine) has been described in the literature: Kawagishi, Toshio; Sato, Tadahisa. Preparation of 3-alkoxy-5-aminopyrazoles as materials for photographic couplers and drugs. JP63250368.

### Example 87

### N-[(3-methyl1,2-oxazol-5-yl)methyl]-N′-[5-[(3-morpholin-4-ylphenyl)methoxy]-2H-pyrazol-3-yl]pyrimidine-2,4-diamine

Prepared in an analogous way to example 11 but starting with 5-[(3-morpholin-4-ylphenyl)methoxy]-1H-pyrazol-3-amine (182 mg, 0.66 mmol, 1 eq) and using a 25-45% gradient of acetonitrile in water containing 1% ammonia to purify. The title compound was obtained as a solid (28.4 mg, 9.3% yield).

1H NMR (300.132 MHz, DMSO): δ 2.19 (s, 3H), 3.11 (t, 4H), 3.74 (t, 4H), 4.58 (d, 2H), 5.07 (s, 2H), 5.33 (s, 1H), 6.05 (d, 1H), 6.16 (s, 1H), 6.89 (m, 2H), 7.00 (s, 1H), 7.23 (t, 1H), 7.66 (s, 1H), 7.91 (d, 1H), 9.96 (s, 1H), 11.92 (s, 1H). MS: m/z 463 (MH+).

5-[(3-morpholin-4-ylphenyl)methoxy]-1H-pyrazol-3-amine used as starting material was prepared in a similar manner to 5-[(3-ethylphenyl)methoxy]-2H-pyrazol-3-amine in Example 74a) and taken on crude to the next step.

### Example 88

### N-[(3-methyl1,2-oxazol-5-yl)methyl]-N′-[5-[(3-methylsulfonyloxyphenyl)methoxy]-2H-pyrazol-3-yl]pyrimidine-2,4-diamine

Prepared in an analogous way to example 38, but starting with 5-[(3-methylsulfonyloxyphenyl)methoxy]-2H-pyrazol-3-amine (80 mg, 0.28 mmol, 1 eq) and using a 15-35% gradient of acetonitrile in water containing 1% ammonia to purify. The title compound was obtained as a solid (37.5 mg, 29% yield).

1H NMR (300.132 MHz, DMSO): δ 2.19 (s, 3H), 3.39 (s, 3H), 4.58 (d, 2H), 5.20 (s, 2H), 5.32 (s, 1H), 6.03 (d, 1H), 6.17 (s, 1H), 7.26-7.58 (m, 2H), 7.71 (s, 1H), 7.92 (d, 1H), 10.03 (s, 1H), 11.95 (s, 1H). MS: m/z 472 (MH+).

5-[(3-methylsulfonyloxyphenyl)methoxy]-2H-pyrazol-3-amine, used as starting material was prepared from (3-methylsulfonyloxyphenyl)methanol in an analogous way to 5-[(3-ethylphenyl)methoxy]-2H-pyrazol-3-amine in Example 74a). Isolated as a clear film (80 mg, 9% yield) MS: m/z 284 (MH+).

### Example 89

### tert-Butyl N-[3-[[5-[[2-[(3-methyl1,2-oxazol-5-yl)methylamino]pyrimidin-4-yl]amino]-1H-pyrazol-3-yl]oxymethyl]phenyl]carbamate

3-[[5-[[2-[(3-Methyl 1,2-oxazol-5-yl)methylamino]pyrimidin-4-yl]amino]-1H-pyrazol-3-yl]oxymethyl]benzoic acid (70 mg, 0.17 mmol, 1 eq), diphenylphosphoryl azide (40 μl, 0.18 mmol, 1.1 eq) and diisopropylethylamine (23 μl, 0.18 mmol, 1.1 eq) were dissolved in t-butanol (3 ml) and heated to 150° C. for 20 minutes. After this time the mixture was concentrated and the residue purified by basic prep HPLC. The product containing fraction was concentrated to give the title compound (14 mg, 17%) as a white solid.

1H NMR (300.132 MHz, DMSO) δ 1.48 (s, 9H), 2.19 (s, 3H), 4.58 (d, 2H), 5.06 (s, 2H), 5.29 (s, 1H), 6.02 (d, 1H), 6.17 (s, 1H), 7.02 (d, 1H), 7.21-7.26 (m, 1H), 7.34 (d, 1H), 7.59 (s, 1H), 7.69 (s, 1H), 7.91 (d, 1H), 9.34 (s, 1H), 10.00 (s, 1H), 11.91 (s, 1H). MS: m/z 493

(MH+)

3-[[5-[[2-[(3-Methyl 1,2-oxazol-5-yl)methylamino]pyrimidin-4-yl]amino]-1H-pyrazol-3-yl]oxymethyl]benzoic acid was prepared as outlined in Example 98.

### Example 90

### [3-[[5-[[2-[(3-methyl1,2-oxazol-5-yl)methylamino]pyrimidin-4-yl]amino]-1H-pyrazol-3-yl]oxymethyl]phenyl]-morpholin-4-yl-methanone

To a stirred solution of 3-[[5-[[2-[(3-Methyl1,2-oxazol-5-yl)methylamino]pyrimidin-4-yl]amino]-1H-pyrazol-3-yl]oxymethyl]benzoic acid (60 mg, 0.14 mmol, 1 eq) in DMF (4 ml) was added HATU (60 mg, 0.16 mmol, 1.1 eq) followed by morpholine (25 mg, 0.29 mmol, 2 eq). The reaction was stirred for 24 hours at room temperature, then concentrated and the residue partitioned between water (10 ml) and ethyl acetate (10 ml). The organic layer, in each case, was separated and washed with water (2×10 ml), sat NaHCO₃ (2×10 ml), brine (2×10 ml) and dried over anhydrous Na₂SO₄. The solution was concentrated to yield the title compound (22 mg, 32%) as a white solid.

1H NMR (300.132 MHz, DMSO) δ 2.24 (s, 3H), 3.61-3.68 (m, 8H), 4.63 (d, 2H), 5.25 (s, 2H), 5.36 (s, 1H), 6.08 (d, 1H), 6.22 (s, 1H), 7.40 (d, 1H), 7.49-7.59 (m, 3H), 7.75 (s, 1H), 7.97 (d, 1H), 10.07 (s, 1H), 11.98 (s, 1H). MS: m/z 491 (MH+)

3-[[5-[[2-[(3-Methyl 1,2-oxazol-5-yl)methylamino]pyrimidin-4-yl]amino]-1H-pyrazol-3-yl]oxymethyl]benzoic acid was prepared as outlined in Example 98.

### Example 91

### N-methyl-3-[[5-[[2-[(3-methyl1,2-oxazol-5-yl)methylamino]pyrimidin-4-yl]amino]-1H-pyrazol-3-yl]oxymethyl]benzamide

Prepared using a method analogous to example 90, using methylamine hydrochloride (20 g, 0.29 mmol, 2 eq) and diisopropylethylamine (50μl, 0.29 eq, 2 eq) as starting materials to yield the title compound (45 mg, 74%) as a white solid.

1H NMR (300.132 MHz, DMSO) δ 2.24 (s, 3H), 2.84 (d, 3H), 4.63 (d, 2H), 5.24 (s, 2H), 5.36 (s, 1H), 6.08 (d, 1H), 6.22 (s, 1H), 7.49-7.54 (m, 1H), 7.63 (d, 1H), 7.76 (d, 1H), 7.83 (d, 1H), 7.96 (s, 2H), 8.49 (d, 1H), 10.06 (s, 1H), 11.98 (s, 1H). MS: m/z 435 (MH+)

### Example 92

### 3-[[5-[[2-[(3-methyl1,2-oxazol-5-yl)methylamino]pyrimidin-4-yl]amino]-2H-pyrazol-3-yl]oxymethyl]benzonitrile hydrochloride

Prepared using an analogous method to example 46, but starting with 3-[(5-amino-2H-pyrazol-3-yl)oxymethyl]benzonitrile (77 mg, 0.36 mmol) to give the title compound (27 mg, 17% yield)

1H NMR (300.132 MHz, DMSO) δ 2.19 (s, 3H), 4.71 (s, 2H), 5.19 (s, 2H), 6.25 (s, 1H), 6.38 (s, 1H), 7.61 (t, 1H), 7.75-7.93 (m, 4H). MS: m/z 403 (MH+)

3-[(5-Amino-2H-pyrazol-3-yl)oxymethyl]benzonitrile, used as starting material, was prepared as follows:
a) 3-Amino-5-hydroxypyrazole (2 g, 20.18 mmol, 1 eq) and triphenylphosphine (6.36 g, 24.22 mmol, 1.2 eq) were stirred in DCM (20 ml) for 30 mins. After this time, DIAD (4.77 ml, 24.22 mmol, 1.2 eq) was slowly added, keeping the temp below 20° C. with a water bath, and the resulting mixture stirred for a further 45 mins. A solution of 3-cyanobenzyl alcohol (3.23 g, 24.22 mmol, 1.2 eq) in DCM (10 ml) was added slowly and the reaction left to stir at RT for 24 hours. After this time the solid was filtered off and the solution extracted with 2M HCl solution (3×30 ml). The aqueous layer was back-washed with diethyl ether (2×30 ml), then basified to pH 9 using ammonium hydroxide, cooling the mixture to avoid a strong exotherm. The solution was extracted with DCM (3×30 ml) and the organic fractions combined, dried over magnesium sulphate and concentrated to give 3-[(5-amino-2H-pyrazol-3-yl)oxymethyl]benzonitrile as a colourless gum (321 mg, 7%). MS: m/z 215 (MH+)

### Example 93

### N′-[5-[(3-chlorophenyl)methoxy]-1H-pyrazol-3-yl]-N-[(3-methyl1,2-oxazol-5-yl)methyl]pyrimidine-2,4-diamine hydrochloride

Prepared using an analogous method to example 46, but starting with 5-[(3-chlorophenyl)methoxy]-1H-pyrazol-3-amine (80 mg, 0.36 mmol) to give the title compound (42 mg, 26% yield)

1H NMR (300.132 MHz, DMSO) δ 2.19 (s, 3H), 4.71 (s, 2H), 5.14 (s, 2H), 6.26 (s, 1H), 6.37 (s, 1H), 7.37-7.42 (m, 4H), 7.49 (s, 1H), 7.92 (d, 1H). MS: m/z 412 (MH+)

5-[(3-chlorophenyl)methoxy]-1H-pyrazol-3-amine, used as a starting material, was prepared using an analogous method to example 92a, but starting with (3-chlorophenyl)methanol (3.75 g, 26.2 mmol) to give 5-[(3-chlororophenyl)methoxy]-1H-pyrazol-3-amine (179 mg, 4%) as a white solid. 1H NMR (300.132 MHz, DMSO) δ 4.75 (s, 1H), 4.94 (s, 2H), 5.06 (s, 2H), 7.32-7.41 (m, 3H), 7.44 (s, 1H), 10.43 (s, 1H). MS: m/z 224 (MH+)

### Example 94

### N′-[5-[(3-fluorophenyl)methoxy]-1H-pyrazol-3-yl]-N-[(3-methyl1,2-oxazol-5-yl)methyl]pyrimidine-2,4-diamine hydrochloride

Prepared using an analogous method to to example 46, but starting with 5-[(3-fluorophenyl)methoxy]-1H-pyrazol-3-amine (74 mg, 0.36 mmol) to give the title compound (73 mg, 47% yield)

1H NMR (300.132 MHz, DMSO) δ 2.19 (s, 3H), 4.71 (s, 2H), 5.14 (s, 2H), 6.26 (s, 1H), 6.38 (s, 1H), 7.12-7.19 (m, 1H), 7.22-7.28 (m, 2H), 7.40-7.47 (m, 1H), 7.91 (d, 1H). MS: m/z 396 (MH+)

5-[(3-fluorophenyl)methoxy]-1H-pyrazol-3-amine, used as a starting material, was prepared using an analogous method to example 92a), but starting with (3-fluorophenyl)methanol (3.3 g, 26.2 mmol) to give 5-[(3-fluorophenyl)methoxy]-1H-pyrazol-3-amine (428 mg, 10%) as a white solid. 1H NMR (300.132 MHz, DMSO) δ 4.76 (s, 1H), 4.93 (s, 2H), 5.06 (s, 2H), 7.09-7.15 (m, 1H), 7.18-7.24 (m, 2H), 7.37-7.44 (m, 1H), 10.41 (s, 1H). MS: m/z 208 (MH+)

### Example 95

### N-[(3-methyl1,2-oxazol-5-yl)methyl]-N′-[5-[[3-(trifluoromethyl)phenyl]methoxy]-1H-pyrazol-3-yl]pyrimidine-2,4-diamine hydrochloride

Prepared using an analogous method to example 46, but starting with 5-[[3-(trifluoromethyl)phenyl]methoxy]-1H-pyrazol-3-amine (92 mg, 0.36 mmol) to give the title compound (29 mg, 17% yield)

1H NMR (300.132 MHz, DMSO) δ 2.18 (s, 3H), 4.70 (s, 2H), 5.22 (s, 2H), 6.25 (s, 1H), 6.37 (s, 1H), 7.61-7.75 (m, 3H), 7.78 (s, 1H), 7.90 (d, 1H). MS: m/z 446 (MH+)

5-[[3-(trifluoromethyl)phenyl]methoxy]-1H-pyrazol-3-amine, used as a starting material, was prepared using an analogous method to example 92a, but starting with [3-(trifluoromethyl)phenyl]methanol (4.63 g, 26.2 mmol) to give 5-[[3-(trifluoromethyl)phenyl]methoxy]-1H-pyrazol-3-amine (121 mg, 2.4%) as an off-white solid.

MS: m/z 258 (MH+)

### Example 96

### N-[(3-methyl1,2-oxazol-5-yl)methyl]-N′-[5-[[4-(trifluoromethyl)phenyl]methoxy]-1H-pyrazol-3-yl]pyrimidine-2,4-diamine hydrochloride

Prepared using an analogous method to example 46, but starting with 5-[[4-(trifluoromethyl)phenyl]methoxy]-1H-pyrazol-3-amine (77 mg, 0.36 mmol) to give the title compound (58 mg, 38% yield)

1H NMR (300.132 MHz, DMSO) δ 2.18 (s, 3H), 4.71 (s, 2H), 5.24 (s, 2H), 6.25 (s, 1H), 6.37 (s, 1H), 7.64 (d, 2H), 7.75 (d, 2H), 7.91 (d, 1H). MS: m/z 445 (MH+)

5-[[4-(trifluoromethyl)phenyl]methoxy]-1H-pyrazol-3-amine, used as a starting material, was prepared using an analogous method to example 92a, but starting with [4-(trifluoromethyl)phenyl]methanol (4.27 g, 24.2 mmol) to give 5-[[4-(trifluoromethyl)phenyl]methoxy]-1H-pyrazol-3-amine (177 mg, 3.4%) as a white solid.

1H NMR (399.902 MHz, DMSO) δ 4.77 (s, 1H), 4.95 (s, 2H), 5.16 (s, 2H), 7.61 (d, 2H), 7.73 (d, 2H), 10.42 (s, 1H). MS: m/z 258 (MH+)

### Example 97

### Methyl 3-[[5-[[2-[(3-methyl1,2-oxazol-5-yl)methylamino]pyrimidin-4-yl]amino]-1H-pyrazol-3-yl]oxymethyl]benzoate hydrochloride

Prepared using an analogous method to example 46, but starting with methyl 3-[(5-amino-1H-pyrazol-3-yl)oxymethyl]benzoate (500 mg, 2.02 mmol) to give the title compound (320 mg, 44% yield).

1H NMR (300.132 MHz, DMSO) δ 2.18 (s, 3H), 3.86 (s, 3H), 4.70 (s, 2H), 5.20 (s, 2H), 6.25 (s, 1H), 6.37 (s, 1H), 7.52-7.57 (m, 1H), 7.70 (d, 1H), 7.89-7.94 (m, 2H), 8.03 (s, 1H). MS: m/z 436 (MH+)

Methyl 3-[(5-amino-1H-pyrazol-3-yl)oxymethyl]benzoate, used as a starting material, was prepared using an analogous method to example 92a, but starting with methyl 3-(hydroxymethyl)benzoate (4.5 g, 27.1 mmol) to give Methyl 3-[(5-amino-1H-pyrazol-3-yl)oxymethyl]benzoate (602 mg, 9%) as a brown gum.

1H NMR (300.132 MHz, DMSO) δ 3.86 (s, 3H), 4.77 (s, 1H), 4.93 (s, 2H), 5.12 (s, 2H), 7.49-7.54 (m, 1H), 7.67 (d, 1H), 7.89 (d, 1H), 7.99 (s, 1H), 10.42 (s, 1H) MS: m/z 248 (MH+)

Methyl 3-(hydroxymethyl)benzoate was prepared as follows:

mono-Methylisophthalate (8 g, 44.4 mmol, 1 eq) was dissolved in tetrahydrofuran (250 ml) at room temperature. 1.0M Borane-THF solution (222 ml, 222 mmol, 5 eq) was added slowly and the solution stirred for 24 hours at RT. After this time, methanol (30 ml) was slowly added and the reaction stirred at RT for 1 hour after which it was concentrated. The residue was partitioned between ethyl acetate (50 ml) and 10% aq ammonium hydroxide solution and the organic layer separated. The aqueous layer was washed with ethyl acetate (2×50 ml) and the organic layers combined, washed with 10% aq ammonium hydroxide solution (2×50 ml), 2M hydrochloric acid (2×50 ml), water (2×50 ml), brine (2×50 ml) and dried over anhydrous sodium sulphate. The solution was concentrated to give methyl 3-(hydroxymethyl)benzoate as a colourless oil (6.2 g, 84%).

1H NMR (400.132 MHz, DMSO) δ 3.86 (s, 3H), 4.58 (d, 2H), 5.33 (t, 1H), 7.45-7.49 (m, 1H), 7.59 (d, 1H), 7.84 (d, 1H), 7.96 (s, 1H). MS: N/A

### Example 98

### 3-[[5-[[2-[(3-methyl1,2-oxazol-5-yl)methylamino]pyrimidin-4-yl]amino]-1H-pyrazol-3-yl]oxymethyl]benzoic acid

3-[[5-[[2-[(3-Methyl1,2-oxazol-5-yl)methylamino]pyrimidin-4-yl]amino]-1H-pyrazol-3-yl]oxymethyl]benzoate hydrochloride (30 mg, 0.063 mmol, 1 eq) was dissolved in 2M sodium hydroxide solution (2 ml) with one drop of methanol added. The mixture was heated to 120° C. for 20 mins. After this time, the reaction was cooled to approx 10° C. and neutralised with 2M hydrochloric acid. The precipitate was filtered and washed with cold water, then dried to give 3-[[5-[[2-[(3-methyl1,2-oxazol-5-yl)methylamino]pyrimidin-4-yl]amino]-1H-pyrazol-3-yl]oxymethyl]benzoic acid as a white solid (14 mg, 52%)

1H NMR (300.132 MHz, DMSO) d 2.17 (s, 3H), 4.57 (s, 2H), 5.21 (s, 2H), 5.38 (s, 1H), 6.15 (s, 1H), 7.47-7.52 (m, 1H), 7.67 (d, 1H), 7.87-7.91 (m, 2H), 8.01 (s, 1H)

3-[[5-[[2-[(3-Methyl 1,2-oxazol-5-yl)methylamino]pyrimidin-4-yl]amino]-1H-pyrazol-3-yl]oxymethyl]benzoate was prepared as outlined in Example 97.

### Example 99

### N′-[5-[(4-ethoxy-3-methoxy-phenyl)methoxy]-1H-pyrazol-3-yl]-N-[(3-methyl1,2-oxazol-5-yl)methyl]pyrimidine-2,4-diamine

A mixture of 5-[(4-ethoxy-3-methoxy-phenyl)methoxy]-1H-pyrazol-3-amine (87 mg, 0.33 mmol), 4-chloro-N-[(3-methylisoxazol-5-yl)methyl]pyrimidin-2-amine (also known as 4-chloro-N-[(3-methyl-1,2-oxazol-5-yl)methyl]pyrimidin-2-amine; 75 mg, 0.33 mmol) and ethanol (3 ml) was heated at 80° C. for 24 h. After evaporating under reduced pressure, the crude product was purified by column chromatography on silica in ammonia/methanol/DCM (2:8:90). Fractions containing product were combined and evaporated to yield an off white solid that required additional purification by reverse phase prep. HPLC (acidic) using a 25-45% gradient of acetonitrile in water containing 0.1% trifluoroacetic acid. The clean fractions were taken and evaporated to afford the title compound as a white solid (11 mg, 7%). ¹H NMR (399.9 MHz, DMSO-d₆) δ1.29 (3H, t), 2.18 (3H, s), 3.36 (2H, s), 3.72 (3H, s), 3.94 (2H, q), 4.64-4.66 (2H, m), 6.17 (1H, s), 6.43 (2H, s), 6.77-6.79 (1H, m), 6.93-6.94 (1H, m), 7.42 (1H, s), 7.48 (1H, d), 8.08 (1H, d), 9.56 (1H, s); MS: m/z 452 (MH+).

4-Chloro-N-[(3-methyl-1,2-oxazol-5-yl)methyl]pyrimidin-2-amine was prepared as outlined in Example 13.

5-[(4-ethoxy-3-methoxy-phenyl)methoxy]-1H-pyrazol-3-amine used as starting material was prepared using an analogous procedure to 82a), starting from 3-methoxy-4-ethoxybenzylalcohol (4.74 g, 26 mmol) as starting material. 5-[(4-Ethoxy-3-methoxy-phenyl)methoxy]-1H-pyrazol-3-amine was obtained as a solid (90 mg, 1.3%); MS: m/z 264 (MH+).

### Example 100

### N′-[5-[(4-fluoro-3-methoxy-phenyl)methoxy]-1H-pyrazol-3-yl]-N-[(3-methyl1,2-oxazol-5-yl)methyl]pyrimidine-2,4-diamine hydrochloride

Prepared using an analogous method to example 46, but starting with 5-[(4-fluoro-3-methoxy-phenyl)methoxy]-N-methyl-1H-pyrazol-3-amine (85 mg, 0.36 mmol) to give the title compound (55 mg, 33% yield)

1H NMR (300.132 MHz, DMSO) δ 2.18 (s, 3H), 3.85 (s, 3H), 4.72 (s, 2H), 5.06 (s, 2H), 6.27 (s, 1H), 6.37 (s, 1H), 6.97-7.03 (m, 1H), 7.16-7.26 (m, 2H), 7.91 (d, 1H). MS: m/z 426 (MH+)

5-[(4-Fluoro-3-methoxy-phenyl)methoxy]-N-methyl-1H-pyrazol-3-amine, used as a starting material, was prepared using an analogous method to example 92a, but starting with methyl (4-fluoro-3-methoxy-phenyl)methanol (3.79 g, 24.2 mmol) to give 5-[(4-Fluoro-3-methoxy-phenyl)methoxy]-N-methyl-1H-pyrazol-3-amine (258 mg, 5.4%) as a white solid.

1H NMR (300.132 MHz, DMSO) δ 4.75 (s, 1H), 4.91 (s, 2H), 4.99 (s, 2H), 6.93-6.98 (m, 1H), 7.15 (d, 1H), 7.19 (d, 1H), 10.41 (s, 1H). MS: m/z 238 (MH+)

### Example 101

### N-[(3-methyl1,2-oxazol-5-yl)methyl]-N′-[5-(2-phenoxyethoxy)-2H-pyrazol-3-yl]pyrimidine-2,4-diamine

A mixture of 5-(2-phenoxyethoxy)-2H-pyrazol-3-amine (0.483 g, 2.20 mmol), 4-chloro-N-[(3-methyl1,2-oxazol-5-yl)methyl]pyrimidin-2-amine (0.495 g, 2.20 mmol) and ethanol (10 ml) was stirred and heated at 80° C. for 18 h. The mixture was filtered and the precipitate washed with ice cold ethanol and then washed with ether to give product (0.355 g, 40% yield).

¹H NMR (399.9 MHz, DMSO-d₆) δ 2.20 (3H, s), 4.30 (2H, t), 4.37 (2H, s), 4.76 (2H, s), 5.9 (1H, s), 6.22-6.43 (2H, d), 6.39 (1H, s), 6.95-6.99 (3H, m), 7.29-7.34 (2H, m), 7.94 (1H, d), 8.80-8.95 (1H, s), 11.2-11.4 (1H, s), 12.5-13.2 (1H, s); MS: m/z 408 (MH⁺)

5-(2-phenoxyethoxy)-2H-pyrazol-3-amine, used as starting material was prepared as follows:

A mixture of 2-cyanoacetohydrazide (2.34 g, 24.12 mmol), 4-methylbenzenesulfonic acid (9.18 g, 48.24 mmol), 2-phenoxyethanol (10.00 g, 72.37 mmol) and toluene (15 ml) was stirred under reflux (Dean and Stark conditions) for 5 hours. Ethyl acetate (20 ml) was added and stirred, and the mixture allowed to cool. After cooling, the mixture was filtered and the obtained sulfonate of 5-(2-phenoxyethoxy)-2H-pyrazol-3-amine was neutralised with 10% aqueous sodium hydroxide solution. The precipitated 5-(2-phenoxyethoxy)-2H-pyrazol-3-amine was then filtered, washed with ethyl acetate and brine, and dried with magnesium sulphate to give the final product (1215 mg, 23%).

### Example 102

### N-[(3-cyclobutyl-1,2-oxazol-5-yl)methyl]-N′-(5-propan-2-yloxy-1H-pyrazol-3-yl)pyrimidine-2,4-diamine

A mixture of 2-chloro-N-(5-propan-2-yloxy-2H-pyrazol-3-yl)pyrimidin-4-amine (254 mg, 1.00 mmol), (3-cyclobutyl-1,2-oxazol-5-yl)methanamine (153 mg, 1.00 mmol) and ethanol (3 ml) was heated at 150° C. in the microwave for 30 mins. After cooling, the crystalline solid was filtered off, washed with cold ethanol and the crude product was purified by reverse phase prep. HPLC (basic) using a 31-51% gradient of acetonitrile in water containing 1% ammonium hydroxide. The desired fractions were collected and evaporated to afford the title compound as a white solid (78 mg, 22%). ¹H NMR (399.9 MHz, DMSO-d₆) δ 1.28 (6H, d), 1.83-1.92 (1H, m), 1.95-2.04 (1H, m), 2.12-2.19 (1H, m), 2.24-2.32 (1H, m), 3.50-3.58 (1H, m), 4.60 (2H, d), 7.71 (1H, s), 7.92 (2H, d), 9.99 (1H, m), 11.89 (1H, m),

MS: m/z 370 (MH+).

2-chloro-N-(5-propan-2-yloxy-2H-pyrazol-3-yl)pyrimidin-4-amine, used as starting material was prepared as in Example 77.

(3-cyclobutyl-1,2-oxazol-5-yl)methanamine, used as starting material was prepared as in Example 23.

### Example 103

### N-[(3-cyclopropyl-1,2-oxazol-5-yl)methyl]-N′-(5-phenylmethoxy-2H-pyrazol-3-yl)pyrimidine-2,4-diamine

To a reaction tube was added 4-chloro-N-[(3-cyclopropyl-1,2-oxazol-5-yl)methyl]pyrimidin-2-amine (100 mg, 0.40 mmoles), ethanol (2 ml), and 5-phenylmethoxy-2H-pyrazol-3-amine (80 mg, 0.42 mmoles). The mixture was heated overnight at 80° C. The cooled mixture was filtered and the solid was washed with ethanol. The solid was suspended in water and to this was added a few drops of conc. ammonia and the resulting solid was filtered off. The resulting gum was combined with the aqueous filtrate and the mixture was diluted with methanol to dissolve any solid. The mixture was poured onto a SCX-2 column and washed with methanol. The product was eluted with 2N ammonia in methanol to give crude product as a yellow gum. The crude product was purified by reverse phase prep. HPLC (basic) using a 10-95% gradient of acetonitrile in water containing 1% ammonium hydroxide. The product was obtained as a solid (15 mg, 9%).

1H NMR (DMSO 400.13 MHz) δ 0.71 (m, 2H), 0.95 (m, 2H), 1.94 (m, 1H), 4.55 (d, 2H), 5.13 (s, 2H), 5.28 (bs, 1H), 6.01 (d, 1H), 6.05 (s, 1H), 7.3-7.45 (m, 5H), 7.56 (bs, 1H), 7.92 (d, 1H), 9.97 (bs, 1H), 11.9 (bs, 1H)

MS: m/z 404 (MH+).

4-chloro-N-[(3-cyclopropyl-1,2-oxazol-5-yl)methyl]pyrimidin-2-amine, used as starting material was prepared as in Example 19.

5-Phenylmethoxy-2H-pyrazol-3-amine (also named as 5-benzyloxy-1H-pyrazol-3-amine), used as starting material was prepared as in Example 72.

### Example 131

### N′-[5-[(3-methoxy-5-methyl-phenyl)methoxy]-1H-pyrazol-3-yl]-N-[(3-methyl-1,2-oxazol-5-yl)methyl]pyrimidine-2,4-diamine

2-chloro-N-[5-[(3-methoxy-5-methyl-phenyl)methoxy]-2H-pyrazol-3-yl]pyrimidin-4-amine (73 mg, 0.2 mmol), (3-methyl-1,2-oxazol-5-yl)methanamine. hydrochloride (38 mg, 0.25 mmol) and N-ethyl-N-propan-2-yl-propan-2-amine (112 uL, 0.63 mmol) in ethanol (4 ml) were heated at 180° C. in a microwave reactor for 45 mins. The reaction mixture was cooled and the solution concentrated. The crude product was purified by reverse-phase prep. HPLC (basic) using a 35-55% gradient of acetonitrile in water containing 1% ammonium hydroxide solution. The clean fractions were taken and evaporated to afford the title compound as a gum. (8 mg, 9% yield). H NMR (500.13 MHz, DMSO-d₆) δ 2.17 (3H, m), 2.27 (3H, s), 3.72 (3H, s) 4.50-4.59 (2H, m), 5.03, (2H, s), 5.30 (1H, s), 5.99 (1H, s), 6.13 (1H, s), 6.68 (1H, s), 6.75 (1H, s), 6.80 (1H, s), 7.67 (1H, s), 7.89 (1H, d), 10.08 (1H, s), 11.95 (1H, s). MS: m/z 422 (MH+).

(3-methyl-1,2-oxazol-5-yl)methanamine hydrochloride, used as starting material, was prepared as outlined in Example 1.

2-chloro-N-[5-[(3-methoxy-5-methyl-phenyl)methoxy]-2H-pyrazol-3-yl]pyrimidin-4-amine used as starting material was prepared as follows:

5-[(3-methoxy-5-methyl-phenyl)methoxy]-2H-pyrazol-3-amine mono hydrochloride (256 mg, 0.95 mmol), 2,4-dichloropyrimidine (170 mg, 1. 14 mmol) and N-ethyl-N-propan-2-yl-propan-2-amine (423 μL, 2.38 mmol) in ethanol (15 ml) were heated at 80° C. for 144 h. The reaction mixture was cooled and the solution concentrated. The crude product was purified by normal phase chromatography on silica, using a 0-5% gradient of methanol in DCM. The clean fractions were taken and evaporated to afford the title compound as a oil. (75 mg, 23% yield). MS: m/z346(MH+).

5-[(3-methoxy-5-methyl-phenyl)methoxy]-2H-pyrazol-3-amine mono hydrochloride used as starting material was prepared as follows:

To a stirred solution of triphenylphosphine (4.095 g, 15.6 mmol) in DCM (20 ml) was added 5-amino-2H-pyrazol-3-ol (1.43 g, 14.4 mmol) and the suspension stirred for 1 h at room temperature and then cooled to 5-10° C. Propan-2-yl (NZ)-N-propan-2-yloxycarbonyliminocarbamate (3.08 ml, 15.6 mmol) was added over 30 mins and the mixture allowed to warm to room temperature and stirred for 1 hr. A solution of (3-methoxy-5-methyl-phenyl)methanol (1.83 g, 12 mmol) in DCM (10 ml) was added and the mixture stirred for 24 h. The mixture was filtered and the organic layer extracted with 2M HCl (3×100 ml). The aqueous layer was extracted with DCM (2×20 ml). Upon standing, a solid crystallised out from the DCM liquors. This was filtered off to give 5-[(3-methoxy-5-methyl-phenyl)methoxy]-1H-pyrazol-3-amine mono hydrochloride as a white solid (259 mg, 18.2%).

1H NMR (399.9 MHz, DMSO-d₆) δ 2.30 (3H, s), 3.70-3.75 (3H, m), 5.19 (2H, s), 5.28 (1H, s), 6.78 (1H, s), 6.83 (2H, t), 7.54-7.58 (1H, m), 7.62-7.66 (1H, m). MS: m/z 233 (MH+).

(3-methoxy-5-methyl-phenyl)methanol used as starting material was prepared as follows:—

1M solution of Lithium aluminium hydride in tetrahydrofuran (22.4 ml, 22.4 mmol) was added over 10 mins at −4° C. under nitrogen to a stirred solution of methyl 3-methoxy-5-methyl-benzoate (2.525 g, 14 mmol) in anhydrous tetrahydrofuran (25 ml). The reaction mixture was stirred at room temperature for 4 h. The reaction mixture was cooled to 0° C. and quenched with 5N hydrochloric acid and adjusted to pH7. The reaction mixture was evaporated to dryness and the residue partitioned between ether and water (50 ml each). This was extracted with diethyl ether (3×40 ml), washed with saturated brine solution, dried (MgSO4), filtered and evaporated to give (3-methoxy-5-methyl-phenyl)methanol as an oil (1.864 g, 87.6%). 1H NMR (399.9 MHz, DMSO-d₆) δ 2.27 (3H, d), 3.73 (3H, s), 4.44 (2H, d), 5.10 (1H, t), 6.62 (1H, s), 6.69-6.71 (2H, m). MS: m/z 175 (M+Na)+

Methyl 3-methoxy-5-methyl-benzoate used as starting material was prepared as follows:

A solution of methyl 3-hydroxy-5-methyl-benzoate (4.16 g, 25 mmol) in anhydrous N,N dimethylformamide (20 ml) was added drop wise at 20° C. to a stirred suspension of sodium hydride (60% dispersion in mineral oil, 1.51 g, 37.5 mmol). The reaction mixture was stirred for 20 mins at 20° C. and iodomethane (2.36 ml, 37.5 mmol) was added in one portion. The suspension stirred for 18 h. The reaction mixture was quenched by pouring onto a mixture of ice and water (50 g and 100 ml). The product was extracted with ethyl acetate (4×25 ml) and the extracts were washed with water and saturated brine solution. The organic layers were dried (MgSO4), filtered and evaporated to give crude methyl 3-methoxy-5-methyl-benzoate as a oil (4.93 g, >100%).

1H NMR (399.9 MHz, DMSO-d₆) δ 2.35 (3H, d), 3.80 (3H, s), 3.85 (3H, s), 7.05-7.06 (1h, m), 7.25-7.27 (1H, m), 7.38-7.39 (1H, m)

3-hydroxy-5-methyl-benzoate used as starting material was prepared by the method described in the literature (Fred A. Turner and James E Gearien - Journal of Organic Chemistry 1959, Volume 24, p 1952- Synthesis of Reserpine Analogs).

### Example 135

### N′-[5-[(5-fluoro-2-methoxy-pyridin-4-yl)methoxy]-1H-pyrazol-3-yl]-N-[(3-methyl-1,2-oxazol-5-yl)methyl]pyrimidine-2,4-diamine

(5-Fluoro-2-methoxy-pyridin-4-yl)methoxy]-1H-pyrazol-3-amine (130 mg, 0.546 mmol) was heated with 4-chloro-N-[(3-methyl-1,2-oxazol-5-yl)methyl]pyrimidin-2-amine (1 24 mg, 0.546 mmol) in ethanol (8 ml) in a microwave reactor at 120° C. for 1.5 h. The reaction mixture was allowed to stand at 5° C. for 2 days. The precipitated solid was collected by filtration, washed with ethanol and dried under vacuum. The crude solid was purified by preparative HPLC using decreasingly polar mixtures of water (containing 1% NH₃) and MeCN as eluents. Fractions containing the desired compound were evaporated to dryness to afford N′-[5-[(5-fluoro-2-methoxy-pyridin-4-yl)methoxy]-1H-pyrazol-3-yl]-N-[(3-methyl-1,2-oxazol-5-yl)methyl]pyrimidine-2,4-diamine as a white solid (45 mg, 18% yield).

1H NMR (399.902 MHz, DMSO) δ 2.19 (3H, s), 3.83 (3H, s), 4.58 (2H, d), 5.25 (2H, s), 5.35 (1H, bs), 6.03 (1H, d), 6.17 (1H, s), 6.89 (1H, d), 7.69 (1H, bs), 7.93 (1H, d), 8.15 (1H, s), 10.05 (1H, bs), 11.98 (1H, bs); m/z (ES+) [M+H]+=427.

4-Chloro-N-[(3-methyl-1,2-oxazol-5-yl)methyl]pyrimidin-2-amine was prepared as outlined in Example 13.

5-[(5-Fluoro-2-methoxy-pyridin-4-yl)methoxy]-1H-pyrazol-3-amine, used as starting material, was prepared as follows:—

3-Amino-5-hydroxypyrazole (0.56 g, 5.65 mmol) and triphenylphosphine (1.78 g, 6.78 mmol) were stirred in DCM (I6 ml) under nitrogen and the reaction mixture was cooled in an ice-bath. Diisopropylazodicarboxylate (1.34 ml, 6.78 mmol) was added dropwise over a period of 10 min. The reaction mixture was then stirred in the ice-bath for 1 h. (5-Fluoro-2-methoxy-pyridin-4-yl)methanol (1.07 g, 6.78 mmol) in THF (15 ml) was added slowly over 5-10 min. The reaction mixture was stirred and allowed to warm to room temperature over 1 h. This was then stirred for a further 18 h. The mixture was filtered and washed through with DCM (10 ml). The filtrate was extracted with 2M HCl(aq) (3×8 ml) and the combined extracts were basified with 6N NaOH(aq). The basified aqueous phase was extracted with DCM (3×20 ml). The combined extracts were filtered, dried over MgSO₄, filtered and evaporated. The crude product was purified by silica column chromatography, eluting with 0-3% MeOH in DCM, to afford 5-[(5-fluoro-2-methoxy-pyridin-4-yl)methoxy]-1H-pyrazol-3-amine as a white solid (354 mg, 26% yield).

¹H NMR (399.902 MHz, DMSO) δ 3.75 (s, 3H), 4.70 (s, 1H), 4.91 (s, 2H), 5.06 (s, 2H), 6.76 (d, 1H), 8.04 (d, 1H), 10.37 (s, 1H); m/z (ES+) [M+H]+=239.

(5-Fluoro-2-methoxy-pyridin-4-yl)methanol, used as starting material, was prepared as follows:—

Borane-tetrahydrofuran complex (IM solution in THF, 52.6 ml, 52.6 mmol) was added slowly to a solution of 5-fluoro-2-methoxy-pyridine-4-carboxylic acid (2 g, 11.7 mmol) in THF (100 ml) under nitrogen. The reaction mixture was stirred at room temperature for 2.5 h. The solvent was evaporated and the residue was stirred in methanol (40 ml) for 18 h. The solvent was evaporated and the crude product was purified by silica column chromatography, eluting with 0-1% MeOH in DCM. Pure product fractions were combined and evaporated to afford (5-fluoro-2-methoxypyridin-4-yl)methanol as a white solid (1.42 g, 77%).

¹H NMR (399.902 MHz, CDCl₃) δ 3.90 (s, 3H), 4.76 (s, 2H), 6.84-6.87 (m, 1H), 7.92 (d, 1H); m/z (ES+) [M+H]+=158.

### Example 137

### N′-[5-[(4-methoxypyridin-2-yl)methoxy]-1H-pyrazol-3-yl]-N-[(3-methyl-1,2-oxazol-5-yl)methyl]pyrimidine-2,4-diamine

A solution of 5-((4-methoxypyridin-2-yl)methoxy)-1H-pyrazol-3-amine (50 mg, 0.23 mmol) and 4-chloro-N-[(3-methyl-1,2-oxazol-5-yl)methyl]pyrimidin-2-amine (51.0 mg, 0.23 mmol) in ethanol (1.5 ml) was stirred at 80° C. for 3 days. The solution was cooled to room temperature and allowed to stand overnight. A small amount of crystallised solid was removed by filtration and the filtrate was evaporated to dryness. The crude product from the filtrate was purified by preparative HPLC using decreasingly polar mixtures of water (containing 0.1% TFA) and MeCN as eluents, then further purified by preparative HPLC using decreasingly polar mixtures of water (containing 1% NH₃) and MeCN as eluents. Fractions containing the desired compound were evaporated to dryness to afford N′-[5-[(4-methoxypyridin-2-yl)methoxy]-1H-pyrazol-3-yl]-N-[(3-methyl-1,2-oxazol-5-yl)methyl]pyrimidine-2,4-diamine (25 mg, 27%) as a white solid.

1H NMR (399.902 MHz, DMSO) δ 2.24 (3H, s), 3.89 (3H, s), 4.64 (2H, d), 5.21 (2H, s), 5.39 (1H, bs), 6.08 (1H, d), 6.22 (1H, s), 6.94-6.99 (1H, m), 7.07 (1H, d), 7.76 (1H, bs), 7.97 (1H, d), 8.42 (1H, d), 10.10 (1H, bs), 12.01 (1H, bs); m/z (ES+) [M+H]+=409

4-Chloro-N-[(3-methyl-1,2-oxazol-5-yl)methyl]pyrimidin-2-amine was prepared as outlined in Example 13.

5-((4-Methoxypyridin-2-yl)methoxy)-1H-pyrazol-3-amine, used as starting material, was prepared as follows:—

3-Amino-5-hydroxypyrazole (1 g, 10.09 mmol) and triphenylphosphine (3.18 g, 12.22 mmol) were stirred in DCM (25 ml) under nitrogen and the reaction mixture was cooled in an ice-bath. Diisopropylazodicarboxylate (2.38 ml, 12.11 mmol) was added dropwise over a period of 10 min. The reaction mixture was then stirred in the ice-bath for 1 h. (4-Methoxypyridin-2-yl)methanol (1.495 g, 12.11 mmol) in DCM (10 ml) was added over 5 min. The reaction mixture was then stirred at room temperature for 18 h. The mixture was filtered and washed through with DCM (10 ml). The filtrate was extracted with 2M HCl(aq) (3×8 ml) and the combined extracts were basified with 6N NaOH(,q). The basified aqueous phase was then extracted with DCM (3×20 ml). The combined DCM extracts from the basic phase were dried over MgSO₄, filtered, evaporated and purified by silica column chromatography, eluting with 0-7% MeOH in DCM. Product fractions were combined and evaporated to afford the product, 5-((4-methoxypyridin-2-yl)methoxy)-1H-pyrazol-3-amine, as a yellow gum (220 mg, 67% purity), used for subsequent reaction without further purification.

1H NMR (399.902 MHz, DMSO) δ 3.83 (3H, s), 4.79 (1H, s), 4.96 (2H, s), 5.05 (2H, s), 6.87-6.92 (1H, m), 6.97 (1H, d), 8.35 (1H, d), 10.41 (1H, s); m/z (ES+) [M+H]+=221.

### Example 144

### N-[(3-propan-2-yl-1,2-oxazol-5-yl)methyl]-N′-(5-propan-2-yloxy-2H-pyrazol-3-yl)pyrimidine-2,4-diamine

2-chloro-N-(5-propan-2-yloxy-2H-pyrazol-3-yl)pyrimidin-4-amine (100 mg, 0.39 mmol), (3-propan-2-yl-1,2-oxazol-5-yl)methanamine (83 mg, 0.59 mmol) and N-ethyl-N-propan-2-yl-propan-2-amine (0.171 ml, 0.99 mmol) were dissolved in 2-methoxyethanol (2 ml) and sealed into a microwave tube. The reaction was heated to 160° C. for 1 h then 200° C. for 2 h in the microwave reactor and cooled to room temperature. The crude product was purified by ion exchange chromatography, using an SCX column. The crude product was eluted from the column using 7M NH3/MeOH and then was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford the title compound (13.00 mg, 9.23%) as a yellow solid.

1H NMR (400.13 MHz, DMSO-d6) δ 1.20 (6H, d), 1.27 (6H, d), 2.93-2.99 (1H, m), 4.59 (2H, d), 4.66 (1H, q), 5.20 (1H, s), 6.02 (1H, d), 6.25 (1H, s), 7.68 (1H, s), 7.92 (1H, d), 9.97 (1H, s), 11.88 (1H, s) MS m/z 358 (MH+).

2-Chloro-N-(5-propan-2-yloxy-2H-pyrazol-3-yl)pyrimidin-4-amine, used as starting material, was prepared as in Example 77.

(3-Propan-2-yl-1,2-oxazol-5-yl)methanamine, used as starting material, was prepared in an analogous manner to that outlined for 3-cyclopropyl-1,2-oxazol-5-yl)methanamine hydrochloride in Example 3, except using 2-methylpropanal as starting material.

### Example 145

### N-[[3-(3-methyloxetan-3-yl)-1,2-oxazol-5-yl]methyl]-N′-(5-propan-2-yloxy-2H-pyrazol-3-yl)pyrimidine-2,4-diamine

N-Ethyl-N-propan-2-yl-propan-2-amine (0.388 mL, 2.23 mmol), [3-(3-methyloxetan-3-yl)-1,2-oxazol-5-yl]methanamine (250 mg, 1.49 mmol) and 2-chloro-N-(5-propan-2-yloxy-2H-pyrazol-3-yl)pyrimidin-4-amine (189 mg, 0.74 mmol) were dissolved in 2-methoxy ethanol (4 mL) and sealed into a microwave tube. The reaction was heated to 180° C. for 4 h in the microwave reactor and cooled to room temperature. The crude product was purified by preparative HPLC using decreasingly polar mixtures of water (containing 1% NH3) and MeCN as eluents. Fractions containing the desired compound were evaporated to dryness to afford the title compound (7.00 mg, 2.444%) as a white solid.

1H NMR (399.9 MHz, DMSO-d6) δ1.25 (6H, d), 1.61 (3H, s), 4.49 (2H, d), 4.63 (2H, d), 4.65 (1H, m), 4.74 (2H, d), 5.23 (1H, s), 6.00 (1H, d), 6.49 (1H, s), 7.68 (1H, s), 7.94 (1H, d), 9.98 (1H, s), 11.75 (1H, s) MS: m/z 386 (MH+)

[3-(3-methyloxetan-3-yl)-1,2-oxazol-5-yl]methanamine, used as starting material, was prepared in an analogous manner to that outlined for 3-cyclopropyl-1,2-oxazol-5-yl)methanamine hydrochloride in Example 3, except using (NE)-N-[(3-methyloxetan-3-yl)methylidene]hydroxylamine as starting material.

2-Chloro-N-(5-propan-2-yloxy-2H-pyrazol-3-yl)pyrimidin-4-amine, used as starting material, was prepared as in Example 77.

### Example 146

### N-[[3-(1-methylcyclopropyl)-1,2-oxazol-5-yl]methyl]-N′-(5-propan-2-yloxy-2H-pyrazol-3-yl)pyrimidine-2,4-diamine

2-Chloro-N-(5-propan-2-yloxy-2H-pyrazol-3-yl)pyrimidin-4-amine (100 mg, 0.39 mmol, 1 eq), [3-(1-methylcyclopropyl)-1,2-oxazol-5-yl]methanamine (120 mg, 0. 79 mmol, 2 eq) and N-ethyl-N-propan-2-yl-propan-2-amine A (0.103 ml, 0.59 mmol, 1.5 eq) were dissolved in 2-methoxyethanol (1.5 ml) and sealed into a microwave tube. The reaction was heated to 200° C. for 75 mins in the microwave reactor, before being cooled to room temperature. The crude product solution was purified by reverse-phase prep. HPLC (basic) using a 31-51% gradient of acetonitrile in water containing 1% ammonium hydroxide solution. The clean fractions were taken and evaporated to afford the title compound as a cream-coloured solid. (31.0 mg, 21.29% yield)

¹H NMR (399.902 MHz, DMSO) δ 0.82 (2H, m), 0.91 (2H, m), 1.28 (6H, d), 1.37 (3H, s), 4.56 (2H, d), 4.67 (1H, bs), 5.21 (1H, bs), 6.03 (1H, bs), 6.08 (1H, bs), 7.66 (1H, bs), 7.91 (1H, bs), 9.98 (1H, bs), 11.78 (1H, bd).

MS: m/z 370 (MH+)

[3-(1-methylcyclopropyl)-1,2-oxazol-5-yl]methanamine, used as starting material, was prepared as follows:—

A stirred solution of 1-methylcyclopropanecarbaldehyde oxime (3.90 g, 39.34 mmol, 1 eq) and tert-butyl prop-2-ynylcarbamate (13.43 g, 86.55 mmol, 2.2 eq) in dichloromethane (70 ml) was cooled to <5° C. (ice bath) under nitrogen. Aqueous sodium hypochlorite solution (13% active chlorine) (37.6 ml, 165.43 mmol, 4.2 eq) was added over a period of 2 h to the stirred solution, keeping the temperature below <10° C. (under nitrogen). The resulting mixture was then stirred under nitrogen, for 64 h, before being diluted with dichloromethane (160 ml) and water (160 ml), and being separated. The organic layer was washed with saturated brine (107 ml×2), dried with magnesium sulphate, filtered, and evaporated under reduced pressure to afford a pale yellow oil (15.22 g), which was dissolved in methanol (25 ml). 5N aqueous hydrochloric acid (26.0 ml, 129.82 mmol, 3.3 eq), and water (8 ml) were added, and the resulting solution was stirred at 50° C. for 3 h, before being allowed to cool to room temperature overnight. The methanol was then removed by evaporation under reduced pressure and the remaining aqueous solution was washed with dichloromethane (52 ml×3), before being adjusted to pH12 with 40% w/w aqueous sodium hydroxide solution, and extracted into dichloromethane (105 ml×4). The dichloromethane extracts were then washed with saturated brine (157 ml×2), dried with magnesium sulphate and filtered, before being evaporated under reduced pressure to give [3-(1-methylcyclopropyl)-1,2-oxazol-5-yl]methanamine as a brown oil (2.91 g, 48.6% yield).

¹H NMR (399.902 MHz, DMSO) δ 0.83 (2H, m), 0.91 (2H, m), 1.38 (3H, s), 1.99 (2H, bs), 3.73 (2H, s), 6.07 (1H, s).

MS: m/z 153 (MH+)

2-Chloro-N-(5-propan-2-yloxy-2H-pyrazol-3-yl)pyrimidin-4-amine, used as starting material, was prepared as in Example 77.

### Example 147

### N′-(5-methoxy-2H-pyrazol-3-yl)-N-[(3-methyl-1,2-oxazol-5-yl)methyl]pyrimidine-2,4-diamine

4-chloro-N-[(3-methyl-1,2-oxazol-5-yl)methyl]pyrimidin-2-amine (0.225 g, 1.00 mmol) and 3-methoxy-1H-pyrazol-5-amine (0. 113 g, 1 mmol) in ethanol were sealed into a microwave tube. The reaction was heated to 100° C. for 2 h in the microwave reactor and cooled to room temperature. The reaction mixture was evaporated to dryness. The crude product was purified by preparative HPLC (Waters XBridge Prep C18 OBD column, 5μ silica, 19 mm diameter, 100 mm length), using decreasingly polar mixtures of water (containing 1% TFA) and MeCN as eluents. Fractions containing the desired compound were evaporated to dryness to afford the title compound (0.065 g, 21.57%) as a yellow solid.

1HNMR (399.9 MHz, DMSO-d6) δ 2.19 (3H, d), 3.89 (3H, s), 4.73 (2H, d), 5.60-5.81 (1H, bs), 6.29-6.45 (2H, 2bs), 7.92 (1H, d), 8.85 (1H, bs), 11.10 (1H, bs)

MS: m/z 302 (MH+)

4-Chloro-N-[(3-methyl-1,2-oxazol-5-yl)methyl]pyrimidin-2-amine was prepared as outlined in Example 13.

TABLE 5

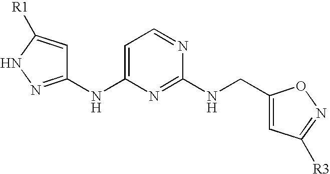

| Example | R1 | R3 |
|---|---|---|
| 104 | 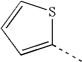 | Me |
| 105 | 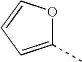 | Me |
| 106 | 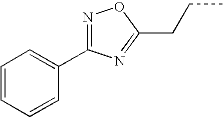 | Me |
| 107 | 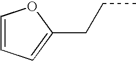 | Me |
| 108 | 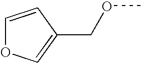 | Me |
| 109 | 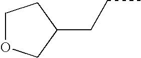 | Me |
| 110 | 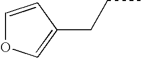 | Me |
| 111 | 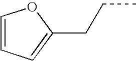 | 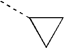 |
| 112 | 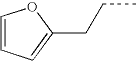 | 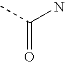 |
| 113 | 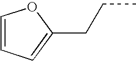 | 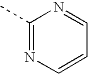 |
| 114 | 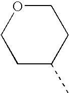 | Me |
| 115 | 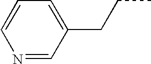 | Me |
| 116 | 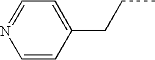 | Me |
| 117 | 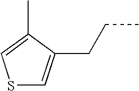 | Me |
| 118 | 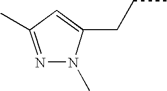 | Me |
| 119 | 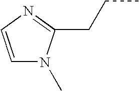 | Me |
| 120 | 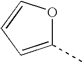 | 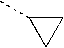 |
| 121 | 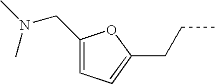 | Me |
| 129 | 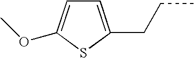 | Me |
| 130 | 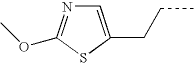 | Me |

### Example 104

### N-[(3-methyl1,2-oxazol-5-yl)methyl]-N′-(5-thiophen-2-yl-1H-pyrazol-3-yl)pyrimidine-2,4-diamine

4-chloro-N-[(3-methyl1,2-oxazol-5-yl)methyl]pyrimidin-2-amine (100mg, 0.45 mmol, 1 eq) and the 5-amino-3-(2-thienyl)pyrazole (0.47 mmol, 1.05 eq) were combined in ethanol (5 ml) and heated to 80° C. for 24 h. After this time the precipitate was filtered and washed with cold ethanol (20 ml). The solid was taken up into water (8 ml) and basified to pH9 using ammonium hydroxide solution, added dropwise. The resultant solid was filtered and washed with cold water (20 ml), then dried under vacuum to yield the title compound (71 mg, 45%) as a white solid.

1H NMR (500.133 MHz, DMSO) δ 2.17 (s, 3H), 4.59 (s, 2H), 6.11 (s, 1H), 6.27 (s, 2H), 6.54 (s, 1H), 6.70 (s, 1H), 7.63 (s, 1H), 7.89 (d, 1H). MS: m/z 354 (MH+).

4-Chloro-N-[(3-methyl-1,2-oxazol-5-yl)methyl]pyrimidin-2-amine was prepared as outlined in Example 13.

### Example 105

### N′-[5-(2-furyl)-1H-pyrazol-3-yl]-N-[(3-methyl1,2-oxazol-5-yl)methyl]pyrimidine-2,4-diamine

Made using the method in example 104 from 4-chloro-N-[(3-methyl1,2-oxazol-5-yl)methyl]pyrimidin-2-amine (100 mg, 0.45 mmol, 1 eq) and 5-(2-furyl)-1H-pyrazol-3-amine (70 mg, 0.47 mmol, 1.05 eq) to give the title compound (1 19 mg, 78%) as a white solid.

MS: m/z 337 (MH+).

4-Chloro-N-[(3-methyl-1,2-oxazol-5-yl)methyl]pyrimidin-2-amine was prepared as outlined in Example 13.

### Example 106

### N-[(3-methyl1,2-oxazol-5-yl)methyl]-N′-[5-[2-(3-phenyl-1,2,4-oxadiazol-5-yl)ethyl]-2H-pyrazol-3-yl]pyrimidine-2,4-diamine

A mixture of 5-[2-(3-phenyl-1,2,4-oxadiazol-5-yl)ethyl]-2H-pyrazol-3-amine (77 mg, 0.30 mmol, 1 eq) and 4-chloro-N-[(3-methyl1,2-oxazol-5-yl)methyl]pyrimidin-2-amine (67 mg. 0.30 mmol, 1 eq) in ethanol (5 ml) containing a few drops of 4M HCl in dioxane was heated at reflux for 18 hours before allowing to cool. The precipitated solid was filtered, washed with cold ethanol then dried. The solid was suspended in water and basified by the addition of 2M sodium hydroxide. The solid was then filtered, washed with water then 50% ether/hexane and dried overnight in the vacuum dessicator at 60° C.

1H NMR (300.132 MHz, DMSO): δ 2.17 (s, 3H), 3.12 (t, 2H), 3.36 (t, 2H), 4.52 (d, 2H), 6.11 (s, 1H), 6.11-6.46 (m, 2H), 7.19 (s, 1H), 7.53-7.63 (m, 3H), 7.83 (d, 1H), 7.98-8.03 (m, 2H), 9.38 (s, 1H), 12.04 (s, 1H). MS: m/z 444 (MH+).

4-Chloro-N-[(3-methyl-1,2-oxazol-5-yl)methyl]pyrimidin-2-amine was prepared as outlined in Example 13.

5-[2-(3-phenyl-1,2,4-oxadiazol-5-yl)ethyl]-2H-pyrazol-3-amine, used as starting material, was prepared from methyl 3-(3-phenyl-1,2,4-oxadiazol-5-yl)propanoate in a similar manner example 24a). An orange solid was obtained (336 mg, 13% yield).

1H NMR (300.132 MHz, DMSO) δ 2.98 (t, 2H), 3.27 (t, 2H), 4.26-4.78 (m, 1H), 5.19 (s, 1H), 7.53-7.60 (m, 3H), 7.97-8.05 (m, 3H), 11.15 (s, 2H). MS: m/z 256 (MH+).

### Example 107

### N′-[5-[2-(2-furyl)ethyl]-2H-pyrazol-3-yl]-N-[(3-methyl1,2-oxazol-5-yl)methyl]pyrimidine-2,4-diamine

A mixture of 2-chloro-N-[5-[2-(2-furyl)ethyl]-2H-pyrazol-3-yl]pyrimidin-4-amine (100 mg, 0.35 mmol, 1 eq), (3-methyl-1,2-oxazol-5-yl)methanamine hydrochloride (62 mg, 0.42 mmol, 1.5 eq) and diisopropylethylamine (159[l, 0.91 mmol, 3 eq) in methoxyethanol (3 ml) was heated in the microwave at 190° C. for 240 mins before evaporating solvent under reduced pressure. The crude product was purified on the acidic reverse phase hplc using a 20-40% gradient of acetonitrile in water containing 0.2% TFA. The clean fractions were taken and loaded onto a SCX-3 column pre-wet with methanol. After washing through three times with methanol the product was finally eluted with 10% ammonia solution in methanol. After evaporation to low volume a white solid was obtained. (68.7 mg, 48% yield)

1H NMR (300.132 MHz, DMSO): δ 2.17 (s, 3H), 2.80-2.99 (m, 4H), 4.54 (d, 2H), 6.11 (d, 2H), 6.22-6.33 (m, 2H), 6.34 (dd, 1H), 7.23 (s, 1H), 7.51 (d, 1H), 7.82 (d, 1H), 9.41 (s, 1H), 11.95 (s, 1H). MS: m/z 366 (MH+).

(3-Methyl-1,2-oxazol-5-yl)methanamine was synthesized as outlined in Example 1.

2-chloro-N-[5-[2-(2-furyl)ethyl]-2H-pyrazol-3-yl]pyrimidin-4-amine, used as starting material was prepared from 4-[2-(2-furyl)ethyl]-1H-pyrazol-3-amine in a similar way to the synthesis of 2-chloro-N-[5-[2-(3-methoxyphenyl)ethyl]-1H-pyrazol-3-yl]pyrimidin-4-amine used in example 27b). (2.26 g, 78% yield, beige solid)

1H NMR (300.132 MHz, DMSO): δ 2.87-2.99 (m, 4H), 6.03-6.21 (m, 2H), 6.35 (dd, 1H), 6.91-7.44 (m, 1H), 7.52 (m, 1H), 8.16 (d, 1H), 10.27 (s, 1H), 12.23 (s, 1H). MS: m/z 289 (MH+).

4-[2-(2-furyl)ethyl]-1H-pyrazol-3-amine (2.19 g, 31% over 2steps) was prepared in an analogous manner to example 24a) starting from ethyl 3-(2-furyl)propanoate.

1H NMR (300.132 MHz, DMSO): δ 2.70-2.88 (m, 4H), 4.43 (s, 1H), 5.18 (s, 1H), 6.09 (d, 1H), 6.34 (t, 1H), 7.50 (s, 1H), 11.10 (s, 1H).

### Alternative Method for Synthesis of Example 107

### N′-[5-[2-(2-furyl)ethyl]-2H-pyrazol-3-yl]-N-[(3-methyl1,2-oxazol-5-yl)methyl]pyrimidine-2,4-diamine

Prepared in an analogous way to example 11 but starting with 5-[2-(2-furyl)ethyl]-2H-pyrazol-3-amine (1 12 mg, 0.50 mmol, 1 eq). The title compound was isolated as a solid by the method used in example. (95 mg, 52% yield).

1H NMR (300.132 MHz, DMSO): δ2.17 (s, 3H), 2.81-2.98 (m, 4H), 4.53 (d, 2H), 6.11 (s, 1H), 6.12 (d, 1H), 6.24-6.30 (m, 2H), 6.34 (dd, 1H), 7.18 (s, 1H), 7.51 (dd, 1H), 7.83 (d, 1H), 9.35 (s, 1H), 11.94 (s, 1H). MS: m/z 366 (MH+).

4-[2-(2-furyl)ethyl]-2H-pyrazol-3-amine, used as starting material was prepared as follows:
a) A mixture of ethyl 2-(triphenylphosphoranylidene)acetate (34.84 g, 100 mmol, 1 eq) and furan-2-carbaldehyde (9609 mg, 100 mmol, 1 eq) in anhydrous tetrahydrofuran (200 ml) was stirred at room temperature overnight for 24 hours. The solvent was evaporated under reduced pressure and the residue triturated with ether to produce a brown solution and a precipitate. The solid was filtered, washed and removed. The filtrate was then evaporated and dry loaded onto silica using dichloromethane. The product was purified on a 120 g silica column eluting with 0-20% ethyl acetate in hexane. The clean fractions were taken and evaporated to yield a cis/trans mixture of ethyl-3-(2-furyl)prop-2-enoate as a pale yellow oil. (NMR suggested mainly trans product) (15.5 g, 93%).
b) A cis/trans mixture of ethyl-3-(2-furyl)prop-2-enoate (15.5 g, 93.27 mmol, 1 eq) was stirred in ethanol (120 ml) containing 10% palladium on charcoal (775 mg, 5% by w). The reaction was stirred under a hydrogen balloon for 4 hours. A further quantity of 10% palladium on charcoal (775 mg, 5% by w) was then added. The reaction was stirred under a hydrogen balloon for an additional 95 minutes until no starting material was indicated. The reaction was filtered to remove the palladium residues and evaporated under reduced pressure. NMR suggested a mixture of product and over-reduced product. The crude product was purified by silica chromatography on a 120 g column, eluting with 20% ethyl acetate in hexane. The clean fractions were evaporated under reduced pressure and ethyl 3-(2-furyl)propanoate obtained as a clear oil. (3.69 g, 24% yield)

1H NMR (300.132 MHz, CDCl3): δ 1.25 (t, 3H), 2.64 (t, 2H), 2.97 (t, 2H), 4.15 (q, 2H), 6.02 (td, 1H), 6.27 (dd, 1H), 7.30 (dd, 1H).

5-[2-(2-furyl)ethyl]-2H-pyrazol-3-amine (2.09 g, 72% over 2steps) was then prepared in an analogous manner to that previously shown starting from ethyl-3-(2-furyl)propanoate.

1H NMR (300.132 MHz, DMSO): δ 2.69-2.90 (m, 4H), 4.45 (s, 2H), 5.18 (s, 1H), 6.09 (dd, 1H), 6.34 (dd, 1H), 7.50 (dd, 1H), 11.10 (s, 1H). MS: m/z 178 (MH+).

### Example 108

### N′-[5-(3-furylmethoxy)-1H-pyrazol-3-yl]-N-[(3-methyl1,2-oxazol-5-yl)methyl]pyrimidine-2,4-diamine

A mixture of 5-(3-furylmethoxy)-1H-pyrazol-3-amine (117 mg, 0.65 mmol), 4-chloro-N-[(3-methylisoxazol-5-yl)methyl]pyrimidin-2-amine (also known as 4-chloro-N-[(3-methyl-1,2-oxazol-5-yl)methyl]pyrimidin-2-amine; 147 mg, 0.65 mmol) and ethanol (5 ml) was heated at 100° C. in the microwave for 15 mins. After cooling, the crystalline solid was filtered off, washed with ethanol and diethyl ether to afford the title compound as a white solid (42 mg, 19%). ¹H NMR (399.9 MHz, DMSO-d₆) δ2.20 (3H, s), 4.75 (2H, d), 4.98 (2H, s), 5.96 (1H, s), 6.49 (1H, s), 6.57 (1H, d), 7.68 (1H, s), 7.78 (1H, s), 7.94 (1H, d), 8.82 (1H, s); MS: m/z 368 (MH+).

4-Chloro-N-[(3-methyl-1,2-oxazol-5-yl)methyl]pyrimidin-2-amine was prepared as outlined in Example 13.

5-(3-furylmethoxy)-1H-pyrazol-3-amine, used as starting material was prepared as follows:

A mixture of triphenylphosphine (6.82 g, 26 mmol), 3-amino-5-hydroxypyrazole (1.49 g, ml 5 mmol) in dichloromethane (40 ml) was treated portion wise at 0° C. with DTAD (5.99 g, 26 mmol). Stirred for 15 mins at 0° C. and a solution of 3-furanmethanol (1.915 g, 19.5 mmol) in dichloromethane (20 ml) was added at 0° C. Stirred at ambient temperature for 18 h. After filtration, the organic layer was extracted with 2N HCl solution (2×20 ml). The aqueous layer was neutralised with 40% sodium hydroxide to pH 8, extracted with diethyl ether (3×25 ml), washed with water and then brine and finally dried over magnesium sulphate. After evaporating under reduced pressure, the crude product was purified by reverse phase prep. HPLC (acidic) using a 2-40% gradient of acetonitrile in water containing 0.1% trifluoroacetic acid. The desired fractions were taken and evaporated to afford 5-(3-furylmethoxy)-1H-pyrazol-3-amine as a purple solid (121 mg, 3.5%). ¹H NMR (500.13 MHz, DMSO-d₆) δ5.09 (2H, s), 5.22 (1H, s), 6.58-6.58 (1H, m), 7.70 (1H, t), 7.83 (1H, s). MS: m/z 180 (MH+).

### Example 109

### N-[(3-methyl1,2-oxazol-5-yl)methyl]-N′-[5-[2-(oxolan-3-yl)ethyl]-1H-pyrazol-3-yl]pyrimidine-2,4-diamine

Prepared in an analogous way to example 107, but starting with 5-[2-(oxolan-3-yl)ethyl]-1H-pyrazol-3-amine (112 mg, 0.50 mmol, 1 eq). The HCl salt precipitated out of the reaction mixture on cooling and was filtered and dried. The product was suspended in water and basified by the addition of ammonium hydroxide solution before extraction into ethyl acetate. The organic layer was separated, washed again with ammonium hydroxide solution and then brine. Dried with magnesium sulphate, filtered and evaporated to afford the title compound as a solid. (84 mg, 45% yield).

1H NMR (300.132 MHz, DMSO): δ 1.47 (dq, 1H), 1.64 (q, 2H), 1.93-2.17 (m, 2H), 2.17 (s, 3H), 2.49-2.56 (m, 2H), 3.18-3.38 (m, 1H), 3.61 (qd, 1H), 3.69-3.76 (m, 1H), 3.78 (t, 1H), 4.53 (d, 2H), 6.10 (s, 1H), 6.16-6.37 (m, 2H), 7.19 (s, 1H), 7.82 (d, 1H), 9.35 (s, 1H), 11.87 (s, 1H). MS: m/z 370 (MH+).

5-[2-(oxolan-3-yl)ethyl]-1H-pyrazol-3-amine used as starting material was prepared as follows:
a) Ethyl 2-(triphenylphosphoranylidene)acetate (32.4 g, 02.83 mol, 1 eq) was added to a stirred solution of 3-furaldehyde (9.82 g, 92.83 mmol,1 eq) in anhydrous tetrahydrofuran (93 ml). The reaction was stirred at room temperature overnight. The solvent was evaporated under reduced pressure and the residue triturated with ether to produce a brown solution and a precipitate. The solid was filtered. The filtrate was then evaporated. The filtrate was evaporated and dry loaded onto silica in dichloromethane. The product was purified on a 120 g silica column eluting with 0-25% ethyl acetate in hexane. The clean fractions were taken and evaporated to afford ethyl (E)-3-(3-furyl)prop-2-enoate as an orange oil (11.88 g, 77% yield as mainly trans product)

1H NMR (300.132 MHz, DMSO): δ 1.24 (t, 3H), 4.16 (q, 2H), 6.36 (d, 1H), 6.96 (d, 1H), 7.56 (d, 1H), 7.73 (dd, 1H), 8.10 (d, 1H). MS: m/z 167 (MH+).
b) Ethyl (E)-3-(3-furyl)prop-2-enoate (11.88 g, 71.50 mmol, 1 eq) was stirred under a hydrogen balloon in ethanol (150 ml) containing 10% palladium on charcoal (1.2 g) for 6 hours. The reaction was filtered to remove the palladium residues and evaporated under reduced pressure. NMR suggested product and over reduced product. The crude product was combined with the product from a smaller scale reaction and purified by column chromatography using a silica column and eluting with hexane then 0-20% ethyl acetate/hexane. Desired fractions were combined and evaporated to afford ethyl 3-(oxolan-3-yl)propanoate as a clear oil. (6.46 g).
c) Acetonitrile (2.4 ml, 45.0 mmol, 1.2 eq) was added to a slurry of sodium hydride (1.805 g, 45.0 mmol, 1.2 eq) in anhydrous 1,4-dioxane (40 ml) followed by ethyl 3-(oxolan-3-yl)propanoate (6.46 g, 37.51 mmol, 1 eq) in anhydrous 1,4-dioxane (40 ml). The reaction was then heated at 1l0degc for 24 hours then cooled. Ethanol (10 ml) was added followed by hydrazine hydrochloride (5.14 g, 75.0 mmol, 2 eq) and the reaction heated at 100 degC. for 18 hours. The solvent was decanted to remove the insoluble inorganics. The solvent was then evaporated under reduced pressure. The residue was extracted into ethyl acetate and washed twice with water. The organic layer was then washed three times with 2M HCl and the aqueous layers combined. After basifying with ammonium hydroxide solution the aqueous layer was extracted twice with ethyl acetate. The organic layers were combined, washed with brine then dried over magnesium sulphate. After filtering the solvent was evaporated under reduced pressure to yield 786 mg as a brown oil. LC/MS indicated a molecular ion ES(+ve)=182, 54% by hplc. This was dissolved in acetonitrile and purified on the basic reverse phase hplc machine in several batches using a 5-25% gradient of acetonitrile in water containing 1% ammonium hydroxide solution. The fractions containing the desired product were combined and evaporated under reduced pressure to afford 5-[2-(oxolan-3-yl)ethyl]-1H-pyrazol-3-amine as an orange oil. (478 mg, 73% by hplc).

### Example 110

### N′-[5-[2-(3-furyl)ethyl]-1H-pyrazol-3-yl]-N-[(3-methyl1,2-oxazol-5-yl)methyl]pyrimidine-2,4-diamine

Prepared in an analogous way to example 107, but starting with 5-[2-(3-furyl)ethyl]-1H-pyrazol-3-amine (112 mg, 0.50 mmol, 1 eq). The title compound was isolated as a solid (105.7 mg, 58% yield).

1H NMR (300.132 MHz, DMSO): δ 2.17 (s, 3H), 2.66-2.83 (m, 4H), 4.53 (d, 2H), 6.10 (s, 1H), 6.22-6.34 (m, 2H), 6.38 (s, 1H), 7.18 (s, 1H), 7.44 (s, 1H), 7.55 (t, 1H), 7.83 (d, 1H), 9.35 (s, 1H), 11.91 (s, 1H). MS: m/z 366 (MH+).

5-[2-(3-furyl)ethyl]-1H-pyrazol-3-amine used as starting material was prepared in an analogous manner to example 24a), from ethyl 3-(3-furyl)propanoate. Isolated as an orange solid (3.94 g, 59% yield).

1H NMR (300.132 MHz, CDCl3): δ 2.70-2.83 (m, 4H), 5.47 (s, 1H), 6.24 (d, 1H), 7.21 (s, 1H), 7.35 (t, 1H). MS: m/z 178 (MH+).

Ethyl 3-(3-furyl)propanoate was obtained as a clear oil. (6.33 g, 47% yield)

1H NMR (300.132 MHz, CDCl3): δ 1.25 (t, 3H), 2.55 (t, 2H), 2.76 (t, 2H), 4.14 (q, 2H), 6.27 (s, 1H), 7.24 (td, 1H), 7.34 (t, 1H).

### Example 111

### N-[(3-cyclopropyl1,2-oxazol-5-yl)methyl]-N′-[5-[2-(2-furyl)ethyl]-2H-pyrazol-3-yl]pyrimidine-2,4-diamine

Prepared in an analogous manner to example 107, but starting with (3-cyclopropyl1,2-oxazol-5-yl)methanamine hydrochloride (73 mg, 0.42 mmol, 1.5 eq). Purified on the acidic reverse phase hplc using a 25-45% gradient of acetonitrile in water containing 0.2% TFA to give the title compound (15.6 mg, 11% yield)

1H NMR (300.132 MHz, DMSO): δ 0.69 (m, 2H), 0.96 (m, 2H), 1.95 (ddd, 1H), 2.82-2.97 (m, 4H), 4.56 (d, 2H), 6.06 (s, 1H), 6.11 (d, 1H), 6.15-6.40 (m, 3H), 7.51 (s, 1H), 7.74 (s, 1H), 7.85 (d, 1H), 10.05 (s, 1H), 12.13 (s, 1H). MS: m/z 392 (MH+).

(3-Cyclopropyl1,2-oxazol-5-yl)methanamine hydrochloride was synthesized as outlined in Example 3.

### Example 112

### 5-[[[4-[[5-[2-(2-furyl)ethyl]-2H-pyrazol-3-yl]amino]pyrimidin-2-yl]amino]methyl]1,2-oxazole-3-carboxamide

Prepared in an analogous manner to example 107, but starting with 5-(aminomethyl)-1,2-oxazole-3-carboxamide trifluoroacetic acid salt (84 mg, 0.33 mmol, 1 eq). Purified on the acidic reverse phase hplc using a 15-35% gradient of acetonitrile in water containing 0.2% TFA to give the title compound (8.3 mg, 6% yield)

1H NMR (300.132 MHz, DMSO): δ 2.82-2.97 (m, 4H), 4.66 (d, 2H), 6.11 (d, 1H), 6.15-6.42 (m, 3H), 6.57 (s, 1H), 7.00 (s, 1H), 7.50 (d, 1H), 7.74 (s, 1H), 7.86 (d, 1H), 8.03 (s, 1H), 9.85 (s, 1H), 12.08 (s, 1H). MS: m/z 395 (MH+).

5-(Aminomethyl)-1,2-oxazole-3-carboxamide, used as starting material, can be prepared as outlined in Example 4.

### Example 113

### N′-[5-[2-(2-furyl)ethyl]-2H-pyrazol-3-yl]-N-[(3-pyrimidin-2-yl1,2-oxazol-5-yl)methyl]pyrimidine-2,4-diamine

Prepared in an analogous manner to example 107, but starting with (3-pyrimidin-2-yl1,2-oxazol-5-yl)methanamine trifluoroacetic acid salt (122 mg, 0.42 mmol, 1.2 eq). Purified on the acidic reverse phase hplc using a 20-40% gradient of acetonitrile in water containing 0.2% TFA. The cleaner fractions were trapped onto a 5 g scx-3 column then the column was washed with methanol before the product was eluted with 10% ammonium hydroxide solution in methanol. Evaporation under reduced pressure yielded slightly purer material. This was re-purified on the basic reverse phase prep hplc using a 25-45% gradient. After evaporation this afforded the title compound (8.3 mg, 6% yield)

1H NMR (300.132 MHz, DMSO): δ 2.82-2.97 (m, 4H), 4.66 (d, 2H), 6.11 (d, 1H), 6.15-6.42 (m, 3H), 6.57 (s, 1H), 7.00 (s, 1H), 7.50 (d, 1H), 7.74 (s, 1H), 7.86 (d, 1H), 8.03 (s, 1H), 9.85 (s, 1H), 12.08 (s, 1H). MS: m/z 395 (MH+).

(3-Pyrimidin-2-yl1,2-oxazol-5-yl)methanamine, used as starting material, can be prepared as outlined in Example 32.

### Example 114

### N-[(3-methyl1,2-oxazol-5-yl)methyl]-N′-[5-(oxan-4-yl)-1H-pyrazol-3-yl]pyrimidine-2,4-diamine hydrochloride

Prepared using an analogous method to example 46, but starting with 5-(oxan-4-yl)-1H-pyrazol-3-amine (60 mg, 0.36 mmol) to give the title compound (61 mg, 43% yield)

1H NMR (300.132 MHz, DMSO) δ 1.52-1.65 (m, 2H), 1.78 (d, 2H), 2.18 (s, 3H), 2.81-2.91 (m, 1H), 3.36-3.45 (m, 2H), 3.86-3.91 (m, 2H), 4.72 (s, 2H), 6.27 (s, 1H), 6.31 (bs, 1H), 6.39 (bs, 1H), 7.88 (d, 1H). MS: m/z 356 (MH+)

5-(oxan-4-yl)-1H-pyrazol-3-amine, used as starting material, was prepared using an analogous method to example 24a), but starting with methyl oxane-4-carboxylate (10 g, 69.4 mmol) to give 5-(oxan-4-yl)-1H-pyrazol-3-amine (1.87 g, 16%) as a white solid.

1H NMR (300.132 MHz, CDCl3) δ 1.56-1.82 (m, 4H), 2.64-2.81 (m, 1H), 3.33-3.47 (m, 2H), 3.88-3.99 (m, 2H), 5.38 (s, 1H). MS: m/z 168 (MH+)

### Example 115

### N-[(3-methyl1,2-oxazol-5-yl)methyl]-N′-[5-(2-pyridin-3-ylethyl)-2H-pyrazol-3-yl]pyrimidine-2,4-diamine

Prepared in an analogous way to example 38, but starting with 5-(2-pyridin-3-ylethyl)-2H-pyrazol-3-amine (158.5 mg, 0.84 mmol, 1 eq) and using a 15-35% gradient of acetonitrile in water containing 1% ammonia to purify. The title compound was obtained as a solid (48.7 mg, 15.4% yield).

1H NMR (300.132 MHz, DMSO): δ 2.17 (s, 3H), 2.81-2.98 (m, 4H), 4.53 (d, 2H), 6.11 (s, 1H), 6.22 (s, 2H), 7.24 (s, 1H), 7.30 (dd, 1H), 7.63 (d, 1H), 7.83 (d, 1H), 8.40 (dd, 1H), 8.44 (d, 1H), 9.39 (s, 1H), 11.94 (s, 1H). MS: m/z 377 (MH+).

5-(2-pyridin-3-ylethyl)-2H-pyrazol-3-amine used as starting material was prepared as follows:
a)Acetonitrile (2.90 ml, 55 mmol, 1.3 eq) was added to a slurry of sodium hydride (2.195 g, 54.77 mmol, 1.3 eq) in anhydrous 1,4-dioxane (50 ml). To this was added a solution of methyl 3-(3-pyridyl)propionate (6.96 g, 42.13 mmol, 1 eq) in anhydrous 1,4-dioxane (50 ml). The reaction was heated to reflux and hydrogen gas was evolved. Heating was continued overnight for 18 hours. The reaction was then cooled. Ethanol (5 ml) was added followed by hydrazine.HCl (3181 mg, 46.43 mmol, 1 eq). The reaction was refluxed overnight for 20 hours before leaving to cool. The solvent was evaporated under reduced pressure. The orange residue was dissolved in water and partioned twice with ethyl acetate. The organic layers were combined and washed twice with 2M HCl. The aqueous acidic layers were combined and washed with ethyl acetate. The aqueous layer was then separated and basified by the addition of 8N ammonia solution. The basic layer was then extracted twice with ethyl acetate. After separating, the ethyl acetate layer was washed with brine, dried with magnesium sulphate, filtered and evaporated under reduced pressure to yield 373 mg as an orange oil. LC/MS indicated the desired product with a molecular ion ES(+ve) =189, 77% by hplc. Re-extraction of the basic layer with ethyl acetate as before gave a further 220 mg of product which was 89% pure by hplc. The initial product was dissolved in 10 ml of acetonitrile and purified in two batches on the basic reverse phase hplc using a 2-20% gradient of acetonitrile in water containing 1% ammonia. Fractions 10-14 and 16-20 were taken. The second batch was purified first using a 5-25% gradient. Fractioins 1-4 were taken. All clean fractions were combined and evaporated to yield 5-(2-pyridin-3-ylethyl)-2H-pyrazol-3-amine as product (348 mg, 5% yield)

1H NMR (400.132 MHz, DMSO): δ 2.74 (t, 2H), 2.87 (t, 2H), 4.43 (s, 2H), 5.17 (s, 1H), 7.29 (ddd, 1H), 7.61 (dddd, 1H), 8.39 (dd, 1H), 8.42 (d, 1H), 11.08 (s, 1H). MS: m/z 189 (MH+).

### Example 116

### N-[(3-methyl-1,2-oxazol-5-yl)methyl]-N′-[5-(2-pyridin-4-ylethyl)-2H-pyrazol-3-yl]pyrimidine-2,4-diamine

A mixture of 5-(2-pyridin-4-ylethyl)-2H-pyrazol-3-amine (95 mg, 0.5 mmol, 1.0 eq), 4-chloro-N-[(3-methyl-1,2-oxazol-5-yl)methyl]pyrimidin-2-amine (113 mg, 0.5 mmol, 1.0 eq), and ethanol (2.5 ml) were stirred and heated at 80° C. overnight under an atmosphere of nitrogen. The solution was allowed to cool to room temperature and then evaporated to dryness. The crude product was purified by chromatography on silica column using a 0-10% gradient of methanol containing ammonia (2.0M) in dichloromethane. The clean fractions were taken and evaporated to a yellow solid. This solid was triturated with dichloromethane to afford the title compound as a yellow solid, (95 mg, 50% yield).

¹H NMR (499.8 MHz, DMSO) δ 2.19 (3H, s), 2.90-2.99 (4H, m), 4.58 (2H, d), 6.07 (1H, s), 6.11 (1H, s), 6.28 (1H, d), 6.86 (1H, s), 7.23 (2H, d), 7.87 (1H, d), 8.45 (2H, d), 8.98 (1H, s).

MS: m/z 377 (MH+)

4-Chloro-N-[(3-methyl-1,2-oxazol-5-yl)methyl]pyrimidin-2-amine was prepared as outlined in Example 13.

5-(2-pyridin-4-ylethyl)-2H-pyrazol-3-amine, used as starting material was prepared as follows:

Acetonitrile (0.151 ml, 2.84 mmol, 1.2 eq) was added to a slurry of sodium hydride (114 mg dispersion in mineral oil, 2.84 mmol, 1.2 eq) in anhydrous dioxan (8 ml) and the mixture stirred at room temperature under an atmosphere of nitrogen. Methyl 3-pyridin-4-ylpropanoate (532 mg, 2.37 mmol, 1 eq) was then added and the reaction was refluxed overnight for 18 h. The mixture was cooled to room temperature and ethanol (1 ml) added followed by hydrazine hydrochloride (325 mg, 4.74 mmol, 2.0 eq). The mixture was stirred and heated to reflux and then stirred at this temperature for 1 hour.

After cooling and quenching with a small amount of water the solvent was evaporated under reduced pressure. The residue was dissolved in 2M HCl (25 ml). The acidic solution was then extracted with ethyl acetate (50 ml). The aqueous layer was separated and the ethyl acetate layer was washed with 2M HCl (10 ml). The combined aqueous fraction was basified to pH 9 using concentrated aqueous ammonia. The product was extracted using ethyl acetate (3×50 ml). The aqueous was further basified with 4M NaOH solution and saturated with salt and extracted using ethyl acetate (3×50 ml). Finally it was extracted with 1-BuOH (100 ml). The extracts were evaporated to dryness. The residues were dissolved in dichloromethane containing 10% methanol, filtered to remove inorganics and evaporated to afford the crude product as a golden oil. The crude product was purified by column chromatography using a 0-10% gradient of methanol containing ammonia (2.0M) in dichloromethane. The clean fractions were taken and evaporated to afford the title compound as a clear gum, (209 mg, 47% yield).

MS: m/z 189 (MH+)

Methyl 3-pyridin-4-ylpropanoate was prepared as outlined in EP 0 539 977.

### Example 117

### N-[(3-methyl1,2-oxazol-5-yl)methyl]-N′-[5-[2-(4-methylthiophen-2-yl)ethyl]-2H-pyrazol-3-yl]pyrimidine-2,4-diamine

The mixture of 5-[2-(4-methylthiophen-2-yl)ethyl]-2H-pyrazol-3-amine (0.104 g, 1 mmol), 4-chloro-N-[(3-methyl-1,2-oxazol-5-yl)methyl]pyrimidin-2-amine (0.113 g, 1 mmol), and ethanol (3 ml) were heated in a microwave at 100° C. for 15 mins. The crude product was purified by reverse-phase prep. HPLC (basic) using a 30-40% gradient of acetonitrile in water containing 1% ammonium hydroxide solution, and a thin film of final product was obtained (0.002 g, 1%). MS: m/z 396.29 (MH⁺)

4-Chloro-N-[(3-methyl-1,2-oxazol-5-yl)methyl]pyrimidin-2-amine was prepared as outlined in Example 13.

5-[2-(4-Methylthiophen-2-yl)ethyl]-2H-pyrazol-3-amine, used as starting material, was prepared as follows:

Sodium hydride (60%, 0.236 g, 5.88 mmol) was added to a stirred solution of methyl 3-(4-methylthiophen-2-yl)propanoate (0.903 g, 4.90 mmol) in 1,4 dioxane (25 ml) and dry acetonitrile (0.308 ml, 5.88 mmol) under nitrogen. The mixture was stirred at r.t. for 10 mins and then refluxed under nitrogen o/n. The mixture was cooled to r.t. and ethanol (2 ml) was added, followed by hydrazine monohydrochloride (0.672 g, 9.8 mmol). The mixture was refluxed for 7 h and then left to stir at room temperature for 2d. The reaction mixture was filtered, concentrated in vacuo and partitioned between 2N HCl and ethyl acetate (25 ml each). The aqueous layer was extracted with ethyl acetate, basified with ammonium hydroxide solution to pH 8, extracted with ethyl acetate (2×), washed with water and brine, dried (MgSO4), filtered and evaporated to dryness to give yellow needle-like crystals (223 mg, 22%).

Methyl 3-(4-methylthiophen-2-yl)propanoate, used as starting material was prepared as follows:—

Methyl (E)-3-(4-methylthiophen-2-yl)prop-2-enoate (1.095 g) was hydrogenated under a hydrogen balloon with 10% Pd/C and hydrogen in ethanol (20 ml) overnight. Filtration through celite and evaporation to dryness gave an oil (0.914 g, 82.7%).

Methyl (E)-3-(4-methylthiophen-2-yl)prop-2-enoate used as starting material was prepared as follows:

4-Methyl-thiophene-2-carboxaldehyde (1.01 g, 8 mmol), methyl(triphenyl-phosphoranylidene)acetate (4.01 g, 12 mmol) and dichloromethane (25 ml) were mixed together at r.t. and stirred for 4 h. The reaction mixture was evaporated to dryness and purified by column chromatography on silica, eluting with ethyl acetate/isohexane (2:98 increasing to 10:90). The desired fractions were vaporated to dryness to give a gum (1.095 g, 75.5%).

### Example 118

### N′-[5-[2-(2,5-dimethylpyrazol-3-yl)ethyl]-1H-pyrazol-3-yl]-N-[(3-methyl-1,2-oxazol-5-yl)methyl]pyrimidine-2,4-diamine

Prepared in an analogous procedure to that in Example 57, starting from 5-[2-(2,5-dimethylpyrazol-3-yl)ethyl]-1H-pyrazol-3-amine (124 mg, 0.60 mmol) and 4-chloro-N-[(3-methylisoxazol-5-yl)methyl]pyrimidin-2-amine (also known as 4-chloro-N-[(3-methyl-1,2-oxazol-5-yl)methyl]pyrimidin-2-amine; 135 mg, 0.60 mmol) in ethanol (5 ml). The crystalline solid was filtered off and washed with cold ethanol and diethyl ether to afford the title compound as a white solid (104 mg, 44%).

¹H NMR (399.9 MHz, DMSO-d₆) δ1. 2.07 (3H, s), 2.19 (3H, s), 2.88 (4H, s), 3.63 (3H, s), 4.72 (2H, d), 5.82 (1H, s), 6.28 (1H, s), 6.39 (1H, s), 7.91 (1H, s), 8.87 (1H, s), 11.25 (1H, s), 12.49 (1H, s), 12.74 (1H, s). MS: m/z 394 (MH+).

4-Chloro-N-[(3-methyl-1,2-oxazol-5-yl)methyl]pyrimidin-2-amine was prepared as outlined in Example 13.

5-[2-(2,5-dimethylpyrazol-3-yl)ethyl]-1H-pyrazol-3-amine used as starting material was prepared using the procedure for 5-[2-(3,5-dimethoxy)ethyl]-2H-pyrazol-3-amine) in Example 42, starting from methyl 3-(2,5-dimethylpyrazol-3-yl)propanoate (645 mg, 3.54 mmol), Sodium hydride (171 mg dispersion in mineral oil, 4.26 mmol), acetonitrile (223 uL, 4.26 mmol) and hydrazine monohydrochloride (486 mg, 7.08 mmol). The crude product was purified by normal phase chromatography on silica gel using a 5-10% gradient of methanol in dichloromethane. The clean fractions were taken and evaporated to afford 5-[2-(2,5-dimethylpyrazol-3-yl)ethyl]-1H-pyrazol-3-amine as an oil (270 mg, 37%). MS: m/z 206 (MH+).

3-(2,5-dimethylpyrazol-3-yl)propanoate used as starting material was prepared using the procedure as for methyl 3-[3-(dimethylcarbamoyl)phenyl]propanoate in Example 59, starting from methyl (E)-3-(2,5-dimethylpyrazol-3-yl)prop-2-enoate (612 mg, 3.45 mmol) with 10% Pd/C (60 mg) in ethanol (15 ml) under a hydrogen atmosphere. Filtered through 10 celite, evaporated to afford 3-(2,5-dimethylpyrazol-3-yl)propanoate as an oil (648m g, >100%) ¹H NMR (399.9 MHz, DMSO-d₆) δ2.06 (3H, s), 2.64 (2H, t), 2.80 (2H, d), 3.62 (3H, s), 3.64 (3H, s), 5.79 (1H, s).

Methyl (E)-3-(1-methylimidazol-4-yl)prop-2-enoate was prepared using the procedure as for methyl (E)-3-[3-fluoro-5-(trifluoromethyl)phenyl]prop-2-enoate in Example 49, starting from 1,3-dimethyl-1H-pyrazole-5-carbaldehyde (786 mg, 6.33 mmol) and methyl(triphenyl-phosphoranylidene)acetate (3.17 g, 9.49 mmol) in dichloromethane (25 ml). The crude product was purified by normal phase chromatography on silica gel using a 0-2.5% gradient of methanol in dichloromethane, followed by chromatography on a silica gel column using 25% ethyl acetate in hexanes. The clean fractions were taken and evaporated to afford methyl (E)-3-(1-methylimidazol-4-yl)prop-2-enoate as an oil (614 mg, 54%). ¹H NMR (399.9 MHz, DMSO-d₆) δ2.14 (3H, s), 3.73 (3H, s), 3.85 (3H, s), 6.49 (1H, d), 6.64 (1H, s), 7.54-7.58 (1H, m).

### Example 119

### N′-[5-[2-(1-methylimidazol-4-yl)ethyl]-1H-pyrazol-3-yl]-N-[(3-methyl-1,2-oxazol-5-yl)methyl]pyrimidine-2,4-diamine

Prepared in an analogous procedure to that used in Example 57, starting from 5-[2-(1-methylimidazol-4-yl)ethyl]-1H-pyrazol-3-amine (115 mg, 0.60 mmol) and 4-chloro-N-[(3-methylisoxazol-5-yl)methyl]pyrimidin-2-amine (also known as 4-chloro-N-[(3-methyl-1,2-oxazol-5-yl)methyl]pyrimidin-2-amine; 135 mg, 0.60 mmol). Purified by reverse phase prep. HPLC (basic) using a 18-35% gradient of acetonitrile in water containing 1% ammonia. The clean fractions were taken and evaporated to afford the title compound as a white solid (41 mg, 18%). ¹H NMR (399.9 MHz, DMSO-d₆) 62.18 (3H, s), 2.63-2.87 (4H, m), 3.60 (3H, s), 4.54 (2H, d), 6.12 (1H, s), 6.19-6.44 (2H, m), 6.85 (1H, s), 7.20 (1H, s), 7.51 (1H, s), 7.83 (1H, d), 9.38 (1H, s), 11.96 (1H, s). MS: m/z 380 (MH+).

4-Chloro-N-[(3-methyl-1,2-oxazol-5-yl)methyl]pyrimidin-2-amine was prepared as outlined in Example 13.

5-[2-(1-methylimidazol-4-yl)ethyl]-1H-pyrazol-3-amine used as starting material was prepared using an analogous procedure to that for 5-[2-(3,5-dimethoxy)ethyl]-2H-pyrazol-3-amine) in Example 42, starting from methyl 3-(1-methylimidazol-4-yl)propanoate (732 mg, 4.35 mmol), Sodium hydride (209 mg dispersion in mineral oil, 5.22 mmol), acetonitrile (273 uL, 5.22 mmol) and hydrazine monohydrochloride (597 mg, 8.7 mmol). The crude product was purified by normal phase chromatography on silica gel using a 5-10% gradient of methanol in dichloromethane. The clean fractions were taken and evaporated to afford 5-[2-(1-methylimidazol-4-yl)ethyl]-1H-pyrazol-3-amine as an oil (198 mg, 24%). MS: m/z 192 (MH+).

3-(1-methylimidazol-4-yl)propanoate used as starting material was prepared using the procedure described in Example 59 for methyl 3-[3-(dimethylcarbamoyl)phenyl]propanoate, starting from methyl (E)-3-(1-methylimidazol-4-yl)prop-2-enoate (760 mg, 4.57 mmol) with 10% Pd/C (80 mg) in ethanol (15 ml) under a hydrogen atmosphere. Filtered through celite, evaporated to afford 3-(1-methylimidazol-4-yl)propanoate as an oil (743m g, 97%). ¹H NMR (399.9 MHz, DMSO-d₆) δ2.58-2.60 (2H, m), 2.68-2.72 (2H, m), 3.57 (3H, s), 3.62 (3H, s), 6.82 (1H, d), 7.43 (1H, d).

Methyl (E)-3-(1-methylimidazol-4-yl)prop-2-enoate was prepared using the procedure for Methyl (E)-3-[3-fluoro-5-(trifluoromethyl)phenyl]prop-2-enoate in Example 49, starting from 1-methylimidazole-4-carbaldehyde (1.03 g, 9.35 mmol) and methyl(triphenyl-phosphoranylidene)acetate (4.69 g, 14.03 mmol) in dichloromethane (25 ml). The crude product was purified by normal phase chromatography on silica gel using a 0-2.5% gradient of methanol in dichloromethane, followed by chromatography on a silica gel column using ethyl acetate. The clean fractions were taken and evaporated to afford methyl (E)-3-(1-methylimidazol-4-yl)prop-2-enoate as a solid (760 mg, 49%). ¹H NMR (399.9 MHz, DMSO-d₆) 63.67 (3H, s), 3.69 (3H, s), 6.33 (1H, d), 7.51 (1H, d), 7.57 (1H, s), 7.69 (1H, s).

### Example 120

### N-[(3-cyclopropyl-1,2-oxazol-5-yl)methyl]-N′-[5-(2-furyl)-2H-pyrazol-3-yl]pyrimidine-2,4-diamine

To a reaction tube was added 4-chloro-N-[(3-cyclopropyl-1,2-oxazol-5-yl)methyl]pyrimidin-2-amine (100 mg, 0.40 mmoles), ethanol (2 ml), and 5-(2-furyl)-2H-pyrazol-3-amine (63 mg, 0.42 mmoles). The mixture was heated overnight at 80° C. The cooled mixture was filtered and the solid was washed with ethanol. The sample was dissolved in methanol, poured onto a SCX-2 column and washed with methanol. The product eluted with 2N ammonia in methanol and the solvent was evaporated to give a gum. The gum was triturated with ether, filtered, dried in a vacuum oven at 45° C. overnight to yield the title product as a white solid (62 mg, 43%).

1H NMR (DMSO 400.13MHz d4AcOH at 373K) 0.69 (m, 2H), 0.92 (m, 2H), 1.89 (m, 1H), 4.56 (s, 2H), 5.98 (s, 1H), 6.25 (d, 1H), 6.45 (s, 1H), 6.52 (m, 1H), 6.69 (d, 1H), 7.59 (d, 1H), 7.87 (d, 1H)

MS: m/z 364 (MH+).

4-chloro-N-[(3-cyclopropyl-1,2-oxazol-5-yl)methyl]pyrimidin-2-amine was prepared as in Example 14.

### Example 121

### N′-[5-[2-[5-(dimethylaminomethyl)-2-furyl]ethyl]-1H-pyrazol-3-yl]-N-[(3-methyl-1,2-oxazol-5-yl)methyl]pyrimidine-2,4-diamine

A mixture of 5-(2-{5-[(dimethylamino)methyl]-2-furyl}ethyl)-1H-pyrazol-3-amine (118 mg, 0.5 mmol, 1.0 eq), 4-chloro-N-[(3-methylisoxazol-5-yl)methyl]pyrimidin-2-amine (also known as 4-chloro-N-[(3-methyl-1,2-oxazol-5-yl)methyl]pyrimidin-2-amine; 113 mg, 0.5 mmol, 1.0 eq), hydrogen chloride (2.0M solution in diethyl ether, 0.25 mL, 0.5 mmol, 1.0 eq) and ethanol (2.5 ml) were stirred and heated at 80° C. for 45 mins under an atmosphere of nitrogen. The solution was allowed to cool to room temperature and then evaporated to dryness. The crude product was purified by chromatography on a silica column using a 0-10% gradient of methanol containing ammonia (2.0M) in dichloromethane. The clean fractions were taken and evaporated to a white solid, 108 mg. This material was further purified by reverse-phase prep. HPLC (basic) using a 22-32% gradient of acetonitrile in water containing 1% ammonium hydroxide solution. The clean fractions were taken and evaporated to afford the title compound as a solid. (16 mg, 8% yield)

¹H NMR (499.8 MHz, DMSO-d₆, CD₃CO₂D) δ 2.19 (3H, s), 2.22 (6H, s), 2.87-2.90 (2H, m), 2.91-2.96 (2H, m), 3.46 (2H, s), 4.58 (2H, s), 6.03 (1H, d), 6.09 (2H, d), 6.14 (1H, d), 6.29 (1H, d), 7.86 (1H, d). MS: m/z 423 (MH+)

4-Chloro-N-[(3-methyl-1,2-oxazol-5-yl)methyl]pyrimidin-2-amine was prepared as outlined in Example 13.

5-(2- {5-[(dimethylamino)methyl]-2-furyl} ethyl)-1H-pyrazol-3-amine, used as starting material was prepared as follows:

Acetonitrile (0.258 ml, 4.88 mmol, 1.2 eq) was added to a slurry of sodium hydride (196 mg dispersion in mineral oil, 4.88 mmol, 1.2 eq) in anhydrous dioxan (15 ml) and the mixture stirred at room temperature under an atmosphere of nitrogen for 5 mins. Ethyl 3-{5-[(dimethylamino)methyl]-2-furyl}propanoate (917 mg, 4.07 mmol, 1.0 eq) was then added and the reaction was refluxed overnight for 18 h. The mixture was cooled to room temperature and ethanol (1.9 ml) was added, followed by hydrazine hydrochloride (558 mg, 8.14 mmol, 2.0 eq). The mixture was refluxed for 1 h. After cooling the solvent was evaporated under reduced pressure. The residue was dissolved in dichloromethane containing 10% methanol (50 mL) and the insoluble impurities were filtered off. The filtrate was evaporated to give the crude product as a golden oil, 1.07 g. This material was purified by silica column chromatography eluting with a 0-10% gradient of methanol (containing ammonia at 2M) in dichloromethane. Pure product fractions were combined and evaporated to give a clear oil. (520 mg, 55% yield)

1H NMR (399.9 MHz, DMSO-d6) δ2.16 (6H, s), 2.70-2.74 (2H, m), 2.81-2.85 (2H, m), 3.40 (2H, s), 5.20 (1H, s), 6.03 (1H, d), 6.15 (1H, d). MS: m/z 235 (MH+)

Ethyl 3- {5-[(dimethylamino)methyl]-2-furyl}propanoate, used as starting material was prepared as follows:

A mixture of ethyl 3-(2-furanyl)propionate (12.1lg, 72.0 mmol, 1.0 eq), dimethylammonium chloride (6.76 g, 82.8 mmol, 1.15 eq), 37% aqueous formaldehyde (6.43 g, 79.2 mmol, 1.1 eq) in acetic acid (75 mL) was stirred at room temperature until a solution formed. The solution was allowed to stand for 44 h. The mixture was evaporated to an oil. This was suspended in water and extracted with ethyl acetate (2×250 mL). The aqueous layer (containing the product) was basified to pH11 with 4M sodium hydroxide solution and then extracted into ethyl acetate (2×250 mL). These combined extracts were washed with brine, dried over magnesium sulphate and evaporated to give the crude product as a dark brown oil, 6.5 g. This material was purified by silica column chromatography eluting with a 0-10% gradient of methanol (containing ammonia at 2M) in dichloromethane. Fractions containing the product were combined and evaporated to give a light brown oil. (3.44 g) This material was repurified by silica column chromatography eluting with a 0-5% gradient of methanol (containing ammonia at 2M) in dichloromethane. Fractions containing the product were combined and evaporated to give a light brown oil. (1.36 g, 8% yield)

1H NMR (399.9 MHz, CDCl3) δ 1.24 (3H, t), 2.29 (6H, s), 2.62-2.65 (2H, m), 2.95 (2H, t), 3.47 (2H, s), 4.11-4.15 (2H, m), 5.95 (1H, d), 6.11 (1H, d). MS: m/z 226 (MH+)

### Example 129

### N′-[5-[2-(5-methoxythiophen-2-yl)ethyl]-1H-pyrazol-3-yl]-N-[(3-methyl-1,2-oxazol-5-yl)methyl]pyrimidine-2,4-diamine

5-(2-(5-methoxythiophen-2-yl)ethyl)-1H-pyrazol-3-amine (100 mg, 0.45 mmol, 1 eq) was added to 4-chloro-N-[(3-methyl-1,2-oxazol-5-yl)methyl]pyrimidin-2-amine (101 mg, 0.45 mmol, 1 eq) in ethanol (3 ml). The resulting solution was stirred at 80° C. for 24 h. The resulting mixture was evaporated to dryness and the residue was purified by preparative HPLC using decreasingly polar mixtures of water (containing 1% ammonium hydroxide) and MeCN as eluents. Fractions containing the desired compound were evaporated to dryness to afford N′-[5-[2-(5-methoxythiophen-2-yl)ethyl]-1 H-pyrazol-3-yl]-N-[(3-methyl-1,2-oxazol-5-yl)methyl]pyrimidine-2,4-diamine (60.0 mg, 32.6%) as a white solid.

1H NMR (400.13 MHz, DMSO-d6) δ 2.16 (3H, s), 2.81 (2H, m), 2.95 (2H, t), 3.78 (3.78 (3H, s), 4.52 (2H, d), 6.07 (1H, d), 6.10 (1H, s), 6.45-6.46 (1H, m), 7.23 (1H, s), 7.82 (1H, d), 9.40 (1H, s), 11.94 (1H, s). MS m/z 412 (MH+).

4-chloro-N-[(3-methyl-1,2-oxazol-5-yl)methyl]pyrimidin-2-amine was prepared as outlined in Example 13.

5-(2-(5-Methoxythiophen-2-yl)ethyl)-1H-pyrazol-3-amine, used as starting material, was prepared as follows:—

Acetonitrile (1. 174 ml, 22.47 mmol, 1. 8 eq) was added dropwise to lithium diisopropylamide (1 1.24 ml, 22.47 mmol, 1.8 eq 1M in THF) in THF (80 ml) at −78° C. over a period of 5 mins under nitrogen. The resulting solution was stirred at −78° C. for 10 mins. Methyl 3-(5-methoxythiophen-2-yl)propanoate (2.5 g, 12.48 mmol, 1 eq) was added dropwise and the reaction was stirred for 30 mins before being allowed to warm to 22° C. The reaction mixture was diluted with ethanol (80 ml) and hydrazine monohydrochloride (1.539 g, 22.47 mmol, 1.8 eq) was added. The reaction was heated at 70° C. until formation of pyrazole was complete. The resulting mixture was evaporated to dryness, suspended in DCM and filtered. The filtrate was purified by silica column chromatography, eluting with a gradient of 0-10% MeOH in EtOAc. Pure fractions were evaporated to dryness to afford 5-(2-(5-methoxythiophen-2-yl)ethyl)-1H-pyrazol-3-amine (875 mg, 31.4%)

1H NMR (399.902 MHz, DMSO) δ 2.69 (2H, t), 2.89 (2H, t), 3.80 (3H, s), 4.51 (2H, s), 5.22 (1H, s), 6.07 (1H, d), 6.44 (1H, d), 11.18 (1H, s). MS m/z 224 (MH+).

Methyl 3-(5-methoxythiophen-2-yl)propanoate, used as starting material, was prepared as follows:—

(E)-Methyl 3-(5-methoxythiophen-2-yl)prop-2-enoate (4 g, 2.52 mmol, 1 eq) and Palladium, (5% on Carbon 50% wet) (0.8 g, 0.16 mmol, 0.01 eq) in EtOH (100 mL) were stirred under an atmosphere of hydrogen at 3 bar and 25° C. for 15 h. The reaction mixture was filtered through celite and the solvent evaporated to give crude product as a yellow oil (2.58 g, 63%). 1H NMR (400.13 MHz, DMSO-d6) δ 2.59 (2H, t), 2.86-2.88 (2H, m), 3.59 (3H, t), 3.79 (3H, s), 6.06-6.07 (1H, m), 6.45-6.46 (1H, m). MS m/z 201 (MH+).

(E)- Methyl 3-(5-methoxythiophen-2-yl)prop-2-enoate, used as starting material, was prepared as follows:—

To 5-methoxythiophene-2-carbaldehyde (5.69 g, 40 mmol, 1 eq) in DCM (150 mL) was added methyl (triphenylphosphorylidene) acetate (20. 1 g, 60 mmol, 1.5 eq) portionwise. The reaction was stirred at room temperature overnight and then evaporated to dryness and purified by silica column chromatography, eluting with 2-5% ethyl acetate in isohexane to give product as a yellow solid (5.24 g, 66%).

1H NMR (400.13 MHz CDCl3) δ 3.75 (3H, s), 3.92 (3H, s), 5.93 (1H, d), 6.14 (1H, d), 6.63 (1H, d), 7.63 (1H, d). MS m/z 199 (MH+).

5-Methoxythiophene-2-carbaldehyde, used as starting material, was prepared as follows:—

A solution of n-butyllithium (35.5 mL, 56.93 mmol, 1.3 eq 1.6M in hexanes) was added to a solution of 2-methoxythiophene (5 g, 43.79 mmol, 1 eq) in ethoxyethane (100 mL) at 0° C. under nitrogen. The reaction was stirred for 15 mins and then DMF (4.41 ml, 56.93 mmol, 1.3 eq) was added dropwise. The temperature was allowed to rise to 25° C. over 15 mins. The mixture was heated at 35° C. for 1 h and then allowed to cool to room temperature and poured into water. The mixture was extracted with diethyl ether (×3), the organics were washed with brine, dried (MgSO₄) and evaporated to give crude product as a yellow liquid (7.2 g, >100%).

1H NMR (400.13 MHz CDCl3) δ 3.99 (1H, s), 6.34 (1H, d), 7.51 (1H, d), 9.67 (1H, s).

### Example 130

### N′-[5-[2-(2-methoxy-1,3-thiazol-5-yl)ethyl]-1H-pyrazol-3-yl]-N-[(3-methyl-1,2-oxazol-5-yl)methyl]pyrimidine-2,4-diamine

5-[2-(2-methoxy-1,3-thiazol-5-yl)ethyl]-1H-pyrazol-3-amine (100 mg, 0.45 mmol, 1 eq) was added to 4-chloro-N-[(3-methyl-1,2-oxazol-5-yl)methyl]pyrimidin-2-amine (100 mg, 0.45 mmol, 1 eq) in ethanol (3 ml). The resulting solution was stirred at 80° C. for 18 h. The resulting mixture was evaporated to dryness and the residue was purified by preparative HPLC using decreasingly polar mixtures of water (containing 0.1% TFA) and MeCN as eluents. The crude product was converted to free base by preparative HPLC using decreasingly polar mixtures of water (containing 1% ammonium hydroxide) and MeCN as eluents. Fractions containing the desired compound were evaporated to dryness to afford N′-[5-[2-(2-methoxy-1,3-thiazol-5-yl)ethyl]-1H-pyrazol-3-yl]-N-[(3-methyl-1,2-oxazol-5-yl)methyl]pyrimidine-2,4-diamine (47.0 mg, 25.6%) as a white solid.

1H NMR (400.13 MHz, DMSO-d6) δ 2.17 (3H, s), 2.83 (2H, t), 2.99 (2H, t), 3.95 (3H, s), 4.53 (2H, d), 6.10 (1H, s), 6.29 (1H, s), 6.90 (1H, s), 7.18 (1H, s), 7.83 (1H, s), 9.36 (1H, s), 11.92 (1H, s). MS m/z 413 (MH+).

4-chloro-N-[(3-methyl-1,2-oxazol-5-yl)methyl]pyrimidin-2-amine was prepared as outlined in Example 13.

5-[2-(2-methoxy-1,3-thiazol-5-yl)ethyl]-1H-pyrazol-3-amine, used as starting material, was prepared as follows:—

Acetonitrile (0.29 ml, 5.5 mmol, 2 eq) was added dropwise to a solution of lithium diisopropylamide (1.8 M in THF, 3.05 ml, 5.5 mmol, 2 eq) in THF (20 ml) at −78° C. under a nitrogen atmosphere. After stirring at -78° C. for 10 mins, methyl 3-(2-methoxy-1,3-thiazol-5-yl)propanoate (553 mg, 2.75 mmol, 1 eq) in THF (5 ml) was added dropwise. The reaction was stirred at −78° C. for 20 mins and then warmed to room temperature. Ethanol (20 ml) was added followed by hydrazine monohydrochloride (471 mg, 6.87 mmol, 2.5 eq) and the reaction was refluxed overnight. After cooling to room temperature, the volatiles were removed under reduced pressure and the residue purified by silica column chromatography eluting with 0-10% methanol in dichloromethane to afford the title compound as a pale yellow solid (401 mg, 65% yield). 1H NMR (399.902 MHz, CDCl3) δ 2.83 (2H, t), 2.96 (2H, t), 4.03 (3H, s), 5.46 (1H, s), 6.80 (1H, s). MS: m/z 225 (MH+).

Methyl 3-(2-methoxy-1,3-thiazol-5-yl)propanoate, used as starting material, was prepared as follows:—

Methyl (E)-3-(2-methoxy-1,3-thiazol-5-yl)prop-2-enoate (650 mg, 3.26 mmol, 1 eq) and 5% Pd on barium sulfate (1.63 g, 3.26 mmol) in ethanol (10 mL) were stirred under an atmosphere of hydrogen at 1 atmosphere and 25° C. for 18 h. The reaction mixture was filtered through Celite. The filtrate was evaporated under reduced pressure to afford the title compound as a pale yellow liquid (563 mg, 86% yield). ¹H NMR (399.902 MHz, CDCl3) δ2.61 (2H, t), 2.99 (2H, t), 3.70 (3H, s), 4.02 (3H, s), 6.83 (1H, s). MS: m/z 202 (MH+).

Methyl (E)-3-(2-methoxy-1,3-thiazol-5-yl)prop-2-enoate, used as starting material, was prepared as follows:—

Methyl (E)-3-(2-chloro-1,3-thiazol-5-yl)prop-2-enoate (400 mg, 1.96 mmol, 1 eq), sodium methoxide (319 mg, 5.89 mmol, 3 eq) and dry methanol (12 ml) were added into a microwave vial. The reaction mixture was heated to 120° C. in a microwave reactor for 15 mins. The procedure was repeated on exactly the same scale under exactly the same conditions and the reactions combined for work-up. The combined reactions were evaporated, the residue taken up in water (50 ml), neutralized with 2 N HCl (aq.), extracted with EtOAc (2×50 ml) and the combined organic phases dried over sodium sulfate. After filtering, the solvent was evaporated under reduced pressure to afford the title compound as a pale yellow solid (655 mg, 84% yield). 1H NMR (399.902 MHz, DMSO) 6 4.09 (3H, s), 6.05 (1H, d), 7.68 (1H, s), 7.76 (1H, d). MS: m/z 200 (MH+)

Methyl (E)-3-(2-chloro-1,3-thiazol-5-yl)prop-2-enoate, used as starting material, was prepared as follows:—

Methyl 2-triphenylphosphoranylideneacetate (3.4 g, 10. 16 mmol, 1.5 eq) was added portionwise to a stirred solution of 2-chloro-1,3-thiazole-5-carbaldehyde (1 g, 6.78 mmol, 1 eq) in DCM (20 ml) at ambient temperature and the reaction was allowed to stir overnight. The volatiles were removed under reduced pressure and the residue purified by silica column chromatography to afford the title compound as a colourless solid (1 1 53 g, 84% yield).

¹H NMR (399.902 MHz, DMSO) δ 3.74 (3H, s), 6.42 (1H, d), 7.83 (1H, d), 8.11 (1H, s)

### Kinase Assay

To determine inhibition of FGFR activity, kinase assays were conducted using ELISA (Enzyme-Linked Immunosorbent Assay) technology.

Kinase activity assays were performed in 384-well polypropylene plates (Matrix, 4311) with a total volume of 40 μl in each well. Each well was coated with 2 μg of polyEAY substrate (Sigma, P3899) at 4° C. overnight. The plates were then washed once with 100 μl PBS and once with 100 μl 50 mM HEPES (pH 7.4) prior to the addition of the kinase assay reagents. Each kinase reaction contained 0.1 ng His₆-tagged FGFR kinase domain (FGFR kinase domain (amino acids 458-765, C488A, C584S) N-terminally fused to a His₆-tag and TEV cleavage site encoded by the following sequence; [MHHHHHHEFKGSTSLYKKAGSSENLYFQGA]. The final alanine denotes the start of the FGFR protein sequence. The resultant protein was expressed and purified based on Mohammadi et al, Cell Vol 86, 577-587 (1996)), 50 mM HEPES (pH 7.4), 0.1 mM Na₃VO₄, 0.1 mM DTT, 0.05% (v/v) Triton X100, 20 mM MgCl₂, 160 μM ATP. Various concentrations of test compounds were each added in 5% (v/v) DMSO to yield a final assay DMSO concentration of 1.25% (v/v). The kinase reactions were incubated at room temperature for 45 minutes and stopped by washing the plate three times with 100 μl PBS plus 0.05% Tween. 40 μl of a one in 10000 dilution of 4G10-HRP antibody (Upstate Biotechnology, UBI 16-105) made up in 0.5% (w/v) BSA/PBS was then added to each well and the plates incubated at room temperature for one hour. Following this, the plates were then washed repeatedly with 100 μl PBS plus 0.05% Tween to remove all traces of the antibody solution. 40 μl of 50 μg/ml 3,3′,5,5′-Tetramethylbenzidine (Sigma, T2885), 0.05M phosphate-citrate buffer, containing 0.03% sodium perborate was added to each well and the plates incubated at room temperature for twelve minutes. The colour reaction was stopped by the addition of 20 μl 2M H₂SO₄ and the plates read at 450 nm on a Spectrafluor Plus (Tecan). The mean data values for each test compound concentration, untreated control wells and 100% inhibition control wells were used to determine the test compounds IC₅₀ value. IC₅₀ value is the concentration of test compound that inhibits 50% of FGFR kinase activity.

### Results of FGFR Inhibition Tests for Examples 1-11, 17-22, 24-30, and 66-73

| Example | Activity class |
|---|---|
| 1 | B |
| 2 | B |
| 3 | A |
| 4 | B |
| 5 | C |
| 6 | A |
| 7 | A |
| 8 | A |
| 9 | A |
| 10 | A |
| 11 | A |
| 17 | A |
| 18 | A |
| 19 | B |
| 20 | B |
| 21 | A |
| 22 | B |
| 24 | B |
| 25 | B |
| 26 | B |
| 27 | A |
| 28 | B |
| 29 | A |
| 30 | B |
| 66 | A |
| 67 | A |
| 68 | A |
| 69 | A |
| 70 | A |
| 71 | A |
| 72 | B |
| 73 | A |

Activity:
A less than 0.1 μM
B greater than 0.1 μM and less than 1 μM
C greater than 1 μM and less than 10 μM

### For example, Example 33 was measured to have an IC₅₀ of 92 nM

### Kinase Assay (Using Caliper Technology)

To determine inhibition of FGFR activity, kinase assays were conducted using Caliper technology.

Kinase activity assays were performed in Greiner 384-well low volume plates, with a total reaction volume of 12ul per well. Final concentration of FGFR1 active kinase in each reaction well was 7.2 nM. The substrate for each assay was a custom peptide with fluorescent tag (13 amino acids in length) the sequence of which was specific for FGFR1 kinase.

Compounds were serially diluted in 5% (v/v) DMSO, before being added to assay plates. The Enzyme (at 7.2 nM [final]) and Substrate (at 3.6 uM [final]) were added separately to the compound plates, in reaction buffer [comprising: 50mM MOPS—pH 6.5, 0.004% Triton, 2.4 mM DTT, 12 mM MgCl₂, 408 uM ATP]resulting in a final DMSO concentration in the reaction mix of 0.8%.

Assay plates were incubated at room temperature for 1.5 h, before the reaction was stopped with the addition of buffer [comprising: 100 mM HEPES—pH7.5, 0.033% Brij-35, 0.22% Caliper Coating Reagent #3, 88mM EDTA, 5% DMSO]. Stopped assay plates were then read using the Caliper LabChip® LC₃₀₀₀ (which uses microfludics to measure a shift in mobility between fluorescent labelled peptide and the FGFR1 kinase—phosphorylated form of this peptide).

The mean data values for each compound concentration, untreated control wells and 100% inhibition control wells were used to determine the IC₅₀ for each test compound. The IC₅₀ is the concentration of compound, which inhibits FGFR¹ kinase activity by 50% in the context of this assay.

The following compounds were tested in this assay and exhibited an IC50 of:—
Less than 30 μM 37, 142;
with the following being <10 μM 34, 35, 36, 38, 39, 49, 51, 55, 134, 143, 74, 75, 81, 85, 87, 90, 92, 95, 96, 129, 98, 99, 100, 114, 116, 119;
with the following being <1 μM 23, 24, 25, 26, 31, 32, 40, 45, 47, 48, 50, 53, 54, 57, 58, 59, 60, 62, 64, 122, 123, 127, 136, 138, 80, 83, 88, 89, 93, 94, 101, 137, 104, 105, 106, 109, 115, 117, 118, 121, 130;
with the following being <200 nM 27, 28, 29, 30, 33, 41, 42, 43, 44, 14, 15, 16, 52, 56, 61, 63, 65, 124, 125, 126, 128, 132, 133, 141, 66, 67, 68, 69, 70, 71, 73, 78, 79, 82, 84, 86, 91, 102, 103, 131, 135, 107, 108, 110-113, 120.

### Growth Factor Stimulated Erk Phosphorylation

These and other assays were used to evaluate the ability of a test compound to inhibit growth factor stimulated cellular signalling in mammalian cell lines. This was achieved by measuring the amount of receptor tyrosine kinase regulated Erk phosphorylation within a cell following compound treatment.

NIH 3T3 (ECACC, 93061524) cells were routinely passaged in DMEM (Gibco BRL, 41966) plus 10% foetal calf serum (FCS), 1% L-glutamine (Gibco BRL, 25030) to a confluence not greater than 80%. To undertake the assay, NIH 3T3's were seeded at 1×10⁴ cells/well in DMEM plus 10% foetal calf serum, 1% L-glutamine in 96 well plates (Costar, 3904) and incubated at 37° C. (+5% CO₂) in a humidified incubator. Once the cells had fully adhered (typically following 4-5 hours incubation) the media was removed from each well and the cells gently washed with 100μl warm serum free media. 90μl of serum free DMEM plus 1% L-glutamine was then added to each well and the plates were returned to a humidified 37° C. (+5% CO₂) incubator. The following day, the plates were dosed with 10[l compound (diluted from 10 mM stock in DMSO using serum free DMEM) and the plates were returned to a humidified 37° C. (+5% CO₂) incubator for one hour. NIH 3T3 cells were then stimulated with a final concentration of 3 ng/ml bFGF (Sigma, F029 1) for 20 minutes at 37° C. Following stimulation the cells were fixed by adding formaldehyde (4% v/v final concentration) and incubating at room temperature for 20 minutes. The fixative solution was then removed and the wells were washed twice with 100μl phosphate buffered saline (PBS/A) before permeabilising the cells by the addition of 50 μl/well 0.1% triton/PBS/A for 10 minutes at room temperature. The permeabilisation solution was then removed and the cells washed twice more with 100 μl/well PBS/A before the addition of 50 μl/well anti-phospho p44/42 (Cell Signalling Technology, 9106), diluted 1/500 with PBS/A plus 10% FCS. The anti-phospho p44/42 antibody recognises Erk phosphorylated at threonine 202 and tyrosine 204. Following incubation at room temperature for 2 hours, the antibody solution was removed and the wells were washed twice with 100 μl/well PBS/A. 50 μl/well 1/250 goat anti-mouse alexa fluor 488 secondary antibody (Molecular Probes, A11001) and 1/10000 Hoescht (Molecular Probes, H-3570) diluted with PBS/A plus 10% FCS was added and the plate incubated in the dark at room temperature for one hour. Finally, the plates were washed three times with 100 μl/well PBS/A, leaving the final wash in the wells before sealing the plates. The plates were read at 350 nm and 488 nm using an Arrayscan (Cellomics). The mean average intensity fluorescence values for each test compound concentration, untreated control wells and 100% inhibition control wells were used to determine the test compounds IC₅₀ value. IC₅₀ value is the concentration of test compound that inhibits 50% of Erk phosphorylation.

The following compounds were tested in this assay and exhibited an IC50 of:—
with the following being <30 μM 118;
with the following being <10 μM 31, 34, 37, 46, 48, 51, 55, 79, 80, 81, 83, 85, 87, 88, 90, 95, 96, 98, 100, 109, 112, 113, 114, 115;
with the following being <1 μM 1, 23, 33, 35, 38, 39, 40, 43, 47, 53, 54, 72, 74, 76, 77, 78, 82, 86, 89, 92, 104, 105, 106, 107, 108, 110;
with the following being <200 nM 3, 41, 42, 44, 52, 53, 66, 67, 73, 84, 91, 93, 94, 97, 111.

For example, Example 33 was measured to have an IC₅₀ of 518 nM

### Cell Based Inhibition of Transiently Expressed FGFR1 IIIc Phosphorylation (Measured Using Phospho-Specific Primary and Fluorescent Secondary Antibodies).

This assay is designed to detect inhibitors of transiently expressed FGFR1 phosphorylation by antibody staining of fixed cells detected using ArrayScan technology.

Cos-1 cells were routinely passaged in DMEM (Gibco BRL, 41966) plus 3% foetal calf serum (FCS), 1% L-glutamine (Gibco BRL, 25030) to a confluence of 80%. To undertake the assay, Cos-1 cells were harvested at 90-95% confluence for cell transfection. For each 96-well plate, 24 ul Lipofectamine 2000 was added to 809 ul OptiMEM and incubated at room temperature for 5 minutes. For each 96 well plate, 20 ug 3′ FLAG tagged FGFR1/pcDNA3.1 (In-house clone15, MSD 4793) was diluted with OptiMEM to a total volume of 833 ul. Equal volumes of DNA and Lipofectamine 2000 were combined (DNA: Lipid=1:1.2 ratio) and incubated at room temperature for 20 minutes.

The harvested Cos-1 cells are counted using a coulter counter and diluted further with 1% FCS/DMEM to 2.5×10⁵ cells/ml. For each 96-well, 8.33 ml cells were required. The complexed transfection solution was added to the cell solution and the cells were seeded at 2.5×10⁵ cells/well in DMEM plus 1% foetal calf serum, 1% L-glutamine in 96 well plates (Costar, 3904) and incubated at 37° C. (+5% CO₂) in a humidified incubator overnight (24 hrs). The following day, the plates were dosed with 25 μl compound (diluted from 10 mM stock in DMSO using serum free DMEM) and the plates were returned to a humidified 37° C. (+5% CO₂) incubator for one hour. Media was removed from the wells using vacuum aspiration; cells were fixed by adding 50μl of 100% methanol to each well and incubated at room temperature for 20 minutes. The fixative solution was then removed and the wells were washed once with 200 μl phosphate buffered saline (PBS/A) before permeabilising the cells by the addition of 50 ul/well 0.1% triton/PBS/A for 20 minutes at room temperature. The permeabilisation solution was then removed and the cells washed once more with 200 ul/well PBS/A before the addition of 40μl 1/1000 primary antibody solution (Cell Signalling Technologies #CS3476; mouse anti-phospho FGFR1 diluted in PBS/A with 10% FCS+0.1% Tween20) to each well.

Following incubation at room temperature for 1 hour, the antibody solution was removed and the wells were washed once with 200 ul/well PBS/A. Then 40 μl 1/500 secondary antibody (A11005; goat anti-mouse 594) solution and 1/10000 Hoechst (diluted together in PBS/A with 10% FCS+0.1% Tween 20) were added and the plate incubated in the dark at room temperature for one hour. Finally, the plates were washed once with 200[l/well PBS/A, leaving the final wash in the wells before sealing the plates. The plates were read on an Arrayscan (Cellomics). The Channel 2 (594 nm) values obtained from undosed (max) and reference compound (min) wells within a plate are used to set boundaries for 0% and 100% compound inhibition. Compound data was normalized against these values to determine the dilution range of a test compound that gives 50% inhibition of phosphorylated FGFR1.

The following compounds were tested in this assay and exhibited an IC50 of:—
Less than 30 μM 5, 58, 59, 60, 116, 118, 119, 121;
with the following being <10μM, 29, 31, 34, 38, 39, 40, 43, 45, 46, 48, 49, 51, 63, 64, 65, 78, 88, 95, 100, 105, 108, 109, 113, 128;
with the following being <1 μM 3, 15, 16, 24, 30, 41, 47, 52, 53, 54, 61, 62, 66, 91, 93, 94, 110, 111, 120;
with the following being <200 nM, 13, 14, 27, 28, 42, 56, 57, 67, 73, 97, 102, 103.

### Cell Based Inhibition of Transiently Expressed FGFR1 IIIc Phosphorylation Via Use of ECHO Technology (Measured Using Phospho-Specific Primary and Fluorescent Secondary Antibodies).

This assay is designed to detect inhibitors of transiently expressed FGFR1 phosphorylation by antibody staining of fixed cells detected using ArrayScan technology.

Cos-1 cells were routinely passaged in DMEM (Gibco BRL, 41966) plus 3% foetal calf serum (FCS), 1% L-glutamine (Gibco BRL, 25030) to a confluence of 80%. To undertake the assay, Cos-1 cells were harvested at 90-95% confluence for cell transfection. For each 96-well plate, 24μl Lipofectamine 2000 was added to 809 ul OptiMEM and incubated at room temperature for 5 minutes. For each 96 well plate, 20 ug 3° FLAG tagged FGFR1/pcDNA3.1 (In-house clone15, MSD 4793) was diluted with OptiMEM to a total volume of 833 μl. Equal volumes of DNA and Lipofectamine 2000 were combined (DNA: Lipid=1:1.2 ratio) and incubated at room temperature for 20 minutes.

The harvested Cos-1 cells are counted using a coulter counter and diluted further with 1% FCS/DMEM to 2.5×10⁵ cells/ml. For each 96-well, 8.33 ml cells were required. The complexed transfection solution was added to the cell solution and the cells were seeded at 2.5×10⁵ cells/well in DMEM plus 1% foetal calf serum, 1% L-glutamine in 96 well plates (Costar, 3904) and incubated at 37° C. (+5% CO₂) in a humidified incubator overnight (24 hrs). The following day, compounds from dry weight samples were dissolved in 100% DMSO to give 10 mM concentration. 40μl of the compound was dispensed into the wells of each quadrant across the 384 Labcyte plate (inclusive of a positive control (100% DMSO), a negative control (10 μM) and a reference compound (250 nM)). The 384 Labcyte plate was then transferred to the Hydra to dilute the compounds 1:100 into the remaining wells of the quadrant. 70 μl of media was aspirated from the assay plate using the Quadra before the plate was transferred onto the ECHO 550. The 384 Labcyte compound plate was also transferred onto the ECHO 550. Compound transfer to the assay plate on the ECHO 550 was at concentration ranges 1)10 μM, 2) 3 μM, 3) 1 μM, 4) 0.3 μM, 5) 0.1 μM, 6) 0.01.

The plates were gently tapped to mix compound in with the cell media and left to incubate at 37° C. with 5% CO₂ for 1 hour.

Media was removed from the wells using vacuum aspiration; cells were fixed by adding 50 μl of 100% methanol to each well and incubated at room temperature for 20 minutes. The fixative solution was then removed and the wells were washed once with 200 μl phosphate buffered saline (PBS/A) before permeabilising the cells by the addition of 50 ul/well 0.1% triton/PBS/A for 20 minutes at room temperature. The permeabilisation solution was then removed and the cells washed once more with 200μl/well PBS/A before the addition of 40 μl 1/1000 primary antibody solution (Cell Signalling Technologies #CS3476; mouse anti-phospho FGFR1 diluted in PBS/A with 10% FCS+0.1% Tween20) to each well.

Following incubation at room temperature for 1 hour, the antibody solution was removed and the wells were washed once with 200 ul/well PBS/A. Then 40 μl 1/500 secondary antibody (A11005; goat anti-mouse 594) solution and 1/10000 Hoechst (diluted together in PBS/A with 10% FCS+0.1% Tween 20) were added and the plate incubated in the dark at room temperature for one hour. Finally, the plates were washed once with 200 μl/well PBS/A, leaving the final wash in the wells before sealing the plates. The plates were read on an Arrayscan (Cellomics). The Channel 2 (594 nm) values obtained from undosed (max) and reference compound (min) wells within a plate are used to set boundaries for 0% and 100% compound inhibition. Compound data was normalized against these values to determine the dilution range of a test compound that gives 50% inhibition of phosphorylated FGFR1.

The following compounds were tested in this assay and exhibited an IC50 of:—
Less than 30 μM
5, 19, 22, 36, 58, 59, 127, 134, 137, 139, 143;
with the following being <10 μM
4, 17, 20, 26, 50, 63, 64, 65, 79, 123, 128, 130, 133, 136, 138, 140, 142;
with the following being <1 μM
2, 3, 8, 11, 13, 18, 21, 32, 41, 44, 52, 57, 62, 66, 82, 84, 91, 93, 101, 122, 125, 129, 132, 135, 141;
with the following being <200 nM
6, 7, 10, 14, 15, 16, 25, 28, 42, 56, 67, 68, 69, 70, 71, 73, 94, 97, 102, 103, 111, 120, 124, 126, 131.

### Inhibition of Insulin-like Growth Factor-1 Receptor Phosphorylation

This immunofluorescence end point cell assay measures the ability of a test compound to reduce the measured levels of IGF1R phosphorylation after IGF1 stimulation in R⁺ cells. R⁻ cells were derived by transfection of R⁻ mouse fibroblast cells with human IGF1R. R⁺ cells were routinely cultured in DMEM growth medium (Gibco BRL, 41966) containing 2 mM L-Glutamine (Invitrogen Code no. 25030-024) and 10% (v/v) foetal bovine serum (FBS)) in a 5% CO₂ air incubator at 37° C.

To undertake the assay, the R⁺ cells were seeded at 5×10³ cells/well in DMEM plus 1% foetal calf serum, 1% L-glutamine in 96-well black Packard View plates (PerkinElmer 6005182) and incubated at 37° C. (+5% CO₂) in a humidified incubator. The following day, the plates were dosed with 10μl of 10× concentrated compound (diluted from 10 mM stock in DMSO and DMEM without serum) and the plates were e returned to a humidified 37° C. (+5% CO₂) incubator for 30 minutes. Cells were tested in duplicates in a suitable dose range to accurately measure the compound IC50.

Following the compound treatment, the R⁺ cells were stimulated with a final concentration of 30 nM IGF1 (Gropep 1M001) for 20 minutes at 37° C. The IGF1 was dissolved according to the manufacture's instructions to a 26 μM stock solution and diluted in DMEM without serum. Following stimulation, the cells were fixed by adding formaldehyde (4% v/v final concentration) and incubated at room temperature for 20 minutes. The fixative solution was removed and the wells were washed twice with 100 μl phosphate buffered saline containing 0.05% Tween20 (PBS-Tween 20) before permeabilisation of the cells by the addition of 50 μl/well 0.05% Triton in PBS for 10 minutes at room temperature. The permeabilisation solution was removed and the cells were washed twice with 100μl/well PBS-Tween 20 before addition of 50 μl blocking solution containing 2% BSA (Sigma. A-78888)+2% goat serum (DAKO X0907 ) in PBS. Plates were incubated for 1 hour at room temperature. The blocking solution was aspirated from the wells and 50 μl rabbit dual phospho specific anti-phospho IGF1R/IR (BioSource 44-804) 1/350 diluted in blocking solution was added to the wells. Additionally, in-house antibodies raised against phospho TGF1R were also used at a suitable titre determined for each batch.

Following incubation at room temperature for 1 hour, the antibody solution was removed and the wells washed twice with 100 μl/well PBS-Tween 20. 50 μl/well Alexa Fluor conjugated anti rabbit (Invitrogen/Molecular Probes-A11008) was added to the wells in a dilution of 1/1000 in blocking solution. The plates were incubated at room temperature for one hour. Finally, the plates were washed three times with 100 μl/well PBS-Tween. After addition of 100 μl/well PBS the plates were sealed with a black seal.

The Green Fluorescent phospho IGF1R -associated signal in each well was measured using an Acumen Explorer HTS Reader (TTP Labtech Ltd., Cambridge). Phospho IGF1R-associated fluorescence emission can be detected at 530 nm following excitation at 488 nm. The instrument is a laser-scanning fluorescence microplate cytometer, which samples the well at regular intervals and uses threshold algorithms to identify all fluorescent intensities above the solution background without the need to generate and analyse an image. These fluorescent objects can be quantified and provide a measure of the phospho IGF1R levels in cells. Fluorescence dose response data obtained with each compound was exported into a suitable software package (such as Origin) to perform curve fitting analysis. Phospho-IGF1R levels in response to compound treatment versus stimulated and unstimulated controls were expressed as an IC₅₀ value. This was determined by calculation of the concentration of compound that was required to give a 50% reduction of the maximum phospho-IGF1R signal.

### Results of IGFR Inhibition Tests for Examples 1, 3, 4, 9-11, 17, 18, 27, 66-68 and 70

| Example No. | IGF cell class |
|---|---|
| 1 | D |
| 3 | C |
| 4 | D |
| 6 | B |
| 9 | C |
| 10 | B |
| 11 | C |
| 17 | D |
| 18 | C |
| 24 | D |
| 27 | D |
| 29 | D |
| 30 | D |
| 31 | D |
| 32 | C |
| 33 | D |
| 34 | D |
| 35 | D |
| 36 | D |
| 37 | C |
| 38 | B |
| 39 | D |
| 40 | C |
| 41 | C |
| 42 | C |
| 43 | D |
| 44 | D |
| 46 | C |
| 47 | C |
| 48 | D |
| 50 | D |
| 51 | D |
| 52 | D |
| 53 | D |
| 54 | C |
| 55 | D |
| 56 | C |
| 60 | D |
| 61 | D |
| 62 | D |
| 65 | C |
| 66 | D |
| 67 | C |
| 68 | D |
| 70 | D |
| 73 | C |
| 74 | D |
| 75 | D |
| 76 | D |
| 87 | D |
| 88 | D |
| 89 | C |
| 90 | D |
| 91 | D |
| 92 | D |
| 93 | D |
| 94 | D |
| 95 | D |
| 96 | D |
| 97 | C |
| 99 | D |
| 100 | C |
| 101 | D |
| 102 | B |
| 103 | C |
| 104 | C |
| 105 | D |
| 106 | C |
| 107 | D |
| 109 | D |
| 110 | C |
| 111 | C |
| 113 | C |
| 114 | D |
| 115 | D |
| 116 | D |
| 118 | D |
| 120 | C |

Activity:
A less than 0.1 μM
B greater than 0.1 μM and less than 1 μM
C greater than 1 μM and less than 10 μM
D greater than 10 μM

Conclusion: Although the compounds tested show some inhibition of IGFR in cells, the compounds show reduced potency against IGFR than the much higher levels of potency against FGFR as demonstrated in the enzyme assay results. Reduced inhibition of IGFR is desirable to ameliorate potential effects upon insulin or growth factor production.

### Cytochrome P450 Inhibition Assay

The inhibitory potential (IC₅₀) of test compounds against 5 human cytochrome P450 (CYP) isoforms (IA2, 2C9, 2C19, 3A4 and 2D6) was assessed using an automated fluorescent end point in vitro assay modified from Crespi (Crespi and Stresser, 2000). Microsomal subcellular fractions prepared from Yeast cell lines expressing each human CYP isoform were used as an enzyme source in this assay. The activity of the 5 major human CYPs was determined from the biotransformation of a number of coumarin substrates to fluorescent metabolites, in the presence of NADPH. Inhibition of these CYPs resulted in a decrease in the amount of fluorescent metabolite formed. Comparison of the fluorescence observed in the presence of varying concentrations of test compound with that seen in its absence allowed an IC₅₀ value to be calculated. Initial experiments were performed to optimise the kinetic parameters of the assay and these have been listed in Table 1. Stock solutions of each CYP, with its respective substrate, were prepared in phosphate buffer pH7.4 (see Table 1) and 178 μl was added to the well of a black solid, flat bottom, 300 μl 96 well microtitre plate (Coming Costar). Test compounds were serially diluted in DMSO/acetonitrile and added (2 μl) to the reaction to give final concentrations of 0.1, 0.3, 1, 3 and 10 μM. After pre-incubating at 37° C. for 5 min the reactions were started with addition of NADPH (20 μl, concentration shown in Table 1). The final solvent content in each incubation was <=2% (1% from the test compound and a maximum of 1% from the substrate). The appropriate solvent controls and substrate blanks were included in each experiment to assess control activity and identify any inherent fluorescence due to the test compounds. In addition, known inhibitors of each CYP were included as positive controls (see Table 3 for inhibitor concentrations and expected IC₅₀ ranges). The reactions were stopped at defined timepoints (see Table 1) by quenching with 100 μl of solvent (acetonitrile:0.5M Tris buffer 80:20 v/v). The plates were read on a fluorimeter (Spectrafluor Plus) at the appropriate excitation and emission wavelengths (listed in Table 2) and the percent activity, corrected for control, was plotted against the test compound concentration. The IC₅₀ (the concentration of test compound required to cause 50% inhibition of metabolic activity) for each CYP was then determined from the slope of these plots.

TABLE 1
Concentrations of assay reagents and assay conditions.

| | CYP | | | Phos- | | Incu- |
|---|---|---|---|---|---|---|
| | solution | | Sub- | phate | | bation |
| | (pmol/ | | strate | Buffer | NADPH | time |
| CYP | 200 μl) | Substrate | (uM) | (M) | (μM) | (min) |
| 1A2 | 1 | 3-cyano-7- | 3 | 0.1 | 250 | 20 |
| | | ethoxy-coumarin | | | | |
| | | (CEC) | | | | |
| 2C9 | 3 | 7-methoxy-4- | 50 | 0.025 | 250 | 40 |
| | | trifluoromethyl- | | | | |
| | | coumarin (MFC) | | | | |
| 2C19 | 5 | 7-methoxy-4- | 50 | 0.05 | 250 | 60 |
| | | trifluoromethyl- | | | | |
| | | coumarin (MFC) | | | | |
| 2D6 | 3 | 7-methoxy-4- | 20 | 0.1 | 60 | 35 |
| | | (aminomethyl)- | | | | |
| | | coumarin | | | | |
| | | (MAMC) | | | | |
| 3A4 | 5 | 7-benzyloxy-4- | 15 | 0.1 | 250 | 35 |
| | | (trifluoromethyl)- | | | | |
| | | coumarin (BFC) | | | | |

TABLE 2
Excitation and emission wavelengths used by Spectrafluor
Plus Fluorimeter to detect fluorometric metabolites. CEC
and HFC were obtained from Ultrafine Chemicals; CHC was
obtained from Molecular Probes; MFC, MAMC, HAMC and
BFC were obtained from Gentest Corporation.

| | | | Excitation | Emission |
|---|---|---|---|---|
| CYP | Substrate | Metabolite | λ (nm) | λ (nm) |
| 1A2 | 3-cyano-7-ethoxy- | 3-cyano-7-hydroxy- | 405 | 460 |
| | coumarin (CEC) | coumarin (CHC) | | |
| 2C9 | 7-methoxy-4- | 7-hydroxy-4- | 405 | 535 |
| | trifluoromethyl- | trifluoromethyl- | | |
| | coumarin (MFC) | coumarin (HFC) | | |
| 2C19 | 7-methoxy-4- | 7-hydroxy-4- | 405 | 535 |
| | trifluoromethyl- | trifluoromethyl- | | |
| | coumarin (MFC) | coumarin (HFC) | | |
| 2D6 | 7-methoxy-4- | 7-hydroxy-4- | 390 | 460 |
| | (aminomethyl)- | (aminomethyl)- | | |
| | coumarin (MAMC) | coumarin (HAMC) | | |
| 3A4 | 7-benzyloxy-4- | 7-hydroxy-4- | 405 | 535 |
| | (trifluoromethyl)- | trifluoromethyl- | | |
| | coumarin (BFC) | coumarin (HFC) | | |

TABLE 3
Known inhibitors and optimised experimental conditions
for each of the 5 human CYP isoforms. Fluvoxamine was
obtained from Tocris Cookson Ltd; Sulphaphenazole and
Quinidine were obtained from Sigma; Omeprazole
was obtained from AstraZeneca; Ketoconazole was
obtained from Ultrafine Chemicals.

| | | Range of standard | |
|---|---|---|---|
| | Substrate | inhibitor concentrations | IC₅₀ range |
| CYP | (μM) | (μM) | (μM) |
| 1A2 | 3 | Fluvoxamine | 0.01-0.07 |
| | | 1, 0.3, 0.1, 0.03, 0.01 | |
| 2C9 | 50 | Sulphaphenazole | 0.1-1.0 |
| | | 10, 3, 1, 0.3, 0.1 | |
| 2C19 | 50 | Omeprazole | 1.5-4.6 |
| | | 10, 3, 1, 0.3, 0.1 | |
| 2D6 | 20 | Quinidine | 0.003-0.03 |
| | | 0.1, 0.03, 0.01, 0.003, | |
| | | 0.001 | |
| 3A4 | 15 | Ketoconazole | 0.005-0.015 |
| | | 0.25, 0.075, | |
| | | 0.025, 0.0075, | |
| | | 0.0025 | |

### REFERENCE

Crespi C L, Stresser, D M., Fluorometric screening for metabolism-based drug-drug interactions. J Pharmacol Toxicol Methods. 2000, 44 (1): 325-31.

### Comparative Testing of Examples 1 and 9.

| | Fgf | Ic50 | Ic50 | Ic50 | Ic50 | Ic50 |
|---|---|---|---|---|---|---|
| | Ic50 | 1A2 | 2C9 | 2C19 | 2D6 | 3A4 |
| Comparative | | | | | | |
| Example | | | | | | |
| (a) | 0.14 | 0.79 | 10 | 10 | 10 | 3.31 |
| (b) | 0.36 | 0.46 | 1.12 | 10 | 10 | 4.40 |
| (c) | 0.03 | 0.1 | 1.98 | 9.06 | 10 | 0.22 |
| (d) | 0.09 | 0.1 | 3.08 | 2.88 | 10 | 0.37 |
| Examples | | | | | | |
| 1 | 0.21 | 10 | 10 | 10 | 10 | 5.70 |
| 9 | 0.04 | 2.19 | 10 | 10 | 10 | 10 |

Compounds described in Examples 1 and 9 were tested against compounds known IGFR inhibitors (as described in WO03/048133).

Comparative Example (a) is 5-bromo-N-[(3-methylisoxazol-5-yl)methyl]-N′-(5-methyl-2H-pyrazol-3-yl)pyrimidine-2,4-diamine (WO03/048133, Example 1)

Comparitive Example (b) is 5-chloro-N-[(3-methylisoxazol-5-yl)methyl]-N′-(5-methyl-2H-pyrazol-3-yl)pyrimidine-2,4-diamine (WO03/048133, Example 2)

Comparative Example (c) is 5-bromo-N′-(5-cyclopropyl-2H-pyrazol-3-yl)-N-[(3-methylisoxazol-5-yl)methyl]pyrimidine-2,4-diamine (WO03/048133, Example 3)

Comparative Example (d) is 5-bromo-N-[(3-methylisoxazol-5-yl)methyl]-N′-(5-propyl-2H-pyrazol-3-yl)pyrimidine-2,4-diamine (WO03/048133, Example 47)

Conclusion: Compounds of the present invention (Example 1 and 9) while showing good FGFR inhibition, also show decreased Cytochrome P₄₅₀ inhibition when compared to known IGF inhibitors. Low inhibition of Cytochrome P₄₅₀ is desirable to ameliorate potential drug:drug interactions.

1. A compound of formula (I): 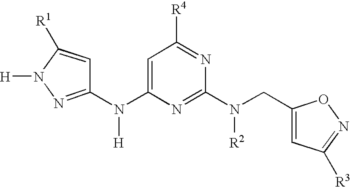
wherein
R¹ represents a C₁-C₆alkyl group optionally substituted by one or more substituents selected from C₁-C₆alkoxy, C₃-C₆cycloalkyl, C₁-C₆alkylthio, —NR⁵R⁶, —C(O)NR⁷R⁸, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), cyano and hydroxyl, a C₃-C₅cycloalkyl group optionally substituted by one or more substituents selected from C₁-C₆alkoxy, C₃-C₆cycloalkyl, C₁-C₆alkylthio, —NR⁹R¹⁰, —C(O)NR¹¹R¹² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a C₂-C₆alkenyl group optionally substituted by one or more substituents selected from C₁-C₆alkoxy, C₃-C₆cycloalkyl, C₁-C₆alkylthio, —NR¹³R¹⁴, —C(O)NR¹⁵R¹⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a 4- to 6-membered heterocyclyl group optionally substituted with by one or more substituents selected from C₁-C₆alkyl, C₁-C₆alkoxy, C₃-C₆cycloalkyl, C₁-C₆alkylthio, —NR¹⁷R¹⁸, —C(O)NR¹⁹R²⁰, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₘC₁-C₆alkyl, —NR²¹R²², —C(O)NR²³R²⁴, —SO₂NR²⁵R²⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₁-C₆alkoxy group optionally substituted by one or more substituents selected from C₁-C₆alkoxy, C₆-aryloxy, C₃-C₆cycloalkyl, —NR²⁷R²⁸, —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —OSO₂C₁₋₆alkyl, —NR³¹R³², —C(O)NR³³R³⁴, —NHC(O)OC₁₋₆alkyl, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₃-C₁₂carbocyclyloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₚC₁-C₆alkyl, —NR³⁷R³⁸, —C(O)NR³⁹R⁴⁰, —SO₂NR⁴¹R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a 5- to 6-membered heterocyclyloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl, a —S(O)ₓR⁴⁹ group, a —S(O)₂NR⁵⁰R⁵¹ group, or -A-B;
R² represents hydrogen or a C₁-C₃alkyl group optionally substituted by one or more substituents selected from C₁-C₃alkoxy, cyano, hydroxyl, amino (—NH₂), mono-C₁-C₃alkylamino and di-(C₁-C₃alky)amino;
R⁴ represents hydrogen, a C₁-C₆alkyl group optionally substituted by one or more substituents selected from C₁-C₃alkoxy, hydroxyl, amino (—NH₂), mono-C₁-C₃alkylamino and di-(C₁-C₃alkyl)amino, a C₁-C₆alkenyl group optionally substituted with C₁-C₃alkoxy, a C₁-C₆alkynyl group optionally substituted with C₁-C₃alkoxy, a C₃-C₅cycloalkyl group optionally substituted with C₁-C₃alkoxy, a C₁-C₆alkoxy group optionally substituted with C₁-C₃alkoxy, hydroxyl, amino (—NH₂), mono-C₁-C₃alkylamino and di-(C₁-C₃alkyl)amino, —C(O)NR⁵²R⁵³, —NR⁵⁴R⁵⁵, —S(O)_{y}R⁵⁶;
A represents a C₂-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or a C₁-alkyleneoxy optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), 25 mono- and di-C₁-C₆alkylamino, hydroxyl and trifluoromethyl), and hydroxyl, or an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be 30 optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl;
B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, C₁-C₆alkyloxycarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₃₋₅cycloalkyl, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring;
m is 0, 1 or 2;
n is 0, 1 or 2;
p is 0, 1 or 2;
r is 0, 1 or 2;
s is 0, 1 or 2
x is 0, 1 or 2;
y is 0, 1 or 2;
R⁵ and R⁶ each independently represent hydrogen, C₁-C₄alkyl or C₃-C₆cycloalkyl, or R⁵ and R⁶ together with the nitrogen atom to which they are attached form a 4- to 6-membered saturated heterocycle;
R⁷ and R⁸ each independently represent hydrogen, C₁-C₄alkyl or C₃-C₆cycloalkyl, or R⁷ and R⁸ together with the nitrogen atom to which they are attached form a 4- to 6-membered saturated heterocycle;
R⁹ and R¹⁰ each independently represent hydrogen, C₁-C₄alkyl or C₃-C₆cycloalkyl, or R⁹ and R¹⁰ together with the nitrogen atom to which they are attached form a 4- to 6-membered saturated heterocycle;
R¹¹ and R¹² each independently represent hydrogen, C₁-C₄alkyl or C₃-C₆cycloalkyl, or R¹¹ and R¹² together with the nitrogen atom to which they are attached form a 4- to 6-membered saturated heterocycle;
R¹³ and R¹⁴ each independently represent hydrogen, C₁-C₄alkyl or C₃-C₆cycloalkyl, or R¹³ and R¹⁴ together with the nitrogen atom to which they are attached form a 4- to 6-membered saturated heterocycle;
R¹⁵ and R¹⁶ each independently represent hydrogen, C₁-C₄alkyl or C₃-C₆cycloalkyl, or R¹⁵ and R¹⁶ together with the nitrogen atom to which they are attached form a 4- to 6-membered saturated heterocycle;
R¹⁷ and R¹⁸ each independently represent hydrogen, C₁-C₄alkyl or C₃-C₆cycloalkyl, or R¹⁷ and R¹⁸ together with the nitrogen atom to which they are attached form a 4- to 6-membered saturated heterocycle;
R¹⁹ and R²⁰ each independently represent hydrogen, C₁-C₄alkyl or C₃-C₆cycloalkyl, or R¹⁹ and R²⁰ together with the nitrogen atom to which they are attached form a 4- to 6-membered saturated heterocycle;
R²¹ and R²² each independently represent hydrogen, C₁-C₄alkyl or C₃-C₆cycloalkyl, or R²¹ and R²² together with the nitrogen atom to which they are attached form a 4- to 6-membered saturated heterocycle;
R²³ and R²⁴ each independently represent hydrogen, C₁-C₄alkyl or C₃-C₆cycloalkyl, or R²³ and R²⁴ together with the nitrogen atom to which they are attached form a 4- to 6-membered saturated heterocycle;
R²⁵ and R²⁶ each independently represent hydrogen, C₁-C₄alkyl or C₃-C₆cycloalkyl, or R²⁵ and R²⁶ together with the nitrogen atom to which they are attached form a 4- to 6-membered saturated heterocycle;
R²⁷ and R²⁸ each independently represent hydrogen, C₁-C₄alkyl or C₃-C₆cycloalkyl, or R²⁷ and R²⁸ together with the nitrogen atom to which they are attached form a 4- to 6-membered saturated heterocycle;
R²⁹ and R³⁰ each independently represent hydrogen, C₁-C₄alkyl or C₃-C₆cycloalkyl, or R²⁹ andR³⁰ together with the nitrogen atom to which they are attached form a 4- to 6-membered saturated heterocycle;
R³¹ and R³² each independently represent hydrogen, C₁-C₆alkyl or C₃-C₆cycloalkyl, or R³¹ and R³² together with the nitrogen atom to which they are attached form a 4- to 6-membered saturated heterocycle optionally comprising an additional heteratom selected from oxygen, sulphur or nitrogen;
R³³ and R³⁴ each independently represent hydrogen, C₁-C₄alkyl or C₃-C₆cycloalkyl, or R³³ and R³⁴ together with the nitrogen atom to which they are attached form a 4- to 6-membered saturated heterocycle optionally comprising an additional heteratom selected from oxygen, sulphur or nitrogen;
R³⁵ and R³⁶ each independently represent hydrogen, C₁-C₄alkyl or C₃-C₆cycloalkyl, or R³⁵ and R³⁶ together with the nitrogen atom to which they are attached form a 4- to 6-membered saturated heterocycle;
R³⁷ and R³⁸ each independently represent hydrogen, C₁-C₄alkyl or C₃-C₆cycloalkyl, or R³⁷ and R³⁸ together with the nitrogen atom to which they are attached form a 4- to 6-membered saturated heterocycle;
R³⁹ and R⁴⁰ each independently represent hydrogen, C₁-C₄alkyl or C₃-C₆cycloalkyl, or R³⁹ and R⁴⁰ together with the nitrogen atom to which they are attached form a 4- to 6-membered saturated heterocycle;
R⁴¹ and R⁴² each independently represent hydrogen, C₁-C₄alkyl or C₃-C₆cycloalkyl, or R⁴¹ and R⁴² together with the nitrogen atom to which they are attached form a 4- to 6-membered saturated heterocycle;
R⁴³ and R⁴⁴ each independently represent hydrogen, C₁-C₄alkyl or C₃-C₆cycloalkyl, or R⁴³ and R⁴⁴ together with the nitrogen atom to which they are attached form a 4- to 6-membered saturated heterocycle;
R⁴⁵ and R⁴⁶ each independently represent hydrogen, C₁-C₄alkyl or C₃-C₆cycloalkyl, or R⁴⁵ and R⁴⁶ together with the nitrogen atom to which they are attached form a 4- to 6-membered saturated heterocycle;
R⁴⁷ and R⁴⁸ each independently represent hydrogen, C₁-C₄alkyl or C₃-C₆cycloalkyl, or R⁴⁷ and R⁴⁸ together with the nitrogen atom to which they are attached form a 4- to 6-membered saturated heterocycle;
R⁴⁹ represents C₁-C₆alkyl, C₃-C₆cycloalkyl or -CH₂Ar wherein Ar represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring;
R⁵⁰ and R⁵¹ each independently represent hydrogen, C₁-C₄alkyl or C₃-C₆cycloalkyl, or R⁵⁰ and R⁵¹ together with the nitrogen atom to which they are attached form a 4- to 6-membered saturated heterocycle;
R⁵² and R⁵³ each independently represent hydrogen, C₁-C₄alkyl or C₃-C₆cycloalkyl, or R⁵² and R⁵³ together with the nitrogen atom to which they are attached form a 4- to 6-membered saturated heterocycle;
R⁵⁴ and R⁵⁵ each independently represent hydrogen, C₁-C₄alkyl or C₃-C₆cycloalkyl, or R⁵⁴ and R⁵⁵ together with the nitrogen atom to which they are attached form a 4- to 6-membered saturated heterocycle;
R⁵⁶ represents C₁-C₆alkyl or C₃-C₆cycloalkyl;
R⁵⁷ and R⁵⁸ each independently represent hydrogen, C₁-C₄alkyl or C₃-C₆cycloalkyl, or R⁵⁷ and R⁵⁸ together with the nitrogen atom to which they are attached form a 4- to 6-membered saturated heterocycle;
R⁵⁹ and R⁶⁰ each independently represent hydrogen, C₁-C₄alkyl or C₃-C₆cycloalkyl, or R⁵⁹ and R⁶⁰ together with the nitrogen atom to which they are attached form a 4- to 6-membered saturated heterocycle;
R⁶¹ and R⁶² each independently represent hydrogen, C₁-C₄alkyl or C₃-C₆cycloalkyl, or R⁶¹ and R⁶² together with the nitrogen atom to which they are attached form a 4- to 6-membered saturated heterocycle optionally comprising an additional heteratom selected from oxygen, sulphur or nitrogen;
R⁶³ and R⁶⁴ each independently represent hydrogen, C₁-C₄alkyl or C₃-C₆cycloalkyl, or R⁶³ and R⁶⁴ together with the nitrogen atom to which they are attached form a 4- to 6-membered saturated heterocycle;
R⁶⁵ and R⁶⁶ each independently represent hydrogen, C₁-C₄alkyl or C₃-C₆cycloalkyl, or R⁶⁵ and R⁶⁶ together with the nitrogen atom to which they are attached form a 4- to 6-membered saturated heterocycle; and
wherein
(i) when R¹ is an optionally substituted C₂-C₆alkenyl, 4- to 6-membered heterocyclyl group, C₁-C₆alkoxy group, C₃-C₁₂carbocyclyloxy group, a 5- to 6-membered heterocyclyloxy, —S(O)ₓR⁴⁹, —S(O)₂NR⁵⁰R⁵¹or -A-B group,
R³ represents a C₁-C₅alkyl group optionally substituted by one or more substituents selected from C₁-C₃alkoxy, cyano, hydroxyl, amino (—NH₂), mono-C₁-C₃alkylamino and di-(C₁-C₃alkyl)amino, a C₃-C₅cycloalkyl group optionally substituted by one or more substituents selected from C₁-C₃alkyl and C₁-C₃alkoxy, a 3- to 5-membered saturated heterocyclyl group optionally substituted with by one or more substituents selected from C₁-C₃alkyl, C₁-C₃alkoxy and C₃cycloalkyl, a 5- or 6-membered aromatic ring optionally comprising at least one ring heteroatom selected from nitrogen, oxygen and sulphur, a mono-C₁-C₃alkylaminocarbonyl group, a di-(C₁-C₃alkyl)aminocarbonyl group, a C₁-C₃alkoxy carbonyl group, a —CONH₂ group, a —CN group, or a —CO₂H group;
or (ii) when R¹ is an optionally substituted C₁-C₆alkyl or a C₃-C₅cycloalkyl group,
R³ represents a C₁-C₅alkyl group optionally substituted by one or more substituents selected from C₁-C₃alkoxy, cyano, hydroxyl, amino (—NH₂), mono-C₁-C₃alkylamino and di-(C₁-C₃alkyl)amino, a C₃-C₅cycloalkyl group optionally substituted with C₁-C₃alkoxy, a 3- to 5-membered saturated heterocyclyl group optionally substituted with by one or more substituents selected from C₁-C₃alkyl, C₁-C₃alkoxy and C₃cycloalkyl, a —CONH₂ group, a —CN group, or a —CO₂H group;
or a pharmaceutically acceptable salt thereof,
provided that the compound of Formula 1 is not
N′-(5-cyclopropyl-1H-pyrazol-3-yl)-6-methyl-N-[(3-propan-2-yl-1,2-oxazol-5-yl)methyl]pyrimidine-2,4-diamine,
N′-(5-cyclopropyl-1H-pyrazol-3-yl)-N-[(3-propan-2-yl-1,2-oxazol-5-yl)methyl]pyrimidine-2,4-diamine,
N-[(3-cyclohexyl-1,2-oxazol-5-yl)methyl]-N′-(5-cyclopropyl-1H-pyrazol-3-yl)-6-methyl-pyrimidine-2,4-diamine,
N-[(3-cyclohexyl-1,2-oxazol-5-yl)methyl]-N′-(5-cyclopropyl-1H-pyrazol-3-yl)pyrimidine-2,4-diamine,
6-methyl-N-[(3-propan-2-yl-1,2-oxazol-5-yl)methyl]-N′-(5-propan-2-yl-1H-pyrazol-3-yl)pyrimidine-2,4-diamine,
N4-(5-cyclopropyl-1H-pyrazol-3-yl)-N6-(3-diethylaminopropyl)-N2-[(3-propan-2-yl-1,2-oxazol-5-yl)methyl]pyrimidine-2,4,6-triamine,
N4-(5-cyclopropyl-1H-pyrazol-3-yl)-N6-(2-diethylaminoethyl)-N2-[(3-propan-2-yl-1,2-oxazol-5-yl)methyl]pyrimidine-2,4,6-triamine,
N′-(5-cyclopropyl-1H-pyrazol-3-yl)-6-(2-dimethylaminoethoxy)-N-[(3-propan-2-yl-1,2-oxazol-5-yl)methyl]pyrimidine-2,4-diamine,
6-(2-diethylaminoethoxy)-N-[(3-propan-2-yl-1,2-oxazol-5-yl)methyl]-N′-(5-propan-2-yl-1H-pyrazol-3-yl)pyrimidine-2,4-diamine,
N-[(3-propan-2-yl-1,2-oxazol-5-yl)methyl]-N′-(5-propan-2-yl- I H-pyrazol-3-yl)pyrimidine-2,4-diamine,
6-(2-dimethylaminoethoxy)-N-[(3-propan-2-yl-1,2-oxazol-5-yl)methyl]-N′-(5-propan-2-yl-1H-pyrazol-3-yl)pyrimidine-2,4-diamine,
N′-(5-cyclopropyl-1H-pyrazol-3-yl)-6-methyl-N-[(3-methyl-1,2-oxazol-5-yl)methyl]pyrimidine-2,4-diamine,
N′-(5-cyclopropyl-1H-pyrazol-3-yl)-6-(2-diethylaminoethoxy)-N-[(3-propan-2-yl-1,2-oxazol-5-yl)methyl]pyrimidine-2,4-diamine,
N′-(5-cyclopropyl-1H-pyrazol-3-yl)-6-(2-diethylaminoethoxy)-N-[(3-methyl-1,2-oxazol-5-yl)methyl]pyrimidine-2,4-diamine,
N′-(5-cyclopropyl-1H-pyrazol-3-yl)-6-(2-dimethylaminoethoxy)-N-[(3-methyl-1,2-oxazol-5-yl)methyl]pyrimidine-2,4-diamine,
6-(2-dimethylaminoethoxy)-N-[(3-methyl-1,2-oxazol-5-yl)methyl]-N′-(5-methyl-1H-pyrazol-3-yl)pyrimidine-2,4-diamine,
6-(2-diethylaminoethoxy)-N-[(3-methyl-1,2-oxazol-5-yl)methyl]-N′-(5-methyl-1H-pyrazol-3-yl)pyrimidine-2,4-diamine,
N′-(5-cyclopropyl-1H-pyrazol-3-yl)-6-(2-dimethylaminoethoxy)-N-[(3-ethyl-1,2-oxazol-5-yl)methyl]pyrimidine-2,4-diamine
N′-(5-cyclopropyl-1H-pyrazol-3-yl)-6-(2-diethylaminoethoxy)-N-[(3-ethyl-1,2-oxazol-5-yl)methyl]pyrimidine-2,4-diamine, or
N′-(5-cyclopropyl-1H-pyrazol-3-yl)-N-[(3-ethyl-1,2-oxazol-5-yl)methyl]-6-(2-pyrrolidin-1-ylethoxy)pyrimidine-2,4-diamine.

2. A compound of formula (I) according to claim 1 wherein:
R¹ represents a C₁-C₆alkyl group optionally substituted by one or more substituents selected from C₁-C₆alkoxy, C₃-C₆cycloalkyl, C₁-C₆alkylthio, —NR⁵R⁶, —C(O)NR⁷R⁸, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, cyano, hydroxyl and trifluoromethyl), cyano and hydroxyl, a C₃-C₅cycloalkyl group optionally substituted by one or more substituents selected from C₁-C₆alkoxy, C₃-C₆cycloalkyl, C₁-C₆alkylthio, —NR⁹R¹⁰, —C(O)NR¹¹R¹² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a C₂-C₆alkenyl group optionally substituted by one or more substituents selected from C₁-C₆alkoxy, C₃-C₆cycloalkyl, C₁-C₆alkylthio, —NR¹³R¹⁴, -C(O)NR¹⁵R¹⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a 4- to 6-membered heterocyclyl group optionally substituted with by one or more substituents selected from C₁-C₆alkyl, C₁-C₆alkoxy, C₃-C₆cycloalkyl, C₁-C₆alkylthio, —NR¹⁷R¹⁸, —C(O)NR¹⁹R²⁰, (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₘC₁-C₆alkyl, —NR²¹R²², —C(O)NR²³R²⁴, —SO₂NR²⁵R²⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₁-C₆alkoxy group optionally substituted by one or more substituents selected from C₁-C₆alkoxy, C₆-aryloxy, C₃-C₆cycloalkyl, —NR²⁷R²⁸, —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —OSO₂C₁₋₆alkyl, —NR³¹R³², —C(O)NR³³R³⁴, —NHC(O)OC₁₋₆alkyl, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a C₆aryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₚC₁-C₆alkyl, —NR³⁷R³⁸, —C(O)NR³⁹R⁴⁰, —SO₂NR⁴¹R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, a 5- to 6-membered heteroaryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl, a —S(O)ₓR⁴⁹ group, a —S(O)₂NR⁵⁰R⁵¹ group, or -A-B;
R² represents hydrogen or a C₁-C₃alkyl group optionally substituted by one or more substituents selected from C₁-C₃alkoxy, cyano, hydroxyl, amino (—NH₂), mono-C₁-C₃alkylamino and di-(C₁-C₃alky)amino;
R⁴ represents hydrogen, a C₁-C₆alkyl group optionally substituted by one or more substituents selected from C₁-C₃alkoxy, hydroxyl, amino (—NH₂), mono-C₁-C₃alkylamino and di-(C₁-C₃alkyl)amino, a C₁-C₆alkenyl group optionally substituted with C₁-C₃alkoxy, a C₁-C₆alkynyl group optionally substituted with C₁-C₃alkoxy, a C₃-C₅cycloalkyl group optionally substituted with C₁-C₃alkoxy, a C₁-C₆alkoxy group optionally substituted with C₁-C₃alkoxy, hydroxyl, amino (—NH₂), mono-C₁-C₃alkylamino and di-(C₁-C₃alkyl)amino,
-C(O)NR⁵²R⁵³, —NR⁵⁴R⁵⁵, —S(O)_{y}R⁵⁶;
A represents a C₂-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or a C₁-alkyleneoxy optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; ‘B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, C₁-C₆alkyloxycarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₃₋₅cycloalkyl, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring;
m is 0, 1 or 2;
n is 0, 1 or 2;
p is 0, 1 or 2;
r is 0, 1 or 2;
s is 0, 1 or 2
x is 0, 1 or 2;
y is 0, 1 or 2;
R⁵ and R⁶ each independently represent hydrogen, C₁-C₄alkyl or C₃-C₆cycloalkyl, or R⁵ and R⁶ together with the nitrogen atom to which they are attached form a 4- to 6-membered saturated heterocycle;
R⁷ and R⁸ each independently represent hydrogen, C₁-C₄alkyl or C₃-C₆cycloalkyl, or R⁷ and R⁸ together with the nitrogen atom to which they are attached form a 4- to 6-membered saturated heterocycle;
R⁹ and R¹⁰ each independently represent hydrogen, C₁-C₄alkyl or C₃-C₆cycloalkyl, or R⁹ and R¹⁰ together with the nitrogen atom to which they are attached form a 4- to 6-membered saturated heterocycle;
R¹¹ and R¹² each independently represent hydrogen, C₁-C₄alkyl or C₃-C₆cycloalkyl, or R¹¹ and R¹² together with the nitrogen atom to which they are attached form a 4- to 6-membered saturated heterocycle;
R¹³ and R¹⁴ each independently represent hydrogen, C₁-C₄alkyl or C₃-C₆cycloalkyl, or R¹³ and R¹⁴ together with the nitrogen atom to which they are attached form a 4- to 6-membered saturated heterocycle;
R¹⁵ and R¹⁶ each independently represent hydrogen, C₁-C₄alkyl or C₃-C₆cycloalkyl, or R¹⁵ and R¹⁶ together with the nitrogen atom to which they are attached form a 4- to 6-membered saturated heterocycle;
R¹⁷ and R¹⁸ each independently represent hydrogen, C₁-C₄alkyl or C₃-C₆cycloalkyl, or R¹⁷ and R¹⁸ together with the nitrogen atom to which they are attached form a 4- to 6-membered saturated heterocycle;
R¹⁹ and R²⁰ each independently represent hydrogen, C₁-C₄alkyl or C₃-C₆cycloalkyl, or R¹⁹ and R²⁰ together with the nitrogen atom to which they are attached form a 4- to 6-membered saturated heterocycle;
R²¹ and R²² each independently represent hydrogen, C₁-C₄alkyl or C₃-C₆cycloalkyl, or R²¹ and R²² together with the nitrogen atom to which they are attached form a 4- to 6-membered saturated heterocycle;
R²³ and R²⁴ each independently represent hydrogen, C₁-C₄alkyl or C₃-C₆cycloalkyl, or R²³ and R²⁴ together with the nitrogen atom to which they are attached form a 4- to 6-membered saturated heterocycle;
R²⁵ and R²⁶ each independently represent hydrogen, C₁-C₄alkyl or C₃-C₆cycloalkyl, or R²⁵ and R²⁶ together with the nitrogen atom to which they are attached form a 4- to 6-membered saturated heterocycle;
R²⁷ and R²⁸ each independently represent hydrogen, C₁-C₄alkyl or C₃-C₆cycloalkyl, or R²⁷ and R²⁸ together with the nitrogen atom to which they are attached form a 4- to 6-membered saturated heterocycle;
R²⁹ and R³⁰ each independently represent hydrogen, C₁-C₄alkyl or C₃-C₆cycloalkyl, or R²⁹ and R³⁰ together with the nitrogen atom to which they are attached form a 4- to 6-membered saturated heterocycle;
R³¹ and R³² each independently represent hydrogen, C₁-C₆alkyl or C₃-C₆cycloalkyl, or R³¹ and R³² together with the nitrogen atom to which they are attached form a 4- to 6-membered saturated heterocycle optionally comprising an additional heteratom selected from oxygen, sulphur or nitrogen;
R³³ and R³⁴ each independently represent hydrogen, C₁-C₄alkyl or C₃-C₆cycloalkyl, or R³³ and R³⁴ together with the nitrogen atom to which they are attached form a 4- to 6-membered saturated heterocycle optionally comprising an additional heteratom selected from oxygen, sulphur or nitrogen;
R³⁵ and R³⁶ each independently represent hydrogen, C₁-C₄alkyl or C₃-C₆cycloalkyl, or R³⁵ and R³⁶ together with the nitrogen atom to which they are attached form a 4- to 6-membered saturated heterocycle;
R³⁷ and R³⁸ each independently represent hydrogen, C₁-C₄alkyl or C₃-C₆cycloalkyl, or R³⁷ and R³⁸ together with the nitrogen atom to which they are attached form a 4- to 6-membered saturated heterocycle;
R³⁹ and R⁴⁰ each independently represent hydrogen, C₁-C₄alkyl or C₃-C₆cycloalkyl, or R³⁹ and R⁴⁰ together with the nitrogen atom to which they are attached form a 4- to 6-membered saturated heterocycle;
R⁴¹ and R⁴² each independently represent hydrogen, C₁-C₄alkyl or C₃-C₆cycloalkyl, or R⁴¹ and R⁴² together with the nitrogen atom to which they are attached form a 4- to 6-membered saturated heterocycle;
R⁴³ and R⁴⁴ each independently represent hydrogen, C₁-C₄alkyl or C₃-C₆cycloalkyl, or R⁴³ and R⁴⁴ together with the nitrogen atom to which they are attached form a 4- to 6-membered saturated heterocycle;
R⁴⁵ and R⁴⁶ each independently represent hydrogen, C₁-C₄alkyl or C₃-C₆cycloalkyl, or R⁴⁵ and R⁴⁶ together with the nitrogen atom to which they are attached form a 4- to 6-membered saturated heterocycle;
R⁴⁷ and R⁴⁸ each independently represent hydrogen, C₁-C₄alkyl or C₃-C₆cycloalkyl, or R⁴⁷ and R⁴⁸ together with the nitrogen atom to which they are attached form a 4- to 6-membered saturated heterocycle;
R⁴⁹ represents C₁-C₆alkyl, C₃-C₆cycloalkyl or —CH₂Ar wherein Ar represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, -S(O),C i-C₆alkyl, -OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), —CH₂OCO₂H, halogen, nitro, cyano, carboxyl and hydroxyl, and optionally wherein two or more adjacent substituents together with the atoms to which they are attached form a partially or fully unsaturated 4- to 6-membered ring;
R⁵⁰ and R⁵¹ each independently represent hydrogen, C₁-C₄alkyl or C₃-C₆cycloalkyl, or R⁵⁰ and R⁵¹ together with the nitrogen atom to which they are attached form a 4- to 6-membered saturated heterocycle;
R⁵² and R⁵³ each independently represent hydrogen, C₁-C₄alkyl or C₃-C₆cycloalkyl, or R⁵² and R⁵³ together with the nitrogen atom to which they are attached form a 4- to 6-membered saturated heterocycle;
R⁵⁴ and R⁵⁵ each independently represent hydrogen, C₁-C₄alkyl or C₃-C₆cycloalkyl, or R⁵⁴ and R⁵⁵ together with the nitrogen atom to which they are attached form a 4- to 6-membered saturated heterocycle;
R⁵⁶ represents C₁-C₆alkyl or C₃-C₆cycloalkyl;
R⁵⁷ and R⁵⁸ each independently represent hydrogen, C₁-C₄alkyl or C₃-C₆cycloalkyl, or R⁵⁷ and R⁵⁸ together with the nitrogen atom to which they are attached form a 4- to 6-membered saturated heterocycle;
R⁵⁹ and R⁶⁰ each independently represent hydrogen, C₁-C₄alkyl or C₃-C₆cycloalkyl, or R⁵⁹ and R⁶⁰ together with the nitrogen atom to which they are attached form a 4- to 6-membered saturated heterocycle;
R⁶¹ and R⁶² each independently represent hydrogen, C₁-C₄alkyl or C₃-C₆cycloalkyl, or R⁶¹ and R⁶² together with the nitrogen atom to which they are attached form a 4- to 6-membered saturated heterocycle optionally comprising an additional heteratom selected from oxygen, sulphur or nitrogen;
R⁶³ and R⁶⁴ each independently represent hydrogen, C₁-C₄alkyl or C₃-C₆cycloalkyl, or R⁶³ and R⁶⁴ together with the nitrogen atom to which they are attached form a 4- to 6-membered saturated heterocycle;
R⁶⁵ and R⁶⁶ each independently represent hydrogen, C₁-C₄alkyl or C₃-C₆cycloalkyl, or R⁶⁵ and R⁶⁶ together with the nitrogen atom to which they are attached form a 4- to 6-membered saturated heterocycle; and
wherein
(i) when R¹ is an optionally substituted C₂-C₆alkenyl, 4- to 6-membered heterocyclyl group, C₁-C₆alkoxy group, C₆aryloxy group, 5- to 6-membered heteroaryloxy, —S(O)ₓR⁴⁹, —S(O)₂NR⁵⁰R⁵¹ or -A-B group,
R³ represents a C₁-C₅alkyl group optionally substituted by one or more substituents selected from C₁-C₃alkoxy, cyano, hydroxyl, amino (—NH₂), mono-C₁-C₃alkylamino and di-(C₁-C₃alkyl)amino, a C₃-C₅cycloalkyl group optionally substituted by one or more substituents selected from C₁-C₃alkyl and C₁-C₃alkoxy, a 3- to 5-membered saturated heterocyclyl group optionally substituted with by one or more substituents selected from C₁-C₃alkyl, C₁-C₃alkoxy and C₃cycloalkyl, a 5- or 6-membered aromatic ring optionally comprising at least one ring heteroatom selected from nitrogen, oxygen and sulphur, a mono-C₁-C₃alkylaminocarbonyl group, a di-(C₁-C₃alkyl)aminocarbonyl group, a C₁-C₃alkoxy carbonyl group, a —CONH₂ group, a —CN group, or a —CO₂H group;
or (ii) when R¹ is an optionally substituted C₁-C₆alkyl or a C₃-C₅cycloalkyl group,
R³ represents a C₁-C₅alkyl group optionally substituted by one or more substituents selected from C₁-C₃alkoxy, cyano, hydroxyl, amino (—NH₂), mono-C₁-C₃alkylamino and di-(C₁-C₃alkyl)amino, a C₃-C₅cycloalkyl group optionally substituted by one or more substituents selected from C₁-C₃alkyl and C₁-C₃alkoxy, a 3- to 5-membered saturated heterocyclyl group optionally substituted with by one or more substituents selected from C₁-C₃alkyl, C₁-C₃alkoxy and C₃cycloalkyl, a —CONH₂ group, a —CN group, or a —CO₂H group;
or a pharmaceutically acceptable salt thereof,
provided that the compound of Formula 1 is not
N′-(5-cyclopropyl-1H-pyrazol-3-yl)-6-methyl-N-[(3-propan-2-yl-1,2-oxazol-5-yl)methyl]pyrimidine-2,4-diamine,
N′-(5-cyclopropyl-1H-pyrazol-3-yl)-N-[(3-propan-2-yl-1,2-oxazol-5-yl)methyl]pyrimidine-2,4-diamine,
N-[(3-cyclohexyl-1,2-oxazol-5-yl)methyl]-N′-(5-cyclopropyl-1H-pyrazol-3-yl)-6-methyl-pyrimidine-2,4-diamine,
N-[(3-cyclohexyl-1,2-oxazol-5-yl)methyl]-N′-(5-cyclopropyl-1H-pyrazol-3-yl)pyrimidine-2,4-diamine,
6-methyl-N-[(3-propan-2-yl-1,2-oxazol-5-yl)methyl]-N′-(5-propan-2-yl-1H-pyrazol-3-yl)pyrimidine-2,4-diamine,
N4-(5-cyclopropyl-1H-pyrazol-3-yl)-N6-(3-diethylaminopropyl)-N2-[(3-propan-2-yl-1,2-oxazol-5-yl)methyl]pyrimidine-2,4,6-triamine,
N4-(5-cyclopropyl-1H-pyrazol-3-yl)-N6-(2-diethylaminoethyl)-N2-[(3-propan-2-yl-1,2-oxazol-5-yl)methyl]pyrimidine-2,4,6-triamine,
N′-(5-cyclopropyl-1H-pyrazol-3-yl)-6-(2-dimethylaminoethoxy)-N-[(3-propan-2-yl-1,2-oxazol-5-yl)methyl]pyrimidine-2,4-diamine,
6-(2-diethylaminoethoxy)-N-[(3-propan-2-yl-1,2-oxazol-5-yl)methyl]-N′-(5-propan-2-1H-pyrazol-3-yl)pyrimidine-2,4-diamine,
N-[(3-propan-2-yl-1,2-oxazol-5-yl)methyl]-N′-(5-propan-2-yl-1H-pyrazol-3-yl)pyrimidine-2,4-diamine,
6-(2-dimethylaminoethoxy)-N-[(3-propan-2-yl-1,2-oxazol-5-yl)methyl]-N′-(5-propan-2-yl-1H-pyrazol-3-yl)pyrimidine-2,4-diamine,
N′-(5-cyclopropyl-1H-pyrazol-3-yl)-6-methyl-N-[(3-methyl-1,2-oxazol-5-yl)methyl]pyrimidine-2,4-diamine,
N′-(5-cyclopropyl-1H-pyrazol-3-yl)-6-(2-diethylaminoethoxy)-N-[(3-propan-2-yl-1,2-oxazol-5-yl)methyl]pyrimidine-2,4-diamine,
N′-(5-cyclopropyl-1H-pyrazol-3-yl)-6-(2-diethylaminoethoxy)-N-[(3-methyl-1,2-oxazol-5-yl)methyl]pyrimidine-2,4-diamine,
N′-(5-cyclopropyl-1H-pyrazol-3-yl)-6-(2-dimethylaminoethoxy)-N-[(3-methyl-1,2-oxazol-5-yl)methyl]pyrimidine-2,4-diamine,
6-(2-dimethylaminoethoxy)-N-[(3-methyl-1,2-oxazol-5-yl)methyl]-N′-(5-methyl-1H-pyrazol-3-yl)pyrimidine-2,4-diamine,
6-(2-diethylaminoethoxy)-N-[(3-methyl-1,2-oxazol-5-yl)methyl]-N′-(5-methyl-1H-pyrazol-3-yl)pyrimidine-2,4-diamine,
N′-(5-cyclopropyl-1H-pyrazol-3-yl)-6-(2-dimethylaminoethoxy)-N-[(3-ethyl-1,2-oxazol-5-yl)methyl]pyrimidine-2,4-diamine
N′-(5-cyclopropyl-1H-pyrazol-3-yl)-6-(2-diethylaminoethoxy)-N-[(3-ethyl-1,2-oxazol-5-yl)methyl]pyrimidine-2,4-diamine, or
N′-(5-cyclopropyl-1H-pyrazol-3-yl)-N-[(3-ethyl-1,2-oxazol-5-yl)methyl]-6-(2-pyrrolidin-1-ylethoxy)pyrimidine-2,4-diamine.

3. A compound according to claim 1 wherein R⁴represents hydrogen, a C₁-C₆alkyl group; a C₃-C₅cycloalkyl; a C₁-C₆alkoxy group.

4. A compound according to claim 1 wherein R⁴ represents hydrogen, methyl or methoxy.

5. A compound according to claim 1 wherein R⁴ represents hydrogen.

6. A compound according to claim 1 wherein R² represents hydrogen or a C₁-C₃alkyl group.

7. A compound according to claim 1 wherein R² represents hydrogen or methyl.

8. A compound according to claim 1 wherein R² represents hydrogen.

9. A compound according to claim 1 wherein R³ represents a C₁-C₅alkyl group; a C₃-C₅cycloalkyl group; a oxolan-2-yl group; a CH₂N(CH₃)₂ group; a —CONHMe group or a —CONH₂ group.

10. A compound according to claim 1 wherein R³ represents a C₁-C₅alkyl group; a C₃-C₅cycloalkyl group; a oxolan-2-yl group; or a —CONH₂ group.

11. A compound according to claim 1 wherein R³ represents methyl, ethyl, propyl, i-propyl, cyclopropyl, cyclobutyl or —CONH₂.

12. A compound according to claim 1 wherein R³ represents methyl, cyclopropyl or —CONH₂.

13. A compound according to claim 1 wherein R³ represents methyl or cyclopropyl.

14. A compound according to claim 1 wherein R⁴represents hydrogen, a C₁-C₆alkyl group, a C₃-C₅cycloalkyl, or a C₁-C₆alkoxy group; R² represents hydrogen or a C₁-C₃alkyl group; and R³ represents a C₁-C₅alkyl group, a C₃-C₅cycloalkyl group, an oxolan-2-yl group, a CH₂N(CH₃)₂ group, a —CONHMe group or a —CONH₂ group.

15. A compound according to claim 1 wherein R⁴ represents hydrogen, methyl or methoxy; R² represents hydrogen or methyl; and R³ represents a C₁-C₅alkyl group, a C₃-C₅cycloalkyl group, a oxolan-2-yl group or a —CONH₂ group.

16. A compound according to claim 1 wherein R⁴ represents hydrogen; R² represents hydrogen; and R³ represents methyl, ethyl, propyl, i-propyl, cyclopropyl, cyclobutyl or —CONH₂.

17. A compound according to claim 1 wherein R⁴ represents hydrogen; R² represents hydrogen; and R³ represents methyl, cyclopropyl or —CONH₂.

18. A compound according to claim 1 wherein R⁴ represents hydrogen; R² represents hydrogen; and R³ represents methyl or cyclopropyl.

19. A compound according to claim 1 wherein
R¹ represents a C₁-C₆alkoxy group optionally substituted by one or more substituents selected from C₁-C₆alkoxy, C₆-aryloxy, C₃-C₆cycloalkyl, —NR²⁷R²⁸, —C(O)NR²⁹R³⁰ (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), hydroxyl and a 5- or 6-membered aromatic ring optionally comprising at least one ring heteroatom selected from nitrogen, oxygen and sulphur, the ring being optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₙC₁-C₆alkyl, —OSO₂C₁₋₆alkyl, —NR³¹R³², —C(O)NR³³R³⁴, —NHC(O)OC₁₋₆alkyl, —SO₂NR³⁵R³⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl;
a C₆aryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ₚC₁-C₆alkyl, —NR³⁷R³⁸, —C(O)N³⁹R⁴⁰, —SO₂NR⁴¹R⁴² (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl; or a 5- to 6-membered heteroaryloxy group optionally substituted by one or more substituents selected from C₁-C₆alkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, —S(O)ᵣC₁-C₆alkyl, —NR⁴³R⁴⁴, —C(O)NR⁴⁵R⁴⁶, —SO₂NR⁴⁷R⁴⁸ (each of which may be optionally substituted by one or more substituents selected from halogen, C₁-C₆alkyl, C₁-C₆alkoxy, C₁-C₆alkylthio, amino (—NH₂), mono-C₁-C₆alkylamino, di-(C₁-C₆alky)amino, hydroxyl and trifluoromethyl), halogen, nitro, cyano, carboxyl and hydroxyl.

20. A compound according to claim 1 wherein R¹ represents a C₁-C₆alkoxy group optionally substituted by one or more substituents selected from C₁-C₆alkoxy.

21. A compound according to claim 1 wherein R¹ represents a C₁-C₆alkoxy group

22. A compound according to claim 1 wherein R¹ represents a C₁-C₃alkoxy group

23. A compound according to claim 1 wherein R¹ represents a i-propoxy group

24. A compound according to claim 19 wherein R⁴ represents hydrogen, a C₁-C₆alkyl group, a C₃-C₅cycloalkyl, or a C₁-C₆alkoxy group; R² represents hydrogen or a C₁-C₃alkyl group; and R³ represents a C₁-C₅alkyl group, a C₃-C₅cycloalkyl group, an oxolan-2-yl group, a CH₂N(CH₃)₂ group, a —CONHMe group or a —CONH₂ group.

25. A compound according to claim 20 wherein R⁴ represents hydrogen, methyl or methoxy; R² represents hydrogen or methyl; and R³ represents a C₁-C₅alkyl group, a C₃-C₅cycloalkyl group, a oxolan-2-yl group or a —CONH₂ group.

26. A compound according to claim 24 wherein R⁴ represents hydrogen, methyl or methoxy; R² represents hydrogen or methyl; and R³ represents a C₁-C₅alkyl group, a C₃-C₅cycloalkyl group, a oxolan-2-yl group or a —CONH₂ group.

27. A compound according to claim 25 wherein R⁴ represents hydrogen; R² represents hydrogen; and R³ represents methyl, ethyl, propyl, i-propyl, cyclopropyl, cyclobutyl or —CONH₂.

28. A compound according to claim 26 wherein R⁴ represents hydrogen; R² represents hydrogen; and R³ represents methyl, ethyl, propyl, i-propyl, cyclopropyl, cyclobutyl or —CONH₂.

29. A compound according to claim 1 wherein R¹ represents -A-B wherein
A represents a C₂-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, a C₁-alkyleneoxy optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl, or an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; and
B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, C₁-C₆alkyloxycarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring.

30. A compound according to claim 1 wherein R¹ represents -A-B wherein
A represents a C₂-alkylene optionally substituted by one or more substituents selected from C₁-C₆alkyl, C₁-C₆alkoxy, C₃-C₆cycloalkyl, C₁-C₆alkylthio,- —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; or an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; and
B represents a 5- or 6-membered aromatic ring optionally comprising at least one ring heteroatom selected from nitrogen, oxygen and sulphur, the aromatic ring being optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring.

31. A compound according to claim 1 wherein R¹ represents -A-B wherein
A represents a C₂-alkylene optionally substituted by one or more substituents selected from C₁-C₆alkyl, C₁-C₆alkoxy, C₃-C₆cycloalkyl, C₁-C₆alkylthio,- —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; or an oxyC₁-alkylene optionally substituted by one or more substituents selected from C₁-C₆alkyl, C₁-C₆alkoxy, C₃-C₆cycloalkyl, C₁-C₆alkylthio, —NR⁵⁷R⁵⁸, —C(O)NR⁵⁹R⁶⁰ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), and hydroxyl; and
B represents a phenyl ring or a pyridin-4-yl ring each optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, —OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring.

32. A compound according to claim 1 wherein R¹ represents -A-B wherein
A represents a —CH₂CH₂— or a —OCH₂—; and
B represents a phenyl ring or a pyridin-4-yl ring each optionally substituted by one or more substituents selected from C₁-C₆alkyl, C₃₋₅cycloalkyl, C₁-C₆alkoxy, C₂-C₆alkenyl, C₃-C₆cycloalkyl, C₁-C₆alkoxycarbonyl, C₁-C₆alkylcarbonyl, C₁-C₆alkylcarbonylamino, phenylcarbonyl, phenyl, benzyl, benzyloxy, —S(O)ₛC₁-C₆alkyl, -OS(O)₂C₁-C₆alkyl, —NR⁶¹R⁶², —C(O)NR⁶³R⁶⁴, —SO₂NR⁶⁵R⁶⁶ (each of which may be optionally substituted by one or more substituents selected from halogen, C₁-C₆alkyl, C₁-C₆alkoxy, C₁-C₆alkylthio, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring.

33. A compound according to claim 1 wherein R¹ represents -A-B wherein
A represents a —CH₂CH₂— or a —OCH₂—; and
B represents a phenyl ring or a pyridin-4-yl ring each optionally substituted by one 20 or more substituents selected from C₁-C₆alkyl, C₁-C₆alkoxy, C₁-C₆alkoxycarbonyl, C₁-C₆alkylcarbonylamino, phenyl, —NR⁶¹R⁶², —C(O)NR⁶³R⁶⁴, (each of which may be optionally substituted by one or more substituents selected from halogen, C₁-C₆alkyl, C₁-C₆alkoxy, amino (—NH₂), mono- and di-C₁-C₆alkylamino, hydroxyl and trifluoromethyl), halogen, nitro, cyano, carboxyl and hydroxyl, and optionally wherein two or more adjacent substituents together with the atoms to which they are attached form a partially or fully unsaturated 4- to 6-membered ring.

34. A compound according to any one of claim 29 wherein R⁶¹ and R⁶² each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁶¹ and R⁶² together with the nitrogen atom to which they are attached form a 4- to 6-membered saturated heterocycle (such as pyrrolidinyl, morpholiny or piperidinyl); and R⁶³ and R⁶⁴ each independently represent hydrogen, C₁-C₄, particularly C₁-C₂alkyl (such as methyl, ethyl, n-propyl, isopropyl, n-butyl, isobutyl or tert-butyl) or C₃-C₆cycloalkyl (cyclopropyl, cyclobutyl, cyclopentyl and cyclohexyl), or R⁶³ and R⁶⁴ together with the nitrogen atom to which they are attached form a 4- to 6-membered saturated heterocycle (such as pyrrolidinyl, morpholiny or piperidinyl).

35. A compound according to claim 29 wherein R⁴ represents hydrogen, a C₁-C₆alkyl group, a C₃-C₅cycloalkyl, or a C₁-C₆alkoxy group; R² represents hydrogen or a C₁-C₃alkyl group; and R³ represents a C₁-C₅alkyl group, a C₃-C₅cycloalkyl group, an oxolan-2-yl group, a CH₂N(CH₃)₂ group, a —CONHMe group or a —CONH₂ group.

36. A compound according to claim 30 wherein R⁴ represents hydrogen; R² represents hydrogen; and R³ represents methyl, ethyl, propyl, i-propyl, cyclopropyl, cyclobutyl or —CONH₂.

37. A compound according to claim 31 wherein R⁴ represents hydrogen; R² represents hydrogen; and R³ represents methyl, ethyl, propyl, i-propyl, cyclopropyl, cyclobutyl or —CONH₂.

38. A compound according to claim 33 wherein R⁴ represents hydrogen; R² represents hydrogen; and R³ represents methyl, ethyl, propyl, i-propyl, cyclopropyl, cyclobutyl or —CONH₂.

39. A compound according to claim 35 wherein R⁴ represents hydrogen, methyl or methoxy; R² represents hydrogen or methyl; and R³ represents a C₁-C₅alkyl group, a C₃-C₅cycloalkyl group, a oxolan-2-yl group or a —CONH₂ group.

40. A compound according to claim 39 wherein R⁴ represents hydrogen; R² represents hydrogen; and R³ represents methyl, ethyl, propyl, i-propyl, cyclopropyl, cyclobutyl or —CONH₂.

41. A compound according to claim 1 wherein R¹ represents a methyl, ethyl, propyl, i-propyl, hydroxymethyl, cyclopropyl, methoxypropyl, ethoxypropyl, phenylethyl, p-methoxyphenylethyl, m-methoxyphenylethyl, 3,5-dimethoxyphenylethyl, i-propoxy, benzyloxy, or a (3,5-dimethoxyphenyl)methoxy group.

42. A compound according to claim 1 wherein R¹ represents a hydroxymethyl, methoxypropyl, ethoxypropyl, phenylethyl, 2-(3-methoxyphenyl)ethyl, 2-(3,5-dimethoxyphenyl)ethyl, i-propoxy, benzyloxy, (3,5-dimethoxyphenyl)methoxy, 2-(3-hydroxyphenyl)ethyl, 2-(3,5-dihydroxyphenyl)ethyl, (3-methoxyphenyl)methoxy, [3-(methylcarbamoyl)phenyl]methoxy, [3-methoxy-5-(methylcarbamoyl)phenyl]methoxy, 2-[3-(methylcarbamoyl)phenyl]ethyl, 2-[3-methoxy-5-(methylcarbamoyl)phenyl]ethyl, (3-hydroxyphenyl)methoxy, (3,5-dihydroxyphenyl)methoxy, (3-chloro-5-methoxy-phenyl)methoxy, 2-(2,6-dimethoxypyridin-4-yl)ethyl, (5-fluoro-2-methoxy-pyridin-4-yl)methoxy, 2-(5-fluoro-2-methoxy-pyridin-4-yl)ethyl, (3-methoxy-5-methyl-phenyl)methoxy, (3-fluorophenyl)methoxy, (3-chlorophenyl)methoxy, 2-(3-aminophenyl)ethyl, 2-(5-methoxythiophen-2-yl)ethyl, 2-(2-furyl)ethyl, (2,6-dimethoxypyridin-4-yl)methoxy or a 2-(3-chloro-5-methoxy-phenyl)ethyl group.

43. A compound according to claim 1 wherein R¹ represents a hydroxymethyl, methoxypropyl, ethoxypropyl, phenylethyl, 2-(3-methoxyphenyl)ethyl, 2-(3,5-dimethoxyphenyl)ethyl, i-propoxy, benzyloxy, (3,5-dimethoxyphenyl)methoxy, 2-(3-hydroxyphenyl)ethyl, 2-(3,5-dihydroxyphenyl)ethyl, (3-methoxyphenyl)methoxy, [3-(methylcarbamoyl)phenyl]methoxy, [3-methoxy-5-(methylcarbamoyl)phenyl]methoxy, 2-[3-(methylcarbamoyl)phenyl]ethyl, 2-[3-methoxy-5-(methylcarbamoyl)phenyl]ethyl, (3-hydroxyphenyl)methoxy, (3,5-dihydroxyphenyl)methoxy, (3-chloro-5-methoxy-phenyl)methoxy, 2-(2,6-dimethoxypyridin-4-yl)ethyl, (5-fluoro-2-methoxy-pyridin-4-yl)methoxy, 2-(5-fluoro-2-methoxy-pyridin-4-yl)ethyl, (3-methoxy-5-methyl-phenyl)methoxy, (3-fluorophenyl)methoxy, (3-chlorophenyl)methoxy, 2-(3-aminophenyl)ethyl, 2-(5-methoxythiophen-2-yl)ethyl, 2-(2-furyl)ethyl or a 2-(3-chloro-5-methoxy-phenyl)ethyl group.

44. A compound according to claim 1 wherein R¹ represents a hydroxymethyl, methoxypropyl, ethoxypropyl, phenylethyl, 2-(3-methoxyphenyl)ethyl, 2-(3,5-dimethoxyphenyl)ethyl, i-propoxy, benzyloxy, (3,5-dimethoxyphenyl)methoxy, 2-(3-hydroxyphenyl)ethyl, 2-(3,5-dihydroxyphenyl)ethyl, (3-methoxyphenyl)methoxy, [3-(methylcarbamoyl)phenyl]methoxy, [3-methoxy-5-(methylcarbamoyl)phenyl]methoxy, 2-[3-(methylcarbamoyl)phenyl]ethyl, 2-[3-methoxy-5-(methylcarbamoyl)phenyl]ethyl, (3-hydroxyphenyl)methoxy, (3,5-dihydroxyphenyl)methoxy, (3-chloro-5-methoxy-phenyl)methoxy, or a 2-(3-chloro-5-methoxy-phenyl)ethyl group.

45. A compound according to claim 2 wherein R⁴ represents hydrogen and R¹ represents a C₁-C₃alkyl group (such as methyl, ethyl, propyl and i-propyl) substituted by one or more substituents selected from C₁-C₃alkoxy (such as methoxy, ethoxy, propoxy and i-propoxy) [which may be optionally substituted by one or more substituents selected from halogen (such as fluorine, chlorine, bromine or iodine), C₁-C₃alkyl (such as methyl, ethyl, propyl and i-propyl), C₁-C₃alkoxy (such as methoxy, ethoxy, propoxy and i-propoxy)], and hydroxyl; a C₁-C₃alkoxy group (such as methoxy, ethoxy, propoxy and i-propoxy) optionally substituted by one or more substituents selected from C₁-C₃alkoxy (such as methoxy, ethoxy, propoxy and i-propoxy) and cyclopropyl; a phenyloxy group optionally substituted by one or more substituents selected from C₁-C₃alkyl (such as methyl, ethyl, propyl and i-propyl), C₁-C₃alkoxy(such as methoxy, ethoxy, propoxy and i-propoxy) and cyclopropyl; or -A-B wherein A represents a C₂-alkylene or oxyC₁-alkylene, and B represents a phenyl ring optionally substituted by one or more substituents selected from halogen, C₁-C₃alkyl, C₁-C₃alkoxy or C(O)NR⁶³R⁶⁴.

46. A compound according to claim 45 wherein R¹ represents a hydroxymethyl, methoxypropyl, ethoxypropyl, phenylethyl, 2-(3-methoxyphenyl)ethyl, 2-(3,5-dimethoxyphenyl)ethyl, i-propoxy, benzyloxy, (3,5-dimethoxyphenyl)methoxy, 2-(3-hydroxyphenyl)ethyl, 2-(3,5-dihydroxyphenyl)ethyl, (3-methoxyphenyl)methoxy, [3-(methylcarbamoyl)phenyl]methoxy, [3-methoxy-5-(methylcarbamoyl)phenyl]methoxy, 2-[3-(methylcarbamoyl)phenyl]ethyl, 2-[3-methoxy-5-(methylcarbamoyl)phenyl]ethyl, (3-hydroxyphenyl)methoxy, (3,5-dihydroxyphenyl)methoxy, (3-chloro-5-methoxy-phenyl)methoxy, or a 2-(3-chloro-5-methoxy-phenyl)ethyl group.

47. A compound according to claim 42 wherein R² represents hydrogen.

48. A compound according to claim 42 wherein R³ represents a C₁-C₅alkyl group; a C₃-C₅cycloalkyl group; or a —CONH₂ group.

49. A compound according to claims 42 wherein R² represents hydrogen and R³ represents a C₁-C₅alkyl group; a C₃-C₅cycloalkyl group; or a —CONH₂ group.

50. A compound according to claim 49 wherein R³ represents methyl, cyclopropyl or —CONH₂.

51. A compound according to claim 45 wherein
(i) when R¹ is an optionally substituted 4- to 6-membered heterocyclyl group, C₁-C₆alkoxy group, C₆aryloxy group, 5- to 6-membered heteroaryloxy or -A-B group,
R³ represents methyl, ethyl, propyl, i-propyl, cyclopropyl, cyclobutyl, —CONH₂ or —CONHMe,
or (ii) when R¹ is an optionally substituted C₁-C₆alkyl or a C₃-C₅cycloalkyl group,
R³ represents methyl, ethyl, propyl, i-propyl, cyclopropyl, cyclobutyl or —CONH₂.

52. A compound according to claim 45 wherein R² represents hydrogen and
(i) when R¹ is an optionally substituted 4- to 6-membered heterocyclyl group, C₁-C₆alkoxy group, C₆aryloxy group, 5- to 6-membered heteroaryloxy or -A-B group,
R³ represents methyl, ethyl, propyl, i-propyl, cyclopropyl, cyclobutyl, —CONH₂ or —CONHMe,
or (ii) when R¹ is an optionally substituted C₁-C₆alkyl or a C₃-C₅cycloalkyl group,
R³ represents methyl, ethyl, propyl, i-propyl, cyclopropyl, cyclobutyl or —CONH₂.

53. A compound according to claim 52 wherein R³ represents methyl, cyclopropyl or —CONH₂.

54. A compound according to claim 1 selected from any one of the Examples.

55. A compound according to claim 1 selected from any one of Examples 3, 6, 7, 9, 10, 13, 14, 15, 16, 21, 28, 29, 41, 42, 43, 44, 56, 57, 66, 67, 68, 69, 71, 73, 84, 91, 93, 94, 97, 102, 103, 111, 124, 126, 128, 129, 131, 132, 135, 141, 27, 52, 53, 54, 61, 62, 70, 72, 107, 120, 1,2, 4, 8, 12, 17, 18, 19, 1 20, 23, 24, 25, 26, 31, 32, 33, 34, 35, 37, 38, 39, 40, 45, 46, 47, 48, 49, 50, 51, 55, 63, 64, 65, 74, 76, 77, 78, 79, 80, 81, 82, 83, 85, 86, 88, 89, 90, 92, 95, 96, 98, 100, 104, 105, 106, 108, 109, 110, 112, 113, 114, 115, 116, 117, 121, 122, 123, 125, 130, 133, 136, 137, 138, 139, 140, 142, 143 5, 22, 36, 58, 59, 60, 75, 87, 99, 101, 118, 119, 127 and 134.

56. A compound according to claim 1 selected from any one of Examples 3, 6, 7, 9, 10, 13, 14, 15, 16, 21, 28, 29, 41, 42, 43, 44, 56, 57, 66, 67, 68, 69, 71, 73, 84, 91, 93, 94, 97, 102, 103, 111, 124, 126, 128, 129, 131, 132, 135, 141, 27, 30, 52, 53, 54, 61, 62, 70, 72, 107, 120, 1,2, 4, 8, 12, 17, 18, 19, 1 20, 23, 24, 25, 26, 31, 32, 33, 34, 35, 37, 38, 39, 40, 45, 46, 47, 48, 49, 50, 51, 55, 63, 64, 65, 74, 76, 77, 78, 79, 80, 81, 82, 83, 85, 86, 88, 89, 90, 92, 95, 96, 98, 100, 104, 105, 106, 108, 109, 110, 112, 113, 114, 115, 116, 117, 121, 122, 123, 125, 130, 133, 136, 137, 138, 139, 140, 142 and 143.

57. A compound according to claim 1 selected from any one of Examples 3, 6, 7, 9, 10, 13, 14, 15, 16, 21, 28, 29, 41, 42, 43, 44, 56, 57, 66, 67, 68, 69, 71, 73, 84, 91, 93, 94, 97, 102, 103, 111, 124, 126, 128, 129, 131, 132, 135, 141, 27, 30, 52, 53, 54, 61, 62, 70, 72, 107, and 120.

58. A compound according to claim 1 selected from any one of Examples 3, 6, 7, 9, 10, 13, 14, 15, 16, 21, 28, 29, 41, 42, 43, 44, 56, 57, 66, 67, 68, 69, 71, 73, 84, 91, 93, 94, 97, 102, 103, 111, 124, 126, 128, 129, 131, 132, 135 and 141.

59. A process for the preparation of a compound of formula (I) as claimed in claim 1, or a pharmaceutically acceptable salt thereof, which comprises:
(i) reacting a compound of formula (IV) 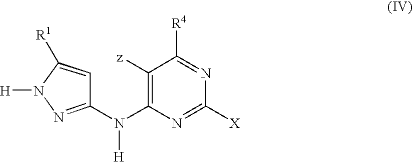
wherein X represents a leaving group (e.g. halogen or sulfanyl such as methanesulfanyl or sulphonyloxy such as methanesulphonyloxy or toluene-4-sulphonyloxy), Z represents hydrogen or a halogen, and R¹ and R⁴ are as hereinbefore defined for a compound formula (I)
with a compound of formula (V) 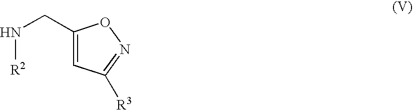
wherein R² and R³ are as defined hereinbefore for a compound of formula (I) to give,
when Z is hydrogen, a compound of formula (I) or,
when Z is halogen, a compound of formula (VI) 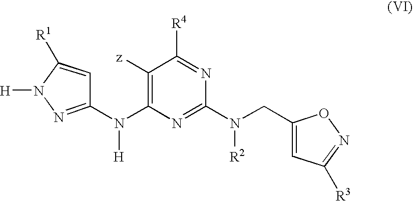
and (ii) when Z is a halogen, optionally reacting a compound of formula (VI) with a de-halogenating reagent to give a compound of formula (I);
and optionally after (i) or (ii) carrying out one or more of the following:
converting the compound obtained to a further compound of the invention
forming a pharmaceutically acceptable salt of the compound.

60. A process for the preparation of a compound of formula (I) as claimed in claim 1, or a pharmaceutically acceptable salt thereof, which comprises:
reacting a compound of formula (IX), 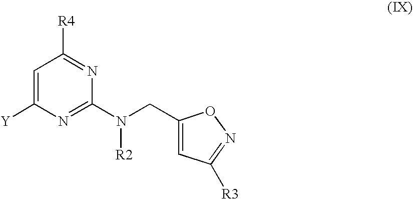
wherein Y is a leaving group such as chloro, and R², R³ and R⁴ are as defined hereinbefore for a compound of formula (I),
with a compound of formula (II) 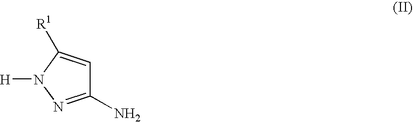
wherein R¹ is as defined hereinbefore for a compound of formula (I)
and optionally carrying out one or more of the following:
converting the compound obtained to a further compound of the invention
forming a pharmaceutically acceptable salt of the compound.

61. A process for the preparation of a compound of formula (I) as claimed in claim 1 wherein R⁴ represent a C₁-C₆alkoxy group optionally substituted with C₁-C₃alkoxy, hydroxyl, amino (—NH₂), mono-C₁-C₃alkylamino and di-(C₁-C₃alky)amino, —NR⁵⁴R⁵⁵, or —S(O)_{y}R⁵⁶, or a pharmaceutically acceptable salt thereof, which comprises:
reacting a compound of formula (XII) 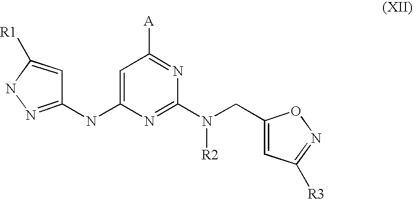
with a compound of formula (XIII)
H—R⁴ (XIII)
wherein R⁴ represents a C₁-C₆alkoxy group optionally substituted with C₁-C₃alkoxy, hydroxyl, amino (—NH₂), mono-C₁-C₃alkylamino and di-(C₁-C₃alky)amino, —NR⁵⁴R⁵⁵, or —S(O)_{y}R⁵⁶ wherein y=0,
and when R⁴ is —S(O)_{y}R⁵⁶ wherein y=0, optionally reacting with an oxidising agent, and optionally carrying out one or more of the following:
converting the compound obtained to a further compound of the invention
forming a pharmaceutically acceptable salt of the compound.

62. A pharmaceutical composition comprising a compound of formula (I), or a pharmaceutically acceptable salt thereof, as claimed in claim 1 in association with a pharmaceutically acceptable adjuvant, diluent or carrier.

63. A process for the preparation of a pharmaceutical composition comprising a compound of formula (I), or a pharmaceutically acceptable salt thereof, as claimed in claim 1 in association with a pharmaceutically acceptable adjuvant, diluent or carrier which comprises mixing a compound of formula (I), or a pharmaceutically acceptable salt thereof, as defined in claim 1, with a pharmaceutically acceptable adjuvant, diluent or carrier.

64. A method of treating cancer which comprises administering to a patient in need thereof a therapeutically effective amount of a compound of formula (I), or a pharmaceutically acceptable salt thereof, as claimed in claim 1.

65. A method of modulating FGFR activity which comprises administering to a patient in need thereof a therapeutically effective amount of a compound of formula (I), or a pharmaceutically acceptable salt thereof, as claimed in claim 1.

66. A method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt as claimed in claim 1.

67. A method of treating cancer which comprises administering to a patient in need thereof a therapeutically effective amount of a compound of formula (I), or a pharmaceutically acceptable salt thereof, as claimed in claim 24.

68. A method of treating cancer which comprises administering to a patient in need thereof a therapeutically effective amount of a compound of formula (I), or a pharmaceutically acceptable salt thereof, as claimed in claim 35.

69. A method of treating cancer which comprises administering to a patient in need thereof a therapeutically effective amount of a compound of formula (I), or a pharmaceutically acceptable salt thereof, as claimed in claim 54.

70. A method of treating cancer which comprises administering to a patient in need thereof a therapeutically effective amount of a compound of formula (I), or a pharmaceutically acceptable salt thereof, as claimed in claim 55.

71. A method of treating cancer which comprises administering to a patient in need thereof a therapeutically effective amount of a compound of formula (I), or a pharmaceutically acceptable salt thereof, as claimed in claim 56.

72. A method of treating cancer which comprises administering to a patient in need thereof a therapeutically effective amount of a compound of formula (I), or a pharmaceutically acceptable salt thereof, as claimed in claim 57.

73. A method of treating cancer which comprises administering to a patient in need thereof a therapeutically effective amount of a compound of formula (I), or a pharmaceutically acceptable salt thereof, as claimed in claim 58.
